SEQUENCE LISTING (228 sequences)

SEQ ID NO: 1 - protein, 1368 aa, synthetic construct [sequence omitted]

SEQ ID NO: 2 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 6 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 7 - protein, 1053 aa, synthetic construct
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 8 - protein, 1053 aa, synthetic construct
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 9 - protein, 984 aa, synthetic construct
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 10 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 11 - protein, 1054 aa, synthetic construct
MENKNLNYILGLDLGIASVGWAVVEIDEKENPLRLIDVGVRTFERAEVPKTGESLALSRRLARSARRLTQRRVARLKKAKRLLKSENILLSTDERLPHQVWQLRVEGLDHKLERQEWAAVLLHLIKHRGYLSQRKNESKSENKELGALLSGVDNNHKLLQQATYRSPAELAVKKFEVEEGHIRNQQGAYTHTFSRLDLLAEMELLFSRQQHFGNPFASEKLLENLTALLMWQKPALSGEAILKMLGKCTFEDEYKAAKNTYSAERFVWITKLNNLRIQENGLERALNDNERLALMEQPYDKNRLFYSQVRSILKLSDEAIFKGLRYSGEDKKAIETKAVLMEMKAYHQIRKVLEGNNLKAEWAELKANPTLLDEIGTAFSLYKTDEDISAYLAGKLSQPVLNALLENLSFDKFIQLSLKALYKLLPLMQQGLRYDEACREIYGDHYGKKTEENHHFLPQIPADEIRNPVVLRTLTQARKVINGVVRLYGSPARIHIETGREVGKSYKDRRELEKRQEENRKQRENAIKEFKEYFPHFAGEPKAKDILKMRLYKQQNAKCLYSGKPIELHRLLEKGYVEVDHALPFSRTWDDSFNNKVLVLANENQNKGNLTPFEWLDGKHNSERWRAFKALVETSAFPYAKKQRILSQKLDEKGFIERNLNDTRYVARFLCNFIADNMHLTGEGKRKVFASNGQITALLRSRWGLAKSREDNDRHHALDAVVVACSTVAMQQKITRFVRFEAGDVFTGERIDRETGEIIPLHFPTPWQFFKQEVEIRIFSDNPKLELENRLPDRPQANHEFVQPLFVSRMPTRKMTGQGHMETVKSAKRLNEGISVIKMPLTKLKLKDLELMVNREREKDLYDTLKARLEAFNDDPAKAFAEPFIKKGGAIVKSVRVEQIQKSGVLVREGNGVADNASMVRVDVFTKGGKYFLVPIYTWQVAKGILPNKAATQYKDEEDWEVMDNSATFKFSLHPNDLVKLVTKKKTILGYFNGLNRATGNIDIKEHDLDKSKGKQGIFEGVGIKLALSFEKYQVDELGKNIRLCKPSKRQPVR

SEQ ID NO: 12 - protein, 1065 aa, synthetic construct
MMMEKFHYVLGLDLGIASVGWAAIEIDKETETSIGLLDCGVRTFERAEVPKTGDSLAKARREARSTRRLIRRRSHRLLRLKRLLKREIFRQPETFKDLPINAWQLRVKGLDSRLNEYEWAAVLLHLVKHRGYLSQRKSEMSETDSKSEMGRLLAGVAENHQLLQQEQYRTPAELALKKFVKHFRNKGGDYAHTFNRLDLQAELHLLFQKQRELGNPFTSPELERQVDDLLMTQRSALQGDAILKMLGHCGFEPEQFKAAKNTFSAERFIWLTKLNNLRIQDQGKERALTADERTKLLDEPYKKSKLTYAQVRKLLSLPQTAIFKGLRYDLEHDKKAENSTLMEMKSYHNIRQTLEKSGLKTEWQSIATQPEILDAIGTAFSIYKTDEDISHELKTCRLPENVLNELLKNINFDGFIQLSLTALRKILPLMEQGYRYDEACTQIYGNHHSGSLQQESKQFLPHIPIDDVRNPVVFRTLTQARKVVNAIIRRYGSPARVHIEMARELGKSKSDRDRIEKQQQKNKKERENAVAKFKEDFPDFVGEPRGKDILKMRLYEQQHGKCLYSGHDIDINRLNEKGYVEIDHALPFSRTWDDSQNNKVLVLGSENQNKRNQTPDEYLDGANNSQRWLEFQARVQTCHFSYGKKQRIQLAKLDDETEKGFLERNLNDTRYIARFMCQFVQENLYLTGKGKRLVFASNGGMTATLRNLWGLRKVREDNDRHHALDAIVVACSTASMQQKITKAFQRHESIEYVDTETGEVKFRIPQPWDFFRQEVMIRVFSDQPCEDLVEKLSARPEALHDNVTPLFVSRAPNRKMSGQGHLETIKSAKRLSEENSMVKKPLTTLKLKDIPEIVGYPSREPQLYAALKTRLETHDDDPIKAFAKPFYKPNKNGELGALVRSVRVKGVQNTGVMVHDGKGIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKK

SEQ ID NO: 13 - protein, 1082 aa, synthetic construct
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 14 - protein, 18 aa, synthetic construct
KRTADGSEFESPKKKRKV

SEQ ID NO: 15 - protein, 32 aa, synthetic construct
SGGSKRTADSQHSTPPKTKRKVEFEPKKKRKV

SEQ ID NO: 16 - protein, 16 aa, synthetic construct
KRPAATKKAGQAKKKK

SEQ ID NO: 17 - protein, 17 aa, synthetic construct
KKTELQTTNAENKTKKL

SEQ ID NO: 18 - protein, 19 aa, synthetic construct
KRGINDRNFWRGENGRKTR

SEQ ID NO: 19 - protein, 16 aa, synthetic construct
RKSGKIAAIVVKRPRK

SEQ ID NO: 20 - protein, 7 aa, synthetic construct
PKKKRKV

SEQ ID NO: 21 - protein, 30 aa, synthetic construct
MDSLLMNRRKFLYQFKNVRWAKGRRETYLC

SEQ ID NO: 22 - protein, 34 aa, synthetic construct
SGGSSGGSKRTADGSEFESPKKKRKVSGGSSGGS

SEQ ID NO: 23 - protein, 33 aa, synthetic construct
SGGSSGGSSGSETPGTSESATPESSGGSSGGSS

SEQ ID NO: 24 - protein, 40 aa, synthetic construct
SGGSSGGSSGGSSGGSSGGSSGGSSGGSSGGSSGGSSGGS

SEQ ID NO: 25 - DNA, 102 bp, synthetic construct
  source 1..102: mol_type = other DNA
tccggcggaagctctggtggcagcaagcggaccgccgacggctctgaattcgagagccctaagaagaaaagaaaggtgagcggaggctctagcggcggaagc

SEQ ID NO: 26 - protein, 47 aa, synthetic construct
LEIEAAFLERENTALETRVAELRQRVQRLRNRVSQYRTRYGPLGGGK

SEQ ID NO: 27 - protein, 47 aa, synthetic construct
LEIRAAFLRQRNTALRTEVAELEQEVQRLENEVSQYETRYGPLGGGK

SEQ ID NO: 28 - protein, 80 aa, synthetic construct
STDHPKYSDMIVAAIQAEKNRAGSSRQSIQKYIKSHYKVGENADSQIKLSIKRLVTTGVLKQTKGVGASGSFRLAKSDEP

SEQ ID NO: 29 - protein, 215 aa, synthetic construct
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 30 - protein, 209 aa, synthetic construct
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 31 - protein, 200 aa, synthetic construct
MAKGDPKKPKGKMSAYAFFVQTCREEHKKKNPEVPVNFAEFSKKCSERWKTMSGKEKSKFDEMAKADKVRYDREMKDYGPAKGGKKKKDPNAPKRPPSGFFLFCSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 32 - protein, 100 aa, synthetic construct
MPKRKVSSAEGAAKEEPKRRSARLSAKPPAKVEAKPKKAAAKDKSSDKKVQTKGKRGAKGKQAEVANQETKEDLPAENGETKTEESPASDEAGEKEAKSD

SEQ ID NO: 33 - protein, 90 aa, synthetic construct
MPKRKAEGDAKGDKAKVKDEPQRRSARLSAKPAPPKPEPKPKKAPAKKGEKVPKGKKGKADAGKEGNNPAENGDAKTDQAQKAEGAGDAK

SEQ ID NO: 34 - protein, 16 aa, synthetic construct
RQIKIYFQNRRMKWKK

SEQ ID NO: 35 - protein, 9 aa, synthetic construct
RKKRRQRRR

SEQ ID NO: 36 - protein, 8 aa, synthetic construct
RRRRRRRR

SEQ ID NO: 37 - protein, 16 aa, synthetic construct
RKKRRRESRKKRRRES

SEQ ID NO: 38 - protein, 17 aa, synthetic construct
GRPRESGKKRKRKRLKP

SEQ ID NO: 39 - protein, 13 aa, synthetic construct
GRRRRRRRRRPPQ

SEQ ID NO: 40 - protein, 18 aa, synthetic construct
LLIILRRRIRKQAHAHSK

SEQ ID NO: 41 - protein, 14 aa, synthetic construct
RVRVFVVWHIPRLT

SEQ ID NO: 42 - protein, 27 aa, synthetic construct
GALFLGFLGAAGSTMGAWSQPKKKRKV

SEQ ID NO: 43 - protein, 27 aa, synthetic construct
GWTLNSAGYLLGKINLKALAALAKKIL

SEQ ID NO: 44 - protein, 5 aa, synthetic construct
VSALK

SEQ ID NO: 45 - protein, 16 aa, synthetic construct
CSIPPEVKFNPFVYLI

SEQ ID NO: 46 - protein, 26 aa, synthetic construct
GIGAVLKVLTTGLPALISWIKRKRQQ

SEQ ID NO: 47 - protein, 20 aa, synthetic construct
HGLASTLTRWAHYNALIRAF

SEQ ID NO: 48 - protein, 8 aa, synthetic construct
DYKDDDDK

SEQ ID NO: 49 - protein, 6 aa, synthetic construct
HHHHHH

SEQ ID NO: 50 - protein, 26 aa, synthetic construct
KRRWKKNFIAVSAANRFKKISSSGAL

SEQ ID NO: 51 - protein, 396 aa, synthetic construct
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 52 - protein, 10 aa, synthetic construct
EQKLISEEDL

SEQ ID NO: 53 - protein, 201 aa, synthetic construct
MKLFYKPGACSLASHITLRESGKDFTLVSVDLMKKRLENGDNYFAVNPKGQVPALLLDDGTLLTEGVAIMQYLADSVPDRQLLAPVNSISRYKTIEWLNYIATELHKGFTPLFRPDTPEEYKSTVRAQLEKKLQYVNEALKDEHWICGQRFTIADAYLFTVLRWAYAVKLNLEGLEHIAAFMQRMAERPEVQDALSAEGLK

SEQ ID NO: 54 - protein, 9 aa, synthetic construct
YPYDVPDYA

SEQ ID NO: 55 - protein, 298 aa, synthetic construct
MAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDR

SEQ ID NO: 56 - protein, 919 aa, synthetic construct
MQRSIMSFFHPKKEGKAKKPEKEASNSSRETEPPPKAALKEWNGVVSESDSPVKRPGRKAARVLGSEGEEEDEALSPAKGQKPALDCSQVSPPRPATSPENNASLSDTSPMDSSPSGIPKRRTARKQLPKRTIQEVLEEQSEDEDREAKRKKEEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTY

SEQ ID NO: 57 - protein, 687 aa, synthetic construct
TPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTY

SEQ ID NO: 58 - protein, 801 aa, synthetic construct
PKRRTARKQLPKRTIQEVLEEQSEDEDREAKRKKEEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTY

SEQ ID NO: 59 - protein, 1009 aa, synthetic construct
MSLAFKIFFPQTLRALSRKELCLFRKHHWRDVRQFSQWSETDLLHGHPLFLRRKPVLSFQGSHLRSRATYLVFLPGLHVGLCSGPCEMAEQRFCVDYAKRGTAGCKKCKEKIVKGVCRIGKVVPNPFSESGGDMKEWYHIKCMFEKLERARATTKKIEDLTELEGWEELEDNEKEQITQHIADLSSKAAGTPKKKAVVQAKLTTTGQVTSPVKGASFVTSTNPRKFSGFSAKPNNSGEAPSSPTPKRSLSSSKCDPRHKDCLLREFRKLCAMVADNPSYNTKTQIIQDFLRKGSAGDGFHGDVYLTVKLLLPGVIKTVYNLNDKQIVKLFSRIFNCNPDDMARDLEQGDVSETIRVFFEQSKSFPPAAKSLLTIQEVDEFLLRLSKLTKEDEQQQALQDIASRCTANDLKCIIRLIKHDLKMNSGAKHVLDALDPNAYEAFKASRNLQDVVERVLHNAQEVEKEPGQRRALSVQASLMTPVQPMLAEACKSVEYAMKKCPNGMFSEIKYDGERVQVHKNGDHFSYFSRSLKPVLPHKVAHFKDYIPQAFPGGHSMILDSEVLLIDNKTGKPLPFGTLGVHKKAAFQDANVCLFVFDCIYFNDVSLMDRPLCERRKFLHDNMVEIPNRIMFSEMKRVTKALDLADMITRVIQEGLEGLVLKDVKGTYEPGKRHWLKVKKDYLNEGAMADTADLVVLGAFYGQGSKGGMMSIFLMGCYDPGSQKWCTVTKCAGGHDDATLARLQNELDMVKISKDPSKIPSWLKVNKIYYPDFIVPDPKKAAVWEITGAEFSKSEAHTADGISIRFPRCTRIRDDKDWKSATNLPQLKELYQLSKEKADFTVVAGDEGSSTTGGSSEENKGPSGSAVSRKAPSKPSASTKKAEGKLSNSNSKDGNMQTAKPSAMKVGEKLATKSSPVKVGEKRKAADETLCQTKVLLDIFTGVRLYLPPSTPDFSRLRRYFVAFDGDLVQEFDMTSATHVLGSRDKNPAAQQVSPEWIWACIRKRRLVAPC

SEQ ID NO: 60 - protein, 949 aa, synthetic construct
MSLAFKIFFPQTLRALSRKELCLFRKHHWRDVRQFSQWSETDLLHGHPLFLRRKPVLSFQGSHLRSRATYLVFLPGLHVGLCSGPCEMAEQRFCVDYAKRGTAGCKKCKEKIVKGVCRIGKVVPNPFSESGGDMKEWYHIKCMFEKLERARATTKKIEDLTELEGWEELEDNEKEQITQHIADLSSKAAGTPKKKAVVQAKLTTTGQVTSPVKGASFVTSTNPRKFSGFSAKPNNSGEAPSSPTPKRSLSSSKCDPRHKDCLLREFRKLCAMVADNPSYNTKTQIIQDFLRKGSAGDGFHGDVYLTVKLLLPGVIKTVYNLNDKQIVKLFSRIFNCNPDDMARDLEQGDVSETIRVFFEQSKSFPPAAKSLLTIQEVDEFLLRLSKLTKEDEQQQALQDIASRCTANDLKCIIRLIKHDLKMNSGAKHVLDALDPNAYEAFKASRNLQDVVERVLHNAQEVEKEPGQRRALSVQASLMTPVQPMLAEACKSVEYAMKKCPNGMFSEIKYDGERVQVHKNGDHFSYFSRSLKPVLPHKVAHFKDYIPQAFPGGHSMILDSEVLLIDNKTGKPLPFGTLGVHKKAAFQDANVCLFVFDCIYFNDVSLMDRPLCERRKFLHDNMVEIPNRIMFSEMKRVTKALDLADMITRVIQEGLEGLVLKDVKGTYEPGKRHWLKVKKDYLNEGAMADTADLVVLGAFYGQGSKGGMMSIFLMGCYDPGSQKWCTVTKCAGGHDDATLARLQNELDMVKISKDPSKIPSWLKVNKIYYPDFIVPDPKKAAVWEITGAEFSKSEAHTADGISIRFPRCTRIRDDKDWKSATNLPQLKELYQLSKEKADFTVVAGDEGSSTTGGSSEENKGPSGSAVSRKAPSKPSASTKKAEGKLSNSNSKDGNMQTAKPSAMKVGEKLATKSSPVKVGEKRKAADETLCQTKRRPASEQRGRTVPAGRR

SEQ ID NO: 61 - protein, 922 aa, synthetic construct
MAEQRFCVDYAKRGTAGCKKCKEKIVKGVCRIGKVVPNPFSESGGDMKEWYHIKCMFEKLERARATTKKIEDLTELEGWEELEDNEKEQITQHIADLSSKAAGTPKKKAVVQAKLTTTGQVTSPVKGASFVTSTNPRKFSGFSAKPNNSGEAPSSPTPKRSLSSSKCDPRHKDCLLREFRKLCAMVADNPSYNTKTQIIQDFLRKGSAGDGFHGDVYLTVKLLLPGVIKTVYNLNDKQIVKLFSRIFNCNPDDMARDLEQGDVSETIRVFFEQSKSFPPAAKSLLTIQEVDEFLLRLSKLTKEDEQQQALQDIASRCTANDLKCIIRLIKHDLKMNSGAKHVLDALDPNAYEAFKASRNLQDVVERVLHNAQEVEKEPGQRRALSVQASLMTPVQPMLAEACKSVEYAMKKCPNGMFSEIKYDGERVQVHKNGDHFSYFSRSLKPVLPHKVAHFKDYIPQAFPGGHSMILDSEVLLIDNKTGKPLPFGTLGVHKKAAFQDANVCLFVFDCIYFNDVSLMDRPLCERRKFLHDNMVEIPNRIMFSEMKRVTKALDLADMITRVIQEGLEGLVLKDVKGTYEPGKRHWLKVKKDYLNEGAMADTADLVVLGAFYGQGSKGGMMSIFLMGCYDPGSQKWCTVTKCAGGHDDATLARLQNELDMVKISKDPSKIPSWLKVNKIYYPDFIVPDPKKAAVWEITGAEFSKSEAHTADGISIRFPRCTRIRDDKDWKSATNLPQLKELYQLSKEKADFTVVAGDEGSSTTGGSSEENKGPSGSAVSRKAPSKPSASTKKAEGKLSNSNSKDGNMQTAKPSAMKVGEKLATKSSPVKVGEKRKAADETLCQTKVLLDIFTGVRLYLPPSTPDFSRLRRYFVAFDGDLVQEFDMTSATHVLGSRDKNPAAQQVSPEWIWACIRKRRLVAPC

SEQ ID NO: 62 - protein, 862 aa, synthetic construct
MAEQRFCVDYAKRGTAGCKKCKEKIVKGVCRIGKVVPNPFSESGGDMKEWYHIKCMFEKLERARATTKKIEDLTELEGWEELEDNEKEQITQHIADLSSKAAGTPKKKAVVQAKLTTTGQVTSPVKGASFVTSTNPRKFSGFSAKPNNSGEAPSSPTPKRSLSSSKCDPRHKDCLLREFRKLCAMVADNPSYNTKTQIIQDFLRKGSAGDGFHGDVYLTVKLLLPGVIKTVYNLNDKQIVKLFSRIFNCNPDDMARDLEQGDVSETIRVFFEQSKSFPPAAKSLLTIQEVDEFLLRLSKLTKEDEQQQALQDIASRCTANDLKCIIRLIKHDLKMNSGAKHVLDALDPNAYEAFKASRNLQDVVERVLHNAQEVEKEPGQRRALSVQASLMTPVQPMLAEACKSVEYAMKKCPNGMFSEIKYDGERVQVHKNGDHFSYFSRSLKPVLPHKVAHFKDYIPQAFPGGHSMILDSEVLLIDNKTGKPLPFGTLGVHKKAAFQDANVCLFVFDCIYFNDVSLMDRPLCERRKFLHDNMVEIPNRIMFSEMKRVTKALDLADMITRVIQEGLEGLVLKDVKGTYEPGKRHWLKVKKDYLNEGAMADTADLVVLGAFYGQGSKGGMMSIFLMGCYDPGSQKWCTVTKCAGGHDDATLARLQNELDMVKISKDPSKIPSWLKVNKIYYPDFIVPDPKKAAVWEITGAEFSKSEAHTADGISIRFPRCTRIRDDKDWKSATNLPQLKELYQLSKEKADFTVVAGDEGSSTTGGSSEENKGPSGSAVSRKAPSKPSASTKKAEGKLSNSNSKDGNMQTAKPSAMKVGEKLATKSSPVKVGEKRKAADETLCQTKRRPASEQRGRTVPAGRR

SEQ ID NO: 63 - protein, 911 aa, synthetic construct
MAASQTSQTVASHVPFADLCSTLERIQKSKGRAEKIRHFREFLDSWRKFHDALHKNHKDVTDSFYPAMRLILPQLERERMAYGIKETMLAKLYIELLNLPRDGKDALKLLNYRTPTGTHGDAGDFAMIAYFVLKPRCLQKGSLTIQQVNDLLDSIASNNSAKRKDLIKKSLLQLITQSSALEQKWLIRMIIKDLKLGVSQQTIFSVFHNDAAELHNVTTDLEKVCRQLHDPSVGLSDISITLFSAFKPMLAAIADIEHIEKDMKHQSFYIETKLDGERMQMHKDGDVYKYFSRNGYNYTDQFGASPTEGSLTPFIHNAFKADIQICILDGEMMAYNPNTQTFMQKGTKFDIKRMVEDSDLQTCYCVFDVLMVNNKKLGHETLRKRYEILSSIFTPIPGRIEIVQKTQAHTKNEVIDALNEAIDKREEGIMVKQPLSIYKPDKRGEGWLKIKPEYVSGLMDELDILIVGGYWGKGSRGGMMSHFLCAVAEKPPPGEKPSVFHTLSRVGSGCTMKELYDLGLKLAKYWKPFHRKAPPSSILCGTEKPEVYIEPCNSVIVQIKAAEIVPSDMYKTGCTLRFPRIEKIRDDKEWHECMTLDDLEQLRGKASGKLASKHLYIGGDDEPQEKKRKAAPKMKKVIGIIEHLKAPNLTNVNKISNIFEDVEFCVMSGTDSQPKPDLENRIAEFGGYIVQNPGPDTYCVIAGSENIRVKNIILSNKHDVVKPAWLLECFKTKSFVPWQPRFMIHMCPSTKEHFAREYDCYGDSYFIDTDLNQLKEVFSGIKNSNEQTPEEMASLIADLEYRYSWDCSPLSMFRRHTVYLDSYAVINDLSTKNEGTRLAIKALELRFHGAKVVSCLAEGVSHVIIGEDHSRVADFKAFRRTFKRKFKILKESWVTDSIDKCELQEENQYLI

SEQ ID NO: 64 - protein, 487 aa, synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 65 - protein, 359 aa, synthetic construct
MMNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKM

SEQ ID NO: 66 - protein, 674 aa, synthetic construct
MTLEAARRRVNELRDLIRYHNYLYYVLDAPEISDAEYDRLLRELKELEERFPELQSPDSPTEQVGARPLESTFRPVRHPTRMYSLDNAFSLDEVRAFEERIERALGRKGPFLYTVEHKVDGLSVNLYYEEGILVFGATRGDGETGEEVTQNLLTIRTIPRRLTGVPDRLEVRGEVYMPIEAFLRLNQELEEAGERIFKNPRNAAAGSLRQKDPRVTARRGLRATFYALGLGLEETGLKSQHDLLLWLRERGFPVEHGFTRALGAEGVEEVYQAWLKERRKLPFEADGVVVKLDDLALWRELGYTARAPRFALAYKFPAEEKETRLLSVAFQVGRTGRITPVGVLEPVFIEGSEVSRVTLHNESFIEELDVRIGDWVLVHKAGGVIPEVLRVLKERRTGEEKPILWPENCPECGHALIKEGKVHRCPNPLCPAKRFEAIRHYASRKAMDIQGLGEKLIEKLLEKGLVRDVADLYRLKKEDLVNLERMGEKSAENLLRQIEESKGRGLERLLYALGLPGVGEVLARNLALRFGHMDRLLEAGLEDLLEVEGVGELTARAILNTLKDPEFRDLVRRLKEAGVEMEAKEREGEALKGLTFVITGELSRPREEVKALLRRLGAKVTDSVSRKTGFLVVGENPGSKLEKARALGVPTLSEEELYRLIEERTGKDPRALTA

SEQ ID NO: 67 - protein, 346 aa, synthetic construct
MNIFNTNPFKAVSFVESAVKKALETSGYLIADCKYDGVRGNIVVDNVAEAAWLSRVSKFIPALEHLNGFDKRWQQLLNDDRCIFPDGFMLDGELMVKGVDFNTGSGLLRTKWVKRDNMGFHLTNVPTKLTPKGREVIDGKFEFHLDPKRLSVRLYAVMPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTSNVKAHGEDFYNGWACQVNYMEATPDGSLRHPSFEKFRGTEDNPQEKM

SEQ ID NO: 68 - protein, 671 aa, synthetic construct
MESIEQQLTELRTTLRHHEYLYHVMDAPEIPDAEYDRLMRELRELETKHPELITPDSPTQRVGAAPLAAFSQIRHEVPMLSLDNVFDEESFLAFNKRVQDRLKNNEKVTWCCELKLDGLAVSILYENGVLVSAATRGDGTTGEDITSNVRTIRAIPLKLHGENIPARLEVRGEVFLPQAGFEKINEDARRTGGKVFANPRNAAAGSLRQLDPRITAKRPLTFFCYGVGVLEGGELPDTHLGRLLQFKKWGLPVSDRVTLCESAEEVLAFYHKVEEDRPTLGFDIDGVVIKVNSLAQQEQLGFVARAPRWAVAFKFPAQEQMTFVRDVEFQVGRTGAITPVARLEPVHVAGVLVSNATLHNADEIERLGLRIGDKVVIRRAGDVIPQVVNVVLSERPEDTREVVFPTHCPVCGSDVERVEGEAVARCTGGLICGAQRKESLKHFVSRRAMDVDGMGDKIIDQLVEKEYVHTPADLFKLTAGKLTGLERMGPKSAQNVVNALEKAKETTFARFLYALGIREVGEATAAGLAAYFGTLEALEAASIEELQKVPDVGIVVASHVHNFFAEESNRNVISELLAEGVHWPAPIVINAEEIDSPFAGKTVVLTGSLSQMSRDDAKARLVELGAKVAGSVSKKTDLVIAGEAAGSKLAKAQELGIEVIDEAEMLRLLGS

SEQ ID NO: 69 - protein, 559 aa, Thermococcus kodakarensis
MRYSELADLYRRLEKTTLKTLKTKFVADFLKKTPDELLEIVPYLILGKVFPDWDERELGVGEKLLIKAVSMATGVPEKEIEDSVRDTGDLGESVALAIKKKKQKSFFSQPLTIKRVYDTFVKIAEAQGEGSQDRKMKYLANLFMDAEPEEGKYLARTVLGTMRTGVAEGILRDAIAEAFRVKPELVERAYMLTSDFGYVAKIAKLEGNEGLSKVRIQIGKPIRPMLAQNAASVKDALIEMGGEAAFEIKYDGARVQVHKDGDKVIVYSRRLENVTRSIPEVIEAIKAALKPEKAIVEGELVAVGENGRPRPFQYVLRRFRRKYNIDEMIEKIPLELNLFDVMFVDGESLIETKFIDRRNKLEEIVKESEKIKLAEQLITKKVEEAEAFYRRALELGHEGLMAKRLDSIYEPGNRGKKWLKIKPTMENLDLVIIGAEWGEGRRAHLLGSFLVAAYDPHSGEFLPVGKVGSGFTDEDLVEFTKMLKPYIVRQEGKFVEIEPKFVIEVTYQEIQKSPKYKSGFALRFPRYVALREDKSPEEADTIERVAELYELQERFKAKK

SEQ ID NO: 70 - protein, 419 aa, African swine fever virus
MLNQFPGQYSNNIFCFPPIESETKSGKKASWIICVQVVQHNTIIPITDEMFSTDVKDAVAEIFTKFFVEEGAVRISKMTRVTEGKNLGKKNATTVVHQAFKDALSKYNRHARQKRGAHTNRGMIPPMLVKYFNIIPKTFFEEETDPIVQRKRNGVRAVACQQGDGCILLYSRTEKEFLGLDNIKKELKQLYLFIDVRVYLDGELYLHRKPLQWIAGQANAKTDSSELHFYVFDCFWSDQLQMPSNKRQQLLTNIFKQKEDLTFIHQVENFSVKNVDEALRLKAQFIKEGYEGAIVRNANGPYEPGYNNYHSAHLAKLKPLLDAEFILVDYTQGKKGKDLGAILWVCELPNKKRFVVTPKHLTYADRYALFQKLTPALFKKHLYGKELTVEYAELSPKTGIPLQARAVGFREPINVLEII

SEQ ID NO: 71 - protein, 552 aa, Vaccinia virus
MTSLREFRKLCCDIYHASGYKEKSKLIRDFITDRDDKYLIIKLLLPGLDDRIYNMNDKQIIKLYSIIFKQSQEDMLQDLGYGYIGDTIRTFFKENTEIRPRDKSILTLEDVDSFLTTLSSVTKESHQIKLLTDIASVCTCNDLKCVVMLIDKDLKIKAGPRYVLNAISPNAYDVFRKSNNLKEIIENASKQNLDSISISVMTPINPMLAESCDSVNKAFKKFPSGMFAEVKYDGERVQVHKNNNEFAFFSRNMKPVLSHKVDYLKEYIPKAFKKATSIVLDSEIVLVDEHNVPLPFGSLGIHKKKEYKNSNMCLFVFDCLYFDGFDMTDIPLYERRSFLKDVMVEIPNRIVFSELTNISNESQLTDVLDDALTRKLEGLVLKDINGVYEPGKRRWLKIKRDYLNEGSMADSADLVVLGAYYGKGAKGGIMAVFLMGCYDDESGKWKTVTKCSGHDDNTLRVLQDQLTMVKINKDPKKIPEWLVVNKIYIPDFVVEDPKQSQIWEISGAEFTSSKSHTANGISIRFPRFTRIREDKTWKESTHLNDLVNLTKS

SEQ ID NO: 72 - protein, 552 aa, synthetic construct
MTSLREFRKLCCDIYHASGYKEKSKLIRDFITDRDDKYLIIKLLLPGLDDRIYNMNDKQIIKLYSIIFKQSQEDMLQDLGYGYIGDTIRTFFKENTEIRPRDKSILTLEEVDSFLTTLSSVTKESHQIKLLTDIASVCTCNDLKCVVMLIDKDLKIKAGPRYVLNAISPHAYDVFRKSNNLKEIIENASKQNLDSISISVMTPINPMLAESCDSVNKAFKKFPSGMFAEVKYDGERVQVHKNNNEFAFFSRNMKPVLSHKVDYLKEYIPKAFKKATSIVLDSEIVLVDEHNVPLPFGSLGIHKKKEYKNSNMCLFVFDCLYFDGFDMTDIPLYERRSFLKDVMVEIPNRIVFSELTNISNESQLTDVLDDALTRKLEGLVLKDINGVYEPGKRRWLKIKRDYLNEGSMADSADLVVLGAYYGKGAKGGIMAVFLMGCYDDESGKWKTVTKCSGHDDNTLRELQDQLKMIKINKDPKKIPEWLVVNKIYIPDFVVEDPKQSQIWEISGAEFTSSKSHTANGISIRFPRFTRIREDKTWKESTHLNDLVNLTKS

SEQ ID NO: 73 - protein, 691 aa, Burkholderia pseudomallei
MARSPVEPPASQPAKRAAWLRAELERANYAYYVLDQPDLPDAEYDRLFVELQRIEAEHPDLVTPDSPTQRVGGEAASGFTPVVHDKPMLSLNNGFADEDVIAFDKRVADGLDKATDLAGTVTEPVEYACELKFDGLAISLRYENGRFVQASTRGDGTTGEDVTENIRTIRAIPLTLKGKRIPRMLDVRGEVLMFKRDFARLNERQRAAGQREFANPRNAAAGSLRQLDSKITASRPLSFFAYGIGVLDGADMPDTHSGLLDWYETLGLPVNRERAVVRGAAGLLAFFHSVGERRESLPYDIDGVVYKVNRRDEQDRLGFVSRAPRFALAHKFPAQEALTKLIAIDVQVGRTGAITPVARLEPVFVGGATVTNATLHNEDEVRRKDIRIGDTVIVRRAGDVIPEVVSAVLDRRPADAQEFVMPTECPECGSRIERLPDEAIARCTGGLFCPAQRKQALWHFAQRRALDIDGLGEKIIDQLVEQNLVRTPADLFNLGFSTLVGLDRFAEKSARNLIDSLEKAKHTTLARFIYALGIRHVGESTAKDLAKHFGSLDPIMDAPIDALLEVNDVGPIVAESIHQFFAEEHNRTVIEQLRARGKVTWPEGPPAPRAPQGVLAGKTVVLTGTLPTLTREAAKEMLEAAGAKVAGSVSKKTDYVVAGADAGSKLAKAEELGIPVLDEAGMHTLLEGHAR

SEQ ID NO: 74 - protein, 317 aa, synthetic construct
MQFFLTVFCLLLITAVTHVNAEDKLDIVDGLQLAKQYSHSRQDINIAEYWVSEKLDGIRARWDGTELRTRNNNKIAAPAWFTANWPKATIDGELWIARGQFERTASIVLSKLTSVAPHSVAGSLPRTESTVGAMTATHSLPSKRWAKIRFMAFDMPVAGQSFDSRLNMLNNLKEATPNPTFAVVSQFTLSSVNALEEKLEQVTLSGGEGLMLHHKKAFYHSGRSDKLLKVKQFEDAEAKVLAHLPGKGKFKGMMGSLLVETPAGVQFKLGTGFSEKERQAPPAIGSWVTFKFYGVTKNGKPRFASFLRVRPPSDLPK

SEQ ID NO: 75 - protein, 755 aa, synthetic construct
MRRLLTGCLLSSARPLKSRLPLLMSSSLPSSAGKKPKQATLARFFTSMKNKPTEGTPSPKKSSKHMLEDRMDNVSGEEEYATKKLKQTAVTHTVAAPSSMGSNFSSIPSSAPSSGVADSPQQSQRLVGEVEDALSSNNNDHYSSNIPYSEVCEVFNKIEAISSRLEIIRICSDFFIKIMKQSSKNLIPTTYLFINRLGPDYEAGLELGLGENLLMKTISETCGKSMSQIKLKYKDIGDLGEIAMGARNVQPTMFKPKPLTVGEVFKNLRAIAKTQGKDSQLKKMKLIKRMLTACKGIEAKFLIRSLESKLRIGLAEKTVLISLSKALLLHDENREDSPDKDVPMDVLESAQQKIRDAFCQVPNYEIVINSCLEHGIMNLDKYCTLRPGIPLKPMLAKPTKAINEVLDRFQGETFTSEYKYDGERAQVHLLNDGTMRIYSRNGENMTERYPEINITDFIQDLDTTKNLILDCEAVAWDKDQGKILPFQVLSTRKRKDVELNDVKVKVCLFAFDILCYNDERLINKSLKERREYLTKVTKVVPGEFQYATQITTNNLDELQKFLDESVNHSCEGLMVKMLEGPESHYEPSKRSRNWLKLKKDYLEGVGDSLDLCVLGAYYGRGKRTGTYGGFLLGCYNQDTGEFETCCKIGTGFSDEMLQLLHDRLTPTIIDGPKATFVFDSSAEPDVWFEPTTLFEVLTADLSLSPIYKAGSATFDKGVSLRFPRFLRIREDKGVEDATSSDQIVELYENQSHMQN

SEQ ID NO: 76 - protein, 944 aa, synthetic construct
MISALDSIPEPQNFAPSPDFKWLCEELFVKIHEVQINGTAGTGKSRSFKYYEIISNFVEMWRKTVGNNIYPALVLALPYRDRRIYNIKDYVLIRTICSYLKLPKNSATEQRLKDWKQRVGKGGNLSSLLVEEIAKRRAEPSSKAITIDNVNHYLDSLSGDRFASGRGFKSLVKSKPFLHCVENMSFVELKYFFDIVLKNRVIGGQEHKLLNCWHPDAQDYLSVISDLKVVTSKLYDPKVRLKDDDLSIKVGFAFAPQLAKKVNLSYEKICRTLHDDFLVEEKMDGERIQVHYMNYGESIKFFSRRGIDYTYLYGASLSSGTISQHLRFTDSVKECVLDGEMVTFDAKRRVILPFGLVKGSAKEALSFNSINNVDFHPLYMVFDLLYLNGTSLTPLPLHQRKQYLNSILSPLKNIVEIVRSSRCYGVESIKKSLEVAISLGSEGVVLKYYNSSYNVASRNNNWIKVKPEYLEEFGENLDLIVIGRDSGKKDSFMLGLLVLDEEEYKKHQGDSSEIVDHSSQEKHIQNSRRRVKKILSFCSIANGISQEEFKEIDRKTRGHWKRTSEVAPPASILEFGSKIPAEWIDPSESIVLEIKSRSLDNTETNMQKYATNCTLYGGYCKRIRYDKEWTDCYTLNDLYESRTVKSNPSYQAERSQLGLIRKKRKRVLISDSFHQNRKQLPISNIFAGLLFYVLSDYVTEDTGIRITRAELEKTIVEHGGKLIYNVILKRHSIGDVRLISCKTTTECKALIDRGYDILHPNWVLDCIAYKRLILIEPNYCFNVSQKMRAVAEKRVDCLGDSFENDISETKLSSLYKSQLSLPPMGELEIDSEVRRFPLFLFSNRIAYVPRRKISTEDDIIEMKIKLFGGKITDQQSLCNLIIIPYTDPILRKDCMNEVHEKIKEQIKASDTIPKIARVVAPEWVDHSINENCQVPEEDFPVVNY

SEQ ID NO: 77 - protein, 487 aa, synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAHPENSKVKDFTEVAESRTASNGIANKSLKGTISEKEAQCMKFQVWDYVPLVEVYGLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 78 - protein, 916 aa, synthetic construct
MQRSIMSFFQPTKEGKAKKPEKETPSSIREKEPPPKVALKERNQVVPESDSPVKRTGRKVAQVLSCEGEDEDEAPGTPKVQKPVSDSEQSSPPSPDTCPENSPVFNCSSPMDISPSGFPKRRTARKQLPKRTIQDTLEEQNEDKTKTAKKRKKEEETPKESLAEAEDIKQKEEKEGDQLIVPSEPTKSPESVTLTKTENIPVCKAGVKLKPQEEEQSKPPARGAKTLSSFFTPRKPAVKTEVKQEESGTLRKEETKGTLDPANYNPSKNNYHPIEDACWKHGQKVPFLAVARTFEKIEEVSARLKMVETLSNLLRSVVALSPPDLLPVLYLSLNRLGPPQQGLELGVGDGVLLKAVAQATGRQLESIRAEVAEKGDVGLVAENSRSTQRLMLPPPPLTISGVFTKFCDIARLTGSASMAKKMDIIKGLFVACRHSEARYIARSLSGRLRLGLAEQSVLAALAQAVSLTPPGQEFPTVVVDAGKGKTAEARKMWLEEQGMILKQTFCEVPDLDRIIPVLLEHGLERLPEHCKLSPGVPLKPMLAHPTRGVSEVLKRFEEVDFTCEYKYDGQRAQIHVLEGGEVKIFSRNQEDNTGKYPDIISRIPKIKHPSVTSFILDTEAVAWDREKKQIQPFQVLTTRKRKEVDASEIQVQVCLYAFDLIYLNGESLVRQPLSRRRQLLRENFVETEGEFVFTTSLDTKDTEQIAEFLEQSVKDSCEGLMVKTLDVDATYEIAKRSHNWLKLKKDYLDGVGDTLDLVVIGAYLGRGKRAGRYGGFLLAAYDEESEELQAICKLGTGFSDEELEEHHQSLQALVLPTPRPYVRIDGAVAPDHWLDPSIVWEVKCADLSLSPIYPAARGLVDKEKGISLRFPRFIRVRKDKQPEQATTSNQVASLYRKQSQIQNQQSSDLDSDVEDY

SEQ ID NO: 79 - protein, 911 aa, synthetic construct
MASSQTSQTVAAHVPFADLCSTLERIQKGKDRAEKIRHFKEFLDSWRKFHDALHKNRKDVTDSFYPAMRLILPQLERERMAYGIKETMLAKLYIELLNLPREGKDAQKLLNYRTPSGARTDAGDFAMIAYFVLKPRCLQKGSLTIQQVNELLDLVASNNSGKKKDLVKKSLLQLITQSSALEQKWLIRMIIKDLKLGISQQTIFSIFHNDAVELHNVTTDLEKVCRQLHDPSVGLSDISITLFSAFKPMLAAVADVERVEKDMKQQSFYIETKLDGERMQMHKDGALYRYFSRNGYNYTDQFGESPQEGSLTPFIHNAFGTDVQACILDGEMMAYNPTTQTFMQKGVKFDIKRMVEDSGLQTCYSVFDVLMVNKKKLGRETLRKRYEILSSTFTPIQGRIEIVQKTQAHTKKEVVDALNDAIDKREEGIMVKHPLSIYKPDKRGEGWLKIKPEYVSGLMDELDVLIVGGYWGKGSRGGMMSHFLCAVAETPPPGDRPSVFHTLCRVGSGYTMKELYDLGLKLAKYWKPFHKKSPPSSILCGTEKPEVYIEPQNSVIVQIKAAEIVPSDMYKTGSTLRFPRIEKIRDDKEWHECMTLGDLEQLRGKASGKLATKHLHVGDDDEPREKRRKPISKTKKAIRIIEHLKAPNLSNVNKVSNVFEDVEFCVMSGLDGYPKADLENRIAEFGGYIVQNPGPDTYCVIAGSENVRVKNIISSDKNDVVKPEWLLECFKTKTCVPWQPRFMIHMCPSTKQHFAREYDCYGDSYFVDTDLDQLKEVFLGIKPSEQQTPEEMAPVIADLECRYSWDHSPLSMFRHYTIYLDLYAVINDLSSRIEATRLGITALELRFHGAKVVSCLSEGVSHVIIGEDQRRVTDFKIFRRMLKKKFKILQESWVSDSVDKGELQEENQYLL

SEQ ID NO: 80 - protein, 790 aa, Arabidopsis sp.
MLAIRSSNYLRCIPSLCTKTQISQFSSVLISFSRQISHLRLSSCHRAMSSSRPSAFDALMSNARAAAKKKTPQTTNLSRSPNKRKIGETQDANLGKTIVSEGTLPKTEDLLEPVSDSANPRSDTSSIAEDSKTGAKKAKTLSKTDEMKSKIGLLKKKPNDFDPEKMSCWEKGERVPFLFVALAFDLISNESGRIVITDILCNMLRTVIATTPEDLVATVYLSANEIAPAHEGVELGIGESTIIKAISEAFGRTEDHVKKQNTELGDLGLVAKGSRSTQTMMFKPEPLTVVKVFDTFRQIAKESGKDSNEKKKNRMKALLVATTDCEPLYLTRLLQAKLRLGFSGQTVLAALGQAAVYNEEHSKPPPNTKSPLEEAAKIVKQVFTVLPVYDIIVPALLSGGVWNLPKTCNFTLGVPIGPMLAKPTKGVAEILNKFQDIVFTCEYKYDGERAQIHFMEDGTFEIYSRNAERNTGKYPDVALALSRLKKPSVKSFILDCEVVAFDREKKKILPFQILSTRARKNVNVNDIKVGVCIFAFDMLYLNGQQLIQENLKIRREKLYESFEEDPGYFQFATAVTSNDIDEIQKFLDASVDVGCEGLIIKTLDSDATYEPAKRSNNWLKLKKDYMDSIGDSVDLVPIAAFHGRGKRTGVYGAFLLACYDVDKEEFQSICKIGTGFSDAMLDERSSSLRSQVIATPKQYYRVGDSLNPDVWFEPTEVWEVKAADLTISPVHRAATGIVDPDKGISLRFPRLLRVREDKKPEEATSSEQIADLYQAQKHNHPSNEVKGDDD

SEQ ID NO: 81 - protein, 1219 aa, Arabidopsis sp.
MTEEIKFSVLVSLFNWIQKSKTSSQKRSKFRKFLDTYCKPSDYFVAVRLIIPSLDRERGSYGLKESVLATCLIDALGISRDAPDAVRLLNWRKGGTAKAGANAGNFSLIAAEVLQRRQGMASGGLTIKELNDLLDRLASSENRAEKTLVLSTLIQKTNAQEMKWVIRIILKDLKLGMSEKSIFQEFHPDAEDLFNVTCDLKLVCEKLRDRHQRHKRQDIEVGKAVRPQLAMRIGDVNAAWKKLHGKDVVAECKFDGDRIQIHKNGTDIHYFSRNFLDHSEYAHAMSDLIVQNILVDKCILDGEMLVWDTSLNRFAEFGSNQEIAKAAREGLDSHKQLCYVAFDVLYVGDTSVIHQSLKERHELLKKVVKPLKGRLEVLVPEGGLNVHRPSGEPSWSIVVHAAADVERFFKETVENRDEGIVLKDLESKWEPGDRSGKWMKLKPEYIRAGADLDVLIIGGYYGSGRRGGEVAQFLVALADRAEANVYPRRFMSFCRVGTGLSDDELNTVVSKLKPYFRKNEHPKKAPPSFYQVTNHSKERPDVWIDSPEKSIILSITSDIRTIRSEVFVAPYSLRFPRIDKVRYDKPWHECLDVQAFVELVNSSNGTTQKQKESESTQDNPKVNKSSKRGEKKNVSLVPSQFIQTDVSDIKGKTSIFSNMIFYFVNVPRSHSLETFHKMVVENGGKFSMNLNNSVTHCIAAESSGIKYQAAKRQRDVIHFSWVLDCCSRNKMLPLLPKYFLHLTDASRTKLQDDIDEFSDSYYWDLDLEGLKQVLSNAKQSEDSKSIDYYKKKLCPEKRWSCLLSCCVYFYPYSQTLSTEEEALLGIMAKRLMLEVLMAGGKVSNNLAHASHLVVLAMAEEPLDFTLVSKSFSEMEKRLLLKKRLHVVSSHWLEESLQREEKLCEDVYTLRPKYMEESDTEESDKSEHDTTEVASQGSAQTKEPASSKIAITSSRGRSNTRAVKRGRSSTNSLQRVQRRRGKQPSKISGDETEESDASEEKVSTRLSDIAEETDSFGEAQRNSSRGKCAKRGKSRVGQTQRVQRSRRGKKAAKIGGDESDENDELDGNNNVSADAEEGNAAGRSVENEETREPDIAKYTESQQRDNTVAVEEALQDSRNAKTEMDMKEKLQIHEDPLQAMLMKMFPIPSQKTTETSNRTTGEYRKANVSGECESSEKRKLDAETDNTSVNAGAESDVVPPLVKKKKVSYRDVAGELLKDW

SEQ ID NO: 82 - protein, 1396 aa, Arabidopsis sp.
MASDSAGATISGNFSNSDNSETLNLNTTKLYSSAISSISPQFPSPKPTSSCPSIPNSKRIPNTNFIVDLFRLPHQSSSVAFFLSHFHSDHYSGLSSSWSKGIIYCSHKTARLVAEILQVPSQFVFALPMNQMVKIDGSEVVLIEANHCPGAVQFLFKVKLESSGFEKYVHTGDFRFCDEMRFDPFLNGFVGCDGVFLDTTYCNPKFVFPSQEESVGYVVSVIDKISEEKVLFLVATYVVGKEKILVEIARRCKRKIVVDARKMSMLSVLGCGEEGMFTEDENESDVHVVGWNVLGETWPYFRPNFVKMNEIMVEKGYDKVVGFVPTGWTYEVKRNKFAVRFKDSMEIHLVPYSEHSNYDELREFIKFLKPKRVIPTVGVDIEKFDCKEVNKMQKHFSGLVDEMANKKDFLLGFYRQSYQKNEKSDVDVVSHSAEVYEEEEKNACEDGGENVPSSRGPILHDTTPSSDSRLLIKLRDSLPAWVTEEQMLDLIKKHAGNPVDIVSNFYEYEAELYKQASLPTPSLNNQAVLFDDDVTDLQPNPVKGICPDVQAIQKGFDLPRKMNLTKGTISPGKRGKSSGSKSNKKAKKDPKSKPVGPGQPTLFKFFNKVLDGGSNSVSVGSETEECNTDKKMVHIDASEAYKEVTDQFIDIVNGSESLRDYAASIIDEAKGDISRALNIYYSKPREIPGDHAGERGLSSKTIQYPKCSEACSSQEDKKASENSGHAVNICVQTSAEESVDKNYVSLPPEKYQPKEHACWREGQPAPYIHLVRTFASVESEKGKIKAMSMLCNMFRSLFALSPEDVLPAVYLCTNKIAADHENIELNIGGSLISSALEEACGISRSTVRDMYNSLGDLGDVAQLCRQTQKLLVPPPPLLVRDVFSTLRKISVQTGTGSTRLKKNLIVKLMRSCREKEIKFLVRTLARNLRIGAMLRTVLPALGRAIVMNSFWNDHNKELSESCFREKLEGVSAAVVEAYNILPSLDVVVPSLMDKDIEFSTSTLSMVPGIPIKPMLAKIAKGVQEFFNLSQEKAFTCEYKYDGQRAQIHKLLDGTVCIFSRNGDETTSRFPDLVDVIKQFSCPAAETFMLDAEVVATDRINGNKLMSFQELSTRERGSKDALITTESIKVEVCVFVFDIMFVNGEQLLALPLRERRRRLKEVFPETRPGYLEYAKEITVGAEEASLNNHDTLSRINAFLEEAFQSSCEGIMVKSLDVNAGYCPTKRSDSWLKVKRDYVDGLGDTLDLVPIGAWYGNGRKAGWYSPFLMACFNPETEEFQSVCRVMSGFSDAFYIEMKEFYSEDKILAKKPPYYRTGETPDMWFSAEVVWEIRGADFTVSPVHSASLGLVHPSRGISVRFPRFISKVTDRNPEECSTATDIAEMFHAQTRKMNITSQH

SEQ ID NO: 83 - protein, 668 aa, Bacillus subtilis
MDKETAKQRAEELRRTINKYSYEYYTLDEPSVPDAEYDRLMQELIAIEEEHPDLRTPDSPTQRVGGAVLEAFQKVTHGTPMLSLGNAFNADDLRDFDRRVRQSVGDDVAYNVELKIDGLAVSLRYEDGYFVRGATRGDGTTGEDITENLKTIRNIPLKMNRELSIEVRGEAYMPKRSFEALNEERIKNEEEPFANPRNAAAGSLRQLDPKIAAKRNLDIFVYSIAELDEMGVETQSQGLDFLDELGFKTNQERKKCGSIEEVITLIDELQAKRADLPYEIDGIVIKVDSLDQQEELGFTAKSPRWAIAYKFPAEEVVTKLLDIELNVGRTGVITPTAILEPVKVAGTTVSRASLHNEDLIKEKDIRILDKVVVKKAGDIIPEVVNVLVDQRTGEEKEFSMPTECPECGSELVRIEGEVALRCINPECPAQIREGLIHFVSRNAMNIDGLGERVITQLFEENLVRNVADLYKLTKERVIQLERMGEKSTENLISSIQKSKENSLERLLFGLGIRFIGSKAAKTLAMHFESLENLKKASKEELLAVDEIGEKMADAVITYFHKEEMLELLNELQELGVNTLYKGPKKVKAEDSDSYFAGKTIVLTGKLEELSRNEAKAQIEALGGKLTGSVSKNTDLVIAGEAAGSKLTKAQELNIEVWNEEQLMGELKK

SEQ ID NO: 84 - protein, 670 aa, Bacillus sp.
MDRQQAERRAAELRELLNRYGYEYYVLDRPSVPDAEYDRLMQELIAIEEQYPELKTSDSPTQRIGGPPLEAFRKVAHRVPMMSLANAFGEGDLRDFDRRVRQEVGEAAYVCELKIDGLAVSVRYEDGYFVQGATRGDGTTGEDITENLKTIRSLPLRLKEPVSLEARGEAFMPKASFLRLNEERKARGEELFANPRNAAAGSLRQLDPKVAASRQLDLFVYGLADAEALGIASHSEALDYLQALGFKVNPERRRCANIDEVIAFVSEWHDKRPQLPYEIDGIVIKVDSFAQQRALGATAKSPRWAIAYKFPAEEVVTTLIGIEVNVGRTGVVTPTAILEPVRVAGTTVQRATLHNEDFIREKDIRIGDAVIIKKAGDIIPEVVGVVVDRRDGDETPFAMPTHCPECESELVRLEGEVALRCLNPNCPAQLRERLIHFASRAAMNIEGLGEKVVTQLFNAGLVRDVADLYCLTKEQLVGLERMGEKSAANLLAAIEASKQNSLERLLFGLGIRYVGAKAAQLLAEHFETMERLERATKEELMAVPEIGEKMADAITAFFAQPEATELLQELRAYGVNMAYKGPKRSAEAPADSAFAGKTVVLTGKLASMSRNEAKEQIERLGGRVTGSVSRSTDLVIAGEDAGSKLEKAQQLGIEIWDESRFLQEINRGKR

SEQ ID NO: 85 - protein, 268 aa, Haemophilus influenzae
MKFYRTLLLFFASSFAFANSDLMLLHTYNNQPIEGWVMSEKLDGVRGYWNGKQLLTRQGQRLSPPAYFIKDFPPFAIDGELFSERNHFEEISTITKSFKGDGWEKLKLYVFDVPDAEGNLFERLAKLKAHLLEHPTTYIEIIEQIPVKDKTHLYQFLAQVENLQGEGVVVRNPNAPYERKRSSQILKLKTARGEECTVIAHHKGKGQFENVMGALTCKNHRGEFKIGSGFNLNERENPPPIGSVITYKYRGITNSGKPRFATYWREKK

SEQ ID NO: 86 - protein, 672 aa, Pseudoalteromonas haloplanktis
MSSSISEQVNHLRIILEQHNYNYYVLDTPSIPDSEYDRLLRELSALETEHPEFLTADSPTQKVGGAALSKFEQVAHQVPMLSLDNAFSEEEFTAFNRRIKERLMSTDELTFCCEPKLDGLAVSIIYRDGVLVQAATRGDGFTGENITQNVKTIRNVPLKLRGDYPKELEVRGEVFMDSAGFDKLNTEAEKRGEKVFVNPRNAAAGSLRQLDSKITAKRPLMFYAYSTGLVADGNIPEDHYQQLEKLTDWGLPLCPETKLVEGPKAALAYYRDILTRRSELKYEIDGVVIKINQKTLQERLGFVARAPRWAIAYKFPAQEEITQLLDVDFQVGRTGAITPVARLKPVFVGGVTVSNATLHNSDEVARLGVKVGDTVIIRRAGDVIPQITQVVLERRPDDARDIEFPTTCPICDSHVEKVEGEAVARCTGGLVCPAQRKQAIKHFASRKALDIDGLGDKIVDQLVDRELIKTPADLFILKQGHFESLERMGPKSAKNLVTALEEAKGTTLAKFLYSLGIREAGEATAQNLANHFLTLENVINASIDSLTQVSDVGEIVAAHVRGFFDEEHNLAVVNALIDQGVNWPALSAPSEEEQPLAGLTYVLTGTLNTLNRNDAKARLQQLGAKVSGSVSAKTDALVAGEKAGSKLTKAQDLGIDILTEDELIELLIKHNG

SEQ ID NO: 87 - protein, 712 aa, Rhodothermus marinus
METHTAPQTAEARLLEATHTLLQTVRQRDLEAIDRKEAEALAARLREVLNQHAYRYYVLDNPLIPDADYDLLMQALRKLEARFPELVTPDSPTQRVGGPPLGRFEKVRHPEPLLSLNNAFGEEDVRVWYERCCRMLAERLGQPVQPAVTAELKIDGLAMALTYENGVLSVGATRGDGIEGENVTQNVRTIPAIPLRIPVDPAVGPPPTRLEVRGEVYMRKRDFERLNEQLQARGERPFANPRNAAAGSVRQLNPQVTALRPLSFFAYGIGPVEGAEVPDSQYEVLQWLGRLGFPVNEHARRFEHLDDVLEYCRYWTEHRDELDYEIDGVVLKIDHRPWQALLGAISNAPRWAVAYKFPAREAITRLLDIMVSVGRTGVVKPVAVLEPVEVGGVTVSQATLHNEDYVRSRDIRIGDLVVVIRAGDVIPQVVRPVVEARTGNERPWRMPERCPSCGSQLVRLPGEADYYCVASDCPAQFVRLLEHFAGRDAMDIEGMGSQVARQLAESGLVRPLSDLYRLKLEDLLKLEGFAETRARNLLRAIEASKQRPLSRLLFGLGIRHVGKTTAELLVQRFASIDELAAATIDELAALEGVGPITAESIANWFRVEDNRRLIEELKELGVNTQRLPEEAPAAESPVRGKTFVLTGALPHLTRKEAEELIKRAGGRVASSVSRNTDYVVVGENPGSKYDRARQLGIPMLDEDGLLRLLGMK

SEQ ID NO: 88 - protein, 670 aa, Thermus filiformis
MTREEARRRINELRDLIRYHNYRYYVLADPEISDAEYDRLLRELKELEERFPEFKSPDSPTEQVGARPLEPTFRPVRHPTRMYSLDNAFTYEEVLAFEERLERALGRKRPFLYTVEHKVDGLSVNLYYEEGVLVFGATRGDGEVGEEVTQNLLTIPTIPRRLKGVPDRLEVRGEVYMPIEAFLRLNEELEERGEKVFKNPRNAAAGSLRQKDPRVTAKRGLRATFYALGLGLEESGLKSQYELLLWLKEKGFPVEHGYEKALGAEGVEEVYRRFLAQRHALPFEADGVVVKLDDLALWRELGYTARAPRFALAYKFPAEEKETRLLDVVFQVGRTGRVTPVGVLEPVFIEGSEVSRVTLHNESYIEELDIRIGDWVLVHKAGGVIPEVLRVLKERRTGEERPIRWPETCPECGHRLVKEGKVHRCPNPLCPAKRFEAIRHYASRKAMDIEGLGEKLIERLLEKGLVRDVADLYHLRKEDLLGLERMGEKSAQNLLRQIEESKHRGLERLLYALGLPGVGEVLARNLARRFGTMDRLLEASLEELLEVEEVGELTARAILETLKDPAFRDLVRRLKEAGVSMESKEEVSDLLSGLTFVLTGELSRPREEVKALLQRLGAKVTDSVSRKTSYLVVGENPGSKLEKARALGVAVLTEEEFWRFLKEKGAPVPA

SEQ ID NO: 89 - protein, 674 aa, Thermus scotoductus
MTLEEARKRVNELRDLIRYHNYRYYVLADPEISDAEYDRLLRELKELEERFPELKSPDSPTEQVGAKPLEATFRPIRHPTRMYSLDNAFNFDELKAFEERIGRALGREGPFAYTVEHKVDGLSVNLYYEDGVLVWGATRGDGEVGEEVTQNLLTIPTIPRRVKGVPERLEVRGEVYMPIEAFLRLNEELEEKGEKIFKNPRNAAAGSLRQKDPRITARRGLRATFYALGLGLEESGLKTQLDLLHWLREKGFPVEHGFARAEGAEGVERIYQGWLKERRSLPFEADGVVVKLDELSLWRELGYTARAPRFAIAYKFPAEEKETRLLQVVFQVGRTGRVTPVGILEPVFIEGSVVSRVTLHNESYIEELDVRIGDWVLVHKAGGVIPEVLRVLKEKRTGEERPIRWPETCPECGHRLVKEGKVHRCPNPLCPAKRFEAIRHYASRKAMDIGGLGEKLIEKLLEKGLVKDVADLYRLKKEDLLGLERMGEKSAQNLLRQIEESKGRGLERLLYALGLPGVGEVLARNLAAHFGTMDRLLEASLEELLQVEEVGELTARGIYETLQDPAFRDLVRRLKEAGVVMEAKERGEEALKGLTFVITGELSRPREEVKALLRRLGAKVTDSVSRKTSYLVVGENPGSKLEKARALGVPTLTEEELYRLIEERTGKPVETLAS

SEQ ID NO: 90 - protein, 674 aa, Thermus sp.
MTLEEARRRVNELRDLIRYHNYLYYVLDAPEISDAEYDRLLRELKELEERFPELKSPDSPTEQVGARPLEATFRPVRHPTRMYSLDNAFSLDEVRAFEERIERALGRKGPFLYTVERKVDGLSVNLYYEEGILVFGATRGDGETGEEVTQNLLTIPTIPRRLTGVPDRLEVRGEVYMPIEAFLRLNQELEEAGERIFKNPRNAAAGSLRQKDPRVTARRGLRATFYALGLGLEETGLKSQHDLLLWLRERGFPVEHGFTRALGAEGVEEVYQAWLKERRKLPFEADGVVVKLDDLALWRELGYTARTPRFALAYKFPAEEKETRLLSVAFQVGRTGRITPVGVLEPVFIEGSEVSRVTLHNESFIEELDVRIGDWVLVHKAGGVIPEVLRVLKERRTGEEKPIIWPENCPECGHALIKEGKVHRCPNPLCPAKRFEAIRHYASRKAMDIQGLGEKLIEKLLEKGLVRDVADLYRLKKEDLVNLERMGEKSAENLLRQIEESKGRGLERLLYALGLPGVGEVLARNLALRFGHMDRLLEAGLEDLLEVEGVGELTARAILNTLKDPEFRDLVRRLKEAGVEMEAKEREGEALKGLTFVITGELSRPREEVKALLRRLGAKVTDSVSRKTSFLVVGENPGSKLEKARALGVPTLSEEELYRLIEERTGKDPRALTA

SEQ ID NO: 91 - protein, 676 aa, Thermus thermophilus
MTLEEARKRVNELRDLIRYHNYRYYVLADPEISDAEYDRLLRELKELEERFPELKSPDSPTLQVGARPLEATFRPVRHPTRMYSLDNAFNLDELKAFEERIERALGRKGPFAYTVEHKVDGLSVNLYYEEGVLVYGATRGDGEVGEEVTQNLLTIPTIPRRLKGVPERLEVRGEVYMPIEAFLRLNEELEERGERIFKNPRNAAAGSLRQKDPRITAKRGLRATFYALGLGLEEVEREGVATQFALLHWLKEKGFPVEHGYARAVGAEGVEAVYQDWLKKRRALPFEADGVVVKLDELALWRELGYTARAPRFAIAYKFPAEEKETRLLDVVFQVGRTGRVTPVGILEPVFLEGSEVSRVTLHNESYIEELDIRIGDWVLVHKAGGVIPEVLRVLKERRTGEERPIRWPETCPECGHRLLKEGKVHRCPNPLCPAKRFEAIRHFASRKAMDIQGLGEKLIERLLEKGLVKDVADLYRLRKEDLVGLERMGEKSAQNLLRQIEESKKRGLERLLYALGLPGVGEVLARNLAARFGNMDRLLEASLEELLEVEEVGELTARAILETLKDPAFRDLVRRLKEAGVEMEAKEKGGEALKGLTFVITGELSRPREEVKALLRRLGAKVTDSVSRKTSYLVVGENPGSKLEKARALGVPTLTEEELYRLLEARTGKKAEELV

SEQ ID NO: 92 - protein, 731 aa, Zymomonas mobilis
MNADIDLFSYLNPEKQDLSALAPKDLSREQAVIELERLAKLISHYDHLYHDKDNPAVPDSEYDALVLRNRRIEQFFPDLIRPDSPSKKVGSRPNSRLPKIAHRAAMLSLDNGFLDQDVEDFLGRVRRFFNLKENQAVICTVEPKIDGLSCSLRYEKGILTQAVTRGDGVIGEDVTPNVRVIDDIPKTLKGDNWPEIIEIRGEVYMAKSDFAALNARQTEENKKLFANPRNAAAGSLRQLDPNITARRSLRFLAHGWGEATSLPADTQYGMMKVIESYGLSVSNLLARADDIGQMLDFYQKIEAERADLDFDIDGVVYKLDQLDWQQRFGFSARAPRFALAHKFPAEKAQTTLLDIEIQVGRTGVLTPVAKLEPVTVGGVVVSSATLHNSDEIERLGVRPGDRVLVQRAGDVIPQIVENLTPDVDRPIWRFPHRCPVCDSVARREEGEVAWRCTGGLICPAQRVERLCHFVSRTAFEIEGLGKSHIESFFADKLIETPADIFRLFQKRQLLIEREGWGELSVDNLISAIDKRRKVPFDRFLFALGIRHVGAVTARDLAKSYQTWDNFKAAIDEAAHLRTILQPSSEESEEKYQKRVDKELISFFHIPNMGGKIIRSLLDFFAETHNSDVVSDLLQEVQIEPLYFELASSPLSGKIIVFTGSLQKITRDEAKRQAENLGAKVASSVSKKTNLVVAGEAAGSKLSKAKELDISIIDEDRWHRIVENDGQDSIKI

SEQ ID NO: 93 - protein, 647 aa, Campylobacter jejuni
MKKEEYLEKVALANLWMRAYYEKDEPLASDEEYDVLIRELRVFEEQNKDEISKDSPTQKIAPTIQSEFKKIAHLKRMWSMEDVFDESELRAWAKRAKCEKNFFIEPKFDGASLNLLYENGKLVSGATRGDGEVGEDITLNVFEIENIPKNIAYKERIEIRGEVVILKDDFEKINEKRALLNQSLFANPRNAASGSLRQLDTSITKERNLKFYPWGVGENTLNFTKHSEVMQFIRELGFLKDDFIKLCANLDEVLKAYDELLALREKKPMMMDGMVVRIDDLALCEELGYTVKFPKFMAAFKFPALEKTTRLIGVNLQVGRSGVITPVAVLEPVNLDGVVVKSATLHNFDEIARLDVKINDFVSVIRSGDVIPKITKVFKDRREGLEMEISRPKLCPTCQSELLDEGTLIKCQNIDCEDRLVNSIIHFVSKKCLNIDGLGENIVELLYKHKKITTLESIFHLKFSDFEGLEGFKEKKINNLLNAIEQARECELFRFITALGIEHIGEVAAKKLSLSFGKEWHKQSFEAYANLEGFGEQMALSLCEFTRVNHVRIDEFYKLLNLKIEKLEIKSDGVIFGKTFVITGTLSRPRDEFKALIEKLGGKVSSSVSKKTDYVLFGEEAGSKLIKAKELEVKCIDESAFNELVKE

SEQ ID NO: 94 - protein, 691 aa, Mycobacterium tuberculosis
MSSPDADQTAPEVLRQWQALAEEVREHQFRYYVRDAPIISDAEFDELLRRLEALEEQHPELRTPDSPTQLVGGAGFATDFEPVDHLERMLSLDNAFTADELAAWAGRIHAEVGDAAHYLCELKIDGVALSLVYREGRLTRASTRGDGRTGEDVTLNARTIADVPERLTPGDDYPVPEVLEVRGEVFFRLDDFQALNASLVEEGKAPFANPRNSAAGSLRQKDPAVTARRRLRMICHGLGHVEGFRPATLHQAYLALRAWGLPVSEHTTLATDLAGVRERIDYWGEHRHEVDHEIDGVVVKVDEVALQRRLGSTSRAPRWAIAYKYPPEEAQTKLLDIRVNVGRTGRITPFAFMTPVKVAGSTVGQATLHNASEIKRKGVLIGDTVVIRKAGDVIPEVLGPVVELRDGSEREFIMPTTCPECGSPLAPEKEGDADIRCPNARGCPGQLRERVFHVASRNGLDIEVLGYEAGVALLQAKVIADEGELFALTERDLLRTDLFRTKAGELSANGKRLLVNLDKAKAAPLWRVLVALSIRHVGPTAARALATEFGSLDAIAAASTDQLAAVEGVGPTIAAAVTEWFAVDWHREIVDKWRAAGVRMVDERDESVPRTLAGLTIVVTGSLTGFSRDDAKEAIVARGGKAAGSVSKKTNYVVAGDSPGSKYDKAVELGVPILDEDGFRRLLADGPASRT

SEQ ID NO: 95 - protein, 735 aa, Emiliania huxleyi virus 86
MEAMCTECEDRDARLDVIDIQLFHALNPKSCNRTTWEQVPKIMGKQGDFVAEGKLDGERDASHLYGESMEDVLCECVREDVTSLLLDGEMMVVDLETGRYLPFGENRSLKDFGTSMRHCFVAFDLLLYNGRSMTGATLAERSELLRKAVRTKQHALTLIERFEVGERGAGATTAVMRQLDVMMSRGLEGVVFKSLSSKYDPGSRDKSWIKLKPDFVDGMGDTLDLLILGGYYGEGRRRSGAVSTFLMGVRAPPEAAKRVGGAAHPLFYPFCKVGTGYSLPQLRELRERLMPASLTRRRGNALGHGASLTAVSCEQVSHEWKNSRRPAHLCHWEPSKRDDIPDYWFEPEASVVLELTAFEIITPRESFLPANYTLRFPRVKRVRYDKGWEGAETFERVVELFKECDGRLSANKRRAEEIAASRASAGPAAKKRAAGVAPTVGVPWHLKLSADLANTAVECYALDGVVAVVKGTLSRRPGVETQIKRLGGKVHKNMTSLTTHLVDAPGAEVLAEVERARRGGGSFEVVTAAWVDECSRVHARVTLEPRYVRHVSEATREQIEAIMDEWGDNYTIAADPESLVDSMRLVREQRSAGGNCGDSPLAREAHVADALRDLDDETAVALRTRYAMLRGVVAYVPRGSVALRLRLRLLGAQTVDEPSADSTHAVLSASTSADERQRLRDKFTEDRVRDGRPSCGRHIVSDRWLAECERRGQREPEAQEDAWFGDRVGIRDRAL

SEQ ID NO: 96 - protein, 548 aa, Lymantria dispar multiple nucleopolyhedrovirus
MENHDSFYKFCQLCQSLYDADDHQEKRDALERHFADFRGSAFMWRELLAPAESDAAADRELTLIFETILSIERTEQENVTRNLKCTIDGAAVPLSRESRITVPQVYEFINDLRGSGSRQERLRLIGQFAAGCTDEDLLTVFRVVSDHAHAGLSAEDVMELVEPWERFQKPVPPALAQPCRRLASVLVKHPEGALAEVKYDGERVQVHKAGSRFKFFSRTLKPVPEHKVAGCREHLTRAFPRARNFILDAEIVMVDGSGEALPFGTLGRLKQMEHADGHVCMYIFDCLRYNGVSYLNATPLDFRRRVLQDEIVPIEGRVVLSAMERTNTLSELRRFVHRTLATGAEGVVLKGRLSSYAPNKRDWFKMKKEHLCDGALVDTLDLVVLGAYYGTGRNCRKMSVFLMGCLDRESNVWTTVTKVHSGLADAALTALSKELRPLMAAPRDDLPEWFDCNESMVPHLLAADPEKMPVWEIACSEMKANIGAHTAGVTMRFPRVKRFRPDKDWSTATDLQEAEQLIRNSQENTKKTFARLATTYDGPSPNKKLKLN

SEQ ID NO: 97 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgccggtgccctggcccaccctcgtgaccaccctgacccatggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 98 - RNA, 149 nt, synthetic construct
  source 1..149: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 146^147: mod_base = OTHER; note = phosphorothioate bond
  modified_base 147^148: mod_base = OTHER; note = phosphorothioate bond
  modified_base 148^149: mod_base = OTHER; note = phosphorothioate bond
  modified_base 146: mod_base = OTHER; note = 2-O-methyl nucleotide
  modified_base 147: mod_base = OTHER; note = 2-O-methyl nucleotide
  modified_base 148: mod_base = OTHER; note = 2-O-methyl nucleotide
ctgaagttcatctgcaccacgtttaagagctatgctggaaacagcatagcaagtttaaataaggctagtccgttatcaacttgaaaaagtggcaccgagtcggtgcgtgggccagggcaccggcagcttgccggtggtgcagatgaact

SEQ ID NO: 99 - RNA, 149 nt, synthetic construct
  source 1..149: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 146: mod_base = OTHER; note = 2-O-methyl nucleotide
  modified_base 147: mod_base = OTHER; note = 2-O-methyl nucleotide
  modified_base 148: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 146^147: mod_base = OTHER; note = phosphorothioate bond
  modified_base 147^148: mod_base = OTHER; note = phosphorothioate bond
  modified_base 148^149: mod_base = OTHER; note = phosphorothioate bond
gacgtagccttcgggcatgggtttaagagctatgctggaaacagcatagcaagtttaaataaggctagtccgttatcaacttgaaaaagtggcaccgagtcggtgctgaagcagcacgacttcttcaagtccgccatgcccgaaggcta

SEQ ID NO: 100 - RNA, 149 nt, synthetic construct
  source 1..149: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 146: mod_base = OTHER; note = 2-O-methyl nucleotide
  modified_base 147: mod_base = OTHER; note = 2-O-methyl nucleotide
  modified_base 148: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 146^147: mod_base = OTHER; note = phosphorothioate bond
  modified_base 147^148: mod_base = OTHER; note = phosphorothioate bond
  modified_base 148^149: mod_base = OTHER; note = phosphorothioate bond
ctgaagttcatctgcaccacgtttaagagctatgctggaaacagcatagcaagtttaaataaggctagtccgttatcaacttgaaaaagtggcaccgagtcggtgcgtgggccagggcaccggcagcttgccggttgtgcagatgaact

SEQ ID NO: 101 - RNA, 149 nt, synthetic construct
  source 1..149: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 146: mod_base = OTHER; note = 2-O-methyl nucleotide
  modified_base 147: mod_base = OTHER; note = 2-O-methyl nucleotide
  modified_base 148: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 146^147: mod_base = OTHER; note = phosphorothioate bond
  modified_base 147^148: mod_base = OTHER; note = phosphorothioate bond
  modified_base 148^149: mod_base = OTHER; note = phosphorothioate bond
gacgtagccttcgggcatgggtttaagagctatgctggaaacagcatagcaagtttaaataaggctagtccgttatcaacttgaaaaagtggcaccgagtcggtgctgaagcagcacgacttcttcaagtcagccatgcccgaaggcta

SEQ ID NO: 102 - DNA, 86 bp, synthetic construct
  source 1..86: mol_type = other DNA
  modified_base 1: mod_base = OTHER; note = 5' phosphate
caccggcaagctgccggtgccctggcccaccctcgtgaccaccctgacctacggcgtgcagtgcttcagccgctaccccgaccaca

SEQ ID NO: 103 - DNA, 86 bp, synthetic construct
  source 1..86: mol_type = other DNA
  modified_base 1: mod_base = OTHER; note = 5' Phosphate
tggcggacttgaagaagtcgtgctgcttcatgtggtcggggtagcggctgaagcactgcacgccgtaggtcagggtggtcacgagg

SEQ ID NO: 104 - DNA, 86 bp, synthetic construct
  source 1..86: mol_type = other DNA
  modified_base 1: mod_base = OTHER; note = 5' Phosphate
caccggcaagctgccggtgccctggcccactcttgtgaccaccttgacctacggtgtccagtgttttagcaggtatccggatcaca

SEQ ID NO: 105 - DNA, 86 bp, synthetic construct
  source 1..86: mol_type = other DNA
  modified_base 1: mod_base = OTHER; note = 5' Phosphate
tggcggacttgaagaagtcgtgctgcttcatgtgatccggatacctgctaaaacactggacaccgtaggtcaaggtggtcacaaga

SEQ ID NO: 106 - DNA, 86 bp, synthetic construct
  source 1..86: mol_type = other DNA
  modified_base 1: mod_base = OTHER; note = 5' Phosphate
aaccggcaagctgccggtgccctggcccactcttgtgaccaccttgacctacggtgtccagtgttttagcaggtatccggatcaca

SEQ ID NO: 107 - DNA, 86 bp, synthetic construct
  source 1..86: mol_type = other DNA
  modified_base 1: mod_base = OTHER; note = 5' Phosphate
tggctgacttgaagaagtcgtgctgcttcatgtgatccggatacctgctaaaacactggacaccgtaggtcaaggtggtcacaaga

SEQ ID NO: 108 - DNA, 86 bp, synthetic construct; identical to SEQ ID NO: 104
  source 1..86: mol_type = other DNA
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 83^84: mod_base = OTHER; note = phosphorothioate bond
  modified_base 84^85: mod_base = OTHER; note = phosphorothioate bond
  modified_base 85^86: mod_base = OTHER; note = phosphorothioate bond
  modified_base 1: mod_base = OTHER; note = 5' Phosphate

SEQ ID NO: 109 - DNA, 86 bp, synthetic construct; identical to SEQ ID NO: 105
  source 1..86: mol_type = other DNA
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 83^84: mod_base = OTHER; note = phosphorothioate bond
  modified_base 84^85: mod_base = OTHER; note = phosphorothioate bond
  modified_base 85^86: mod_base = OTHER; note = phosphorothioate bond
  modified_base 1: mod_base = OTHER; note = 5' Phosphate

SEQ ID NO: 110 - RNA, 126 nt, synthetic construct
  source 1..126: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 123: mod_base = OTHER; note = 2-O-methyl nucleotide
  modified_base 124: mod_base = OTHER; note = 2-O-methyl nucleotide
  modified_base 125: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 123^124: mod_base = OTHER; note = phosphorothioate bond
  modified_base 124^125: mod_base = OTHER; note = phosphorothioate bond
  modified_base 125^126: mod_base = OTHER; note = phosphorothioate bond
ctgaagttcatctgcaccacgtttaagagctatgctggaaacagcatagcaagtttaaataaggctagtccgttatcaacttgaaaaagtggcaccgagtcggtgcctacggcaagctgaccctga

SEQ ID NO: 111 - RNA, 126 nt, synthetic construct
  source 1..126: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 123: mod_base = OTHER; note = 2-O-methyl nucleotide
  modified_base 124: mod_base = OTHER; note = 2-O-methyl nucleotide
  modified_base 125: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 123^124: mod_base = OTHER; note = phosphorothioate bond
  modified_base 124^125: mod_base = OTHER; note = phosphorothioate bond
  modified_base 125^126: mod_base = OTHER; note = phosphorothioate bond
gacgtagccttcgggcatgggtttaagagctatgctggaaacagcatagcaagtttaaataaggctagtccgttatcaacttgaaaaagtggcaccgagtcggtgcagatggtgcgctcctggacg

SEQ ID NO: 112 - DNA, 149 bp, synthetic construct
  source 1..149: mol_type = other DNA
  modified_base 1: mod_base = OTHER; note = 5' Phosphate
aaccggcaagctgccggtgccctggcccaccctcgtgaccaccctgacctacggcgtgcagtgcttcagccgctaccccgaccacatgaagcagcacgacttcttcaagtcagccatgcccgaaggctacgtccaggagcgcaccatct

SEQ ID NO: 113 - DNA, 149 bp, synthetic construct
  source 1..149: mol_type = other DNA
  modified_base 1: mod_base = OTHER; note = 5' Phosphate
tggctgacttgaagaagtcgtgctgcttcatgtggtcggggtagcggctgaagcactgcacgccgtaggtcagggtggtcacgagggtgggccagggcaccggcagcttgccggttgtgcagatgaacttcagggtcagcttgccgtag

SEQ ID NO: 114 - DNA, 149 bp, synthetic construct
  source 1..149: mol_type = other DNA
  modified_base 1: mod_base = OTHER; note = 5' Phosphate
aaccggtaagttgccagtcccgtggcctactcttgtgaccaccttgacctacggtgtccagtgttttagcaggtatccggatcacatgaaacagcatgacttctttaaatcagctatgcccgaaggctacgtccaggagcgcaccatct

SEQ ID NO: 115 - DNA, 149 bp, synthetic construct
  source 1..149: mol_type = other DNA
  modified_base 1: mod_base = OTHER; note = 5' Phosphate
tagctgatttaaagaagtcatgctgtttcatgtgatccggatacctgctaaaacactggacaccgtaggtcaaggtggtcacaagagtaggccacgggactggcaacttaccggttgtgcagatgaacttcagggtcagcttgccgtag

SEQ ID NO: 116 - DNA, 149 bp, synthetic construct
  source 1..149: mol_type = other DNA
  modified_base 1: mod_base = OTHER; note = 5' Phosphate
caccggcaagctgccggtgccctggcccaccctcgtgaccaccctgacctacggcgtgcagtgcttcagccgctaccccgaccacatgaagcagcacgacttcttcaagtccgccatgcccgaaggctacgtccaggagcgcaccatct

SEQ ID NO: 117 - DNA, 149 bp, synthetic construct
  source 1..149: mol_type = other DNA
  modified_base 1: mod_base = OTHER; note = 5' Phosphate
tggcggacttgaagaagtcgtgctgcttcatgtggtcggggtagcggctgaagcactgcacgccgtaggtcagggtggtcacgagggtgggccagggcaccggcagcttgccggtggtgcagatgaacttcagggtcagcttgccgtag

SEQ ID NO: 118 - DNA, 149 bp, synthetic construct; identical to SEQ ID NO: 114
  source 1..149: mol_type = other DNA
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 146^147: mod_base = OTHER; note = phosphorothioate bond
  modified_base 147^148: mod_base = OTHER; note = phosphorothioate bond
  modified_base 148^149: mod_base = OTHER; note = phosphorothioate bond
  modified_base 1: mod_base = OTHER; note = 5' Phosphate

SEQ ID NO: 119 - DNA, 149 bp, synthetic construct; identical to SEQ ID NO: 115
  source 1..149: mol_type = other DNA
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 146^147: mod_base = OTHER; note = phosphorothioate bond
  modified_base 147^148: mod_base = OTHER; note = phosphorothioate bond
  modified_base 148^149: mod_base = OTHER; note = phosphorothioate bond
  modified_base 1: mod_base = OTHER; note = 5' Phosphate

SEQ ID NO: 120 - RNA, 126 nt, synthetic construct
  source 1..126: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 123: mod_base = OTHER; note = 2-O-methyl nucleotide
  modified_base 124: mod_base = OTHER; note = 2-O-methyl nucleotide
  modified_base 125: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 123^124: mod_base = OTHER; note = phosphorothioate bond
  modified_base 124^125: mod_base = OTHER; note = phosphorothioate bond
  modified_base 125^126: mod_base = OTHER; note = phosphorothioate bond
gaaagctggcgggcactatggtttaagagctatgctggaaacagcatagcaagtttaaataaggctagtccgttatcaacttgaaaaagtggcaccgagtcggtgcgtcaccctttacaccagaaa

SEQ ID NO: 121 - RNA, 126 nt, synthetic construct
  source 1..126: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 123: mod_base = OTHER; note = 2-O-methyl nucleotide
  modified_base 124: mod_base = OTHER; note = 2-O-methyl nucleotide
  modified_base 125: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 123^124: mod_base = OTHER; note = phosphorothioate bond
  modified_base 124^125: mod_base = OTHER; note = phosphorothioate bond
  modified_base 125^126: mod_base = OTHER; note = phosphorothioate bond
ctttgccctttaccactcgagtttaagagctatgctggaaacagcatagcaagtttaaataaggctagtccgttatcaacttgaaaaagtggcaccgagtcggtgctttaggaggtactccacttt

SEQ ID NO: 122 - DNA, 860 bp, synthetic construct
  source 1..860: mol_type = other DNA
atggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttaggctacggcgtggcctgcttcgcccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgtgctgaagggcatcgacttcaaggaggacggcaacatcctggggcacaagctggagtacaacttcaacagccacaaggtctatatcacggccgacaagcagaagaacggcatcaaggctaacttcaagacccgccacaacgttgaggacggcggcgtgcagctcgccgaccactaccagcagaacacccccatcggcgacggccccgtgctgctgcccgacaaccactacctgagccatcagtccaaactgagcaaagaccccaacgagaagcgcgatcacatggtcctgaaggagagggtgaccgccgccgggattacacatgacatggacgagctgtacaagtctggaggatctagcggaggatccgggaagaaacaaaacaagaagaaagtggaggaggtgctagaagaggaggaagaggaatatgtggtggaaaaagttctcgatcgtcgagtggtaaagggcaaagtggagtacctcctaaa

SEQ ID NO: 123 - DNA, 860 bp, synthetic construct
  source 1..860: mol_type = other DNA
cgacgatcgagaactttttccaccacatattcctcttcctcctcttctagcacctcctccactttcttcttgttttgtttcttcccggatcctccgctagatcctccagacttgtacagctcgtccatgtcatgtgtaatcccggcggcggtcaccctctccttcaggaccatgtgatcgcgcttctcgttggggtctttgctcagtttggactgatggctcaggtagtggttgtcgggcagcagcacggggccgtcgccgatgggggtgttctgctggtagtggtcggcgagctgcacgccgccgtcctcaacgttgtggcgggtcttgaagttagccttgatgccgttcttctgcttgtcggccgtgatatagaccttgtggctgttgaagttgtactccagcttgtgccccaggatgttgccgtcctccttgaagtcgatgcccttcagcacgatgcggttcaccagggtgtcgccctcgaacttcacctcggcgcgggtcttgtaggtaccgtcgtccttgaaagagatggtgcgctcctggacgtagccttcgggcatggcggacttgaagaagtcgtgctgcttcatgtggtcggggtagcgggcgaagcaggccacgccgtagcctaaggtggtcacgagggtgggccagggcacgggcagcttgccggtggtgcagatgaacttcagggtcagcttgccgttggtggcatcgccctcgccctcgccgcggacgctgaacttgtggccgtttacgtcgccgtccagctcgaccaggatgggcaccaccccggtgaacagctcctcgcccttgctcaccatagtgcccgccagctttctggtgtaaagggtgac

SEQ ID NO: 124 - DNA, 177 bp, synthetic construct
  source 1..177: mol_type = other DNA
cagcgtcaccctttacaccagaaagctggcgggcactatggggaaaaaacaaaacaagaagaaagtggaggaggtgctagaagaggaggaagaggaatatgtggtggaaaaagttctcgaccgtcgagtggtaaagggcaaagtggagtacctcctaaagtggaagggattctcaga

SEQ ID NO: 125 - DNA, 6762 bp, synthetic construct
  source 1..6762: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcccgaagcgccggactgcacgaaagcaactgccaaaacggactatacaagaagtcctggaagaacaaagcgaagatgaggatcgcgaagccaagcgcaagaaagaggaagaggaagaagagactccaaaggagtccttgaccgaagcagaagtcgcaacggagaaggaaggtgaggatggggatcagccaacaaccccgcctaaacctctgaaaacctctaaggcggagacaccaactgagagtgtcagcgaaccggaggtagccacgaaacaagagcttcaggaggaagaagaacagacaaagccacctcggcgggctcccaaaacccttagctccttcttcacgcctcgaaagccagcagtgaagaaagaagtgaaggaggaggaacctggcgcccctggaaaggagggcgcagccgagggcccgctggacccttcagggtataacccggcaaaaaataattaccacccggtcgaggacgcttgttggaaaccaggccaaaaggtaccttacctcgccgtcgctaggacctttgagaagatagaggaagttagtgctaggttgagaatggtcgaaacccttagtaaccttctcaggtccgtagtcgcccttagtcccccagacctgcttccggtgctgtacctgtccctgaaccatctcggtcccccccaacagggactggagttgggcgtcggtgacggcgttctcctgaaagcggttgcacaagctacaggaaggcaactggaatctgtccgggctgaggctgcagagaaaggtgacgtggggcttgtggcagagaatagtcggtcaacacagcggctgatgctgccaccgcccccgcttacggctagtggggtattctccaaatttagagatatagcacggctgacgggatcagcttccactgcgaagaagatcgatatcattaagggtttgttcgtggcttgcaggcattccgaagcacgcttcattgcacgctccctttcagggagactcagacttgggctggccgagcaatctgtactggcggccctgtctcaggcggtgagccttacgccgcccgggcaagagttccctcctgcgatggtcgatgctgggaagggaaaaaccgccgaagctcgaaaaacatggctggaggagcaaggaatgattttgaagcagacgttctgtgaagtaccggacttggatcgcatcatacctgtgcttctcgaacatggtttggagcggctccccgagcattgcaaactctctccgggcatccccctcaagccaatgctcgcccaccccacgcgcggaatcagtgaggtactgaaacgctttgaagaggcagcgtttacttgtgaatacaagtacgatggccaaagggcacaaattcatgcacttgaaggcggggaagttaagatattcagcaggaatcaggaggacaacacgggaaaatatcctgacataatatctcgaatccctaaaattaagttgcctagcgtaaccagcttcatcctggataccgaagccgtggcgtgggatagggagaaaaagcaaatacagccatttcaggtgcttacaactagaaaacgaaaagaggtggacgctagtgaaatccaagtccaggtatgtctttatgccttcgatttgatataccttaatggtgagtcccttgtacgggaaccgcttagtaggaggcggcagttgctgagggaaaattttgtcgaaactgagggagagtttgtatttgcaacgtcattggatacaaaggacatagaacaaatagcagaatttctggagcagtcagtaaaagactcctgcgagggcctgatggtgaaaactcttgatgtggacgccacttatgaaatcgcaaaaaggtcacacaattggctgaaacttaaaaaggattacttggacggggtcggggataccctcgatctcgtcgtaatcggagcttatctcggtagggggaagcgagccgggcgatacggaggctttctcttggctagttatgacgaagattccgaagagctgcaggccatatgcaagcttggaacgggtttcagcgatgaggaattggaggagcatcatcagagcttgaaggcactggtgctcccctctcctaggccgtacgttagaatagacggagcagtgatacccgatcattggctcgatccgtcagctgtttgggaggtgaagtgtgcagacctgtccctctctcctatttaccctgcagcacgcggtctggttgactctgacaaagggattagcttgaggttccctagatttattcgggtgcgcgaagataaacagcctgaacaggcgacaacgtccgcgcaggtcgcatgcctttatcgaaaacagagtcagatccagaatcaacaaggagaagattcagggagtgacccggaggacacttatagtggcggctcaaaacgaaccgccgatagtcagcattcaacacctccaaaaactaaaaggaaagtcgagtttgagccaaagaagaagcgcaaagtctaa

SEQ ID NO: 126 - protein, 2253 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSPKRRTARKQLPKRTIQEVLEEQSEDEDREAKRKKEEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTYSGGSKRTADSQHSTPPKTKRKVEFEPKKKRKV

SEQ ID NO: 127 - DNA, 6420 bp, synthetic construct
  source 1..6420: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcacacccaggaaaccagccgtgaaaaaagaggttaaagaagaggaacctggggctccgggaaaggagggagcagcggaaggtccgctcgacccttcaggatacaacccagccaaaaacaactaccaccccgtagaggatgcttgctggaagccaggccaaaaggtgccctatttggccgttgctaggactttcgaaaaaattgaggaggtgagcgcgcgactcagaatggtagagactctgtctaacctccttcgctccgtagtggctctttcacctccagatcttcttccagtgctgtacctgagcctgaaccacttgggccctccccagcagggactggaactgggcgtaggggacggagtattgctgaaggctgttgctcaggcaaccggacgacagctcgagtctgtgcgagcagaagctgcagaaaagggggacgtcgggttggttgccgaaaattcaagatctacccaacgattgatgttgccaccgccgcctctgactgcgtcaggtgtattctccaagttccgggatattgccaggcttacgggtagcgcttccactgctaaaaagatcgacataataaaaggtctgttcgtcgcttgtcgccattcagaggcgaggtttatagccagatccctttccggacgacttcgactcggcttggctgagcagtcagtactggcagctttgtctcaagctgtatcactcacgccccccggacaagaatttccacccgccatggttgacgcaggcaagggtaagactgctgaggcaagaaagacgtggctggaggaacaaggtatgatacttaaacaaacgttttgcgaagttccggacttggaccggatcatacctgtgttgctggagcacggcctcgagcgcttgcccgaacactgtaaactgtctccaggaatacctctcaaacccatgttggctcatcctacgaggggaatctcagaggtacttaaacggtttgaagaagccgctttcacgtgcgaatacaagtatgatggtcagagagcgcaaatccacgcattggaagggggtgaggtaaagattttttcaaggaatcaggaggacaatacagggaagtaccccgatatcatcagtcggattcctaaaattaagcttccatcagtcacgtccttcatactggacactgaggcagtggcttgggaccgagagaagaagcagatacaaccctttcaggtacttacaaccagaaagcgcaaggaagtcgacgcttctgagattcaagtacaagtctgcctttatgcgtttgacctgatctatcttaatggagagagtttggtgagagaacccttgagcagacgacggcagctcttgagagaaaatttcgtagaaactgagggggagttcgtctttgcgactagtctcgacaccaaagacattgagcaaatcgcggaattcctcgaacagtcagttaaagactcctgcgaaggtctgatggttaagactcttgacgtggatgctacctacgagatagctaagcggtcacacaattggctgaaactgaaaaaggactatctggatggagttggggacacgctggatttggtcgttatcggggcctatctgggacgcggtaagcgggcagggagatatggtggattcctcctcgcttcatacgatgaggactctgaagagctgcaggctatatgcaaacttgggacgggtttttccgatgaagaattggaggaacatcatcagtcactgaaggcccttgtattgccaagtccacgcccatacgtacgaatcgatggagcagtaatccctgaccactggcttgacccgtccgccgtctgggaagtaaagtgcgcggatctctctctcagtccgatctacccagccgcacgggggctggttgacagtgacaagggtatcagcctgcgatttcctcgattcatacgcgtccgggaagacaagcaaccggaacaggctacgacctctgcacaggtcgcatgtttgtatagaaaacagagccaaattcagaatcaacaaggcgaagacagtgggtccgatcctgaagatacctactcaggcggcagtaaacggacagctgatagccaacactcaactcctccgaagactaaaaggaaggtagagttcgaaccaaaaaagaaaaggaaagtgtaa

SEQ ID NO: 128 - protein, 2139 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTYSGGSKRTADSQHSTPPKTKRKVEFEPKKKRKV

SEQ ID NO: 129 - DNA, 5253 bp, synthetic construct
  source 1..5253: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcatggcaatcactaagcccctcttggcggcgactttggaaaacatcgaggatgtgcaattcccgtgccttgccacaccaaagatagacgggatccgatcagtgaagcaaacgcagatgctctctagaacgttcaagcctattagaaactcagtgatgaatcggctcttgactgagctgttgccggaaggcagcgatggggaaatatctatcgagggagccacatttcaagacactacgagcgccgtaatgactggacataagatgtataatgctaaattctcctactattggtttgactatgtcactgatgaccctcttaagaaatatatagaccgagtggaggatatgaaaaattatattactgtacacccgcatattctggaacatgcccaagttaagattattcctctcattcccgtcgagattaataatatcacagaactgcttcagtatgagcgcgacgtattgtccaaaggctttgaaggggttatgatacgcaaaccggacggcaagtacaagttcggaagaagcacattgaaagagggtatattgctgaagatgaagcagtttaaggatgctgaggcaacaataatcagcatgacagcactttttaaaaataccaacacgaaaactaaggacaattttggttatagtaagcggtcaacgcacaaaagtgggaaggtagaagaagacgtaatgggtagcattgaggtggattatgacggggtggttttcagcatagggactgggtttgatgcagatcaacggagggacttttggcagaacaaagaatcatatataggcaaaatggtaaagttcaaatacttcgaaatgggaagtaaagactgccccagattccctgtattcattggcatcaggcacgaggaggacaggagtgggggatcaaagcggactgctgatagtcagcatagtactccacccaagaccaagcggaaagttgagtttgagccgaagaaaaagcgaaaagtgtaa

SEQ ID NO: 130 - protein, 1750 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSMAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDRSGGSKRTADSQHSTPPKTKRKVEFEPKKKRKV

SEQ ID NO: 131 - DNA, 5820 bp, synthetic construct
  source 1..5820: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcatgattcttaaaattcttaacgagattgcgagtattggcagcacgaaacaaaagcaggccatattggaaaagaataaggacaatgagttgcttaaacgcgtgtataggctcacttactctcgcggactgcaatactatattaaaaaatggcctaagcccggcatcgctactcaaagcttcggaatgcttacgctgacagatatgctcgacttcatcgagtttactctcgcaacaaggaagttgactggcaacgccgcgattgaagaattgacgggttatatcacggacgggaagaaggatgatgttgaggtgctgaggcgcgttatgatgcgcgacctcgaatgtggtgcctcagtttccatagccaataaagtttggccaggcttgatcccggagcagccacagatgctggccagtagctacgacgagaagggtattaacaaaaatatcaagtttccagcgtttgcacaacttaaagcggatggggcgcggtgtttcgccgaagtccggggtgacgaattggacgatgtgcgccttctgagtcgcgcaggaaatgaatatctggggcttgacctcttgaaggaggagctgattaagatgacagcagaagccaggcagatccatccagagggggtacttattgatggtgaactcgtataccatgagcaggttaagaaggagccagagggtttggatttcctctttgacgcctatcccgagaattcaaaggcaaaggagttcgccgaggttgcagaatcaagaacggcttccaacggcatagcgaataaatcactcaaaggaactatatctgaaaaggaggcacagtgtatgaaattccaagtgtgggactatgtgccgcttgtcgagatttacagcttgcctgctttccgattgaagtacgatgtacggtttagtaagctcgagcaaatgacttcaggttacgataaagtcatcttgattgagaaccaggtcgttaataatcttgacgaggcgaaggtcatatataagaaatatatagatcaagggctcgagggtatcattctgaagaatatagatggcttgtgggaaaacgccaggtccaaaaacctgtataagtttaaggaagtaatagatgtagatttgaaaatagttggaatttacccccatcggaaggaccccacgaaagcgggtgggtttatcctcgagagcgagtgtgggaagataaaagtgaatgccggctccggattgaaggacaaggcaggtgtgaaaagtcatgagctcgatcggacgagaataatggaaaaccagaattactacattggaaagattttggaatgcgagtgtaacggctggttgaagagcgacggacgcaccgattacgtgaaactctttctgccaattgcaatcaggttgagagaggataagactaaggccaatactttcgaggacgtcttcggagactttcacgaagtcactgggctttctgggggtagtaagagaactgcagatagccagcattcaacgccgccaaaaacaaagcgaaaggtagaattcgaaccaaagaaaaagcggaaagtataa

SEQ ID NO: 132 - protein, 1939 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSMILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADSQHSTPPKTKRKVEFEPKKKRKV

SEQ ID NO: 133 - DNA, 4383 bp, synthetic construct
  source 1..4383: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacggctcaaaaagaaccgccgacggcagcgaattcgagcccaagaagaagaggaaagtcggaggaggaggcagtggtgggcgacttgaaattagagccgcgttcctgcgccagaggaatacggctctccgcacggaggtagccgaacttgagcaagaagtacagagattggagaacgaggtttcacagtatgagacacgatatggcccccttggcggcggaaagtaa

SEQ ID NO: 134 - protein, 2352 aa, synthetic construct
KRTADGSEFESPKKKRKVMGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGKSGGSSGGSSGSETPGTSESATPESSGGSSGGSSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSMILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADSQHSTPPKTKRKVEFEPKKKRKV

SEQ ID NO: 135 - protein, 2352 aa, synthetic construct
KRTADGSEFESPKKKRKVMILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSSGGSSGSETPGTSESATPESSGGSSGGSSMGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGKSGGSSGGSKRTADGSEFESPKKKRKVSGGSSGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADSQHSTPPKTKRKVEFEPKKKRKV

SEQ ID NO: 136 - protein, 2352 aa, synthetic construct
KRTADGSEFESPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSMGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGKSGGSSGGSKRTADGSEFESPKKKRKVSGGSSGGSMILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADSQHSTPPKTKRKVEFEPKKKRKV

SEQ ID NO: 137 - protein, 2352 aa, synthetic construct
KRTADGSEFESPKKKRKVMILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSSGGSSGSETPGTSESATPESSGGSSGGSSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSMGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGKSGGSKRTADSQHSTPPKTKRKVEFEPKKKRKV

SEQ ID NO: 138 - protein, 2352 aa, synthetic construct
KRTADGSEFESPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSMILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSSGGSKRTADGSEFESPKKKRKVSGGSSGGSMGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGKSGGSKRTADSQHSTPPKTKRKVEFEPKKKRKV

SEQ ID NO: 139 - protein, 2352 aa, synthetic construct; identical to SEQ ID NO: 135

SEQ ID NO: 140 - protein, 2263 aa, synthetic construct
KRTADGSEFESPKKKRKVMSKSWGKFIEEEEAEMASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQSGGSSGGSSGSETPGTSESATPESSGGSSGGSSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSMILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLSGGSKRTADSQHSTPPKTKRKVEFEPKKKRKV

SEQ ID NO: 141 - DNA, 1236 bp, synthetic construct
  source 1..1236: mol_type = other DNA
atgttggagatagaggctgctttccttgaacgggagaatacggccctcgagactagggttgctgagcttagacagcgagtccaaagactgcgaaaccgggtgtcccaatataggaccagatacggacctctgggtggagggaaatccggtgggagtagcggcgggtctagtggctcagagacacctggcacgagcgagagtgcgactcctgaaagctccggcggcagcagtgggggaagttccatggcaatcactaagcccctcttggcggcgactttggaaaacatcgaggatgtgcaattcccgtgccttgccacaccaaagatagacgggatccgatcagtgaagcaaacgcagatgctctctagaacgttcaagcctattagaaactcagtgatgaatcggctcttgactgagctgttgccggaaggcagcgatggggaaatatctatcgagggagccacatttcaagacactacgagcgccgtaatgactggacataagatgtataatgctaaattctcctactattggtttgactatgtcactgatgaccctcttaagaaatatatagaccgagtggaggatatgaaaaattatattactgtacacccgcatattctggaacatgcccaagttaagattattcctctcattcccgtcgagattaataatatcacagaactgcttcagtatgagcgcgacgtattgtccaaaggctttgaaggggttatgatacgcaaaccggacggcaagtacaagttcggaagaagcacattgaaagagggtatattgctgaagatgaagcagtttaaggatgctgaggcaacaataatcagcatgacagcactttttaaaaataccaacacgaaaactaaggacaattttggttatagtaagcggtcaacgcacaaaagtgggaaggtagaagaagacgtaatgggtagcattgaggtggattatgacggggtggttttcagcatagggactgggtttgatgcagatcaacggagggacttttggcagaacaaagaatcatatataggcaaaatggtaaagttcaaatacttcgaaatgggaagtaaagactgccccagattccctgtattcattggcatcaggcacgaggaggacaggagtgggggatcaaagcggactgctgatagtcagcatagtactccacccaagaccaagcggaaagttgagtttgagccgaagaaaaagcgaaaagtgtaa

SEQ ID NO: 142 - DNA, 1803 bp, synthetic construct
  source 1..1803: mol_type = other DNA
atgcttgagatcgaggcggcgttcctcgaaagagagaacaccgcacttgaaactcgcgtggcagaattgaggcagcgggtgcaaagacttagaaatagagtctctcagtatcggacccgatatggtcctctgggaggcgggaagtctggaggttcaagcggaggcagttccgggagtgagacaccgggaacttctgagagtgcaactcctgagagctctggtggatcatccggaggctccagtatgattcttaaaattcttaacgagattgcgagtattggcagcacgaaacaaaagcaggccatattggaaaagaataaggacaatgagttgcttaaacgcgtgtataggctcacttactctcgcggactgcaatactatattaaaaaatggcctaagcccggcatcgctactcaaagcttcggaatgcttacgctgacagatatgctcgacttcatcgagtttactctcgcaacaaggaagttgactggcaacgccgcgattgaagaattgacgggttatatcacggacgggaagaaggatgatgttgaggtgctgaggcgcgttatgatgcgcgacctcgaatgtggtgcctcagtttccatagccaataaagtttggccaggcttgatcccggagcagccacagatgctggccagtagctacgacgagaagggtattaacaaaaatatcaagtttccagcgtttgcacaacttaaagcggatggggcgcggtgtttcgccgaagtccggggtgacgaattggacgatgtgcgccttctgagtcgcgcaggaaatgaatatctggggcttgacctcttgaaggaggagctgattaagatgacagcagaagccaggcagatccatccagagggggtacttattgatggtgaactcgtataccatgagcaggttaagaaggagccagagggtttggatttcctctttgacgcctatcccgagaattcaaaggcaaaggagttcgccgaggttgcagaatcaagaacggcttccaacggcatagcgaataaatcactcaaaggaactatatctgaaaaggaggcacagtgtatgaaattccaagtgtgggactatgtgccgcttgtcgagatttacagcttgcctgctttccgattgaagtacgatgtacggtttagtaagctcgagcaaatgacttcaggttacgataaagtcatcttgattgagaaccaggtcgttaataatcttgacgaggcgaaggtcatatataagaaatatatagatcaagggctcgagggtatcattctgaagaatatagatggcttgtgggaaaacgccaggtccaaaaacctgtataagtttaaggaagtaatagatgtagatttgaaaatagttggaatttacccccatcggaaggaccccacgaaagcgggtgggtttatcctcgagagcgagtgtgggaagataaaagtgaatgccggctccggattgaaggacaaggcaggtgtgaaaagtcatgagctcgatcggacgagaataatggaaaaccagaattactacattggaaagattttggaatgcgagtgtaacggctggttgaagagcgacggacgcaccgattacgtgaaactctttctgccaattgcaatcaggttgagagaggataagactaaggccaatactttcgaggacgtcttcggagactttcacgaagtcactgggctttctgggggtagtaagagaactgcagatagccagcattcaacgccgccaaaaacaaagcgaaaggtagaattcgaaccaaagaaaaagcggaaagtataa

SEQ ID NO: 143 - DNA, 2403 bp, synthetic construct
  source 1..2403: mol_type = other DNA
atgctcgagatcgaagctgcatttctggagagggagaataccgccctcgaaacccgggtggctgaattgcgacagagagtgcaacggctccggaatagagtatctcaatatcgaacccgctatgggcctctcggagggggtaaatctggcggaagttctggcggtagttcaggaagtgagacaccgggaactagtgaatccgcgactcccgaatcaagtgggggatcatctggagggtcaagcacacccaggaaaccagccgtgaaaaaagaggttaaagaagaggaacctggggctccgggaaaggagggagcagcggaaggtccgctcgacccttcaggatacaacccagccaaaaacaactaccaccccgtagaggatgcttgctggaagccaggccaaaaggtgccctatttggccgttgctaggactttcgaaaaaattgaggaggtgagcgcgcgactcagaatggtagagactctgtctaacctccttcgctccgtagtggctctttcacctccagatcttcttccagtgctgtacctgagcctgaaccacttgggccctccccagcagggactggaactgggcgtaggggacggagtattgctgaaggctgttgctcaggcaaccggacgacagctcgagtctgtgcgagcagaagctgcagaaaagggggacgtcgggttggttgccgaaaattcaagatctacccaacgattgatgttgccaccgccgcctctgactgcgtcaggtgtattctccaagttccgggatattgccaggcttacgggtagcgcttccactgctaaaaagatcgacataataaaaggtctgttcgtcgcttgtcgccattcagaggcgaggtttatagccagatccctttccggacgacttcgactcggcttggctgagcagtcagtactggcagctttgtctcaagctgtatcactcacgccccccggacaagaatttccacccgccatggttgacgcaggcaagggtaagactgctgaggcaagaaagacgtggctggaggaacaaggtatgatacttaaacaaacgttttgcgaagttccggacttggaccggatcatacctgtgttgctggagcacggcctcgagcgcttgcccgaacactgtaaactgtctccaggaatacctctcaaacccatgttggctcatcctacgaggggaatctcagaggtacttaaacggtttgaagaagccgctttcacgtgcgaatacaagtatgatggtcagagagcgcaaatccacgcattggaagggggtgaggtaaagattttttcaaggaatcaggaggacaatacagggaagtaccccgatatcatcagtcggattcctaaaattaagcttccatcagtcacgtccttcatactggacactgaggcagtggcttgggaccgagagaagaagcagatacaaccctttcaggtacttacaaccagaaagcgcaaggaagtcgacgcttctgagattcaagtacaagtctgcctttatgcgtttgacctgatctatcttaatggagagagtttggtgagagaacccttgagcagacgacggcagctcttgagagaaaatttcgtagaaactgagggggagttcgtctttgcgactagtctcgacaccaaagacattgagcaaatcgcggaattcctcgaacagtcagttaaagactcctgcgaaggtctgatggttaagactcttgacgtggatgctacctacgagatagctaagcggtcacacaattggctgaaactgaaaaaggactatctggatggagttggggacacgctggatttggtcgttatcggggcctatctgggacgcggtaagcgggcagggagatatggtggattcctcctcgcttcatacgatgaggactctgaagagctgcaggctatatgcaaacttgggacgggtttttccgatgaagaattggaggaacatcatcagtcactgaaggcccttgtattgccaagtccacgcccatacgtacgaatcgatggagcagtaatccctgaccactggcttgacccgtccgccgtctgggaagtaaagtgcgcggatctctctctcagtccgatctacccagccgcacgggggctggttgacagtgacaagggtatcagcctgcgatttcctcgattcatacgcgtccgggaagacaagcaaccggaacaggctacgacctctgcacaggtcgcatgtttgtatagaaaacagagccaaattcagaatcaacaaggcgaagacagtgggtccgatcctgaagatacctactcaggcggcagtaaacggacagctgatagccaacactcaactcctccgaagactaaaaggaaggtagagttcgaaccaaaaaagaaaaggaaagtgtaa

SEQ ID NO: 144 - DNA, 2745 bp, synthetic construct
  source 1..2745: mol_type = other DNA
atgctcgagatcgaggcggcgttccttgaacgcgagaacactgcgctggaaacgagggtcgcggaactccgccagagggttcaacggttgaggaatcgagtgagtcagtaccgaacccgatatggaccactgggtggcgggaaatcagggggctcatccggcggctccagcgggagcgaaaccccgggtacctcagaatctgcgacgccagaaagctcaggcggatctagcggcggtagttcaccgaagcgccggactgcacgaaagcaactgccaaaacggactatacaagaagtcctggaagaacaaagcgaagatgaggatcgcgaagccaagcgcaagaaagaggaagaggaagaagagactccaaaggagtccttgaccgaagcagaagtcgcaacggagaaggaaggtgaggatggggatcagccaacaaccccgcctaaacctctgaaaacctctaaggcggagacaccaactgagagtgtcagcgaaccggaggtagccacgaaacaagagcttcaggaggaagaagaacagacaaagccacctcggcgggctcccaaaacccttagctccttcttcacgcctcgaaagccagcagtgaagaaagaagtgaaggaggaggaacctggcgcccctggaaaggagggcgcagccgagggcccgctggacccttcagggtataacccggcaaaaaataattaccacccggtcgaggacgcttgttggaaaccaggccaaaaggtaccttacctcgccgtcgctaggacctttgagaagatagaggaagttagtgctaggttgagaatggtcgaaacccttagtaaccttctcaggtccgtagtcgcccttagtcccccagacctgcttccggtgctgtacctgtccctgaaccatctcggtcccccccaacagggactggagttgggcgtcggtgacggcgttctcctgaaagcggttgcacaagctacaggaaggcaactggaatctgtccgggctgaggctgcagagaaaggtgacgtggggcttgtggcagagaatagtcggtcaacacagcggctgatgctgccaccgcccccgcttacggctagtggggtattctccaaatttagagatatagcacggctgacgggatcagcttccactgcgaagaagatcgatatcattaagggtttgttcgtggcttgcaggcattccgaagcacgcttcattgcacgctccctttcagggagactcagacttgggctggccgagcaatctgtactggcggccctgtctcaggcggtgagccttacgccgcccgggcaagagttccctcctgcgatggtcgatgctgggaagggaaaaaccgccgaagctcgaaaaacatggctggaggagcaaggaatgattttgaagcagacgttctgtgaagtaccggacttggatcgcatcatacctgtgcttctcgaacatggtttggagcggctccccgagcattgcaaactctctccgggcatccccctcaagccaatgctcgcccaccccacgcgcggaatcagtgaggtactgaaacgctttgaagaggcagcgtttacttgtgaatacaagtacgatggccaaagggcacaaattcatgcacttgaaggcggggaagttaagatattcagcaggaatcaggaggacaacacgggaaaatatcctgacataatatctcgaatccctaaaattaagttgcctagcgtaaccagcttcatcctggataccgaagccgtggcgtgggatagggagaaaaagcaaatacagccatttcaggtgcttacaactagaaaacgaaaagaggtggacgctagtgaaatccaagtccaggtatgtctttatgccttcgatttgatataccttaatggtgagtcccttgtacgggaaccgcttagtaggaggcggcagttgctgagggaaaattttgtcgaaactgagggagagtttgtatttgcaacgtcattggatacaaaggacatagaacaaatagcagaatttctggagcagtcagtaaaagactcctgcgagggcctgatggtgaaaactcttgatgtggacgccacttatgaaatcgcaaaaaggtcacacaattggctgaaacttaaaaaggattacttggacggggtcggggataccctcgatctcgtcgtaatcggagcttatctcggtagggggaagcgagccgggcgatacggaggctttctcttggctagttatgacgaagattccgaagagctgcaggccatatgcaagcttggaacgggtttcagcgatgaggaattggaggagcatcatcagagcttgaaggcactggtgctcccctctcctaggccgtacgttagaatagacggagcagtgatacccgatcattggctcgatccgtcagctgtttgggaggtgaagtgtgcagacctgtccctctctcctatttaccctgcagcacgcggtctggttgactctgacaaagggattagcttgaggttccctagatttattcgggtgcgcgaagacaaacagcctgaacaggcgacaacgtccgcgcaggtcgcatgcctttatcgaaaacagagtcagatccagaatcaacaaggagaagattcagggagtgacccggaggacacttatagtggcggctcaaaacgaaccgccgatagtcagcattcaacacctccaaaaactaaaaggaaagtcgagtttgagccaaagaagaagcgcaaagtctaa

SEQ ID NO: 145 - DNA, 1725 bp, synthetic construct
  source 1..1725: mol_type = other DNA
atgatccttaagattctcaacgaaatcgctagtatagggtccactaagcagaagcaggccatattggaaaaaaataaggacaatgaacttttgaagagagtctatagactgacgtactctagggggctccagtactacatcaagaaatggcctaaacctggcattgcgacgcagtcattcggtatgctgacattgacggatatgttggatttcattgagtttacgctggccaccagaaaacttacgggtaatgctgcgatagaagaacttacagggtacataacagacgggaagaaagatgacgtggaagtgctcagacgagttatgatgcgcgatctcgagtgcggcgctagcgtgtcaatcgcgaacaaagtctggcccggcctcataccagagcagccacagatgctggcatcttcctatgacgaaaaaggcataaacaagaatattaaattcccggccttcgctcaactcaaagcagatggtgccaggtgttttgccgaagttcggggtgatgaacttgatgacgtgcggctcttgtctagggcaggtaacgagtacctcggcctggacttgcttaaagaggaactgattaaaatgacagctgaggcgcggcagatacaccccgagggcgtccttatcgacggggagctggtgtatcacgaacaagttaaaaaggaaccggagggtcttgattttcttttcgacgcgtatcctgagaacagcaaggcgaaagaatttgcagaagttgcagaaagcaggaccgcaagtaatggaatcgctaataaaagcctcaagggtaccatcagcgaaaaagaagcccagtgcatgaaatttcaagtttgggactatgtccccttggtcgaaatttactccctgcccgcattccggctgaagtatgatgttcgcttcagtaaactggagcaaatgacgagcggttatgataaggttatacttattgagaatcaggtcgtaaataatttggacgaggcgaaagttatatacaaaaagtatatagaccaagggttggaggggatcattttgaagaacatagacggactttgggagaacgcccggtccaagaatttgtataaattcaaagaagtcatagatgttgacctcaagatagtaggtatatatccccacagaaaggacccaaccaaagcaggcggattcattttggagtccgagtgtgggaagataaaggtcaatgctggatctggactcaaggacaaagctggtgtgaagtcacatgaactggaccgaaccaggattatggagaatcagaactattacatcgggaagatattggagtgtgaatgcaacggctggcttaaatcagatggaagaactgattacgttaaattgttcctgcccatagccatacgactccgcgaggacaaaacgaaggctaacacgtttgaagacgtattcggagatttccatgaggtgactggccttagtggaggctccaaacggacagcagactcccaacattcaacacccccaaaaacaaagcggaaggtagagtttgagccaaaaaagaaaagaaaggtcggaggaggaggcagtggtgggcgacttgaaattagagccgcgttcctgcgccagaggaatacggctctccgcacggaggtagccgaacttgagcaagaagtacagagattggagaacgaggtttcacagtatgagacacgatatggcccccttggcggcggaaagtaa

SEQ ID NO: 146 - DNA, 2199 bp, synthetic construct
  source 1..2199: mol_type = other DNA
atgcttgagatcgaggcggcgttcctcgaaagagagaacaccgcacttgaaactcgcgtggcagaattgaggcagcgggtgcaaagacttagaaatagagtctctcagtatcggacccgatatggtcctctgggaggcgggaagtctggaggttcaagcggaggcagttccgggagtgagacaccgggaacttctgagagtgcaactcctgagagctctggtggatcatccggaggctccagtgcagcttctcagacctctcaaacagtagcctctcatgtaccgttcgctgacttgtgttctacgctcgaacgcatccagaaatcaaaggggcgcgccgagaaaatccggcacttcagagaattcttggattcctggaggaagtttcatgatgctctccacaaaaatcacaaagatgtaacggatagtttctaccctgctatgagacttatactgccgcagcttgagagggaacgcatggcgtatggtataaaggagacaatgttggcgaaattgtatattgagctgctgaacttgccaagagatggaaaggacgcgctcaaactgctgaactatagaacacccacgggtacccatggtgacgccggtgactttgccatgatcgcctatttcgtactgaaacctcgatgtcttcaaaaaggttctcttacaattcagcaagtcaacgacctgctggattcaattgcgagtaacaacagcgctaagcgaaaggatctcattaagaaaagcctcctgcagctgataactcagtcctctgcactcgaacaaaaatggctgattcggatgattatcaaggatttgaagttgggggtatctcagcaaactattttcagcgtgtttcacaatgatgcagcagaattgcataatgtcacaacagatcttgagaaagtctgccgacagttgcacgacccctctgtaggcttgagtgacatatctataacacttttttctgcgttcaaacccatgttggctgctattgcggacatagaacacatcgagaaagacatgaaacatcagtcattctatatagagactaaattggacggcgagaggatgcaaatgcacaaagatggtgatgtgtataaatatttttcccgcaacggctacaactacactgatcaattcggagcgtccccaactgaagggtccctcactcctttcatacacaatgcgtttaaggccgatattcagatatgtatcctcgacggcgaaatgatggcgtacaatcccaatacccagaccttcatgcaaaagggaacgaagttcgatattaaacggatggttgaagattccgacctccaaacatgttactgtgtgtttgatgtcctgatggtgaataacaaaaaactcggccatgaaacccttcgaaagcgatacgaaatactcagcagtatatttactccaataccaggccgaatcgagatcgtacagaaaacacaagcccatactaagaatgaagttattgatgcactgaacgaagccatagacaagagggaagaaggcataatggtcaagcagcctctgagtatatataaacctgacaaaaggggggaaggatggctgaagataaagccagaatacgtgtctggtcttatggacgaattggacattctcatcgtcggaggatattggggtaagggttccaggggggggatgatgtcccactttctgtgtgcggttgccgagaaaccgcccccaggggaaaaaccatcagtgttccatacgttgtcacgcgtcggctcaggttgtacgatgaaggaactttacgatctggggttgaaactcgccaaatattggaagccattccatcggaaagcaccgccctctagtatcttgtgtgggacggagaagccagaagtttatatagagccatgtaactcagtaattgttcaaatcaaagccgcagagatcgtcccgtcagacatgtacaagactggatgcacccttagatttcctcgcatcgaaaaaataagagatgataaagagtggcatgagtgcatgactcttgacgaccttgaacagctccgcgggaaggccagcggtaaactggctagtaagcacctctacatcgggggtgacagtggaggctccaaacggacagcagactcccaacattcaacacccccaaaaacaaagcggaaggtagagtttgagccaaaaaagaaaagaaaggtctaa

SEQ ID NO: 147 - DNA, 4461 bp, synthetic construct
  source 1..4461: mol_type = other DNA
atgcttgagatcgaggcggcgttcctcgaaagagagaacaccgcacttgaaactcgcgtggcagaattgaggcagcgggtgcaaagacttagaaatagagtctctcagtatcggacccgatatggtcctctgggaggcgggaagtctggaggttcaagcggaggcagttccgggagtgagacaccgggaacttctgagagtgcaactcctgagagctctggtggatcatccggaggctccagtaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacggctcaaaaagaaccgccgacggcagcgaattcgagtcacccaagaagaagaggaaagtctaa

SEQ ID NO: 148 - DNA, 1158 bp, synthetic construct
  source 1..1158: mol_type = other DNA
atggcgataacgaagcccttgttggcagctacgttggaaaatattgaggacgtacagttcccatgccttgccactccgaagatcgatggaatccgatccgtgaaacagacacaaatgcttagcagaacattcaaacccatcaggaacagcgtaatgaatagattgcttacggaactcttgcccgaagggtctgacggcgagattagcatcgaaggagcgactttccaagatactacctcagcagttatgacgggacacaagatgtataatgctaaattctcatattactggtttgactatgttactgacgatcctttgaagaaatacatagacagggttgaagatatgaaaaattacataactgtccaccctcatatcctggagcatgcacaggtaaagattatcccgctcataccagtagaaattaacaatataaccgaattgttgcagtatgaacgcgatgtgctctctaaaggcttcgagggcgtgatgataaggaagcctgatggcaaatataagttcggtaggtccacattgaaagagggaattctcttgaagatgaaacagtttaaggatgcggaagctactatcattagtatgacggcactgtttaaaaacactaacactaaaaccaaggacaactttggctatagtaaaaggagcacacacaaatcaggaaaagttgaggaggacgtaatgggcagtatagaggtagattacgatggtgtggtgtttagcattggaacgggcttcgacgctgaccagcggagggacttttggcagaataaggaaagttacattggcaagatggttaaattcaaatacttcgagatgggctcaaaagactgtccgagatttcctgtgtttattggaatcagacacgaagaggataggagtggaggctccaaacggacagcagactcccaacattcaacacccccaaaaacaaagcggaaggtagagtttgagccaaaaaagaaaagaaaggtcggaggaggaggcagtggtgggcgacttgaaattagagccgcgttcctgcgccagaggaatacggctctccgcacggaggtagccgaacttgagcaagaagtacagagattggagaacgaggtttcacagtatgagacacgatatggcccccttggcggcggaaagtaa

SEQ ID NO: 149 - DNA, 2124 bp, synthetic construct
  source 1..2124: mol_type = other DNA
atggcagcttctcagacctctcaaacagtagcctctcatgtaccgttcgctgacttgtgttctacgctcgaacgcatccagaaatcaaaggggcgcgccgagaaaatccggcacttcagagaattcttggattcctggaggaagtttcatgatgctctccacaaaaatcacaaagatgtaacggatagtttctaccctgctatgagacttatactgccgcagcttgagagggaacgcatggcgtatggtataaaggagacaatgttggcgaaattgtatattgagctgctgaacttgccaagagatggaaaggacgcgctcaaactgctgaactatagaacacccacgggtacccatggtgacgccggtgactttgccatgatcgcctatttcgtactgaaacctcgatgtcttcaaaaaggttctcttacaattcagcaagtcaacgacctgctggattcaattgcgagtaacaacagcgctaagcgaaaggatctcattaagaaaagcctcctgcagctgataactcagtcctctgcactcgaacaaaaatggctgattcggatgattatcaaggatttgaagttgggggtatctcagcaaactattttcagcgtgtttcacaatgatgcagcagaattgcataatgtcacaacagatcttgagaaagtctgccgacagttgcacgacccctctgtaggcttgagtgacatatctataacacttttttctgcgttcaaacccatgttggctgctattgcggacatagaacacatcgagaaagacatgaaacatcagtcattctatatagagactaaattggacggcgagaggatgcaaatgcacaaagatggtgatgtgtataaatatttttcccgcaacggctacaactacactgatcaattcggagcgtccccaactgaagggtccctcactcctttcatacacaatgcgtttaaggccgatattcagatatgtatcctcgacggcgaaatgatggcgtacaatcccaatacccagaccttcatgcaaaagggaacgaagttcgatattaaacggatggttgaagattccgacctccaaacatgttactgtgtgtttgatgtcctgatggtgaataacaaaaaactcggccatgaaacccttcgaaagcgatacgaaatactcagcagtatatttactccaataccaggccgaatcgagatcgtacagaaaacacaagcccatactaagaatgaagttattgatgcactgaacgaagccatagacaagagggaagaaggcataatggtcaagcagcctctgagtatatataaacctgacaaaaggggggaaggatggctgaagataaagccagaatacgtgtctggtcttatggacgaattggacattctcatcgtcggaggatattggggtaagggttccaggggggggatgatgtcccactttctgtgtgcggttgccgagaaaccgcccccaggggaaaaaccatcagtgttccatacgttgtcacgcgtcggctcaggttgtacgatgaaggaactttacgatctggggttgaaactcgccaaatattggaagccattccatcggaaagcaccgccctctagtatcttgtgtgggacggagaagccagaagtttatatagagccatgtaactcagtaattgttcaaatcaaagccgcagagatcgtcccgtcagacatgtacaagactggatgcacccttagatttcctcgcatcgaaaaaataagagatgataaagagtggcatgagtgcatgactcttgacgaccttgaacagctccgcgggaaggccagcggtaaactggctagtaagcacctctacatcgggggtgacagtggaggctccaaacggacagcagactcccaacattcaacacccccaaaaacaaagcggaaggtagagtttgagccaaaaaagaaaagaaaggtcggaggaggaggcagtggtgggcgacttgaaattagagccgcgttcctgcgccagaggaatacggctctccgcacggaggtagccgaacttgagcaagaagtacagagattggagaacgaggtttcacagtatgagacacgatatggcccccttggcggcggaaagtaa

SEQ ID NO: 150 - RNA, 6120 nt, synthetic construct
  source 1..6120: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggtggctcctcagggggatctaaacgcacggccgatgggtccgagtttgagtctcccaagaagaaaaggaaagtgagtggtggaagtagcggcggtagcgcagcttctcagacctctcaaacagtagcctctcatgtaccgttcgctgacttgtgttctacgctcgaacgcatccagaaatcaaaggggcgcgccgagaaaatccggcacttcagagaattcttggattcctggaggaagtttcatgatgctctccacaaaaatcacaaagatgtaacggatagtttctaccctgctatgagacttatactgccgcagcttgagagggaacgcatggcgtatggtataaaggagacaatgttggcgaaattgtatattgagctgctgaacttgccaagagatggaaaggacgcgctcaaactgctgaactatagaacacccacgggtacccatggtgacgccggtgactttgccatgatcgcctatttcgtactgaaacctcgatgtcttcaaaaaggttctcttacaattcagcaagtcaacgacctgctggattcaattgcgagtaacaacagcgctaagcgaaaggatctcattaagaaaagcctcctgcagctgataactcagtcctctgcactcgaacaaaaatggctgattcggatgattatcaaggatttgaagttgggggtatctcagcaaactattttcagcgtgtttcacaatgatgcagcagaattgcataatgtcacaacagatcttgagaaagtctgccgacagttgcacgacccctctgtaggcttgagtgacatatctataacacttttttctgcgttcaaacccatgttggctgctattgcggacatagaacacatcgagaaagacatgaaacatcagtcattctatatagagactaaattggacggcgagaggatgcaaatgcacaaagatggtgatgtgtataaatatttttcccgcaacggctacaactacactgatcaattcggagcgtccccaactgaagggtccctcactcctttcatacacaatgcgtttaaggccgatattcagatatgtatcctcgacggcgaaatgatggcgtacaatcccaatacccagaccttcatgcaaaagggaacgaagttcgatattaaacggatggttgaagattccgacctccaaacatgttactgtgtgtttgatgtcctgatggtgaataacaaaaaactcggccatgaaacccttcgaaagcgatacgaaatactcagcagtatatttactccaataccaggccgaatcgagatcgtacagaaaacacaagcccatactaagaatgaagttattgatgcactgaacgaagccatagacaagagggaagaaggcataatggtcaagcagcctctgagtatatataaacctgacaaaaggggggaaggatggctgaagataaagccagaatacgtgtctggtcttatggacgaattggacattctcatcgtcggaggatattggggtaagggttccaggggggggatgatgtcccactttctgtgtgcggttgccgagaaaccgcccccaggggaaaaaccatcagtgttccatacgttgtcacgcgtcggctcaggttgtacgatgaaggaactttacgatctggggttgaaactcgccaaatattggaagccattccatcggaaagcaccgccctctagtatcttgtgtgggacggagaagccagaagtttatatagagccatgtaactcagtaattgttcaaatcaaagccgcagagatcgtcccgtcagacatgtacaagactggatgcacccttagatttcctcgcatcgaaaaaataagagatgataaagagtggcatgagtgcatgactcttgacgaccttgaacagctccgcgggaaggccagcggtaaactggctagtaagcacctctacatcgggggtgactaa

SEQ ID NO: 151 - DNA, 5724 bp, synthetic construct
  source 1..5724: mol_type = other DNA
atgatgatccttaagattctcaacgaaatcgctagtatagggtccactaagcagaagcaggccatattggaaaaaaataaggacaatgaacttttgaagagagtctatagactgacgtactctagggggctccagtactacatcaagaaatggcctaaacctggcattgcgacgcagtcattcggtatgctgacattgacggatatgttggatttcattgagtttacgctggccaccagaaaacttacgggtaatgctgcgatagaagaacttacagggtacataacagacgggaagaaagatgacgtggaagtgctcagacgagttatgatgcgcgatctcgagtgcggcgctagcgtgtcaatcgcgaacaaagtctggcccggcctcataccagagcagccacagatgctggcatcttcctatgacgaaaaaggcataaacaagaatattaaattcccggccttcgctcaactcaaagcagatggtgccaggtgttttgccgaagttcggggtgatgaacttgatgacgtgcggctcttgtctagggcaggtaacgagtacctcggcctggacttgcttaaagaggaactgattaaaatgacagctgaggcgcggcagatacaccccgagggcgtccttatcgacggggagctggtgtatcacgaacaagttaaaaaggaaccggagggtcttgattttcttttcgacgcgtatcctgagaacagcaaggcgaaagaatttgcagaagttgcagaaagcaggaccgcaagtaatggaatcgctaataaaagcctcaagggtaccatcagcgaaaaagaagcccagtgcatgaaatttcaagtttgggactatgtccccttggtcgaaatttactccctgcccgcattccggctgaagtatgatgttcgcttcagtaaactggagcaaatgacgagcggttatgataaggttatacttattgagaatcaggtcgtaaataatttggacgaggcgaaagttatatacaaaaagtatatagaccaagggttggaggggatcattttgaagaacatagacggactttgggagaacgcccggtccaagaatttgtataaattcaaagaagtcatagatgttgacctcaagatagtaggtatatatccccacagaaaggacccaaccaaagcaggcggattcattttggagtccgagtgtgggaagataaaggtcaatgctggatctggactcaaggacaaagctggtgtgaagtcacatgaactggaccgaaccaggattatggagaatcagaactattacatcgggaagatattggagtgtgaatgcaacggctggcttaaatcagatggaagaactgattacgttaaattgttcctgcccatagccatacgactccgcgaggacaaaacgaaggctaacacgtttgaagacgtattcggagatttccatgaggtgactggcctttccggtggctcctcagggggatctaaacgcacggccgatgggtccgagtttgagtctcccaagaagaaaaggaaagtgagtggtggaagtagcggcggtagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacaaacggacagccgacggaagcgagttcgagtcaccaaagaagaagcggaaagtctaa

SEQ ID NO: 152 - DNA, 5157 bp, synthetic construct
  source 1..5157: mol_type = other DNA
atgatggcgataacgaagcccttgttggcagctacgttggaaaatattgaggacgtacagttcccatgccttgccactccgaagatcgatggaatccgatccgtgaaacagacacaaatgcttagcagaacattcaaacccatcaggaacagcgtaatgaatagattgcttacggaactcttgcccgaagggtctgacggcgagattagcatcgaaggagcgactttccaagatactacctcagcagttatgacgggacacaagatgtataatgctaaattctcatattactggtttgactatgttactgacgatcctttgaagaaatacatagacagggttgaagatatgaaaaattacataactgtccaccctcatatcctggagcatgcacaggtaaagattatcccgctcataccagtagaaattaacaatataaccgaattgttgcagtatgaacgcgatgtgctctctaaaggcttcgagggcgtgatgataaggaagcctgatggcaaatataagttcggtaggtccacattgaaagagggaattctcttgaagatgaaacagtttaaggatgcggaagctactatcattagtatgacggcactgtttaaaaacactaacactaaaaccaaggacaactttggctatagtaaaaggagcacacacaaatcaggaaaagttgaggaggacgtaatgggcagtatagaggtagattacgatggtgtggtgtttagcattggaacgggcttcgacgctgaccagcggagggacttttggcagaataaggaaagttacattggcaagatggttaaattcaaatacttcgagatgggctcaaaagactgtccgagatttcctgtgtttattggaatcagacacgaagaggataggtccggtggctcctcagggggatctaaacgcacggccgatgggtccgagtttgagtctcccaagaagaaaaggaaagtgagtggtggaagtagcggcggtagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacaaacggacagccgacggaagcgagttcgagtcaccaaagaagaagcggaaagtctaa

SEQ ID NO: 153 - DNA, 6120 bp, synthetic construct
  source 1..6120: mol_type = other DNA
atggcagcttctcagacctctcaaacagtagcctctcatgtaccgttcgctgacttgtgttctacgctcgaacgcatccagaaatcaaaggggcgcgccgagaaaatccggcacttcagagaattcttggattcctggaggaagtttcatgatgctctccacaaaaatcacaaagatgtaacggatagtttctaccctgctatgagacttatactgccgcagcttgagagggaacgcatggcgtatggtataaaggagacaatgttggcgaaattgtatattgagctgctgaacttgccaagagatggaaaggacgcgctcaaactgctgaactatagaacacccacgggtacccatggtgacgccggtgactttgccatgatcgcctatttcgtactgaaacctcgatgtcttcaaaaaggttctcttacaattcagcaagtcaacgacctgctggattcaattgcgagtaacaacagcgctaagcgaaaggatctcattaagaaaagcctcctgcagctgataactcagtcctctgcactcgaacaaaaatggctgattcggatgattatcaaggatttgaagttgggggtatctcagcaaactattttcagcgtgtttcacaatgatgcagcagaattgcataatgtcacaacagatcttgagaaagtctgccgacagttgcacgacccctctgtaggcttgagtgacatatctataacacttttttctgcgttcaaacccatgttggctgctattgcggacatagaacacatcgagaaagacatgaaacatcagtcattctatatagagactaaattggacggcgagaggatgcaaatgcacaaagatggtgatgtgtataaatatttttcccgcaacggctacaactacactgatcaattcggagcgtccccaactgaagggtccctcactcctttcatacacaatgcgtttaaggccgatattcagatatgtatcctcgacggcgaaatgatggcgtacaatcccaatacccagaccttcatgcaaaagggaacgaagttcgatattaaacggatggttgaagattccgacctccaaacatgttactgtgtgtttgatgtcctgatggtgaataacaaaaaactcggccatgaaacccttcgaaagcgatacgaaatactcagcagtatatttactccaataccaggccgaatcgagatcgtacagaaaacacaagcccatactaagaatgaagttattgatgcactgaacgaagccatagacaagagggaagaaggcataatggtcaagcagcctctgagtatatataaacctgacaaaaggggggaaggatggctgaagataaagccagaatacgtgtctggtcttatggacgaattggacattctcatcgtcggaggatattggggtaagggttccaggggggggatgatgtcccactttctgtgtgcggttgccgagaaaccgcccccaggggaaaaaccatcagtgttccatacgttgtcacgcgtcggctcaggttgtacgatgaaggaactttacgatctggggttgaaactcgccaaatattggaagccattccatcggaaagcaccgccctctagtatcttgtgtgggacggagaagccagaagtttatatagagccatgtaactcagtaattgttcaaatcaaagccgcagagatcgtcccgtcagacatgtacaagactggatgcacccttagatttcctcgcatcgaaaaaataagagatgataaagagtggcatgagtgcatgactcttgacgaccttgaacagctccgcgggaaggccagcggtaaactggctagtaagcacctctacatcgggggtgactccggtggctcctcagggggatctaaacgcacggccgatgggtccgagtttgagtctcccaagaagaaaaggaaagtgagtggtggaagtagcggcggtagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacaaacggacagccgacggaagcgagttcgagtcaccaaagaagaagcggaaagtctaa

SEQ ID NO: 154 - protein, 226 aa, synthetic construct
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 155 - protein, 251 aa, synthetic construct
MAVHLLIVDALNLIRRIHAVQGSPCVETCQHALDQLIMHSQPTHAVAVFDDENRSSGWRHQRLPDYKAGRPPMPEELHDEMPALRAAFEQRGVPCWSTSGNEADDLAATLAVKVTQAGHQATIVSTDKGYCQLLSPTLRIRDYFQKRWLDAPFIDKEFGVQPQQLPDYWGLAGISSSKVPGVAGIGPKSATQLLVEFQSLEGIYENLDAVAEKWRKKLETHKEMAFLCRDIARLQTDLHIDGNLQQLRLVR

SEQ ID NO: 156 - protein, 292 aa, Staphylococcus aureus
MPNKILLVDGMALLFRHFYATSLHKQFMYNSQGVPTNGIQGFVRHIFSAIHEIRPTHVAVCWDMGQSTFRNDMFDGYKQNRSAPPEELIPQFDYVKEISEQFGFVNIGVKNYEADDVIGTLAQQYSTDNDVYIITGDKDLLQCINDNVEVWLIKKGFNIYNRYTLHRFNEEYALEPQQLIDIKAFMGDTADGYAGVKGIGEKTAIKLIQQYQSVENVVENIDALSAGQRNKINDNLDELYLSKRLAEIHTQVPIDSEALFEKMSFATTLNHILSICNEHELHVSGKYISSHF

SEQ ID NO: 157 - protein, 756 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 158 - protein, 756 aa, synthetic construct
MSFVAGVIRRLDETVVNRIAAGEVIQRPANAIKAMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 159 - protein, 753 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 160 - protein, 753 aa, synthetic construct
MSFVAGVIRRLDETVVNRIAAGEVIQRPANAIKAMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 161 - protein, 335 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLL

SEQ ID NO: 162 - protein, 335 aa, synthetic construct
MSFVAGVIRRLDETVVNRIAAGEVIQRPANAIKAMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLL

SEQ ID NO: 163 - protein, 934 aa, synthetic construct [sequence omitted]

SEQ ID NO: 164 - protein, 934 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGAKSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 165 - protein, 934 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPIMGGKSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 166 - RNA, 115 nt, synthetic construct
  source 1..115: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 113: mod_base = OTHER; note = 2-O-methyl nucleotide
  modified_base 114: mod_base = OTHER; note = 2-O-methyl nucleotide
  modified_base 115: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 113^114: mod_base = OTHER; note = phosphorothioate bond
  modified_base 114^115: mod_base = OTHER; note = phosphorothioate bond
gctgaagcactgcacgccatgttttagagctagaaatagcaagttaaaataaggctagtccgttatcgacttgaaaaagtcggaccgagtcggtccagctgcggtattgtggcgt

SEQ ID NO: 167 - DNA, 24 bp, synthetic construct
  source 1..24: mol_type = other DNA
  modified_base 1: mod_base = OTHER; note = 5' Phosphate
cgtatgtcagggtggtcacgaggg

SEQ ID NO: 168 - skipped

SEQ ID NO: 169 - DNA, 56 bp, synthetic construct
  source 1..56: mol_type = other DNA
  modified_base 1: mod_base = OTHER; note = Locked Nucleic Acid
  modified_base 3: mod_base = OTHER; note = Locked Nucleic Acid
  modified_base 5: mod_base = OTHER; note = Locked Nucleic Acid
  modified_base 7: mod_base = OTHER; note = Locked Nucleic Acid
  modified_base 9: mod_base = OTHER; note = Locked Nucleic Acid
  modified_base 11: mod_base = OTHER; note = Locked Nucleic Acid
  modified_base 13: mod_base = OTHER; note = Locked Nucleic Acid
  modified_base 15: mod_base = OTHER; note = Locked Nucleic Acid
  modified_base 17: mod_base = OTHER; note = Locked Nucleic Acid
  modified_base 19: mod_base = OTHER; note = Locked Nucleic Acid
  modified_base 21: mod_base = OTHER; note = Locked Nucleic Acid
  modified_base 23: mod_base = OTHER; note = Locked Nucleic Acid
  modified_base 44: mod_base = OTHER; note = Locked Nucleic Acid
  modified_base 46: mod_base = OTHER; note = Locked Nucleic Acid
  modified_base 48: mod_base = OTHER; note = Locked Nucleic Acid
  modified_base 50: mod_base = OTHER; note = Locked Nucleic Acid
  modified_base 52: mod_base = OTHER; note = Locked Nucleic Acid
  modified_base 54: mod_base = OTHER; note = Locked Nucleic Acid
  modified_base 56: mod_base = OTHER; note = Locked Nucleic Acid
  modified_base 1^2: mod_base = OTHER; note = phosphorothioate bond
  modified_base 2^3: mod_base = OTHER; note = phosphorothioate bond
  modified_base 54^55: mod_base = OTHER; note = phosphorothioate bond
  modified_base 55^56: mod_base = OTHER; note = phosphorothioate bond
ccctcgtgaccaccctgacatacggcgtgcagtgcttacgccacaataccgcagct

SEQ ID NO: 170 - RNA, 115 nt, synthetic construct
  source 1..115: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 113: mod_base = OTHER; note = 2-O-methyl nucleotide
  modified_base 114: mod_base = OTHER; note = 2-O-methyl nucleotide
  modified_base 115: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 113^114: mod_base = OTHER; note = phosphorothioate bond
  modified_base 114^115: mod_base = OTHER; note = phosphorothioate bond
caccccggctctggctaaaggttttagagctagaaatagcaagttaaaataaggctagtccgttatcgacttgaaaaagtcggaccgagtcggtccagctgcggtattgtggcgt

SEQ ID NO: 171 - RNA, 115 nt, synthetic construct
  source 1..115: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 113: mod_base = OTHER; note = 2-O-methyl nucleotide
  modified_base 114: mod_base = OTHER; note = 2-O-methyl nucleotide
  modified_base 115: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 114^115: mod_base = OTHER; note = phosphorothioate bond
  modified_base 113^114: mod_base = OTHER; note = phosphorothioate bond
gatgtctgcaggccagatgagttttagagctagaaatagcaagttaaaataaggctagtccgttatcgacttgaaaaagtcggaccgagtcggtccgtggttccgggctgcatga

SEQ ID NO: 172 - RNA, 115 nt, synthetic construct
  source 1..115: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 113: mod_base = OTHER; note = 2-O-methyl nucleotide
  modified_base 114: mod_base = OTHER; note = 2-O-methyl nucleotide
  modified_base 115: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 113^114: mod_base = OTHER; note = phosphorothioate bond
  modified_base 114^115: mod_base = OTHER; note = phosphorothioate bond
ggcccagactgagcacgtgagttttagagctagaaatagcaagttaaaataaggctagtccgttatcgacttgaaaaagtcggaccgagtcggtccgtggttccgggctgcatga

SEQ ID NO: 173 - RNA, 115 nt, synthetic construct
  source 1..115: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 113: mod_base = OTHER; note = 2-O-methyl nucleotide
  modified_base 114: mod_base = OTHER; note = 2-O-methyl nucleotide
  modified_base 115: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 113^114: mod_base = OTHER; note = phosphorothioate bond
  modified_base 114^115: mod_base = OTHER; note = phosphorothioate bond
gcgactcctggaagtggccagttttagagctagaaatagcaagttaaaataaggctagtccgttatcgacttgaaaaagtcggaccgagtcggtccagctgcggtattgtggcgt

SEQ ID NO: 174 - DNA, 56 bp, synthetic construct
  source 1..56: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 54^55: mod_base = OTHER; note = phosphorothioate bond
  modified_base 55^56: mod_base = OTHER; note = phosphorothioate bond
cctttccaaagcccattccatcattagccagagccggacgccacaataccgcagct

SEQ ID NO: 175 - DNA, 56 bp, synthetic construct
  source 1..56: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 53^54: mod_base = OTHER; note = phosphorothioate bond
  modified_base 54^55: mod_base = OTHER; note = phosphorothioate bond
acaatgtgccatctggagcactgatctggcctgcagatcatgcagcccggaaccac

SEQ ID NO: 176 - DNA, 56 bp, synthetic construct
  source 1..56: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 54^55: mod_base = OTHER; note = phosphorothioate bond
  modified_base 55^56: mod_base = OTHER; note = phosphorothioate bond
gcttcctttcctctgccatcacaccgtgctcagtctgtcatgcagcccggaaccac

SEQ ID NO: 177 - DNA, 56 bp, synthetic construct
  source 1..56: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 54^55: mod_base = OTHER; note = phosphorothioate bond
  modified_base 53^54: mod_base = OTHER; note = phosphorothioate bond
agggcttcctttcctctgcaatcacgtgctcagtctgtcatgcagcccggaaccac

SEQ ID NO: 178 - DNA, 54 bp, synthetic construct
  source 1..54: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 42: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 53^54: mod_base = OTHER; note = phosphorothioate bond
  modified_base 54: mod_base = OTHER; note = Locked nucleic acid (LNA)
  modified_base 52^53: mod_base = OTHER; note = phosphorothioate bond
attagcagaagtggcccttggcacccacttccaggacgccacaataccgcagct

SEQ ID NO: 179 - DNA, 54 bp, synthetic construct
  source 1..54: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 42: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 53^54: mod_base = OTHER; note = phosphorothioate bond
  modified_base 52^53: mod_base = OTHER; note = phosphorothioate bond
tccattagcagaagtggccattggccacttccaggacgccacaataccgcagct

SEQ ID NO: 180 - DNA, 24 bp, synthetic construct
  source 1..24: mol_type = other DNA
  modified_base 1: mod_base = OTHER; note = 5' Phosphate
atgatggaatgggctttggaaagg

SEQ ID NO: 181 - DNA, 24 bp, synthetic construct
  source 1..24: mol_type = other DNA
  modified_base 1: mod_base = OTHER; note = 5' Phosphate
tcagtgctccagatggcacattgt

SEQ ID NO: 182 - DNA, 24 bp, synthetic construct
  source 1..24: mol_type = other DNA
  modified_base 1: mod_base = OTHER; note = 5' Phosphate
gtgtgatggcagaggaaaggaagc

SEQ ID NO: 183 - DNA, 24 bp, synthetic construct
  source 1..24: mol_type = other DNA
  modified_base 1: mod_base = OTHER; note = 5' Phosphate
tgattgcagaggaaaggaagccct

SEQ ID NO: 184 - DNA, 24 bp, synthetic construct
  source 1..24: mol_type = other DNA
  modified_base 1: mod_base = OTHER; note = 5' Phosphate
gtgccaagggccacttctgctaat

SEQ ID NO: 185 - DNA, 24 bp, synthetic construct
  source 1..24: mol_type = other DNA
  modified_base 1: mod_base = OTHER; note = 5' Phosphate
ccaatggccacttctgctaatgga

SEQ ID NO: 186 - DNA, 53 bp, synthetic construct
  source 1..53: mol_type = other DNA
acactctttccctacacgacgctcttccgatcttgccgctcactttgatgtct

SEQ ID NO: 187 - DNA, 51 bp, synthetic construct
  source 1..51: mol_type = other DNA
gactggagttcagacgtgtgctcttccgatctggggagagggacacacaga

SEQ ID NO: 188 - DNA, 52 bp, synthetic construct
  source 1..52: mol_type = other DNA
acactctttccctacacgacgctcttccgatctccctggcctgggtcaatcc

SEQ ID NO: 189 - DNA, 51 bp, synthetic construct
  source 1..51: mol_type = other DNA
gactggagttcagacgtgtgctcttccgatctctgaagggccaggtccctc

SEQ ID NO: 190 - DNA, 53 bp, synthetic construct
  source 1..53: mol_type = other DNA
acactctttccctacacgacgctcttccgatctcgccgggaactgccgctggc

SEQ ID NO: 191 - DNA, 52 bp, synthetic construct
  source 1..52: mol_type = other DNA
gactggagttcagacgtgtgctcttccgatctgaggaggccctcatctggcg

SEQ ID NO: 192 - RNA, 115 nt, synthetic construct
  source 1..115: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 113: mod_base = OTHER; note = 2-O-methyl nucleotide
  modified_base 114: mod_base = OTHER; note = 2-O-methyl nucleotide
  modified_base 115: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 113^114: mod_base = OTHER; note = phosphorothioate bond
  modified_base 114^115: mod_base = OTHER; note = phosphorothioate bond
gctggccccccaccgccccagttttagagctagaaatagcaagttaaaataaggctagtccgttatcgacttgaaaaagtcggaccgagtcggtccgtggttccgggctgcatga

SEQ ID NO: 193 - DNA, 56 bp, synthetic construct
  source 1..56: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 55^56: mod_base = OTHER; note = phosphorothioate bond
  modified_base 54^55: mod_base = OTHER; note = phosphorothioate bond
ggagaccgccgtcgtcgacaagcctctggcctgcagatcatgcagcccggaaccac

SEQ ID NO: 194 - DNA, 56 bp, synthetic construct
  source 1..56: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 54^55: mod_base = OTHER; note = phosphorothioate bond
  modified_base 55^56: mod_base = OTHER; note = phosphorothioate bond
ggcggtctccgtcgtcaggatcattagccagagccggacgccacaataccgcagct

SEQ ID NO: 195 - DNA, 24 bp, synthetic construct
  source 1..24: mol_type = other DNA
  modified_base 1: mod_base = OTHER; note = 5' Phosphate
ggcttgtcgacgacggcggtctcc

SEQ ID NO: 196 - DNA, 24 bp, synthetic construct
  source 1..24: mol_type = other DNA
  modified_base 1: mod_base = OTHER; note = 5' Phosphate
atgatcctgacgacggagaccgcc

SEQ ID NO: 197 - DNA, 58 bp, synthetic construct
  source 1..58: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 25: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 58: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 56^57: mod_base = OTHER; note = phosphorothioate bond
  modified_base 57^58: mod_base = OTHER; note = phosphorothioate bond
ccgtctgcacaccccggctctggctatctggcctgcagatcatgcagcccggaaccac

SEQ ID NO: 198 - DNA, 58 bp, synthetic construct
  source 1..58: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 25: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 58: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 56^57: mod_base = OTHER; note = phosphorothioate bond
  modified_base 57^58: mod_base = OTHER; note = phosphorothioate bond
gctcactttgatgtctgcaggccagatagccagagccggacgccacaataccgcagct

SEQ ID NO: 199 - DNA, 26 bp, synthetic construct
  source 1..26: mol_type = other DNA
  modified_base 1: mod_base = OTHER; note = 5' Phosphate
tagccagagccggggtgtgcagacgg

SEQ ID NO: 200 - DNA, 26 bp, synthetic construct
  source 1..26: mol_type = other DNA
  modified_base 1: mod_base = OTHER; note = 5' Phosphate
tctggcctgcagacatcaaagtgagc

SEQ ID NO: 201 - DNA, 52 bp, synthetic construct
  source 1..52: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 40: mod_base = OTHER; note = Locked nucleic acid (LNA)
  modified_base 42: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 51^52: mod_base = OTHER; note = phosphorothioate bond
  modified_base 50^51: mod_base = OTHER; note = phosphorothioate bond
ggagaccgccgtcgtcgacaagccggcggtgggtcatgcagcccggaaccac

SEQ ID NO: 202 - DNA, 54 bp, synthetic construct
  source 1..54: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 42: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 50^51: mod_base = OTHER; note = phosphorothioate bond
  modified_base 51^52: mod_base = OTHER; note = phosphorothioate bond
ggcggtctccgtcgtcaggatcatccacttccaggacgccacaataccgcagct

SEQ ID NO: 203 - DNA, 54 bp, synthetic construct
  source 1..54: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 25: mod_base = OTHER; note = Locked nucleic acid (LNA)
  modified_base 42: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 53^54: mod_base = OTHER; note = phosphorothioate bond
  modified_base 52^53: mod_base = OTHER; note = phosphorothioate bond
cggggcacagcgactcctggaagtggggcggtgggtcatgcagcccggaaccac

SEQ ID NO: 204 - DNA, 56 bp, synthetic construct
  source 1..56: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 21: mod_base = OTHER; note = Locked nucleic acid (LNA)
  modified_base 23: mod_base = OTHER; note = Locked nucleic acid (LNA)
  modified_base 25: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 54: mod_base = OTHER; note = Locked nucleic acid (LNA)
  modified_base 56: mod_base = OTHER; note = Locked nucleic acid (LNA)
  modified_base 55^56: mod_base = OTHER; note = phosphorothioate bond
  modified_base 54^55: mod_base = OTHER; note = phosphorothioate bond
  modified_base 1^2: mod_base = OTHER; note = phosphorothioate bond
  modified_base 2^3: mod_base = OTHER; note = phosphorothioate bond
ggaactgccgctggccccccaccgccccacttccaggacgccacaataccgcagct

SEQ ID NO: 205 - DNA, 26 bp, synthetic construct
  source 1..26: mol_type = other DNA
  modified_base 1: mod_base = OTHER; note = 5' Phosphate
ccacttccaggagtcgctgtgccccg

SEQ ID NO: 206 - DNA, 26 bp, synthetic construct
  source 1..26: mol_type = other DNA
  modified_base 1: mod_base = OTHER; note = 5' Phosphate
ggcggtggggggccagcggcagttcc

SEQ ID NO: 207 - DNA, 20 bp, synthetic construct
  source 1..20: mol_type = other DNA
  modified_base 1: mod_base = OTHER; note = 5' Phosphate
cgtatgtcagggtggtcacg

SEQ ID NO: 208 - DNA, 52 bp, synthetic construct
  source 1..52: mol_type = other DNA
  modified_base 1: mod_base = OTHER; note = Locked nucleic acid (LNA)
  modified_base 3: mod_base = OTHER; note = Locked nucleic acid (LNA)
  modified_base 5: mod_base = OTHER; note = Locked nucleic acid (LNA)
  modified_base 7: mod_base = OTHER; note = Locked nucleic acid (LNA)
  modified_base 9: mod_base = OTHER; note = Locked nucleic acid (LNA)
  modified_base 11: mod_base = OTHER; note = Locked nucleic acid (LNA)
  modified_base 13: mod_base = OTHER; note = Locked nucleic acid (LNA)
  modified_base 15: mod_base = OTHER; note = Locked nucleic acid (LNA)
  modified_base 17: mod_base = OTHER; note = Locked nucleic acid (LNA)
  modified_base 19: mod_base = OTHER; note = Locked nucleic acid (LNA)
  modified_base 40: mod_base = OTHER; note = Locked nucleic acid (LNA)
  modified_base 42: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 51^52: mod_base = OTHER; note = phosphorothioate bond
  modified_base 50^51: mod_base = OTHER; note = phosphorothioate bond
cgtgaccaccctgacatacggcgtgcagtgcttacgccacaataccgcagct

SEQ ID NO: 209 - DNA, 52 bp, synthetic construct; identical to SEQ ID NO: 208
  misc_feature 3: note = RNA
  misc_feature 17: note = RNA
  source 1..52: mol_type = other DNA
  modified_base 1: mod_base = OTHER; note = 2-O-methyl nucleotide
  modified_base 3: mod_base = OTHER; note = 2-O-methyl nucleotide
  modified_base 5: mod_base = OTHER; note = 2-O-methyl nucleotide
  modified_base 7: mod_base = OTHER; note = 2-O-methyl nucleotide
  modified_base 9: mod_base = OTHER; note = 2-O-methyl nucleotide
  modified_base 11: mod_base = OTHER; note = 2-O-methyl nucleotide
  modified_base 13: mod_base = OTHER; note = 2-O-methyl nucleotide
  modified_base 15: mod_base = OTHER; note = 2-O-methyl nucleotide
  modified_base 17: mod_base = OTHER; note = 2-O-methyl nucleotide
  modified_base 19: mod_base = OTHER; note = 2-O-methyl nucleotide
  modified_base 40: mod_base = OTHER; note = Locked nucleic acid (LNA)
  modified_base 42: mod_base = OTHER; note = Locked nucleic acid (LNA)
  modified_base 44: mod_base = OTHER; note = Locked nucleic acid (LNA)
  modified_base 46: mod_base = OTHER; note = Locked nucleic acid (LNA)
  modified_base 48: mod_base = OTHER; note = Locked nucleic acid (LNA)
  modified_base 50: mod_base = OTHER; note = Locked nucleic acid (LNA)
  modified_base 52: mod_base = OTHER; note = Locked nucleic acid (LNA)
  modified_base 1^2: mod_base = OTHER; note = phosphorothioate bond
  modified_base 2^3: mod_base = OTHER; note = phosphorothioate bond
  modified_base 51^52: mod_base = OTHER; note = phosphorothioate bond
  modified_base 50^51: mod_base = OTHER; note = phosphorothioate bond

SEQ ID NO: 210 - protein, 9 aa, synthetic construct
YAYDVPDYA

SEQ ID NO: 211 - protein, 9 aa, synthetic construct
YDVPDYASL

SEQ ID NO: 212 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 213 - protein, 333 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQ

SEQ ID NO: 214 - protein, 373 aa, synthetic construct
MAETREEETVSAEASGFSDLSDSEFLEFLDLEDAQESKALVNMPGPSSESLGKDDKPISLQNWKRGLDILSPMERFHLKYLYVTDLATQNWCELQTAYGKELPGFLAPEKAAVLDTGASIHLARELELHDLVTVPVTTKEDAWAIKFLNILLLIPTLQSEGHIREFPVFGEGEGVLLVGVIDELHYTAKGELELAELKTRRRPMLPLEAQKKKDCFQVSLYKYIFDAMVQGKVTPASLIHHTKLCLEKPLGPSVLRHAQQGGFSVKSLGDLMELVFLSLTLSDLPVIDILKIEYIHQETATVLGTEIVAFKEKEVRAKVQHYMAYWMGHREPQGVDVEEAWKCRTCTYADICEWRKGSGVLSSTLAPQVKKAK

SEQ ID NO: 215 - protein, 291 aa, synthetic construct
MSKSWGKFIEEEEAEMASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQ

SEQ ID NO: 216 - protein, 300 aa, synthetic construct
MALLDLKQFYELREGCDDKGILVMDGDWLVFQAMSAAEFDASWEEEIWHRCCDHAKARQILEDSIKSYETRKKAWAGAPIVLAFTDSVNWRKELVDPNYKANRKAVKKPVGYFEFLDALFEREEFYCIREPMLEGDDVMGVIASNPSAFGARKAVIISCDKDFKTIPNCDFLWCTTGNILTQTEESADWWHLFQTIKGDITDGYSGIAGWGDTAEDFLNNPFITEPKTSVLKSGKNKGQEVTKWVKRDPEPHETLWDCIKSIGAKAGMTEEDIIKQGQMARILRFNEYNFIDKEIYLWRP

SEQ ID NO: 217 - protein, 866 aa, synthetic construct
MSTKPLFLLRKAKKSSGEPDVVLWASNDFESTCATLDYLIVKSGKKLSSYFKAVATNFPVVNDLPAEGEIDFTWSERYQLSKDSMTWELKPGAAPDNAHYQGNTNVNGEDMTEIEENMLLPISGQELPIRWLAQHGSEKPVTHVSRDGLQALHIARAEELPAVTALAVSHKTSLLDPLEIRELHKLVRDTDKVFPNPGNSNLGLITAFFEAYLNADYTDRGLLTKEWMKGNRVSHITRTASGANAGGGNLTDRGEGFVHDLTSLARDVATGVLARSMDLDIYNLHPAHAKRIEEIIAENKPPFSVFRDKFITMPGGLDYSRAIVVASVKEAPIGIEVIPAHVTEYLNKVLTETDHANPDPEIVDIACGRSSAPMPQRVTEEGKQDDEEKPQPSGTTAVEQGEAETMEPDATEHHQDTQPLDAQSQVNSVDAKYQELRAELHEARKNIPSKNPVDDDKLLAASRGEFVDGISDPNDPKWVKGIQTRDCVYQNQPETEKTSPDMNQPEPVVQQEPEIACNACGQTGGDNCPDCGAVMGDATYQETFDEESQVEAKENDPEEMEGAEHPHNENAGSDPHRDCSDETGEVADPVIVEDIEPGIYYGISNENYHAGPGISKSQLDDIADTPALYLWRKNAPVDTTKTKTLDLGTAFHCRVLEPEEFSNRFIVAPEFNRRTNAGKEEEKAFLMECASTGKTVITAEEGRKIELMYQSVMALPLGQWLVESAGHAESSIYWEDPETGILCRCRPDKIIPEFHWIMDVKTTADIQRFKTAYYDYRYHVQDAFYSDGYEAQFGVQPTFVFLVASTTIECGRYPVEIFMMGEEAKLAGQQEYHRNLRTLSDCLNTDEWPAIKTLSLPRWAKEYAND

SEQ ID NO: 218 - protein, 303 aa, synthetic construct
EHPHNENAGSDPHRDCSDETGEVADPVIVEDIEPGIYYGISNENYHAGPGISKSQLDDIADTPALYLWRKNAPVDTTKTKTLDLGTAFHCRVLEPEEFSNRFIVAPEFNRRTNAGKEEEKAFLMECASTGKTVITAEEGRKIELMYQSVMALPLGQWLVESAGHAESSIYWEDPETGILCRCRPDKIIPEFHWIMDVKTTADIQRFKTAYYDYRYHVQDAFYSDGYEAQFGVQPTFVFLVASTTIECGRYPVEIFMMGEEAKLAGQQEYHRNLRTLSDCLNTDEWPAIKTLSLPRWAKEYAND

SEQ ID NO: 219 - protein, 324 aa, synthetic construct
VQIPQNPLILVDGSSYLYRAYHAFPPLTNSAGEPTGAMYGVLNMLRSLIMQYKPTHAAVVFDAKGKTFRDELFEHYKSHRPPMPDDLRAQIEPLHAMVKAMGLPLLAVSGVEADDVIGTLAREAEKAGRPVLISTGDKDMAQLVTPNITLINTMTNTILGPEEVVNKYGVPPELIIDFLALMGDSSDNIPGVPGVGEKTAQALLQGLGGLDTLYAEPEKIAGLSFRGAKTMAAKLEQNKEVAYLSYQLATIKTDVELELTCEQLEVQQPAAEELLGLFKKYEFKRWTADVEAGKWLQAKGAKPAAKPQETSVADEAPEVTATVI

SEQ ID NO: 220 - protein, 577 aa, synthetic construct
MKQQIQLRRREVDETADLPAELPPLLRRLYASRGVRSAQELERSVKGMLPWQQLSGVEKAVEILYNAFREGTRIIVVGDFDADGATSTALSVLAMRSLGCSNIDYLVPNRFEDGYGLSPEVVDQAHARGAQLIVTVDNGISSHAGVEHARSLGIPVIVTDHHLPGDTLPAAEAIINPNLRDCNFPSKSLAGVGVAFYLMLALRTFLRDQGWFDERNIAIPNLAELLDLVALGTVADVVPLDANNRILTWQGMSRIRAGKCRPGIKALLEVANRDAQKLAASDLGFALGPRLNAAGRLDDMSVGVALLLCDNIGEARVLANELDALNQTRKEIEQGMQIEALTLCEKLERSRDTLPGGLAMYHPEWHQGVVGILASRIKERFHRPVIAFAPAGDGTLKGSGRSIQGLHMRDALERLDTLYPGMMLKFGGHAMAAGLSLEEDKFKLFQQRFGELVTEWLDPSLLQGEVVSDGPLSPAEMTMEVAQLLRDAGPWGQMFPEPLFDGHFRLLQQRLVGERHLKVMVEPVGGGPLLDGIAFNVDTALWPDNGVREVQLAYKLDINEFRGNRSLQIIIDNIWPI

SEQ ID NO: 221 - RNA, 115 nt, synthetic construct; identical to SEQ ID NO: 172
  source 1..115: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 113: mod_base = OTHER; note = 2-O-methyl nucleotide
  modified_base 114: mod_base = OTHER; note = 2-O-methyl nucleotide
  modified_base 115: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 114^115: mod_base = OTHER; note = phosphorothioate bond
  modified_base 113^114: mod_base = OTHER; note = phosphorothioate bond

SEQ ID NO: 222 - RNA, 115 nt, synthetic construct; identical to SEQ ID NO: 173
  source 1..115: mol_type = other RNA
  modified_base 1: mod_base = OTHER; note = 2-O-methyl nucleotide
  modified_base 2: mod_base = OTHER; note = 2-O-methyl nucleotide
  modified_base 3: mod_base = OTHER; note = 2-O-methyl nucleotide
  modified_base 113: mod_base = OTHER; note = 2-O-methyl nucleotide
  modified_base 114: mod_base = OTHER; note = 2-O-methyl nucleotide
  modified_base 115: mod_base = OTHER; note = 2-O-methyl nucleotide
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 113^114: mod_base = OTHER; note = phosphorothioate bond
  modified_base 114^115: mod_base = OTHER; note = phosphorothioate bond

SEQ ID NO: 223 - DNA, 53 bp, synthetic construct; identical to SEQ ID NO: 186
  source 1..53: mol_type = other DNA

SEQ ID NO: 224 - DNA, 51 bp, synthetic construct; identical to SEQ ID NO: 187
  source 1..51: mol_type = other DNA

SEQ ID NO: 225 - DNA, 52 bp, synthetic construct; identical to SEQ ID NO: 188
  source 1..52: mol_type = other DNA

SEQ ID NO: 226 - DNA, 51 bp, synthetic construct; identical to SEQ ID NO: 189
  source 1..51: mol_type = other DNA

SEQ ID NO: 227 - DNA, 53 bp, synthetic construct; identical to SEQ ID NO: 190
  source 1..53: mol_type = other DNA

SEQ ID NO: 228 - DNA, 52 bp, synthetic construct; identical to SEQ ID NO: 191
  source 1..52: mol_type = other DNA

(19) United States
(12) Patent Application Publication
(10) Pub. No.: US 2025/0179468 A1
(43) Pub. Date: Jun. 5, 2025

(54) DIRECT REPLACEMENT GENOME EDITING

(71) Applicant: Transit Therapeutics, Inc., Woodside, CA (US)

(72) Inventors: Schaked Omer HALPERIN, Emeryville, CA (US); Michael CHICKERING, San Francisco, CA (US); Parbir GREWAL, Alameda, CA (US); Leonard CHAVEZ, Oakland, CA (US)

(21) Appl. No.: 19/037,641

(22) Filed: Jan. 27, 2025

Related U.S. Application Data
(63) Continuation of application No. 17/984,045, filed on Nov. 9, 2022, now abandoned.
(60) Provisional application No. 63/278,886, filed on Nov. 12, 2021.
(60) Provisional application No. 63/341,200, filed on May 12, 2022.

(51) Int. Cl. C12N 15/10; C12N 9/22; C12N 15/113

(52) U.S. Cl. CPC C12N 15/102; C12N 9/22; C12N 15/113; C07K 2319/80; C12N 2310/20; C12N 2810/40

(57) ABSTRACT

Described herein are compositions, systems, and methods for nucleic acid editing. The editing may be accomplished using a ligase coupled to an endonuclease. The nucleic acid editing may include ligation of an integrating nucleic acid to a target nucleic acid. The nucleic acid editing may include replacement of a portion of the target nucleic acid with the integrating nucleic acid.

### CROSS-REFERENCE

This application is a continuation of U.S. patent application Ser. No. 17/984,045 filed Nov. 9, 2022, which claims the benefit of U.S. Provisional Application Ser. No. 63/278,886 filed on Nov. 12, 2021, and U.S. Provisional Application Ser. No. 63/341,200 filed on May 12, 2022, the entireties of which are hereby incorporated by reference.

### SEQUENCE LISTING

The instant application contains a Sequence Listing which has been submitted electronically in XML file format and is hereby incorporated by reference in its entirety. Said XML copy, created on Nov. 7, 2022, is named J4040_03015SL.xml and is 703,885 bytes in size.”

### BACKGROUND

Improved gene editing methods are needed for modifying nucleic acids.

### SUMMARY

Disclosed herein, in some aspects, are systems or compositions comprising: a DNA-binding protein coupled to a DNA ligase. The DNA-binding protein may include an endonuclease. The endonuclease may include an RNA-guided endonuclease. In some aspects, the coupling is covalent. Some aspects include a fusion protein comprising the DNA-binding protein (e.g. endonuclease such as an RNA-guided endonuclease) and the DNA ligase. Some aspects include a composition comprising: a cell containing a DNA-binding protein (e.g. endonuclease such as an RNA-guided endonuclease) and a DNA ligase, both of which are heterologous to the cell. In some aspects, the DNA-binding protein is amino (N)-terminal relative to the DNA ligase within the fusion protein. In some aspects, the DNA-binding protein is carboxy (C)-terminal relative to the DNA ligase within the fusion protein. In some aspects, the connection comprises a linker comprising 1-100 amino acids. In some aspects, the coupling is non-covalent. In some aspects, the composition comprises a first polypeptide comprising at least part of the DNA-binding protein, and a second polypeptide comprising at least part of the DNA ligase, wherein the first and second polypeptides are non-covalently coupled. In some aspects, the first polypeptide comprises a first heterodimerization domain that binds a second heterodimerization domain, and wherein the second polypeptide comprises the second heterodimerization domain. In some aspects, the heterodimer domains comprise a leucine zipper, PDZ domain, streptavidin, streptavidin binding protein, foldon domain, hydrophobic moiety, or a functional binding fragment thereof. In some aspects, the first polypeptide comprises a first intein that binds a second intein, and wherein the second polypeptide comprises the second intein. In some aspects, the ligase comprises a hairpin binding motif, and wherein the DNA-binding protein and the DNA ligase are coupled with a nucleic acid comprising a scaffold that binds to the DNA-binding protein and a hairpin that binds to the hairpin binding motif. In some aspects, the hairpin binding motif comprises an MS2 coat protein (MCP) peptide, and wherein the hairpin comprises an MS2 hairpin. In some aspects, the DNA-binding protein and the DNA ligase are coupled with a heterobifunctional molecule comprising an endonuclease binding domain and a DNA ligase binding domain. In some aspects, the heterobifunctional molecule comprises a small molecule. In some aspects, the DNA-binding protein comprises a class II CRISPR/Cas endonuclease. In some aspects, the DNA-binding protein comprises a Cas9 endonuclease. In some aspects, the DNA-binding protein comprises a nickase. In some aspects, the DNA-binding protein comprises an amino acid sequence at least 80% identical to the amino acid sequence of any one of SEQ ID NOS: 1-13, or a functional fragment thereof. In some aspects, the DNA ligase ligates DNA strands base paired to a DNA splint. In some aspects, the DNA ligase ligates DNA strands base paired to an RNA splint. In some aspects, the DNA ligase comprises an amino acid sequence at least 80% identical to the amino acid sequence of any one of SEQ ID NOS: 55-96, or a functional fragment thereof. In some aspects, the DNA-binding protein or the DNA ligase comprises a nuclear localization signal, chromatin modifying domain, cell penetrating peptide, or tag polypeptide. Some aspects include a guide RNA and an integrating nucleic acid. Some aspects include one or more nucleic acids encoding the composition. Some aspects include a cell comprising the composition, or comprising the one or more nucleic acids.

Disclosed herein, in some aspects, are editing methods, comprising: contacting a target nucleic acid in a cell with an endonuclease at a predetermined locus of the target nucleic acid, thereby introducing a nick at the predetermined locus of the target nucleic acid; introducing a pre-synthesized integrating nucleic acid to the cell; and ligating a 5′ end of the pre-synthesized integrating nucleic acid to a 3′ end of the nick at the predetermined locus of the target nucleic acid. In some aspects, the endonuclease comprises a class II CRISPR/Cas endonuclease. In some aspects, the endonuclease comprises Cas9 nickase. Some aspects include contacting the endonuclease and the predetermined locus of the target nucleic acid with a guide nucleic acid. In some aspects, said ligating is performed by a ligase coupled to the endonuclease. In some aspects, the pre-synthesized integrating nucleic acid comprises a mutation in relation to the target nucleic acid. In some aspects, the nick comprises a single phosphodiester strand break in the otherwise double stranded target nucleic acid. In some aspects, the nick comprises a non-sticky, non-blunt end of a strand of the target nucleic acid. In some aspects, the target nucleic acid comprises a chromosome of the cell. In some aspects, the cell is eukaryotic.

Disclosed herein, in some aspects, are editing systems, comprising: a ligase; an endonuclease that introduces a nick at a predetermined locus of a target nucleic acid; and a pre-synthesized integrating nucleic acid comprising a 5′ end that is ligated by the ligase to a 3′ end of the nick at the predetermined locus of the target nucleic acid. In some aspects, the endonuclease comprises a class II CRISPR/Cas endonuclease. In some aspects, the endonuclease comprises Cas9 nickase. Some aspects include a guide nucleic acid that brings the endonuclease into proximity with the predetermined locus of the target nucleic acid. In some aspects, the ligase is coupled to the endonuclease. In some aspects, the pre-synthesized integrating nucleic acid comprises a mutation in relation to the target nucleic acid. In some aspects, the nick comprises a single phosphodiester strand break in the otherwise double stranded target nucleic acid. In some aspects, the nick comprises a non-sticky, non-blunt end of a strand of the target nucleic acid. In some aspects, the target nucleic acid comprises a chromosome of a cell. In some aspects, the cell is eukaryotic.

Disclosed herein, in some aspects, are systems of nucleic acids comprising: a guide nucleic acid comprising: (a) a spacer complementary to a region of a genomic locus of a genomic strand, (b) a scaffold for complexing with a DNA-binding protein, (c) an optional donor binding site that is at least partially complementary to an integrating nucleic acid, and (d) a flap binding site that is at least partially identical or complementary to a genomic flap at or adjacent to the genomic locus; and an integrating nucleic acid comprising a 5′ end to be ligated to a 3′ terminus of the genomic strand generated by a DNA-binding protein. The DNA-binding protein may include an endonuclease. The endonuclease may include an RNA-guided endonuclease. Disclosed herein, in some aspects, are systems of nucleic acids comprising: a guide nucleic acid comprising: (a) a spacer complementary to a region of a genomic locus of a genomic strand, (b) a scaffold for complexing with a DNA-binding protein, and (c) an optional donor binding site that is at least partially complementary to a splinting nucleic acid; an integrating nucleic acid comprising a 5′ end to be ligated to a 3′ terminus of the genomic strand generated by a DNA-binding protein; and a splinting nucleic acid comprising a flap binding site that is at least partially identical or complementary to a genomic flap at or adjacent to the genomic locus, and comprising an optional guide binding site that is at least partially complementary to a guide nucleic acid. In some aspects, the genomic strand is in a cell. In some aspects, the splinting nucleic acid further comprises a donor binding site that is at least partially identical or complementary to a portion of the integrating nucleic acid. In some aspects, the guide nucleic acid comprises a sequence of linking nucleic acids between the scaffold and the donor binding site. In some aspects, the guide nucleic acid or the integrating nucleic acid comprises a modified internucleoside linkage. In some aspects, the modified internucleoside linkage comprises a phosphorothioate linkage. In some aspects, the modified internucleoside linkage is between any of the 4 terminal nucleosides at a 5′ end or at a 3′ end of the guide nucleic acid or the integrating nucleic acid. In some aspects, the guide nucleic acid or the integrating nucleic acid comprises a modified nucleoside. In some aspects, the modified nucleoside comprises a locked nucleic acid (LNA), a 2′ fluoro, a 2′ O-alkyl, or a combination thereof. In some aspects, the modified nucleoside is any of the 3 terminal nucleosides at a 5′ end or at a 3′ end of the guide nucleic acid or the integrating nucleic acid. The modified nucleoside may include an LNA, a 2′fluoro, a 2′ O-alkyl, a methylated cytosine, an inverted thymidine, or a combination thereof.

Disclosed herein, in some aspects, are compositions, comprising: a DNA-binding protein connected to a DNA ligase. The DNA-binding protein may include an endonuclease. The endonuclease may include an RNA-guided endonuclease. In some aspects, the connection between the DNA-binding protein and the DNA ligase is covalent. Some aspects include a fusion protein comprising the DNA-binding protein upstream of the DNA ligase. Some aspects include a fusion protein comprising the DNA-binding protein downstream of the DNA ligase. In some aspects, the connection comprises a linker comprising 1-100 amino acids. In some aspects, the composition comprises a first polypeptide comprising at least part of the DNA-binding protein, and a second polypeptide comprising at least part of the DNA ligase, wherein the first and second polypeptides are bound together covalently or non-covalently. In some aspects, the first polypeptide comprises a first heterodimerization domain that binds a second heterodimerization domain, and wherein the second polypeptide comprises the second heterodimerization domain. In some aspects, the heterodimer domains comprise a leucine zipper, PDZ domain, streptavidin, streptavidin binding protein, foldon domain, hydrophobic moiety, or a functional binding fragment thereof. In some aspects, the first polypeptide comprises a first intein that binds a second intein, and wherein the second polypeptide comprises the second intein. In some aspects, the DNA-binding protein and the DNA ligase are bound together by a small molecule. In some aspects, the DNA-binding protein comprises a class II CRISPR/Cas endonuclease. In some aspects, the DNA-binding protein comprises a Cas9 endonuclease. In some aspects, the DNA-binding protein comprises a nickase. In some aspects, the DNA-binding protein comprises an amino acid sequence at least 80% identical to the amino acid sequence of any one of SEQ ID NOS: 1-13, or a functional fragment thereof. In some aspects, the DNA ligase ligates DNA strands base paired to a DNA splint. In some aspects, the DNA ligase ligates DNA strands base paired to an RNA splint. In some aspects, the DNA ligase comprises an amino acid sequence at least 80% identical to the amino acid sequence of any one of SEQ ID NOS: 55-96, or a functional fragment thereof. In some aspects, the DNA-binding protein or the DNA ligase comprises a nuclear localization signal, chromatin modifying domain, cell penetrating peptide, or tag polypeptide. Some aspects include a guide RNA and an integrating nucleic acid. Some aspects relate to a cell comprising the composition. Some aspects include a nucleic acid encoding the composition. Some aspects include one or more nucleic acids encoding the first or second polypeptides. Some aspects include an editing method (e.g. nucleic acid) which uses the composition. Some aspects include a method of treatment using the composition. Some aspects include administering the composition to a subject.

Disclosed herein, in some aspects, are fusion proteins, comprising: a DNA-binding protein fused to a DNA ligase. The DNA-binding protein may include an endonuclease. The endonuclease may include an RNA-guided endonuclease. Disclosed herein, in some aspects, are protein complexes, comprising: a DNA-binding protein bound to a DNA ligase. In some aspects, the endonuclease and the DNA ligase are bound together through heterodimerization domains. In some aspects, the heterodimerization domains comprise leucine zippers, PDZ domains, streptavidin and streptavidin binding protein, foldon domains, hydrophobic polypeptides, an antibody that binds the Cas nickase, or an antibody that binds the DNA ligase, or one or more binding fragments thereof. Disclosed herein, in some aspects, are cells comprising the fusion protein or the protein complex. Disclosed herein, in some aspects, are cells comprising a heterologous DNA-binding protein and a DNA ligase that was introduced into the cell. Some aspects include a nuclease that is different from the DNA-binding protein. Disclosed herein, in some aspects, are guide nucleic acids, comprising: a spacer at least partially reverse complementary to a first region of a target nucleic acid; a scaffold configured to bind to an endonuclease; and a flap binding site at least partially reverse complementary to a nucleic acid flap, and an integrating nucleic acid binding site. Disclosed herein, in some aspects, are integrating nucleic acids, comprising: a single or double-stranded DNA region to be inserted into a target nucleic acid, wherein the single or double-stranded DNA region is flanked by at least one additional single-stranded region comprising a guide binding site. Disclosed herein, in some aspects, are editing systems, comprising a DNA-binding protein, the guide nucleic acid, and the integrating nucleic acid. Disclosed herein, in some aspects, are editing methods, comprising: contacting a target nucleic acid with the editing system and a DNA ligase.

Disclosed herein, in some aspects, are systems comprising: at least one DNA-binding protein; at least one guide nucleic acid comprising: a spacer at least partially complementary to a genomic locus in a cell; a scaffold for complexing with the at least one DNA-binding protein; and an optional donor binding site that is at least partially complementary to an integrating nucleic acid; and at least one DNA ligase; and the integrating nucleic acid, comprising a flap binding site at least partially reverse complementary to a nucleic acid flap and optionally comprising a guide binding site that is at least partially complementary to the at least one guide nucleic acid, wherein the at least one DNA-binding protein cleaves or nicks at least one strand of the genomic locus, and wherein the at least one DNA ligase ligates an end of the integrating nucleic acid to the genomic flap site, thereby replacing a region of the genomic locus with the integrating nucleic acid in the cell. The DNA-binding protein may include an endonuclease. The endonuclease may include an RNA-guided endonuclease. In some aspects, the integrating nucleic acid comprises a single-stranded DNA. In some aspects, the integrating nucleic acid comprises a double-stranded DNA.

Disclosed herein, in some aspects, are systems comprising: at least one DNA-binding protein comprising a first DNA-binding protein and an optional second DNA-binding protein; at least one guide nucleic acid comprising a first guide nucleic acid and a second guide nucleic acid, the first guide nucleic acid comprising: a first spacer complementary to a first region of a genomic locus in a cell; a first scaffold for complexing with the first DNA-binding protein; and an optional first donor binding site that at least partially complementary to an integrating nucleic acid; and a first flap binding site that is at least partially identical or complementary to a first genomic flap at or adjacent to the genomic locus; and the second guide nucleic acid comprising: a second spacer complementary to a second region of the genomic locus in the cell; a second scaffold for complexing with the first or second DNA-binding protein; an optional second donor binding site that at least partially complementary to the integrating nucleic acid; and a second flap binding site that is at least partially identical or complementary to a second genomic flap at or adjacent to the genomic locus; at least one DNA ligase comprising a first DNA ligase and an optional second DNA ligase; and at least one integrating nucleic acid comprising a first strand and a second strand: wherein the first strand comprises an optional first guide binding site that is at least partially complementary to the first guide nucleic acid; and wherein the second strand comprises an optional second guide binding site that is at least partially complementary to the second guide nucleic acid, wherein the first DNA-binding protein and/or the second DNA-binding protein each cleaves or nicks at least one strand of the genomic locus in the cell; and wherein the first DNA ligase ligates an end of the first strand of the integrating nucleic acid to the first genomic flap; and the first or second DNA ligase ligates an end of the second strand of the integrating nucleic acid to the second genomic flap, thereby replacing a region of the genomic locus with the integrating nucleic acid in the cell. In some aspects, the integrating nucleic acid comprises a double-stranded DNA duplex region. The DNA-binding protein may include an endonuclease. The endonuclease may include an RNA-guided endonuclease. In some aspects, the integrating nucleic acid comprises a 5′ overhang optionally comprising the first guide binding site. In some aspects, the integrating nucleic acid comprises a 5′ overhang optionally comprising the second guide binding site.

Disclosed herein, in some aspects, are systems comprising: at least one DNA-binding protein; at least one guide nucleic acid comprising: a spacer complementary to a genomic locus in a cell; a scaffold for complexing with the at least one DNA-binding protein; and an optional donor binding site that is at least partially complementary to an integrating nucleic acid; at least one DNA ligase; and the integrating nucleic acid that: comprises an optional guide binding site that is at least partially complementary to the at least one guide nucleic acid; and comprises a flap binding site that is at least partially identical or complementary to a genomic flap at or adjacent to the genomic locus, wherein the at least one DNA-binding protein cleaves or nicks at least one strand of the genomic locus; and wherein the at least one DNA ligase ligates an end of the integrating nucleic acid to the genomic flap, thereby replacing a region of the genomic locus with the integrating nucleic acid in the cell. The DNA-binding protein may include an endonuclease. The endonuclease may include an RNA-guided endonuclease. In some aspects, the integrating nucleic acid comprises a DNA comprising a 3′ overhang. In some aspects, the 3′ overhang comprises the guide binding site. In some aspects, the 3′ overhang comprises the flap binding site. In some aspects, the at least one DNA ligase ligates a strand of the integrating nucleic acid to the genomic nucleic acid sequence.

Disclosed herein, in some aspects, are systems comprising: at least one DNA-binding protein comprising a first DNA-binding protein and an optional second DNA-binding protein; at least one guide nucleic acid comprising a first guide nucleic acid and a second guide nucleic acid, the first guide nucleic acid comprising: a first spacer complementary to a first region of a genomic locus in a cell; a first scaffold for complexing with the first DNA-binding protein; and an optional first donor binding site that at least partially complementary to an integrating nucleic acid; and the second guide nucleic acid comprising: a second spacer complementary to a second region of the genomic locus in the cell; a second scaffold for complexing with the first or second DNA-binding protein; and an optional second donor binding site that at least partially complementary to the integrating nucleic acid; and at least one DNA ligase comprising a first DNA ligase and an optional second DNA ligase; and the integrating nucleic acid comprising a first strand and a second strand: wherein the first strand comprises an optional first guide binding site that is at least partially complementary to the first guide nucleic acid; wherein the second strand comprises an optional second guide binding site that is at least partially complementary to the second guide nucleic acid; wherein the first strand comprises a first flap binding site that is at least partially identical or complementary to a first genomic flap at or adjacent to the genomic locus; and wherein the second strand comprises a second flap binding site that is at least partially identical or complementary to a second genomic flap at or adjacent to the genomic locus; wherein the first DNA-binding protein and/or the second DNA-binding protein each cleaves or nicks at least one strand of the genomic locus in the cell; and wherein the first DNA ligase ligates an end of the first strand of the integrating nucleic acid to the first genomic flap; and the first or second DNA ligase ligates an end of the second strand of the integrating nucleic acid to the second genomic flap, thereby replacing a region of the genomic locus with the integrating nucleic acid in the cell. The DNA-binding protein may include an endonuclease. The endonuclease may include an RNA-guided endonuclease. In some aspects, the integrating nucleic acid comprises a double-stranded DNA duplex region. In some aspects, the double-stranded DNA comprises a 3′ overhang optionally comprising the first guide binding site, and comprising the first flap binding site. In some aspects, the double stranded DNA comprises a 3′ overhang optionally comprising the second guide binding site, and comprising the second flap binding site.

The DNA-binding protein may include an endonuclease. The endonuclease may include an RNA-guided endonuclease. In some aspects, the at least one DNA-binding protein comprises a Cas protein or a functional fragment thereof. In some aspects, the Cas protein or the functional fragment thereof comprises nickase activity. In some aspects, the at least one DNA-binding protein comprises a Cas9 nickase or a functional fragment thereof. In some aspects, the at least one DNA ligase ligates nucleic acids bound to DNA. In some aspects, the at least one DNA ligase ligates nucleic acids bound to RNA. In some aspects, the at least one DNA ligase comprises a PBCV-1 DNA ligase. In some aspects, the at least one DNA ligase is operatively coupled to the at least one DNA-binding protein. In some aspects, the at least one DNA ligase is fused to the at least one DNA-binding protein as a fusion polypeptide. In some aspects, the at least one DNA-binding protein and the at least one DNA ligase each comprises a heterodimer domain. In some aspects, the at least one DNA-binding protein and the at least one DNA ligase forms a heterodimer via the heterodimer domain. In some aspects, the at least one DNA-binding protein comprises a linker. In some aspects, the linker connects the Cas protein or a functional fragment thereof to the heterodimer domain. In some aspects, the at least one DNA-binding protein comprises a localization signal sequence. In some aspects, the at least one DNA ligase comprises a localization signal sequence. In some aspects, the localization signal sequence comprises a nuclear localization sequence (NLS). In some aspects, the a least one DNA-binding protein or the at least one DNA ligase are directed to nucleus of the cell by the NLS. In some aspects, the at least one integrating nucleic acid corrects at least one genetic mutation in the at least one genomic locus. In some aspects, the at least one integrating nucleic acid inserts a coding sequence. In some aspects, the coding sequence encodes a full length protein. In some aspects, the at least one integrating nucleic acid inserts a non-coding sequence. In some aspects, the non-coding sequence knocks out an endogenous gene. In some aspects, the non-coding sequence comprises a regulatory element. Some aspects further include a nuclease. In some aspects, the nuclease comprises an exonuclease for digesting the genomic flap. In some aspects, the nuclease comprises a human flap endonuclease 1 (hFEN1), a human exonuclease 5 (hEXO5), a T5 exonuclease, a T7 exonuclease, an exonuclease VIII, a flap endonuclease domain of E. coli Poll, a RecJF, a Lambda exonuclease, a Xni (ExoIXI), a SaFEN (Staphylococcus aureus FEN), a nuclease BAL-31, or a fragment thereof. In some aspects, the heterologous nuclease comprises an endonuclease for digesting the genomic flap, and the endonuclease is different from the at least one DNA-binding protein. In some aspects, the at least one DNA-binding protein comprises at least one additional functional domain. In some aspects, the at least one additional functional domain comprises a chromatin modifying domain. In some aspects, the at least one additional functional domain comprises a cell penetrating peptide. In some aspects, the at least one guide nucleic acid comprises at least one nucleic acid modification. In some aspects, the at least one nucleic acid modification comprises a modification to a backbone, a sugar, a base, or a combination thereof. In some aspects, the at least one DNA-binding protein is complexed with the at least one guide nucleic acid. In some aspects, the at least one guide nucleic acid is complexed with the integrating nucleic acid. In some aspects, the at least one DNA-binding protein, the at least one guide nucleic acid, the at least one at least one DNA ligase, the integrating nucleic acid, or a combination thereof is encoded by a polynucleotide. In some aspects, the polynucleotide comprises mRNA. In some aspects, the polynucleotide comprises a vector. In some aspects, the vector comprises a viral vector. In some aspects, the at least one DNA-binding protein, the at least one guide nucleic acid, the at least one at least one DNA ligase, the integrating nucleic acid, or a combination thereof is encapsulated by at least one lipid nanoparticle. In some aspects, the cell comprises a bacterial cell, an eukaryotic cell, or a plant cell. In some aspects, the eukaryotic cell comprises a mammalian cell. Some aspects include a composition comprising the system. Some aspects include a cell comprising the system. Some aspects include a cell line comprising the cell. Some aspects include a pharmaceutical composition comprising the system. Some aspects include a pharmaceutical composition comprising the composition. Some aspects include a pharmaceutical composition comprising the cell. Some aspects include a pharmaceutically acceptable: excipient, carrier, or diluent. In some aspects, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. Some aspects include a kit comprising: the system, the composition, or the pharmaceutical composition and a container. In some aspects, include method for modifying a cell comprising contacting a cell with the system. In some aspects, include method for modifying a cell comprising contacting a cell with the composition. In some aspects, include method for modifying a cell comprising contacting a cell with the pharmaceutical composition. In some aspects, the cell is not a dividing cell. In some aspects, the integrating nucleic acid is inserted into the genomic locus of the cell independent of endogenous non-homologous end joining (NHEJ) and independent of endogenous homology-directed repair (HDR). Some aspects include a method for treating a disease or condition in subject in need thereof comprising: contacting the cell or the subject with the system, the composition, or the pharmaceutical composition; replacing a genomic locus in a cell with an integrating nucleic acid, thereby treating the disease or condition in the subject. In some aspects, the cell is not a dividing cell. In some aspects, the integrating nucleic acid is inserted into the genomic locus of the cell independent of endogenous non-homologous end joining (NHEJ) and independent of endogenous homology-directed repair (HDR).

Disclosed herein, in some aspects, are guide nucleic acids comprising: a spacer that is at least partially complementary to a genomic locus in a cell; a scaffold for complexing with a DNA-binding protein; and a donor binding site that is at least partially complementary to an integrating nucleic acid. The DNA-binding protein may include an endonuclease. The endonuclease may include an RNA-guided endonuclease. In some aspects, the guide nucleic acid comprises a flap binding site that is at least partially complementary to a genomic sequence of the genomic locus. In some aspects, the guide nucleic acid comprises at least one nucleic acid modification. In some aspects, the at least one nucleic acid modification comprises a modification to a backbone, a sugar, a base, or a combination thereof. In some aspects, the guide nucleic acid comprises RNA sequence.

### BRIEF DESCRIPTION OF THE DRAWINGS

FIG. 1A illustrates a guide nucleic acid, an endonuclease, a ligase, and a donor strand at a genomic locus.

FIG. 1B follows sequentially from FIG. 1A, and illustrates a donor strand incorporated into one side of a genomic locus, the donor strand having displaced a genomic flap.

FIG. 1C follows sequentially from FIG. 1B, and illustrates a donor strand incorporated into one side of a genomic locus, and a nick appearing where a genomic flap has been removed.

FIG. 2A illustrates 2 guide nucleic acids, 2 endonucleases, 2 ligases, and a donor strand at a genomic locus.

FIG. 2B follows sequentially from FIG. 2A, and illustrates a donor strand incorporated into a genomic locus, the donor strand having displaced 2 genomic flaps.

FIG. 2C follows sequentially from FIG. 2B, and illustrates a donor strand incorporated into a genomic locus, and 2 nicks appearing where genomic flaps have been removed.

FIG. 3A illustrates a guide nucleic acid, an endonuclease, a ligase, and a donor strand at a genomic locus.

FIG. 3B follows sequentially from FIG. 3A, and illustrates a donor strand incorporated into one side of a genomic locus, the donor strand having displaced a genomic flap.

FIG. 3C follows sequentially from FIG. 3B, and illustrates a donor strand incorporated into one side of a genomic locus, and a nick appearing where a genomic flap has been removed.

FIG. 4A illustrates 2 guide nucleic acids, 2 endonucleases, 2 ligases, and a donor strand at a genomic locus.

FIG. 4B follows sequentially from FIG. 4A, and illustrates a donor strand incorporated into a genomic locus, the donor strand having displaced 2 genomic flaps.

FIG. 4C follows sequentially from FIG. 4B, and illustrates a donor strand incorporated into a genomic locus, and 2 nicks appearing where genomic flaps have been removed.

FIG. 5A illustrates a guide nucleic acid, an endonuclease, a ligase, and a donor strand at a genomic locus.

FIG. 5B follows sequentially from FIG. 5A, and illustrates a donor strand incorporated into a genomic locus, the donor strand having displaced a genomic flap.

FIG. 5C follows sequentially from FIG. 5B, and illustrates a donor strand incorporated into one side of a genomic locus, and a nick appearing where a genomic flap has been removed.

FIG. 6A illustrates 2 guide nucleic acids, 2 endonucleases, 2 ligases, and a donor strand at a genomic locus.

FIG. 6B follows sequentially from FIG. 6A, and illustrates a donor strand incorporated into a genomic locus, the donor strand having displaced 2 genomic flaps.

FIG. 6C follows sequentially from FIG. 6B, and illustrates a donor strand incorporated into a genomic locus, and 2 nicks appearing where genomic flaps have been removed.

FIG. 7 illustrates some examples of fusion protein arrangements.

FIG. 8A illustrates an exemplary nicking and ligation pattern of an integrating nucleic acid.

FIG. 8B illustrates a DNA gel showing a pattern associated with 1-Sided Replacer 2 performed in vitro using 30 nt GBS/DBS and thermostable T4 ligase. Using a 30 nt GBS/DBS combination, a donor containing a protospacer adjacent motif (PAM) mutation, and a thermostable T4 ligase (Hi-T4, NEB), we were able to produce a final Replacer product (Lane 3) corresponding to the size of our control product (Lane 1). Replacer products were not detected in the absence of nicking Cas9 (Cas9n) (Lane 2), or in the absence of the bottom donor which serves as the splint (Lanes 4 & 5).

FIG. 8C illustrates an exemplary nucleic acid gel showing pattern associated with in vitro 1-Sided Replacer 2 using variable length GBS/DBS combinations and T4 ligase. Using regular T4 ligase (NEB), we were to produce a final Replacer product corresponding to the size of the control when using multiple GBS/DBS combinations, including no GBS/DBS, 20 nt GBS/DBS, and 30 nt GBS/DBS. Additionally, in this experiment, recoded dsDNA donors containing PAM mutation were more efficient at producing final Replacer products compared to PAM mutant dsDNA donors that were not recoded.

FIG. 9 illustrates measurement of a percentage of cells expressing green fluorescent protein (GFP), indicating gene editing from BFP to GFP by a 1-sided Replacer 2 with nicking Cas9 and DNA ligase.

FIG. 10 illustrates sequencing reads merged and aligned to an amplicon of interest and a percentage of total reads that matched an intended edit via a 1-sided replacer 2 with a nicking Cas9 and a T4 DNA ligase.

FIG. 11 illustrates sequencing reads merged and aligned to an amplicon of interest and a percentage of total reads that matched an intended edit via a 2-sided replacer 2 with a nicking Cas9 and a T4 DNA ligase.

FIG. 12 illustrates measurement of a percentage of cells expressing green fluorescent protein (GFP), indicating gene editing from BFP to GFP via a 1-Sided Replacer 2 with a nicking Cas9 and a T4 DNA Ligase.

### DETAILED DESCRIPTION

### Introduction

Recent advances in gene editing tools have enabled precision editing of genomes for therapeutic, agricultural, industrial, and research purposes. Some nuclease-based tools such as CRISPR-Cas9 use a guide RNA to target the Cas9 protein to a specific DNA sequence specified by the spacer sequence in the guide RNA. Cas9 nuclease activity then cleaves the DNA resulting in a double-stranded break (DSB). DSBs are typically repaired through endogenous DNA repair mechanisms including non-homologous end joining (NHEJ) or homology-directed repair (HDR). However, NHEJ results in a spectrum of nucleotide insertions and deletions (indels) that hinder its utility for precision editing. HDR efficiency is very low in nondividing cells and may require DNA replication. Even when HDR editing is detectable, DSB-induced indels are often prevalent, meaning that HDR may not be feasible when precision editing is desired.

Homology-independent targeted insertion (HITI) utilizes NHEJ DNA repair mechanisms active in nondividing cells for CRISPR-guided transgene integration in nondividing cells such as primary neurons, retinal pigment epithelial cells, and HSPCs. However, due to the generation of DSBs from Cas9, HITI generates high frequencies of indels, resulting in unintended mutations in addition to DSB associated toxicity.

Other methods for gene editing have additional limitations. Tools employing fusions of nicking Cas nucleases with nucleotide deaminases (e.g. base editors) can perform certain nucleotide mutations, e.g. cytosine base editors can convert C to T. While some base editors can perform precision editing at high efficiency, they are inherently limited to specific edits determined by the deaminase variant so they are only applicable to specific substitution mutations and further cannot perform precise insertion or deletion edits. Moreover, base editors are generally limited to a small editing window within a subset of the protospacer region and are therefore significantly limited by protospacer adjacent motif (PAM) availability. Finally, base editors can exhibit bystander mutations within the editing region (e.g. if two C's are present) and have demonstrated DNA and RNA off-target deaminase activity.

Existing precision editing technologies have limitations that hamper their practical applicability in a variety of ways. In particular, they may rely on endogenous cellular machinery for editing, for example HDR machinery for nuclease-based editing and mismatch repair for base editing. No system has been reported that is independent of all endogenous factors. Reliance on endogenous factors is problematic because different cell types have different activity levels of these endogenous factors, and in many cases the activity is not sufficient to provide useful levels of editing. An example where this reliance is particularly problematic is nondividing cells, which comprise the majority of cells in adults and therefore are not amenable to many existing precision editing tools.

Accordingly, there remains a need for a system or a method for effective gene editing or for modifying gene expression by gene editing. Particularly, there remains a need for the system or method for gene editing or modifying gene expression, where the system or the method do not rely on the endogenous components or mechanism of a cell. There also remains a need for a system or a method for correcting genetic mutations in a cell. In some cases, the correction of genetic mutation can treat a disease or condition in subject in need thereof. As will be seen below, the systems, methods, and compositions disclosed herein may be useful for addressing these needs or limitations.

### Overview

Described herein are self-contained gene editing systems. In some such self-contained systems, every aspect of gene editing may be controlled. Some such systems do not rely on host cell machinery to perform an editing function, or to replace or repair any aspect of a target nucleic acid such as a genomic locus. Some such systems are unaffected by a cell's nucleotide triphosphate (dNTP) concentration because the editing may be performed without use of a polymerase. For example, an integrating nucleic acid may be delivered and inserted into a genetic locus without transcribing a template. The editing may exclude a need to rely on a cell repair system such as HDR or NHEJ. The editing may be performed without cell cycling. The gene editing may take place in a cell, or may even be performed in vitro. For example, the gene editing may even be performed in a test tube or outside of a cell.

Described herein are systems and methods for editing DNA with a donor strand without generating a double-stranded break in the genome using CRISPR-guided DNA ligases and guide nucleic acids targeting the genomic region of interest. DNA ligases are enzymes which chemically join two DNA molecules via a phosphodiester bond. DNA ligases may or may not require hybridization of the DNA molecules to a DNA or RNA backbone or “splint” which is reverse complementary to the DNA sequences that are to be ligated. Targeting of ligases to genomic nicks generated by CRISPR nucleases enables precise replacement of genomic DNA with donor strands optionally recruited by guide nucleic acids into targeted loci. The CRISPR-guided DNA ligases can be composed of DNA ligases that are fused, recruited, or unfused to the RNA-guided endonuclease by utilizing peptide linkers, heterodimerization domains, or two separate peptides, respectively.

Some aspects include a cell containing or comprising an RNA-guided endonuclease and a DNA ligase, both of which are introduced into the cell. The endonuclease or ligase may be heterologous to the cell. The endonuclease and ligase may be heterologous to the cell. The ligase may be endogenous to the cell. In some aspects, a cell comprises an RNA-guided endonuclease and a DNA ligase, both of which are heterologous to the cell. The cell may include a composition or system described herein. The cell may be used or included in a system, composition, or method described herein.

A system described herein may include a heterologous endonuclease comprising an RNA-guided endonuclease such as nicking Cas9 as well as a heterologous ligase (e.g., a DNA ligase) that can utilize an RNA splint. The guide nucleic acid optionally recruits a donor strand to the site targeted by the endonuclease (e.g., a targeted genomic locus) and also generates a splint across from the donor strand (donor strand) and genomic flap generated by the nicking Cas9, resulting in ligation of the donor strand and the genomic flap by the DNA ligase. In some embodiments, the ligase is or comprises an endogenous ligase. The system can utilize one or more guide nucleic acids that together can comprise the following components, optionally in the following order: 5′ spacer—scaffold—donor binding site (optional)—flap binding site 3′. The donor strand (donor strand) can comprise the following sequence components: 5′ guide binding site—donor strand 3′. The guide binding site of the donor strand is at least partially reverse complementary to the donor binding site of the guide nucleic acid such that the donor hybridizes to the guide and is localized to the target site of the RNA guided endonuclease. The 5′ end of the donor sequence and the 3′ end of the genomic flap generated by nuclease nicking activity are ligated by the DNA ligase, splinted by the donor binding site and a flap binding site of the guide nucleic acid(s).

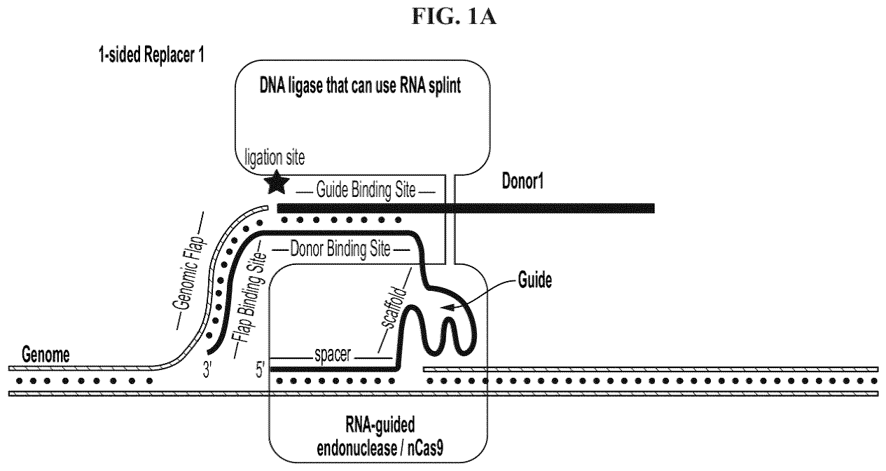

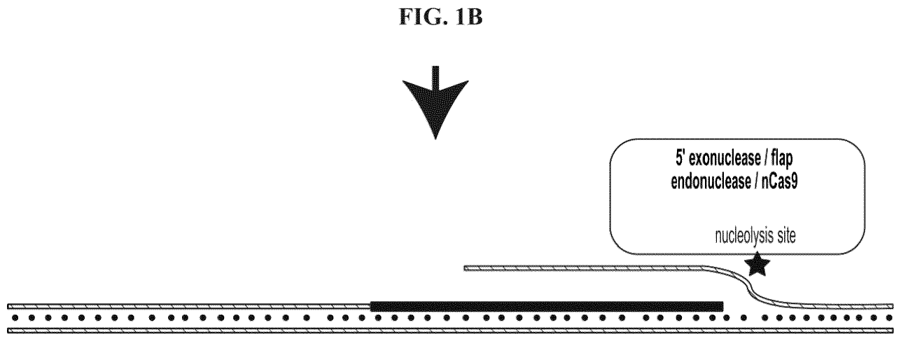

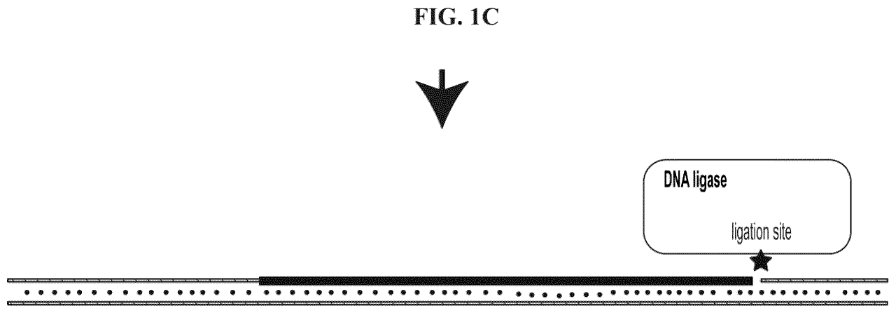

FIG. 1A-1C illustrate a non-limiting example of a system (1-sided Replacer 1). The example includes a guide nucleic acid comprising: a spacer for targeting a genomic locus; a scaffold for complexing and recruiting an endonuclease described herein; a donor binding site for complexing with a donor strand; and a flap binding site for complexing with a genomic flap of the genomic locus. The guide nucleic acid is shown complexed with an endonuclease (e.g., a Cas9 nickase, nCas9) operatively coupled to a ligase. The guide nucleic acid may direct the endonuclease to a genomic locus that is bound by the spacer of the guide nucleic acid. The guide nucleic acid is also shown as partially complementary to a donor strand (complexing between the donor binding site of the guide nucleic acid and guide binding site of the donor strand). The endonuclease, when directed by the guide nucleic acid, can cleave or nick at least one strand of the genomic locus, and the ligase can ligate one end of the donor strand with the cleaved or nicked end of the genomic locus, thus incorporating the donor strand into the genomic locus. The incorporation of the donor strand into the genomic locus may generate a genomic flap that can be digested and removed by a nuclease.

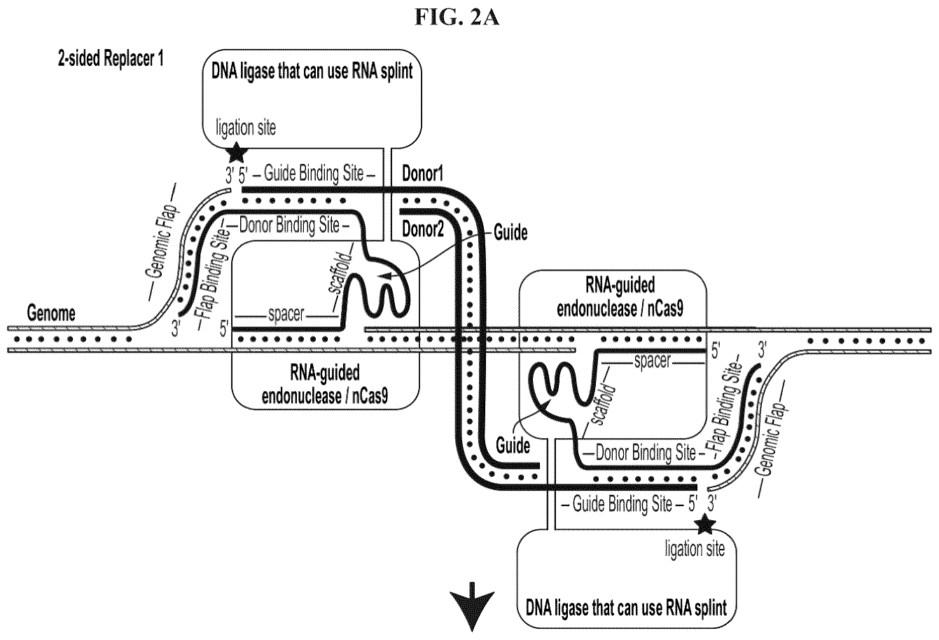

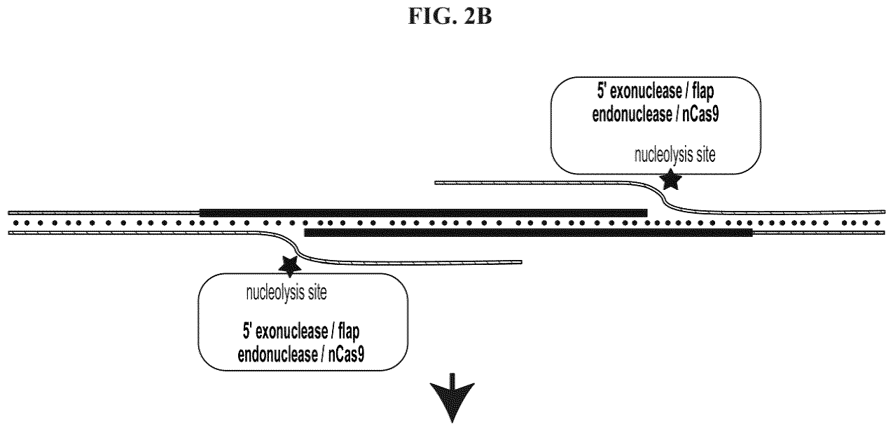

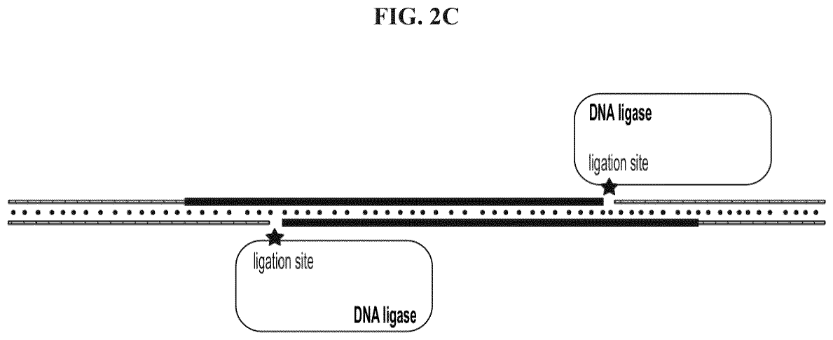

FIG. 2A-2C illustrate a non-limiting example of a system (2-sided Replacer 1). The guide nucleic acid in the example, similar to the guide nucleic acid of FIG. 1A, comprises: a spacer for targeting a genomic locus; a scaffold for complexing and recruiting an endonuclease described herein; a donor binding site for complexing with a donor strand; and a flap binding site for complexing with a genomic flap of the genomic locus. In FIG. 2A, a first guide nucleic acid is shown complexed with a first endonuclease operatively coupled with a first ligase and a second guide nucleic acid is complexed with a second endonuclease operatively coupled with a second ligase. The first endonuclease and the second nuclease may each cleave at least one strand of the genomic locus. The two cleaved ends of the genomic locus can then be ligated to the two ends of the donor strand, thereby incorporating the donor strand into the genomic locus. The insertion of the donor strand at the genomic locus may generate two genomic flaps that can be digested and removed by a nuclease.

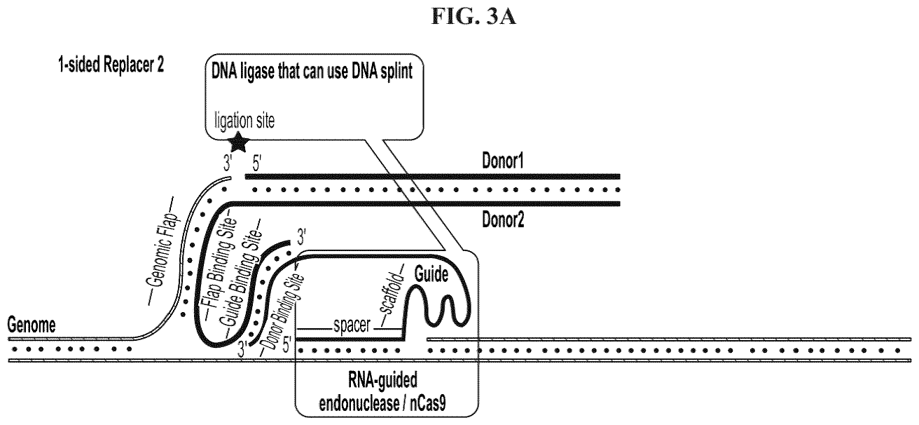

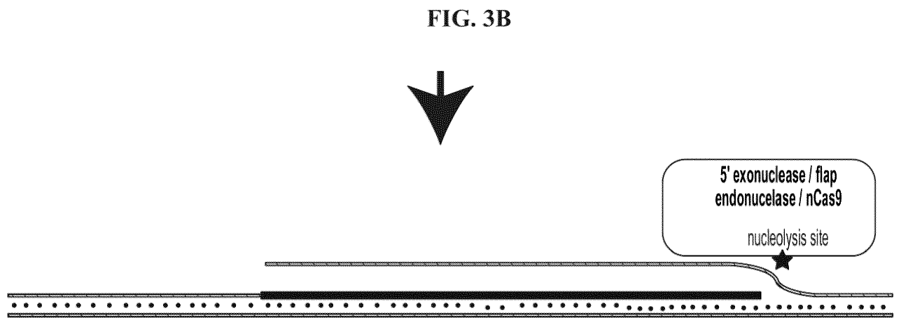

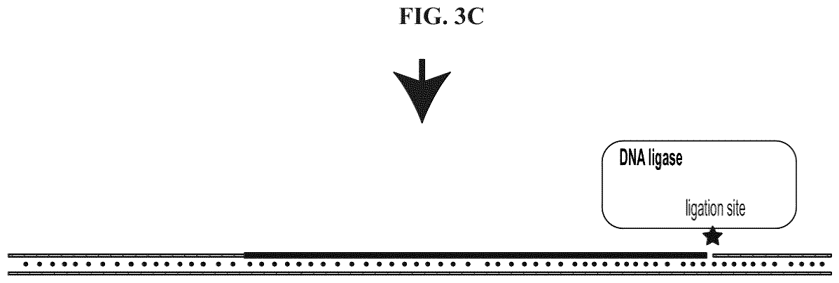

FIG. 3A-3C illustrate a non-limiting example of a system (1-sided Replacer 2). In the example, a guide nucleic acid comprises: a spacer for targeting a genomic locus; a scaffold for complexing and recruiting an endonuclease described herein; and a donor binding site for complexing with a donor strand. Also shown in FIG. 3A is a donor strand comprising at least one overhang, where the overhang comprises: a flap binding site for complexing with a genomic flap of the genomic locus; and a guide binding site for complexing with the guide nucleic acid (via the donor binding site of the guide nucleic acid). The guide nucleic acid can be complexed with an endonuclease (e.g., nCas9) operatively coupled to a ligase. The guide nucleic acid in the example directs the endonuclease and the ligase to a genomic locus that is bound by the spacer of the guide nucleic acid. The guide nucleic acid in the example is also partially complementary to a donor strand (complexing between the donor binding site of the guide nucleic acid and guide binding site of the donor strand). The endonuclease, when directed by the guide nucleic acid, can cleave at least one strand of the genomic locus, and the ligase can ligate one end of the donor strand with the cleaved end of the genomic locus, thus incorporating the donor strand into the genomic locus. The incorporation of the donor strand into the genomic locus may generate a genomic flap that can be digested and removed by a nuclease.

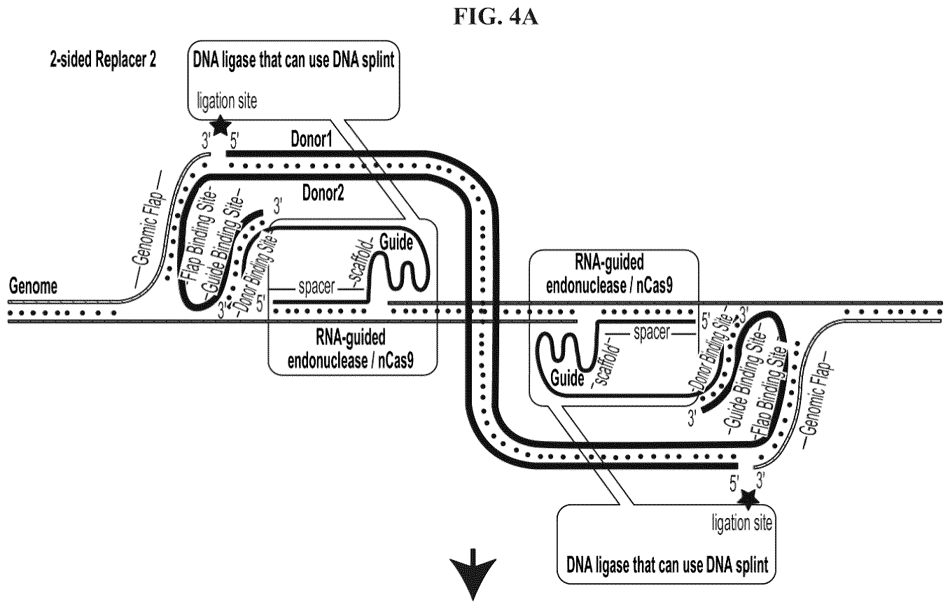

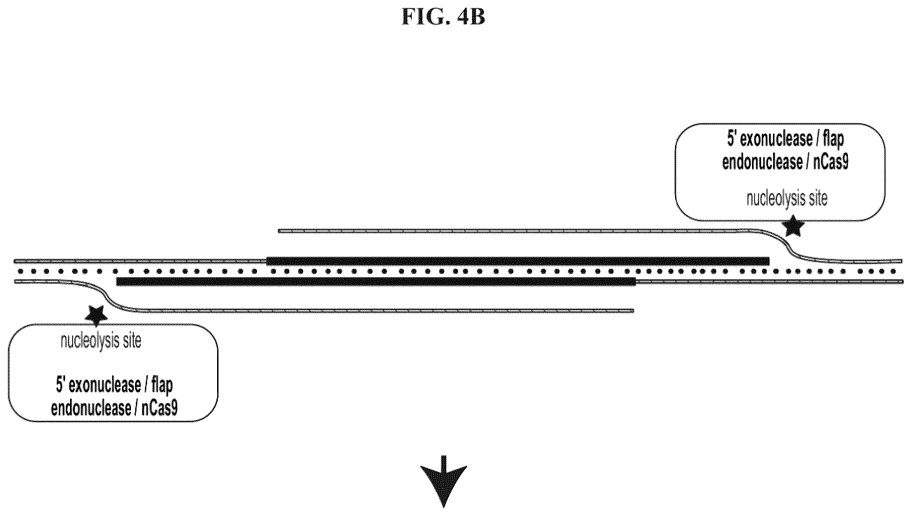

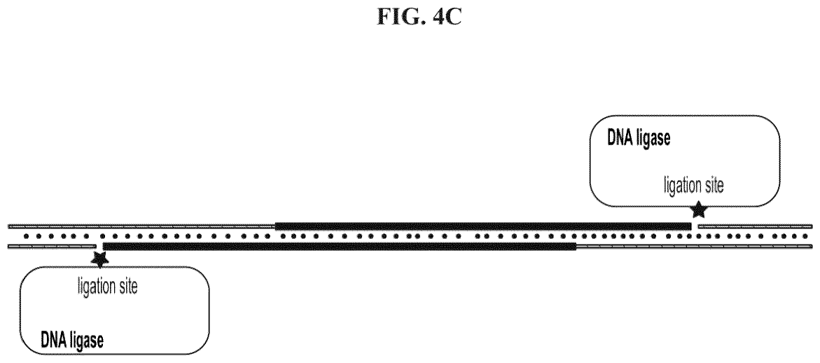

FIG. 4A-4C illustrates a non-limiting example of a system (2-sided Replacer 2). In the example, where the guide nucleic acid, similar to the guide nucleic acid of FIG. 3A, comprises a spacer for targeting a genomic locus; a scaffold for complexing and recruiting an endonuclease described herein; and a donor binding site for complexing with a donor strand. Also shown in FIG. 4A is a donor strand comprising two overhangs, where the overhangs each comprise a flap binding site for complexing with a genomic flap of the genomic locus; and a guide binding site for complexing with a guide nucleic acid (via a donor binding site of the guide nucleic acid). The flap binding site of the donor strand can bring the donor strand in close proximity with the genomic locus after a genomic flap is generated after the endonuclease cleaves at least one strand of the genomic locus. In FIG. 4A, a first guide nucleic acid is shown complexed with a first endonuclease operatively coupled with a first ligase and a second guide nucleic acid is complexed with a second endonuclease operatively coupled with a second ligase. In the example, the first endonuclease and the second nuclease each cleave at least one strand of the genomic locus. The two cleaved ends of the genomic locus can then be ligated to the two ends of the donor strand, thereby incorporating the donor strand into the genomic locus. In the example, the insertion of the donor strand at the genomic locus generates two genomic flaps that can be digested and removed by a nuclease.

A system described herein (Replacer 3) may include a heterologous endonuclease comprising an RNA-guided endonuclease such as nicking Cas9 as well as a ligase (e.g., a DNA ligase) that can utilize a DNA splint. The guide nucleic acid optionally recruits a donor strand to the site targeted by the endonuclease (e.g., a targeted genomic locus) and also generates a splint across from the donor strand (donor strand) and genomic flap generated by the nicking Cas9, resulting in ligation of the donor strand and the genomic flap by the DNA ligase. At least part of the flap binding site and donor binding site on the guide nucleic acid are DNA such that ligases that utilize DNA splints are able to catalyze the intended reaction. The system can utilize one or more guide nucleic acids that together can comprise the following components, optionally in the following order: 5′ spacer—scaffold—donor binding site (optional)—flap binding site 3′. The donor strand (donor strand) can comprise the following sequence components: 5′ guide binding site—donor strand 3′. The guide binding site of the donor strand is at least partially reverse complementary to the donor binding site of the guide nucleic acid such that the donor hybridizes to the guide and is localized to the target site of the RNA guided endonuclease. The 5′ end of the donor sequence and the 3′ end of the genomic flap generated by nuclease nicking activity are ligated by the DNA ligase, splinted by the donor binding site and a flap binding site of the guide nucleic acid(s).

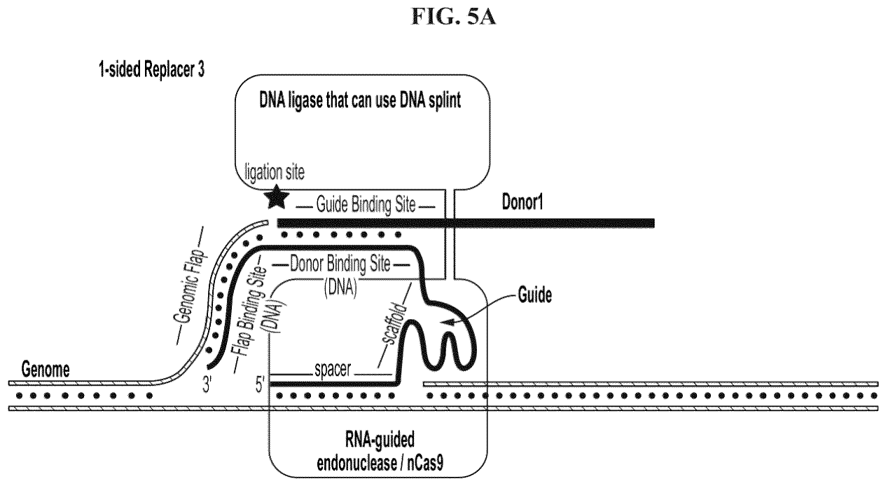

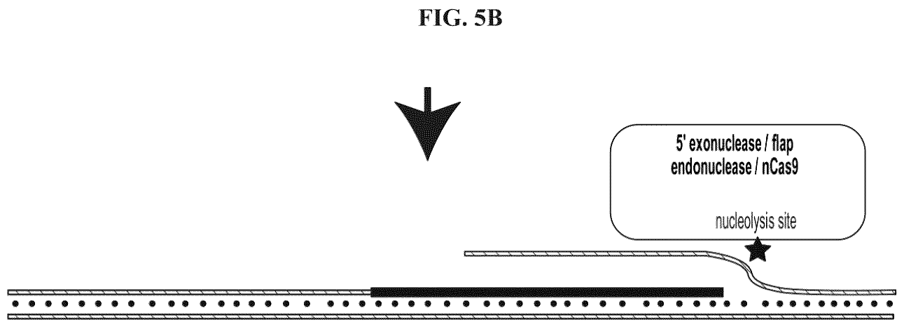

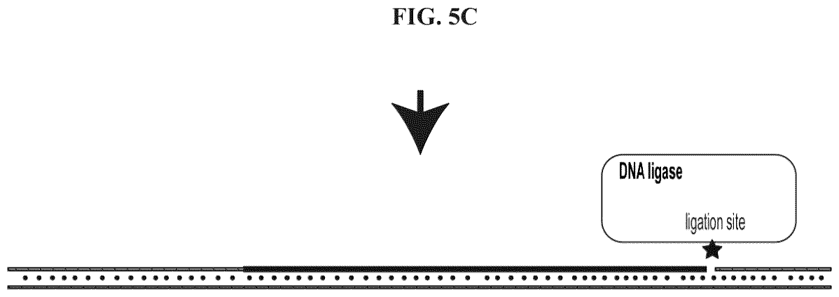

FIG. 5A-5C illustrate a non-limiting example of a system (1-sided Replacer 3). The example includes a guide nucleic acid comprising: a spacer for targeting a genomic locus; a scaffold for complexing and recruiting an endonuclease described herein; a donor binding site for complexing with a donor strand; and a flap binding site for complexing with a genomic flap of the genomic locus, wherein at least part of the flap binding site and donor binding site are comprised of DNA. The guide nucleic acid is shown complexed with an endonuclease (e.g., a Cas9 nickase, nCas9) operatively coupled to a ligase (e.g., an endogenous ligase or an exogenous ligase). The guide nucleic acid may direct the endonuclease to a genomic locus that is bound by the spacer of the guide nucleic acid. The guide nucleic acid is also shown as partially complementary to a donor strand (complexing between the donor binding site of the guide nucleic acid and guide binding site of the donor strand). The endonuclease, when directed by the guide nucleic acid, can cleave at least one strand of the genomic locus, and the ligase can ligate one end of the donor strand with the cleaved end of the genomic locus, thus incorporating the donor strand into the genomic locus. The incorporation of the donor strand into the genomic locus may generate a genomic flap that can be digested and removed by a nuclease.

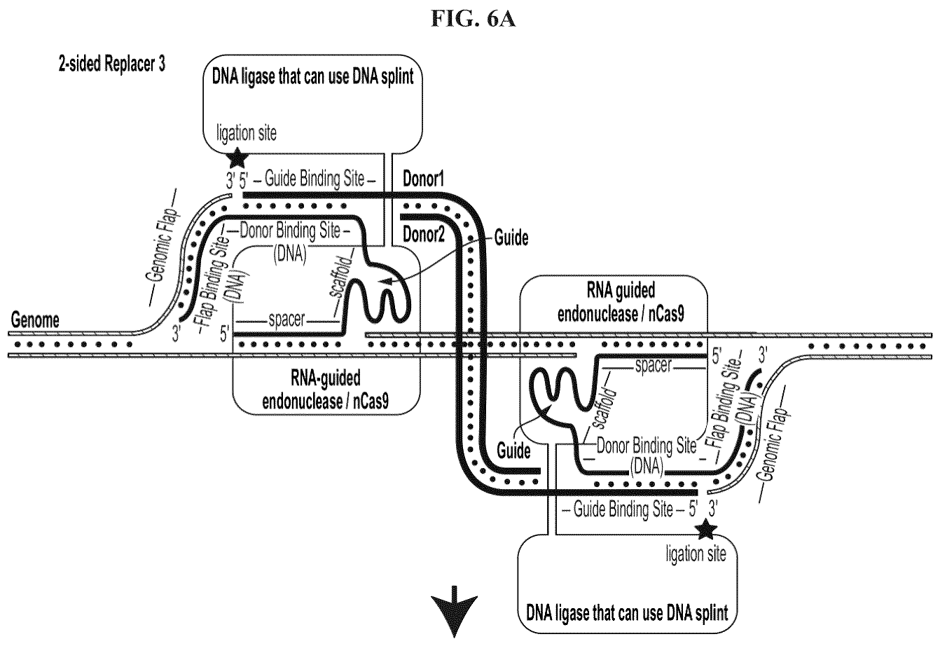

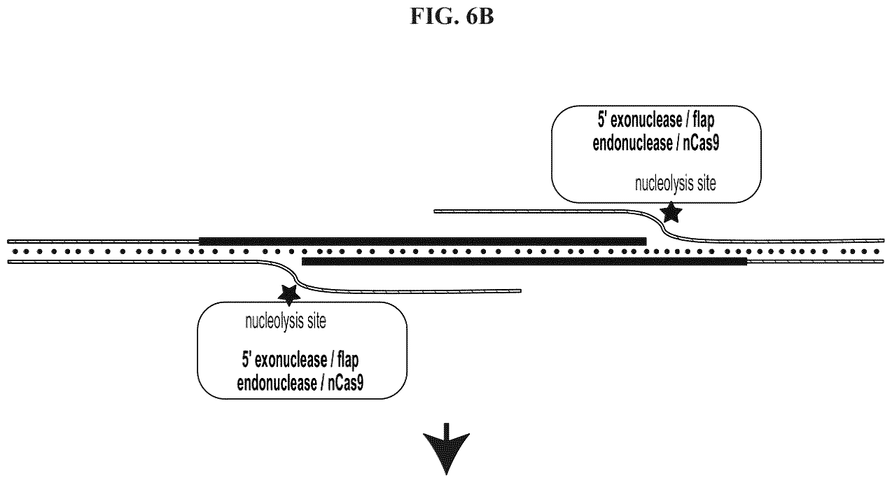

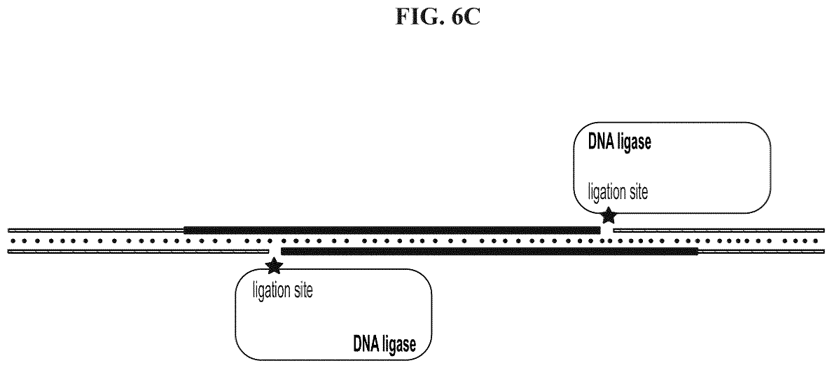

FIG. 6A-6C illustrate a non-limiting example of a system (2-sided Replacer 3). The guide nucleic acid in the example, similar to the guide nucleic acid of FIG. 5A, comprises: a spacer for targeting a genomic locus; a scaffold for complexing and recruiting an endonuclease described herein; a donor binding site for complexing with a donor strand; and a flap binding site for complexing with a genomic flap of the genomic locus, wherein at least part of the flap binding site and donor binding site are comprised of DNA. In FIG. 6A, a first guide nucleic acid is shown complexed with a first endonuclease operatively coupled with a first ligase and a second guide nucleic acid is complexed with a second endonuclease operatively coupled with a second ligase. The first endonuclease and the second nuclease may each cleave at least one strand of the genomic locus. The two cleaved ends of the genomic locus can then be ligated to the two ends of the donor strand, thereby incorporating the donor strand into the genomic locus. The insertion of the donor strand at the genomic locus may generate two genomic flaps that can be digested and removed by a nuclease.

Ligation may be performed using a DNA ligase that can utilize an RNA splint such as SplintR ligase—also known as PBCV-1 DNA Ligase—from Chlorella virus. In some aspects, the system utilizes two guide nucleic acids targeting the CRISPR-guided ligase to target sites on opposite strands flanking the genomic region of interest. In some aspects, each guide nucleic acid interacts with a corresponding donor strand in the manner described above, resulting in ligation of both donor strands which are reverse complementary with each other in the donor strand regions.

A ligase that is fused or recruited to an endonuclease, or supplied in trans, can utilize DNA as a splint, and a donor strand acts as the splint for the genomic flap generated by the endonuclease and another donor strand. In some aspects, the donor strand comprises: 5′ donor strand—flap binding site—guide binding site (optional) 3′. The flap binding site on one donor strand (Donor2) can be reverse complementary to the genomic flap, while the optional guide binding site on Donor2 is reverse complementary to the optional donor binding site of a guide nucleic acid (Guide1), and the donor strand can be at least partially reverse complementary to a different donor strand (Donor1). The 5′ end of this Donor1 and the 3′ end of the genomic flap can be ligated using the flap binding site and donor strand of the Donor2 as a splint. Such 2-sided approach utilizing dual guide nucleic acids with different spacer sequences can be adopted with Donor2, which provides the splint at the first genomic site and can be ligated on its 5′ end to a 3′ end of a different genomic flap at a nick created using a second Replacer2 guide nucleic acid (Guide2) with a spacer sequence that targets a second site. The donor binding site on the second guide nucleic acid system can optionally recruit Donor1 via hybridization with its optional guide binding site, and the Donor1 acts as the DNA splint for ligation of Donor2 to the 3′ end of the genomic flap at the target site of the second guide nucleic acid.

Following ligation, the remaining flaps of native genomic DNA can be excised via exogenously delivered or endogenous flap endonucleases or exonucleases. Examples of exogenous nucleases that can be introduced into the cell include human flap endonuclease 1 (hFEN1), human exonuclease 5 (hEXO5), T5 exonuclease, T7 exonuclease, exonuclease VIII, the flap endonuclease domain of E. coli Poll, RecJF, Lambda exonuclease, Xni (ExoIXI) from Escherichia coli, SaFEN (Staphylococcus aureus FEN), nuclease BAL-31, or fragments thereof. The endonucleases or exonucleases can optionally be fused, recruited, or unfused to the RNA-guided endonuclease or DNA ligase by utilizing peptide linkers, heterodimerization domains, or two separate peptides, respectively.

In some aspects, the system, composition, or method described herein utilizes additional protein that binds to the cleaved or nicked site. For example, the system, composition, or method described herein can include Ku protein or Gam protein from bacteriophage Mu, where the binding of the Ku protein or Gam protein can increase ligation efficiency of the integration nucleic acid at the cleaved or nicked site.

A system or method described herein may use a nicking endonuclease and, therefore, does not generate double stranded breaks. Furthermore, the system described herein addresses the issue of poor editing efficiencies in nondividing cells through a mechanism of action which only depends on the exogenous components delivered to the cells using mRNA, viral vectors, guide nucleic acids, DNA, or peptides, or any other modalities. Therefore, the system does not require the presence of cell cycle-dependent endogenous cell processes or components such as HDR or dNTPS. As such, the system described herein allows efficiency that is not hindered in nondividing cells. Furthermore, the system enables replacement of both strands of a targeted region of the genome, which can increase editing efficiency.

A donor strand may contain a high degree of homology with the replaced genomic DNA. These donors may contain mutations to the genomic DNA such as pathogenic mutation correction, disabling of CRISPR protospacer adjacent motif (PAM) sites, disruption of the guide's spacer sequences, other substitution mutations, or a combination thereof. Additional substitution mutations may be included to increase donor-donor homology versus donor-genome homology to promote hybridization of donor strands and incorporation into the genome. Donor strands may also encode deletions or insertions of nucleotides, or may encode a complex combination of the above which then replaces the target genomic DNA. Optionally, guide and donor strands may be chemically modified using nucleic acid chemistries such as phosphorothioate bonds or 2′-O-methylation. Optionally, guide nucleic acids may include hairpin sequences. Optionally, any combination of guide nucleic acids, donor strands, and proteins can be complexed, using an annealing reaction (gradual reduction in temperature) for example, prior to delivering the editing components to the cell.

Protein components (e.g. nicking Cas9, ligase) may be modified using nuclear localization signals, cell penetrating peptides, or chromatin disrupting peptides in order to improve delivery efficiency to genomic targets.

The predominant cellular DNA repair pathway for resolving small (<13 nt) mismatches between genomic DNA strands is mismatch repair (MMR). For single stranded donor ligation, the ligated donor strand forms a DNA heteroduplex with the reverse complementary genomic DNA strand. This may also occur with competitive hybridization between ligated donor strand strands and genomic DNA strands. In these cases, MMR activity can excise and revert mismatches in the donor strand using the genomic strand as a template, resulting in reduced editing. Expression of dominant negative versions of MMR proteins has been shown to inhibit the MMR pathway and improve editing outcome in cases where similar DNA heteroduplexes are generated. In some aspects, dominant negative MMR peptides such as MSH2 (G674A) and MLH1 (de1754-756) may be delivered as part of the system described herein to improve genomic editing capability, particularly in cells which overexpress the MMR pathway. In some aspects, these dominant negative MMR peptides can be delivered as a fusion (e.g., fused with any component of the system described herein), recruited, or as separate peptides.

### Endonucleases

Disclosed herein are endonucleases. The endonuclease may be included in a composition, system or method disclosed herein. The endonuclease may be recombinant. The endonuclease may be coupled to a ligase. The endonuclease may be coupled directly or indirectly to the ligase. The coupling may be covalent or non-covalent. The endonuclease may be bound or connected to a ligase. The endonuclease may be recruited to, be part of a fusion protein with, or be used in conjunction with the ligase. The endonuclease may be heterologous. Heterologous may indicate a source from without a cell. Where a heterologous endonuclease is described, a non-heterologous (e.g. endogenous) endonuclease may be used in some instances. The endonuclease may be encoded in a cell. The endonuclease may be delivered to the cell in trans. The endonuclease may catalyze cleavage of a phosphate bond within an integrating nucleic acid. The endonuclease may be guided by a guide nucleic acid to cleave or nick a target nucleic acid for ligation of an integrating nucleic acid at the cleavage or nick site. The endonuclease may include any aspect included in FIG. 1A-6C.

The endonuclease may be non-naturally occurring. The endonuclease may be engineered. The endonuclease may be synthetic. The endonuclease may be pre-synthetized. The endonuclease may be added to a subject or a cell. The endonuclease may be encoded by a nucleic acid. The encoding nucleic acid may be engineered, synthetic, or added to a subject or a cell.

At least part of the endonuclease may be included in a first polypeptide. At least part of the endonuclease may be included in a second polypeptide. The endonuclease may be split into two or more polypeptides bound together. The first polypeptide may include an N-terminal portion of the endonuclease. The first polypeptide may include a C-terminal portion of the endonuclease. The second polypeptide may include the N-terminal portion of the endonuclease. The second polypeptide may include the C-terminal portion of the endonuclease. The first or second polypeptide comprising a part of the endonuclease may be fused with at least part, or the whole, of the ligase.

Described herein, in some aspects, is a system comprising at least one endonuclease. In some aspects, the endonuclease is a programmable endonuclease, where the endonuclease can be complexed with and directed by a guide nucleic acid described herein to a genomic locus. The endonuclease may bind DNA. In some aspects, the endonuclease is a RNA-guided endonuclease. In some aspects, the endonuclease can introduce a single-stranded break. Examples of RNA-guided endonucleases can include CRISPR/Cas endonucleases (e.g., class 2 CRISPR/Cas endonucleases such as a type II, type V, or type VI CRISPR/Cas endonucleases). A CRISPR/Cas endonuclease is also referred to as a CRISPR/Cas effector polypeptide. A suitable endonuclease is a CRISPR/Cas endonuclease (e.g., a class 2 CRISPR/Cas endonuclease such as a type II, type V, or type VI CRISPR/Cas endonuclease). In some cases, a suitable RNA-guided endonuclease is a class 2 CRISPR/Cas endonuclease. In some cases, a suitable RNA-guided endonuclease is a class 2 type II CRISPR/Cas endonuclease (e.g., a Cas9 protein). In some cases, an endonuclease includes a class 2 type V CRISPR/Cas endonuclease (e.g., a Cpf1 protein, a C2c1 protein, or a C2c3 protein). In some cases, a suitable RNA-guided endonuclease is a class 2 type VI CRISPR/Cas endonuclease (e.g., a C2c2 protein; also referred to as a “Cas13a” protein). Also suitable for use is a CasX protein. Also suitable for use is a CasY protein. In some aspects, the endonuclease can include any one of the Cas described herein complexed with a guide nucleic acid (e.g., a gRNA) as an RNP complex.

In some cases, the endonuclease is a Type II CRISPR/Cas endonuclease. In some cases, the endonuclease is a Cas9. Cas9 functions as an RNA-guided endonuclease that uses a dual-guide RNA having a crRNA and trans-activating crRNA (tracrRNA) for target recognition and cleavage by a mechanism involving two nuclease active sites in Cas9 that together generate double-stranded DNA breaks (DSBs), or can individually generate single-stranded DNA breaks (SSBs). The Type II CRISPR endonuclease Cas9 and engineered dual- (dgRNA) or single guide RNA (sgRNA) form a ribonucleoprotein (RNP) complex that can be targeted to a desired DNA sequence. Guided by a dual-RNA complex or a chimeric single-guide RNA, Cas9 generates site-specific DSBs or SSBs within double-stranded DNA (dsDNA) target nucleic acids, which are repaired either by non-homologous end joining (NHEJ) or homology-directed recombination (HDR). The Cas9 can be guided to a target site (e.g., stabilized at a target site) within a target nucleic acid sequence by virtue of its association with the RNA-binding segment of the Cas9 to guide RNA. A Cas9 protein can bind and/or modify (e.g., cleave, nick, methylate, demethylate, etc.) a target nucleic acid and/or a polypeptide associated with target nucleic acid (e.g., methylation or acetylation of a histone tail)(e.g., when the Cas9 protein includes a fusion partner with an activity). In some cases, the Cas9 protein is a naturally-occurring protein (e.g., naturally occurs in bacterial and/or archaeal cells). In other cases, the Cas9 protein is not a naturally-occurring polypeptide (e.g., the Cas9 protein is a variant Cas9 protein, a chimeric protein, and the like).

Naturally occurring Cas9 proteins may bind a Cas9 guide RNA, are thereby directed to a specific sequence within a target nucleic acid (a target site), and cleave the target nucleic acid (e.g., cleave dsDNA to generate a double strand break, cleave ssDNA, cleave ssRNA, etc.). A chimeric Cas9 protein may include a fusion protein comprising a Cas9 polypeptide fused to a heterologous protein (referred to as a fusion partner), where the heterologous protein provides an activity (e.g., one that is not provided by the Cas9 protein). The fusion partner can provide an activity, e.g., enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc.). In some cases, a portion of the Cas9 protein (e.g., the RuvC domain and/or the HNH domain) exhibits reduced nuclease activity relative to the corresponding portion of a wild type Cas9 protein (e.g., in some cases the Cas9 protein is a nickase). In some cases, the Cas9 protein is enzymatically inactive, or has reduced enzymatic activity relative to a wild-type Cas9 protein (e.g., relative to Streptococcus pyogenes Cas9). In some cases, the Cas9 is a Cas9 nickase. The Cas9 nickase can be generated by mutating a Cas9 nuclease domain. Non-limiting example of the Cas9 nickase can include SpCas9, SaCas9, CjCas9, GeoCas9, HpaCas9, and NmeCas9. In some aspects, the endonuclease described herein comprises any one of the Cas9 in Table 1. In some aspects, the endonuclease described herein comprises a polypeptide sequence that is at least 70%, at least 75%, at least 80%, at least 85%, at least 90%, at least 95%, at least 99%, or more identical to the polypeptide sequence of any one of the Cas9 in Table 1.

TABLE 1
Non-limiting examples of Cas9 polypeptide sequence

| | | SEQ ID |
|---|---|---|
| Name | Cas9 polypeptide sequence | NO: |
| SpyCas9 | MDKKYSIGLDIGTNSVGWAVITDEYKVPSKKFKVLGNTDRHSIKKNLIGALLFDSG | 1 |
| | ETAEATRLKRTARRRYTRRKNRICYLQEIFSNEMAKVDDSFFHRLEESFLVEEDKK | |
| | HERHPIFGNIVDEVAYHEKYPTIYHLRKKLVDSTDKADLRLIYLALAHMIKFRGHF | |
| | LIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLA | |
| | QIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVK | |
| | LNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKVT | |
| | VKQLKEDYFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRLSRKLINGIRD | |
| | KQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRI | |
| | EEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIE | |
| | TNGETGEIVWDKGRDFATVRKVLSMPQVNIVKKTEVQTGGFSKESILPKRNSDKLI | |
| | ARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANLD | |
| | KVLSAYNKHRDKPIREQAENIIHLFTLTNLGAPAAFKYFDTTIDRKRYTSTKEVLD | |
| | ATLIHQSITGLYETRIDLSQLGGD | |
| Nicking | MDKKYSIGLDIGTNSVGWAVITDEYKVPSKKFKVLGNTDRHSIKKNLIGALLFDSG | 2 |
| SpyCas9 | ETAEATRLKRTARRRYTRRKNRICYLQEIFSNEMAKVDDSFFHRLEESFLVEEDKK | |
| (H840A) | HERHPIFGNIVDEVAYHEKYPTIYHLRKKLVDSTDKADLRLIYLALAHMIKFRGHF | |
| | LIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLA | |
| | QIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVK | |
| | LNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKVT | |
| | VKQLKEDYFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRLSRKLINGIRD | |
| | KQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRI | |
| | EEGIKELGSQILKEHPVENTQLQNEKLYLYYLQNGRDMYVDQELDINRLSDYDVDA | |
| | IVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIE | |
| | TNGETGEIVWDKGRDFATVRKVLSMPQVNIVKKTEVQTGGFSKESILPKRNSDKLI | |
| | ARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANLD | |
| | KVLSAYNKHRDKPIREQAENIIHLFTLTNLGAPAAFKYFDTTIDRKRYTSTKEVLD | |
| | ATLIHQSITGLYETRIDLSQLGGD | |
| Nicking | MDKKYSIGLDIGTNSVGWAVITDEYKVPSKKFKVLGNTDRHSIKKNLIGALLFDSG | 3 |
| SpyCas9 | ETAEATRLKRTARRRYTRRKNRICYLQEIFSNEMAKVDDSFFHRLEESFLVEEDKK | |
| (H840A) | HERHPIFGNIVDEVAYHEKYPTIYHLRKKLVDSTDKADLRLIYLALAHMIKFRGHF | |
| R221K | LIEGDLNPDNSDVDKLFIQLVQTYNQLFEENPINASGVDAKAILSARLSKSRKLEN | |
| N394K | LIAQLPGEKKNGLFGNLIALSLGLTPNFKSNFDLAEDAKLQLSKDTYDDDLDNLLA | |
| | QIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVK | |
| | LKREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKVT | |
| | VKQLKEDYFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRLSRKLINGIRD | |
| | KQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRI | |
| | EEGIKELGSQILKEHPVENTQLQNEKLYLYYLQNGRDMYVDQELDINRLSDYDVDA | |
| | IVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIE | |
| | TNGETGEIVWDKGRDFATVRKVLSMPQVNIVKKTEVQTGGFSKESILPKRNSDKLI | |
| | ARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANLD | |
| | KVLSAYNKHRDKPIREQAENIIHLFTLTNLGAPAAFKYFDTTIDRKRYTSTKEVLD | |
| | ATLIHQSITGLYETRIDLSQLGGD | |
| Nicking | MDKKYSIGLAIGTNSVGWAVITDEYKVPSKKFKVLGNTDRHSIKKNLIGALLFDSG | 4 |
| SpyCas9 | ETAEATRLKRTARRRYTRRKNRICYLQEIFSNEMAKVDDSFFHRLEESFLVEEDKK | |
| (D10A) | HERHPIFGNIVDEVAYHEKYPTIYHLRKKLVDSTDKADLRLIYLALAHMIKFRGHF | |
| | LIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLA | |
| | QIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVK | |
| | LNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKVT | |
| | VKQLKEDYFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRLSRKLINGIRD | |
| | KQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRI | |
| | EEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIE | |
| | TNGETGEIVWDKGRDFATVRKVLSMPQVNIVKKTEVQTGGFSKESILPKRNSDKLI | |
| | ARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANLD | |
| | KVLSAYNKHRDKPIREQAENIIHLFTLTNLGAPAAFKYFDTTIDRKRYTSTKEVLD | |
| | ATLIHQSITGLYETRIDLSQLGGD | |
| SauCas9 | MKRNYILGLDIGITSVGYGIIDYETRDVIDAGVRLFKEANVENNEGRRSKRGARRL | 5 |
| | KRRRRHRIQRVKKLLFDYNLLTDHSELSGINPYEARVKGLSQKLSEEEFSAALLHL | |
| | AKRRGVHNVNEVEEDTGNELSTKEQISRNSKALEEKYVAELQLERLKKDGEVRGSI | |
| | NRFKTSDYVKEAKQLLKVQKAYHQLDQSFIDTYIDLLETRRTYYEGPGEGSPFGWK | |
| | DIKEWYEMLMGHCTYFPEELRSVKYAYNADLYNALNDLNNLVITRDENEKLEYYEK | |
| | FQIIENVFKQKKKPTLKQIAKEILVNEEDIKGYRVTSTGKPEFTNLKVYHDIKDIT | |
| | ARKEIIENAELLDQIAKILTIYQSSEDIQEELTNLNSELTQEEIEQISNLKGYTGT | |
| | HNLSLKAINLILDELWHTNDNQIAIFNRLKLVPKKVDLSQQKEIPTTLVDDFILSP | |
| | VVKRSFIQSIKVINAIIKKYGLPNDIIIELAREKNSKDAQKMINEMQKRNRQTNER | |
| | IEEIIRTTGKENAKYLIEKIKLHDMQEGKCLYSLEAIPLEDLLNNPFNYEVDHIIP | |
| | RSVSFDNSFNNKVLVKQEENSKKGNRTPFQYLSSSDSKISYETFKKHILNLAKGKG | |
| | RISKTKKEYLLEERDINRFSVQKDFINRNLVDTRYATRGLMNLLRSYFRVNNLDVK | |
| | VKSINGGFTSFLRRKWKFKKERNKGYKHHAEDALIIANADFIFKEWKKLDKAKKVM | |
| | ENQMFEEKQAESMPEIETEQEYKEIFITPHQIKHIKDFKDYKYSHRVDKKPNRELI | |
| | NDTLYSTRKDDKGNTLIVNNLNGLYDKDNDKLKKLINKSPEKLLMYHHDPQTYQKL | |
| | KLIMEQYGDEKNPLYKYYEETGNYLTKYSKKDNGPVIKKIKYYGNKLNAHLDITDD | |
| | YPNSRNKVVKLSLKPYRFDVYLDNGVYKFVTVKNLDVIKKENYYEVNSKCYEEAKK | |
| | LKKISNQAEFIASFYNNDLIKINGELYRVIGVNNDLLNRIEVNMIDITYREYLENM | |
| | NDKRPPRIIKTIASKTQSIKKYSTDILGNLYEVKSKKHPQIIKKG* | |
| Nicking | MKRNYILGLDIGITSVGYGIIDYETRDVIDAGVRLFKEANVENNEGRRSKRGARRL | 6 |
| SauCas9 | KRRRRHRIQRVKKLLFDYNLLTDHSELSGINPYEARVKGLSQKLSEEEFSAALLHL | |
| (N580A) | AKRRGVHNVNEVEEDTGNELSTKEQISRNSKALEEKYVAELQLERLKKDGEVRGSI | |
| | NRFKTSDYVKEAKQLLKVQKAYHQLDQSFIDTYIDLLETRRTYYEGPGEGSPFGWK | |
| | DIKEWYEMLMGHCTYFPEELRSVKYAYNADLYNALNDLNNLVITRDENEKLEYYEK | |
| | FQIIENVFKQKKKPTLKQIAKEILVNEEDIKGYRVTSTGKPEFTNLKVYHDIKDIT | |
| | ARKEIIENAELLDQIAKILTIYQSSEDIQEELTNLNSELTQEEIEQISNLKGYTGT | |
| | HNLSLKAINLILDELWHTNDNQIAIFNRLKLVPKKVDLSQQKEIPTTLVDDFILSP | |
| | VVKRSFIQSIKVINAIIKKYGLPNDIIIELAREKNSKDAQKMINEMQKRNRQTNER | |
| | IEEIIRTTGKENAKYLIEKIKLHDMQEGKCLYSLEAIPLEDLLNNPFNYEVDHIIP | |
| | RSVSFDNSFNNKVLVKQEEASKKGNRTPFQYLSSSDSKISYETFKKHILNLAKGKG | |
| | RISKTKKEYLLEERDINRFSVQKDFINRNLVDTRYATRGLMNLLRSYFRVNNLDVK | |
| | VKSINGGFTSFLRRKWKFKKERNKGYKHHAEDALIIANADFIFKEWKKLDKAKKVM | |
| | ENQMFEEKQAESMPEIETEQEYKEIFITPHQIKHIKDFKDYKYSHRVDKKPNRELI | |
| | NDTLYSTRKDDKGNTLIVNNLNGLYDKDNDKLKKLINKSPEKLLMYHHDPQTYQKL | |
| | KLIMEQYGDEKNPLYKYYEETGNYLTKYSKKDNGPVIKKIKYYGNKLNAHLDITDD | |
| | YPNSRNKVVKLSLKPYRFDVYLDNGVYKFVTVKNLDVIKKENYYEVNSKCYEEAKK | |
| | LKKISNQAEFIASFYNNDLIKINGELYRVIGVNNDLLNRIEVNMIDITYREYLENM | |
| | NDKRPPRIIKTIASKTQSIKKYSTDILGNLYEVKSKKHPQIIKKG* | |
| KKH- | MGKRNYILGLDIGITSVGYGIIDYETRDVIDAGVRLFKEANVENNEGRRSKRGARR | 7 |
| SaCas9 | LKRRRRHRIQRVKKLLFDYNLLTDHSELSGINPYEARVKGLSQKLSEEEFSAALLH | |
| | LAKRRGVHNVNEVEEDTGNELSTKEQISRNSKALEEKYVAELQLERLKKDGEVRGS | |
| | INRFKTSDYVKEAKQLLKVQKAYHQLDQSFIDTYIDLLETRRTYYEGPGEGSPFGW | |
| | KDIKEWYEMLMGHCTYFPEELRSVKYAYNADLYNALNDLNNLVITRDENEKLEYYE | |
| | KFQIIENVFKQKKKPTLKQIAKEILVNEEDIKGYRVTSTGKPEFTNLKVYHDIKDI | |
| | TARKEIIENAELLDQIAKILTIYQSSEDIQEELTNLNSELTQEEIEQISNLKGYTG | |
| | THNLSLKAINLILDELWHTNDNQIAIFNRLKLVPKKVDLSQQKEIPTTLVDDFILS | |
| | PVVKRSFIQSIKVINAIIKKYGLPNDIIIELAREKNSKDAQKMINEMQKRNRQTNE | |
| | RIEEIIRTTGKENAKYLIEKIKLHDMQEGKCLYSLEAIPLEDLLNNPFNYEVDHII | |
| | PRSVSFDNSFNNKVLVKQEENSKKGNRTPFQYLSSSDSKISYETFKKHILNLAKGK | |
| | GRISKTKKEYLLEERDINRFSVQKDFINRNLVDTRYATRGLMNLLRSYFRVNNLDV | |
| | KVKSINGGFTSFLRRKWKFKKERNKGYKHHAEDALIIANADFIFKEWKKLDKAKKV | |
| | MENQMFEEKQAESMPEIETEQEYKEIFITPHQIKHIKDFKDYKYSHRVDKKPNRKL | |
| | INDTLYSTRKDDKGNTLIVNNLNGLYDKDNDKLKKLINKSPEKLLMYHHDPQTYQK | |
| | LKLIMEQYGDEKNPLYKYYEETGNYLTKYSKKDNGPVIKKIKYYGNKLNAHLDITD | |
| | DYPNSRNKVVKLSLKPYRFDVYLDNGVYKFVTVKNLDVIKKENYYEVNSKCYEEAK | |
| | KLKKISNQAEFIASFYKNDLIKINGELYRVIGVNNDLLNRIEVNMIDITYREYLEN | |
| | MNDKRPPHIIKTIASKTQSIKKYSTDILGNLYEVKSKKHPQIIKK | |
| Nicking | MGKRNYILGLDIGITSVGYGIIDYETRDVIDAGVRLFKEANVENNEGRRSKRGARR | 8 |
| KKH- | LKRRRRHRIQRVKKLLFDYNLLTDHSELSGINPYEARVKGLSQKLSEEEFSAALLH | |
| SaCas9 | LAKRRGVHNVNEVEEDTGNELSTKEQISRNSKALEEKYVAELQLERLKKDGEVRGS | |
| (N580A) | INRFKTSDYVKEAKQLLKVQKAYHQLDQSFIDTYIDLLETRRTYYEGPGEGSPFGW | |
| | KDIKEWYEMLMGHCTYFPEELRSVKYAYNADLYNALNDLNNLVITRDENEKLEYYE | |
| | KFQIIENVFKQKKKPTLKQIAKEILVNEEDIKGYRVTSTGKPEFTNLKVYHDIKDI | |
| | TARKEIIENAELLDQIAKILTIYQSSEDIQEELTNLNSELTQEEIEQISNLKGYTG | |
| | THNLSLKAINLILDELWHTNDNQIAIFNRLKLVPKKVDLSQQKEIPTTLVDDFILS | |
| | PVVKRSFIQSIKVINAIIKKYGLPNDIIIELAREKNSKDAQKMINEMQKRNRQTNE | |
| | RIEEIIRTTGKENAKYLIEKIKLHDMQEGKCLYSLEAIPLEDLLNNPFNYEVDHII | |
| | PRSVSFDNSFNNKVLVKQEEASKKGNRTPFQYLSSSDSKISYETFKKHILNLAKGK | |
| | GRISKTKKEYLLEERDINRFSVQKDFINRNLVDTRYATRGLMNLLRSYFRVNNLDV | |
| | KVKSINGGFTSFLRRKWKFKKERNKGYKHHAEDALIIANADFIFKEWKKLDKAKKV | |
| | MENQMFEEKQAESMPEIETEQEYKEIFITPHQIKHIKDFKDYKYSHRVDKKPNRKL | |
| | INDTLYSTRKDDKGNTLIVNNLNGLYDKDNDKLKKLINKSPEKLLMYHHDPQTYQK | |
| | LKLIMEQYGDEKNPLYKYYEETGNYLTKYSKKDNGPVIKKIKYYGNKLNAHLDITD | |
| | DYPNSRNKVVKLSLKPYRFDVYLDNGVYKFVTVKNLDVIKKENYYEVNSKCYEEAK | |
| | KLKKISNQAEFIASFYKNDLIKINGELYRVIGVNNDLLNRIEVNMIDITYREYLEN | |
| | MNDKRPPHIIKTIASKTQSIKKYSTDILGNLYEVKSKKHPQIIKK | |
| CjCas9 | MARILAFDIGISSIGWAFSENDELKDCGVRIFTKVENPKTGESLALPRRLARSARK | 9 |
| | RLARRKARLNHLKHLIANEFKLNYEDYQSFDESLAKAYKGSLISPYELRFRALNEL | |
| | LSKQDFARVILHIAKRRGYDDIKNSDDKEKGAILKAIKQNEEKLANYQSVGEYLYK | |
| | EYFQKFKENSKEFTNVRNKKESYERCIAQSFLKDELKLIFKKQREFGFSFSKKFEE | |
| | EVLSVAFYKRALKDFSHLVGNCSFFTDEKRAPKNSPLAFMFVALTRIINLLNNLKN | |
| | TEGILYTKDDLNALLNEVLKNGTLTYKQTKKLLGLSDDYEFKGEKGTYFIEFKKYK | |
| | EFIKALGEHNLSQDDLNEIAKDITLIKDEIKLKKALAKYDLNQNQIDSLSKLEFKD | |
| | HLNISFKALKLVTPLMLEGKKYDEACNELNLKVAINEDKKDFLPAFNETYYKDEVT | |
| | NPVVLRAIKEYRKVLNALLKKYGKVHKINIELAREVGKNHSQRAKIEKEQNENYKA | |
| | KKDAELECEKLGLKINSKNILKLRLFKEQKEFCAYSGEKIKISDLQDEKMLEIDHI | |
| | YPYSRSFDDSYMNKVLVFTKQNQEKLNQTPFEAFGNDSAKWQKIEVLAKNLPTKKQ | |
| | KRILDKNYKDKEQKNFKDRNLNDTRYIARLVLNYTKDYLDFLPLSDDENTKLNDTQ | |
| | KGSKVHVEAKSGMLTSALRHTWGFSAKDRNNHLHHAIDAVIIAYANNSIVKAFSDF | |
| | KKEQESNSAELYAKKISELDYKNKRKFFEPFSGFRQKVLDKIDEIFVSKPERKKPS | |
| | GALHEETFRKEEEFYQSYGGKEGVLKALELGKIRKVNGKIVKNGDMFRVDIFKHKK | |
| | TNKFYAVPIYTMDFALKVLPNKAVARSKKGEIKDWILMDENYEFCFSLYKDSLILI | |
| | QTKDMQEPEFVYYNAFTSSTVSLIVSKHDNKFETLSKNQKILFKNANEKEVIAKSI | |
| | GIQNLKVFEKYIVSALGEVTKAEFRQREDFKK | |
| GeoCas9 | MRYKIGLDIGITSVGWAVMNLDIPRIEDLGVRIFDRAENPQTGESLALPRRLARSA | 10 |
| | RRRLRRRKHRLERIRRLVIREGILTKEELDKLFEEKHEIDVWQLRVEALDRKLNND | |
| | ELARVLLHLAKRRGFKSNRKSERSNKENSTMLKHIEENRAILSSYRTVGEMIVKDP | |
| | KFALHKRNKGENYTNTIARDDLEREIRLIFSKQREFGNMSCTEEFENEYITIWASQ | |
| | RPVASKDDIEKKVGFCTFEPKEKRAPKATYTFQSFIAWEHINKLRLISPSGARGLT | |
| | DEERRLLYEQAFQKNKITYHDIRTLLHLPDDTYFKGIVYDRGESRKQNENIRFLEL | |
| | DAYHQIRKAVDKVYGKGKSSSFLPIDFDTFGYALTLFKDDADIHSYLRNEYEQNGK | |
| | RMPNLANKVYDNELIEELLNLSFTKFGHLSLKALRSILPYMEQGEVYSSACERAGY | |
| | TFTGPKKKQKTMLLPNIPPIANPVVMRALTQARKVVNAIIKKYGSPVSIHIELARD | |
| | LSQTFDERRKTKKEQDENRKKNETAIRQLMEYGLTLNPTGHDIVKFKLWSEQNGRC | |
| | AYSLQPIEIERLLEPGYVEVDHVIPYSRSLDDSYTNKVLVLTRENREKGNRIPAEY | |
| | LGVGTERWQQFETFVLTNKQFSKKKRDRLLRLHYDENEETEFKNRNLNDTRYISRF | |
| | FANFIREHLKFAESDDKQKVYTVNGRVTAHLRSRWEFNKNREESDLHHAVDAVIVA | |
| | CTTPSDIAKVTAFYQRREQNKELAKKTEPHFPQPWPHFADELRARLSKHPKESIKA | |
| | LNLGNYDDQKLESLQPVFVSRMPKRSVTGAAHQETLRRYVGIDERSGKIQTVVKTK | |
| | LSEIKLDASGHFPMYGKESDPRTYEAIRQRLLEHNNDPKKAFQEPLYKPKKNGEPG | |
| | PVIRTVKIIDTKNQVIPLNDGKTVAYNSNIVRVDVFEKDGKYYCVPVYTMDIMKGI | |
| | LPNKAIEPNKPYSEWKEMTEDYTFRFSLYPNDLIRIELPREKTVKTAAGEEINVKD | |
| | VFVYYKTIDSANGGLELISHDHRFSLRGVGSRTLKRFEKYQVDVLGNIYKVRGEKR | |
| | VGLASSAHSKPGKTIRPLQSTRD | |
| HpaCas9 | MENKNLNYILGLDLGIASVGWAVVEIDEKENPLRLIDVGVRTFERAEVPKTGESLA | 11 |
| | LSRRLARSARRLTQRRVARLKKAKRLLKSENILLSTDERLPHQVWQLRVEGLDHKL | |
| | ERQEWAAVLLHLIKHRGYLSQRKNESKSENKELGALLSGVDNNHKLLQQATYRSPA | |
| | ELAVKKFEVEEGHIRNQQGAYTHTFSRLDLLAEMELLFSRQQHFGNPFASEKLLEN | |
| | LTALLMWQKPALSGEAILKMLGKCTFEDEYKAAKNTYSAERFVWITKLNNLRIQEN | |
| | GLERALNDNERLALMEQPYDKNRLFYSQVRSILKLSDEAIFKGLRYSGEDKKAIET | |
| | KAVLMEMKAYHQIRKVLEGNNLKAEWAELKANPTLLDEIGTAFSLYKTDEDISAYL | |
| | AGKLSQPVLNALLENLSFDKFIQLSLKALYKLLPLMQQGLRYDEACREIYGDHYGK | |
| | KTEENHHFLPQIPADEIRNPVVLRTLTQARKVINGVVRLYGSPARIHIETGREVGK | |
| | SYKDRRELEKRQEENRKQRENAIKEFKEYFPHFAGEPKAKDILKMRLYKQQNAKCL | |
| | YSGKPIELHRLLEKGYVEVDHALPFSRTWDDSFNNKVLVLANENQNKGNLTPFEWL | |
| | DGKHNSERWRAFKALVETSAFPYAKKQRILSQKLDEKGFIERNLNDTRYVARFLCN | |
| | FIADNMHLTGEGKRKVFASNGQITALLRSRWGLAKSREDNDRHHALDAVVVACSTV | |
| | AMQQKITRFVRFEAGDVFTGERIDRETGEIIPLHFPTPWQFFKQEVEIRIFSDNPK | |
| | LELENRLPDRPQANHEFVQPLFVSRMPTRKMTGQGHMETVKSAKRLNEGISVIKMP | |
| | LTKLKLKDLELMVNREREKDLYDTLKARLEAFNDDPAKAFAEPFIKKGGAIVKSVR | |
| | VEQIQKSGVLVREGNGVADNASMVRVDVFTKGGKYFLVPIYTWQVAKGILPNKAAT | |
| | QYKDEEDWEVMDNSATFKFSLHPNDLVKLVTKKKTILGYFNGLNRATGNIDIKEHD | |
| | LDKSKGKQGIFEGVGIKLALSFEKYQVDELGKNIRLCKPSKRQPVR | |
| SmuCas9 | MMMEKFHYVLGLDLGIASVGWAAIEIDKETETSIGLLDCGVRTFERAEVPKTGDSL | 12 |
| | AKARREARSTRRLIRRRSHRLLRLKRLLKREIFRQPETFKDLPINAWQLRVKGLDS | |
| | RLNEYEWAAVLLHLVKHRGYLSQRKSEMSETDSKSEMGRLLAGVAENHQLLQQEQY | |
| | RTPAELALKKFVKHFRNKGGDYAHTFNRLDLQAELHLLFQKQRELGNPFTSPELER | |
| | QVDDLLMTQRSALQGDAILKMLGHCGFEPEQFKAAKNTFSAERFIWLTKLNNLRIQ | |
| | DQGKERALTADERTKLLDEPYKKSKLTYAQVRKLLSLPQTAIFKGLRYDLEHDKKA | |
| | ENSTLMEMKSYHNIRQTLEKSGLKTEWQSIATQPEILDAIGTAFSIYKTDEDISHE | |
| | LKTCRLPENVLNELLKNINFDGFIQLSLTALRKILPLMEQGYRYDEACTQIYGNHH | |
| | SGSLQQESKQFLPHIPIDDVRNPVVFRTLTQARKVVNAIIRRYGSPARVHIEMARE | |
| | LGKSKSDRDRIEKQQQKNKKERENAVAKFKEDFPDFVGEPRGKDILKMRLYEQQHG | |
| | KCLYSGHDIDINRLNEKGYVEIDHALPFSRTWDDSQNNKVLVLGSENQNKRNQTPD | |
| | EYLDGANNSQRWLEFQARVQTCHFSYGKKQRIQLAKLDDETEKGFLERNLNDTRYI | |
| | ARFMCQFVQENLYLTGKGKRLVFASNGGMTATLRNLWGLRKVREDNDRHHALDAIV | |
| | VACSTASMQQKITKAFQRHESIEYVDTETGEVKFRIPQPWDFFRQEVMIRVFSDQP | |
| | CEDLVEKLSARPEALHDNVTPLFVSRAPNRKMSGQGHLETIKSAKRLSEENSMVKK | |
| | PLTTLKLKDIPEIVGYPSREPQLYAALKTRLETHDDDPIKAFAKPFYKPNKNGELG | |
| | ALVRSVRVKGVQNTGVMVHDGKGIADNATMVRVDVYTKAGKNYLVPVYVWQVAQGI | |
| | LPNRAVTSGKSEADWDLIDESFEFKFSLSRGDLVEMISNKGRIFGYYNGLDRANGS | |
| | IGIREHDLEKSKGKDGVHRVGVKTATAFNKYHVDPLGKEIHRCSSEPRPTLKIKSK | |
| | K | |
| NmeCas9 | MAAFKPNSINYILGLDIGIASVGWAMVEIDEEENPIRLIDLGVRVFERAEVPKTGD | 13 |
| | SLAMARRLARSVRRLTRRRAHRLLRTRRLLKREGVLQAANFDENGLIKSLPNTPWQ | |
| | LRAAALDRKLTPLEWSAVLLHLIKHRGYLSQRKNEGETADKELGALLKGVAGNAHA | |
| | LQTGDFRTPAELALNKFEKESGHIRNQRSDYSHTFSRKDLQAELILLFEKQKEFGN | |
| | PHVSGGLKEGIETLLMTQRPALSGDAVQKMLGHCTFEPAEPKAAKNTYTAERFIWL | |
| | TKLNNLRILEQGSERPLTDTERATLMDEPYRKSKLTYAQARKLLGLEDTAFFKGLR | |
| | YGKDNAEASTLMEMKAYHAISRALEKEGLKDKKSPLNLSPELQDEIGTAFSLFKTD | |
| | EDITGRLKDRIQPEILEALLKHISFDKFVQISLKALRRIVPLMEQGKRYDEACAEI | |
| | YGDHYGKKNTEEKIYLPPIPADEIRNPVVLRALSQARKVINGVVRRYGSPARIHIE | |
| | TAREVGKSFKDRKEIEKRQEENRKDREKAAAKFREYFPNFVGEPKSKDILKLRLYE | |
| | QQHGKCLYSGKEINLGRLNEKGYVEIDHALPFSRTWDDSFNNKVLVLGSENQNKGN | |
| | QTPYEYFNGKDNSREWQEFKARVETSRFPRSKKQRILLQKFDEDGFKERNLNDTRY | |
| | VNRFLCQFVADRMRLTGKGKKRVFASNGQITNLLRGFWGLRKVRAENDRHHALDAV | |
| | VVACSTVAMQQKITRFVRYKEMNAFDGKTIDKETGEVLHQKTHFPQPWEFFAQEVM | |
| | IRVFGKPDGKPEFEEADTLEKLRTLLAEKLSSRPEAVHEYVTPLFVSRAPNRKMSG | |
| | QGHMETVKSAKRLDEGVSVLRVPLTQLKLKDLEKMVNREREPKLYEALKARLEAHK | |
| | DDPAKAFAEPFYKYDKAGNRTQQVKAVRVEQVQKTGVWVRNHNGIADNATMVRVDV | |
| | FEKGDKYYLVPIYSWQVAKGILPDRAVVQGKDEEDWQLIDDSFNFKFSLHPNDLVE | |
| | VITKKARMFGYFASCHRGTGNINIRIHDLDHKIGKNGILEGIGVKTALSFQKYQID | |
| | ELGKEIRPCRLKKRPPVR | |

Some aspects include an endonuclease such as an RNA-guided endonuclease. The RNA-guided endonuclease may comprise a class II CRISPR/Cas endonuclease. The RNA-guided endonuclease may comprise a Cas9 endonuclease. The RNA-guided endonuclease may comprise a nickase. The RNA-guided endonuclease may comprise an amino acid sequence at least 80% identical to the amino acid sequence of any one of SEQ ID NOS: 1-13, or a functional fragment thereof.

The endonuclease may introduce a single-strand break in a target nucleic acid. The endonuclease may introduce a single-strand break in a target nucleic acid without cleaving a strand opposite the single strand break. The endonuclease may include a nickase. In some instances, the endonuclease may exclude an endonuclease that introduces a double strand break. The endonuclease may exclude a restriction enzyme.

The endonuclease may be included as part of a fusion protein. In some cases, an endonuclease is a fusion protein that is fused to a heterologous polypeptide such as the heterologous ligase described herein. The heterologous polypeptide may include a fusion partner. The fusion protein may include a fusion partner such as a DNA ligase, a nuclear localization signal, chromatin modifying domain, cell penetrating peptide, or tag polypeptide. The fusion protein may include one or more fusion partner. The fusion protein may include a ligase. The fusion protein may include a nuclear localization signal, chromatin modifying domain, cell penetrating peptide, or tag polypeptide.

The fusion partner may be connected to the N-terminus of the endonuclease. The fusion partner may be connected to the C-terminus of the endonuclease. The endonuclease may be connected at an N-terminus or a C-terminus to a linker. The fusion partner may be connected by the fusion partner's N-terminus or C-terminus. The fusion partner may be connected by the fusion partner's N-terminus to the endonuclease. The fusion partner may be connected by the fusion partner's C-terminus to the endonuclease. The fusion partner may be connected at an N-terminus or a C-terminus to a linker.

In some cases, the endonuclease comprises a linker, where the linker covalently connects the endonuclease to the heterologous polypeptide. The linker may connect the endonuclease to any fusion partner. A linker may also connect any fusion partner to another fusion partner. The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Examples of linker polypeptides include glycine polymers (G)n, glycine-serine polymers (including, for example, (GS)n, (GSGGS)n, (GGSGGS)n, and (GGGS)n, where n is an integer of at least one); glycine-alanine polymers; and alanine-serine polymers. Exemplary linkers can comprise amino acid sequences including, but not limited to, GGSG, GGSGG, GSGSG, GSGGG, GGGSG, GSSSG, and the like. Also suitable is a linker having the sequence (GGGGS)n, where n is an integer of from 1 to 10 (e.g., 1, 2, 3, 4, 5, 6, 7, 8, 9, or 10). The ordinarily skilled artisan will recognize that design of a peptide conjugated to any desired element can include linkers that are all or partially flexible, such that the linker can include a flexible linker as well as one or more portions that confer less flexible structure.

One or more linkers may be included in a fusion protein. 1, 2, 3, 4, 5, 6, 7, 8, 9, or 10 linkers, or a range of linkers defined by any two of the aforementioned integers, may be included in the fusion protein. A linker may connect to an N-terminal end of at least part of the endonuclease. A linker may connect to an N-terminal end of at least part of a fusion partner. A linker may connect to an N-terminal end of at least part of a fusion ligase. A linker may connect to an N-terminal end of a nuclear localization signal. A linker may connect to an N-terminal end of a chromatin modifying domain. A linker may connect to an N-terminal end of a cell penetrating peptide. A linker may connect to an N-terminal end of a tag polypeptide. A linker may connect to a C-terminal end of at least part of the endonuclease. A linker may connect to a C-terminal end of at least part of a fusion partner. A linker may connect to a C-terminal end of at least part of a fusion ligase. A linker may connect to a C-terminal end of a nuclear localization signal. A linker may connect to a C-terminal end of a chromatin modifying domain. A linker may connect to a C-terminal end of a cell penetrating peptide. A linker may connect to a C-terminal end of a tag polypeptide.

A linker may comprise a number or range of amino acids or residues. The linker may include at least 1, at least 2, at least 3, at least 4, at least 5, at least 6, at least 7, at least 8, at least 9, at least 10, at least 12, at least 13, at least 14, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acid residues. The linker may, in some aspects, include no more than 1, no more than 2, no more than 3, no more than 4, no more than 5, no more than 6, no more than 7, no more than 8, no more than 9, no more than 10, no more than 12, no more than 13, no more than 14, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 amino acid residues. A linker may include 1-10 amino acids, 1-25 amino acids, or 1-100 amino acids.

Linkers may be included anywhere in a polypeptide chain or protein described herein. For example, a linker may separate an endonuclease from a ligase. A linker may separate an endonuclease from a nuclear localization signal, a chromatin modifying domain, a cell penetrating peptide, or a tag polypeptide.

In some cases, the endonuclease comprises a nuclear localization sequence (e.g., one or more nuclear localization signals or NLSs for targeting to the nucleus). In some aspects, the NLS described herein comprises any one of the NLS in Table 2. In some aspects, the NLS described herein comprises a polypeptide sequence that is at least 70%, at least 75%, at least 80%, at least 85%, at least 90%, at least 95%, at least 99%, or more identical to the polypeptide sequence of any one of NLS in Table 2.

TABLE 2
Non-limiting examples of NLS
polypeptide sequence

| | | SEQ |
|---|---|---|
| | NLS polypeptide | ID |
| Name | sequence | NO: |
| NLS1 | KRTADGSEFESPKKKRKV | 14 |
| NLS2 | SGGSKRTADSQHSTPPKT | 15 |
| | KRKVEFEPKKKRKV | |
| NLS3 | KRPAATKKAGQAKKKK | 16 |
| NLS4 | KKTELQTTNAENKTKKL | 17 |
| NLS5 | KRGINDRNFWRGENGRKTR | 18 |
| NLS6 | RKSGKIAAIVVKRPRK | 19 |
| NLS7 | PKKKRKV | 20 |
| NLS8 | MDSLLMNRRKFLYQFK | 21 |
| | NVRWAKGRRETYLC | |
| SGGSx2-bpNLS- | SGGSSGGSKRTADGSE | 22 |
| SGGSx2 | FESPKKKRKVSGGSSGGS | |
| SGGSx2-XTEN16- | SGGSSGGSSGSETPGT | 23 |
| SGGSx2 | SESATPESSGGSSGGSS | |
| SGGSx10 | SGGSSGGSSGGSSGGS | 24 |
| | SGGSSGGSSGGSSGGS | |
| | SGGSSGGS | |

A polynucleotide encoding an NLS polypeptide may be used. An example of such a polynucleotide may be SGGSx2-bpNLS-SGGSx2:

(SEQ ID NO: 25)
TCCGGCGGAAGCTCTGGTGGCAGCAAGCGGACCGCCGACGGCTCTGAAT
TCGAGAGCCCTAAGAAGAAAAGAAAGGTGAGCGGAGGCTCTAGCGGCGG
AAGC.

In some aspects, the endonuclease comprises a dimerization domain. The dimerization domain can be located at the N-terminus or C-terminus of the endonuclease. In some aspects, the dimerization domain allows the endonuclease to form a heterodimer with another polypeptide (e.g., the heterologous ligase). In some aspects, the dimerization domain allows the endonuclease to be functionally coupled with another polypeptide. Non-limiting examples of the dimerization domains can include a leucine zipper, an FKBP, an FRB, a Calcineurin A, a CyP-Fas, a GyrB, a GAI, a GID1, a SNAP tag, a Halo tag, a Bcl-xL, a Fab, a LOV domain, or SpyTag/SpyCatcher. Other example of dimerization domain can include an antibody such as anyone of heavy chain domain 2 (CH2) of IgM (MHD2) or IgE (EHD2), immunoglobulin Fc region, heavy chain domain 3 (CH3) of IgG or IgA, heavy chain domain 4 (CH4) of IgM or IgE, Fab, Fab2, leucine zipper motifs, barnase-barstar dimers, miniantibodies, or ZIP miniantibodies. In some aspects, the dimerization domain described herein comprises any one of the dimerization domain in Table 3. In some aspects, the dimerization domain described herein comprises a polypeptide sequence that is at least 70%, at least 75%, at least 80%, at least 85%, at least 90%, at least 95%, at least 99%, or more identical to the polypeptide sequence of any one of dimerization domain in Table 3.

TABLE 3
Non-limiting examples of dimerization
domain sequence

| | Dimerization | SEQ |
|---|---|---|
| Name | domain sequence | ID NO: |
| Leucine zipper | LEIEAAFLERENTALETRVAE | 26 |
| EE12RR345L | LRQRVQRLRNRVSQYRTRYGP | |
| | LGGGK | |
| Leucine zipper | LEIRAAFLRQRNTALRTEVAE | 27 |
| RR12EE345L | LEQEVQRLENEVSQYETRYGP | |
| | LGGGK | |

In some aspects, the endonuclease comprises at least one additional domain. In some aspects, the at least one additional domain is a functional domain. For example, the functional domain can comprises a chromatin modifying domain or a cell penetrating peptide. In some aspects, the chromatin modifying domain described herein comprises any one of the chromatin modifying domain in Table 4. In some aspects, the chromatin modifying domain described herein comprises a polypeptide sequence that is at least 70%, at least 75%, at least 80%, at least 85%, at least 90%, at least 95%, at least 99%, or more identical to the polypeptide sequence of any one of chromatin modifying domain in Table 4.

TABLE 4
Non-limiting examples of chromatin
modifying domain polypeptide sequence

| | Chromatin modifying domain | SEQ ID |
|---|---|---|
| Name | polypeptide sequence | NO: |
| H1G | STDHPKYSDMIVAAIQAEKN | 28 |
| (histone | RAGSSRQSIQKYIKSHYKVG | |
| H1 central | ENADSQIKLSIKRLVTTGVL | |
| globular domain) | KQTKGVGASGSFRLAKSDEP | |
| HMGB1 | MGKGDPKKPRGKMSSYAFFV | 29 |
| | QTCREEHKKKHPDASVNFSE | |
| | FSKKCSERWKTMSAKEKGKF | |
| | EDMAKADKARYEREMKTYIP | |
| | PKGETKKKFKDPNAPKRPPS | |
| | AFFLFCSEYRPKIKGEHPGL | |
| | SIGDVAKKLGEMWNNTAADD | |
| | KQPYEKKAAKLKEKYEKDIA | |
| | AYRAKGKPDAAKKGVVKAEK | |
| | SKKKKEEEEDEEDEEDEEEE | |
| | EDEEDEDEEEDDDDE | |
| HMGB2 | MGKGDPNKPRGKMSSYAFFV | 30 |
| | QTCREEHKKKHPDSSVNFAE | |
| | FSKKCSERWKTMSAKEKSKF | |
| | EDMAKSDKARYDREMKNYVP | |
| | PKGDKKGKKKDPNAPKRPPS | |
| | AFFLFCSEHRPKIKSEHPGL | |
| | SIGDTAKKLGEMWSEQSAKD | |
| | KQPYEQKAAKLKEKYEKDIA | |
| | AYRAKGKSEAGKKGPGRPTG | |
| | SKKKNEPEDEEEEEEEEDED | |
| | EEEEDEDEE | |
| HMGB3 | MAKGDPKKPKGKMSAYAFFV | 31 |
| | QTCREEHKKKNPEVPVNFAE | |
| | FSKKCSERWKTMSGKEKSKF | |
| | DEMAKADKVRYDREMKDYGP | |
| | AKGGKKKKDPNAPKRPPSGF | |
| | FLFCSEFRPKIKSTNPGISI | |
| | GDVAKKLGEMWNNLNDSEKQ | |
| | PYITKAAKLKEKYEKDVADY | |
| | KSKGKFDGAKGPAKVARKKV | |
| | EEEDEEEEEEEEEEEEEEDE | |
| HMGN1 | MPKRKVSSAEGAAKEEPKRR | 32 |
| (HN1) | SARLSAKPPAKVEAKPKKAA | |
| | AKDKSSDKKVQTKGKRGAKG | |
| | KQAEVANQETKEDLPAENGE | |
| | TKTEESPASDEAGEKEAKSD | |
| HMGN2 | MPKRKAEGDAKGDKAKVKDE | 33 |
| | PQRRSARLSAKPAPPKPEPK | |
| | PKKAPAKKGEKVPKGKKGKA | |
| | DAGKEGNNPAENGDAKTDQA | |
| | QKAEGAGDAK | |

In some aspects, the cell penetrating peptide described herein comprises any one of the cell penetrating peptide in Table 5. In some aspects, the cell penetrating peptide described herein comprises a polypeptide sequence that is at least 70%, at least 75%, at least 80%, at least 85%, at least 90%, at least 95%, at least 99%, or more identical to the polypeptide sequence of any one of cell penetrating peptide in Table 5.

TABLE 5
Non-limiting examples of cell penetrating
peptide polypeptide sequence

| | Cell penetrating | SEQ ID |
|---|---|---|
| Name | peptide sequence | NO: |
| Penetratin | RQIKIYFQNRRMKWKK | 34 |
| TAT | RKKRRQRRR | 35 |
| R8 | RRRRRRRR | 36 |
| DPV3 | RKKRRRESRKKRRRES | 37 |
| DPV6 | GRPRESGKKRKRKRLKP | 38 |
| R9-TAT | GRRRRRRRRRPPQ | 39 |
| pVEC | LLIILRRRIRKQAHAHSK | 40 |
| ARF(19-31) | RVRVFVVWHIPRLT | 41 |
| MPG | GALFLGFLGAAGSTMGA | 42 |
| | WSQPKKKRKV | |
| Transportan | GWTLNSAGYLLGKINLK | 43 |
| | ALAALAKKIL | |
| Bip4 | VSALK | 44 |
| C105Y | CSIPPEVKFNPFVYLI | 45 |
| Melittin | GIGAVLKVLTTGLPALI | 46 |
| | SWIKRKRQQ | |
| gH625 | HGLASTLTRWAHYNALIRAF | 47 |

In some aspects, the endonuclease comprises a tag, where the tag can be used for increasing expression, identifying, or purifying the endonuclease. In some aspects, the tag described herein comprises any one of the tag sequence in Table 6. In some aspects, the tag described herein comprises a polypeptide sequence that is at least 70%, at least 75%, at least 80%, at least 85%, at least 90%, at least 95%, at least 99%, or more identical to the polypeptide sequence of any one of the tag sequence in Table 6.

TABLE 6
Non-limiting examples of tag
polypeptide sequence

| | Tag polypeptide | SEQ ID |
|---|---|---|
| Name | sequence | NO: |
| FLAG | DYKDDDDK | 48 |
| His-Tag | HHHHHH | 49 |
| CBP | KRRWKKNFIAVSAANRFKK | 50 |
| | ISSSGAL | |
| MBP | MKIKTGARILALSALTTMMF | 51 |
| | SASALAKIEEGKLVIWINGD | |
| | KGYNGLAEVGKKFEKDTGIK | |
| | VTVEHPDKLEEKFPQVAATG | |
| | DGPDIIFWAHDRFGGYAQSG | |
| | LLAEITPDKAFQDKLYPFTW | |
| | DAVRYNGKLIAYPIAVEALS | |
| | LIYNKDLLPNPPKTWEEIPA | |
| | LDKELKAKGKSALMFNLQEP | |
| | YFTWPLIAADGGYAFKYENG | |
| | KYDIKDVGVDNAGAKAGLTF | |
| | LVDLIKNKHMNADTDYSIAE | |
| | AAFNKGETAMTINGPWAWSN | |
| | IDTSKVNYGVTVLPTFKGQP | |
| | SKPFVGVLSAGINAASPNKE | |
| | LAKEFLENYLLTDEGLEAVN | |
| | KDKPLGAVALKSYEEELAKD | |
| | PRIAATMENAQKGEIMPNIP | |
| | QMSAFWYAVRTAVINAASGR | |
| | QTVDEALKDAQTRITK | |
| Myc | EQKLISEEDL | 52 |
| GST | MKLFYKPGACSLASHITLRE | 53 |
| | SGKDFTLVSVDLMKKRLENG | |
| | DNYFAVNPKGQVPALLLDDG | |
| | TLLTEGVAIMQYLADSVPDR | |
| | QLLAPVNSISRYKTIEWLNY | |
| | IATELHKGFTPLFRPDTPEE | |
| | YKSTVRAQLEKKLQYVNEAL | |
| | KDEHWICGQRFTIADAYLFT | |
| | VLRWAYAVKLNLEGLEHIAA | |
| | FMQRMAERPEVQDALSAEGLK | |
| HA | YPYDVPDYA | 54 |
| HA | YAYDVPDYA | 210 |
| HA | YDVPDYASL | 211 |

In some embodiments, the endonuclease can be expressed as split construct as one or more exteins fused to one or more inteins. Intein technology may be used to deliver large proteins into a cell by expressing the protein as two or more shorter peptide segments (exteins). Each extein may be expressed as a fusion with an intein peptide (e.g., an Npu C intein or an Npu N intein). An intein may autocatalyze fusion of two or more exteins and may autocatalyze excision of the intein from its corresponding extein. The result may be a protein complex comprising a first extein fused to a second extein and lacking inteins. An intein may be positioned N-terminal of the extein, or an intein may be positioned C-terminal of the extein. An extein may comprise a cysteine residue positioned adjacent to the intein (e.g., at the C-terminal end of an extein with an intein fused to the C-terminal end of the extein). The Cas nickase may be expressed as two or more segments. A first of the Cas nickase segment may comprise an N-terminal portion of the Cas nickase. A first segment of the Cas nickase may comprise a first intein. A second segment of the Cas nickase may comprise a C-terminal portion of the Cas nickase. A second segment of the Cas nickase may comprise a second intein. An intein may be fused to a C-terminus of an N-terminal portion of the Cas nickase. An intein may be fused to an N-terminus of a C-terminal portion of the Cas nickase. A nucleic acid sequence encoding an extein-intein fusion may fit into a delivery vector (e.g., an adeno-associated virus (AAV) vector).

### DNA Ligases

Disclosed herein are ligases. The ligase may be or include a DNA ligase. The ligase may be included in a composition, system or method disclosed herein. The ligase may be recombinant. The ligase may be coupled to the endonuclease. The ligase may be coupled directly or indirectly to the endonuclease. The coupling may be covalent or non-covalent. The ligase may be bound or connected to the endonuclease. The ligase may be recruited to, be part of a fusion protein with, or be used in conjunction with an endonuclease. The ligase may be heterologous. The ligase may be endogenous. Where a heterologous ligase is described, a non-heterologous (e.g. endogenous) ligase may be used in some cases. The ligase may be encoded in a cell. The ligase may be delivered to the cell in trans. The ligase may form a phosphodiester bond by joining two nucleic acid ends together. The ligase may join an end (e.g. 5′ or 3′ end) of a target nucleic acid to an integrating nucleic acid (e.g. a 3′ or 5′ end of the integrating nucleic acid). The ligase ligates an integrating nucleic acid to a cleaved or nicked end of a target nucleic acid where the cleaved or nicked end has been generated by an endonuclease such as an RNA-guided endonuclease. The ligase may include any aspect included in FIG. 1A-6C.

The ligase may be non-naturally occurring. The ligase may be engineered. The ligase may be synthetic. The ligase may be pre-synthetized. The ligase may be added to a subject or a cell. The ligase may be encoded by a nucleic acid. The encoding nucleic acid may be engineered, synthetic, or added to a subject or a cell.

At least part of the ligase may be included in a first polypeptide. At least part of the ligase may be included in a second polypeptide. The ligase may be split into two polypeptides bound together. The first polypeptide may include an N-terminal portion of the ligase. The first polypeptide may include a C-terminal portion of the ligase. The second polypeptide may include the N-terminal portion of the ligase. The second polypeptide may include the C-terminal portion of the ligase. The first or second polypeptide comprising a part of the ligase may be fused with at least part, or the whole, of the endonuclease.

Examples of DNA ligases are hLIG1, T4 ligase, T7 ligase, and ligases from Aquifex aeolicus VF5, Neisseria meningitidis serogroup A strain Z2491, Neisseria meningitidis serogroup B strain MC58, Pseudomonas aeruginosa PA01, Vibrio cholerae El Tor N1696, Vaccinia virus, and Emiliania huxleyi virus.

The ligase may comprise a ligase that can ligate a substrate comprising DNA. In some aspects, the ligase comprises a ligase that can ligate a substrate comprising a DNA splint. For example, a DNA ligase may ligate a 5′ phosphate to a 3′ hydroxyl of two DNA strands that are hybridized to another DNA strand. The splinting DNA strand may include an RNA portion. For example, a DNA ligase may ligate a 5′ phosphate to a 3′ hydroxyl of two DNA strands that are hybridized across from a DNA portion of an RNA/DNA hybrid strand. In some aspects, the ligase comprises a ligase that can ligate a substrate comprising a DNA/RNA. In some aspects, the ligase comprises a ligase that can ligate a substrate comprising a RNA splint. For example, a DNA ligase may ligate a 5′ phosphate to a 3′ hydroxyl of two DNA strands that are hybridized to an RNA strand. The RNA strand may include a DNA portion. For example, a DNA ligase may ligate a 5′ phosphate to a 3′ hydroxyl of two DNA strands that are hybridized across from an RNA portion of an RNA/DNA hybrid strand.

In some aspects, the ligase described herein comprises any one of the ligase in Table 7. In some aspects, the ligase described herein comprises a polypeptide sequence that is at least 70%, at least 75%, at least 80%, at least 85%, at least 90%, at least 95%, at least 99%, or more identical to the polypeptide sequence of any one of the ligase in Table 7.

TABLE 7
Non-limiting examples of ligase
polypeptide sequence

| | Ligase polypeptide | SEQ ID |
|---|---|---|
| Name | sequence | NO: |
| splintR | MAITKPLLAATLENIEDVQF | 55 |
| (chlorella | PCLATPKIDGIRSVKQTQML | |
| virus DNA | SRTFKPIRNSVMNRLLTELL | |
| ligase PBCV1) | PEGSDGEISIEGATFQDTTS | |
| | AVMTGHKMYNAKFSYYWFDY | |
| | VTDDPLKKYIDRVEDMKNYI | |
| | TVHPHILEHAQVKIIPLIPV | |
| | EINNITELLQYERDVLSKGF | |
| | EGVMIRKPDGKYKFGRSTLK | |
| | EGILLKMKQFKDAEATIISM | |
| | TALFKNTNTKTKDNFGYSKR | |
| | STHKSGKVEEDVMGSIEVDY | |
| | DGVVFSIGTGFDADQRRDFW | |
| | QNKESYIGKMVKFKYFEMGS | |
| | KDCPRFPVFIGIRHEEDR | |
| hLIG1 | MQRSIMSFFHPKKEGKAKKP | 56 |
| | EKEASNSSRETEPPPKAALK | |
| | EWNGVVSESDSPVKRPGRKA | |
| | ARVLGSEGEEEDEALSPAKG | |
| | QKPALDCSQVSPPRPATSPE | |
| | NNASLSDTSPMDSSPSGIPK | |
| | RRTARKQLPKRTIQEVLEEQ | |
| | SEDEDREAKRKKEEEEEETP | |
| | KESLTEAEVATEKEGEDGDQ | |
| | PTTPPKPLKTSKAETPTESV | |
| | SEPEVATKQELQEEEEQTKP | |
| | PRRAPKTLSSFFTPRKPAVK | |
| | KEVKEEEPGAPGKEGAAEGP | |
| | LDPSGYNPAKNNYHPVEDAC | |
| | WKPGQKVPYLAVARTFEKIE | |
| | EVSARLRMVETLSNLLRSVV | |
| | ALSPPDLLPVLYLSLNHLGP | |
| | PQQGLELGVGDGVLLKAVAQ | |
| | ATGRQLESVRAEAAEKGDVG | |
| | LVAENSRSTQRLMLPPPPLT | |
| | ASGVFSKFRDIARLTGSAST | |
| | AKKIDIIKGLFVACRHSEAR | |
| | FIARSLSGRLRLGLAEQSVL | |
| | AALSQAVSLTPPGQEFPPAM | |
| | VDAGKGKTAEARKTWLEEQG | |
| | MILKQTFCEVPDLDRIIPVL | |
| | LEHGLERLPEHCKLSPGIPL | |
| | KPMLAHPTRGISEVLKRFEE | |
| | AAFTCEYKYDGQRAQIHALE | |
| | GGEVKIFSRNQEDNTGKYPD | |
| | IISRIPKIKLPSVTSFILDT | |
| | EAVAWDREKKQIQPFQVLTT | |
| | RKRKEVDASEIQVQVCLYAF | |
| | DLIYLNGESLVREPLSRRRQ | |
| | LLRENFVETEGEFVFATSLD | |
| | TKDIEQIAEFLEQSVKDSCE | |
| | GLMVKTLDVDATYEIAKRSH | |
| | NWLKLKKDYLDGVGDTLDLV | |
| | VIGAYLGRGKRAGRYGGFLL | |
| | ASYDEDSEELQAICKLGTGF | |
| | SDEELEEHHQSLKALVLPSP | |
| | RPYVRIDGAVIPDHWLDPSA | |
| | VWEVKCADLSLSPIYPAARG | |
| | LVDSDKGISLRFPRFIRVRE | |
| | DKQPEQATTSAQVACLYRKQ | |
| | SQIQNQQGEDSGSDPEDTY | |
| hLIG1 | TPRKPAVKKEVKEEEPGAPG | 57 |
| (233-919) | KEGAAEGPLDPSGYNPAKNN | |
| | YHPVEDACWKPGQKVPYLAV | |
| | ARTFEKIEEVSARLRMVETL | |
| | SNLLRSVVALSPPDLLPVLY | |
| | LSLNHLGPPQQGLELGVGDG | |
| | VLLKAVAQATGRQLESVRAE | |
| | AAEKGDVGLVAENSRSTQRL | |
| | MLPPPPLTASGVFSKFRDIA | |
| | RLTGSASTAKKIDIIKGLFV | |
| | ACRHSEARFIARSLSGRLRL | |
| | GLAEQSVLAALSQAVSLTPP | |
| | GQEFPPAMVDAGKGKTAEAR | |
| | KTWLEEQGMILKQTFCEVPD | |
| | LDRIIPVLLEHGLERLPEHC | |
| | KLSPGIPLKPMLAHPTRGIS | |
| | EVLKRFEEAAFTCEYKYDGQ | |
| | RAQIHALEGGEVKIFSRNQE | |
| | DNTGKYPDIISRIPKIKLPS | |
| | VTSFILDTEAVAWDREKKQI | |
| | QPFQVLTTRKRKEVDASEIQ | |
| | VQVCLYAFDLIYLNGESLVR | |
| | EPLSRRRQLLRENFVETEGE | |
| | FVFATSLDTKDIEQIAEFLE | |
| | QSVKDSCEGLMVKTLDVDAT | |
| | YEIAKRSHNWLKLKKDYLDG | |
| | VGDTLDLVVIGAYLGRGKRA | |
| | GRYGGFLLASYDEDSEELQA | |
| | ICKLGTGFSDEELEEHHQSL | |
| | KALVLPSPRPYVRIDGAVIP | |
| | DHWLDPSAVWEVKCADLSLS | |
| | PIYPAARGLVDSDKGISLRF | |
| | PRFIRVREDKQPEQATTSAQ | |
| | VACLYRKQSQIQNQQGEDSG | |
| | SDPEDTY | |
| hLIG1 | PKRRTARKQLPKRTIQEVLE | 58 |
| (119-919) | EQSEDEDREAKRKKEEEEEE | |
| | TPKESLTEAEVATEKEGEDG | |
| | DQPTTPPKPLKTSKAETPTE | |
| | SVSEPEVATKQELQEEEEQT | |
| | KPPRRAPKTLSSFFTPRKPA | |
| | VKKEVKEEEPGAPGKEGAAE | |
| | GPLDPSGYNPAKNNYHPVED | |
| | ACWKPGQKVPYLAVARTFEK | |
| | IEEVSARLRMVETLSNLLRS | |
| | VVALSPPDLLPVLYLSLNHL | |
| | GPPQQGLELGVGDGVLLKAV | |
| | AQATGRQLESVRAEAAEKGD | |
| | VGLVAENSRSTQRLMLPPPP | |
| | LTASGVFSKFRDIARLTGSA | |
| | STAKKIDIIKGLFVACRHSE | |
| | ARFIARSLSGRLRLGLAEQS | |
| | VLAALSQAVSLTPPGQEFPP | |
| | AMVDAGKGKTAEARKTWLEE | |
| | QGMILKQTFCEVPDLDRIIP | |
| | VLLEHGLERLPEHCKLSPGI | |
| | PLKPMLAHPTRGISEVLKRF | |
| | EEAAFTCEYKYDGQRAQIHA | |
| | LEGGEVKIFSRNQEDNTGKY | |
| | PDIISRIPKIKLPSVTSFIL | |
| | DTEAVAWDREKKQIQPFQVL | |
| | TTRKRKEVDASEIQVQVCLY | |
| | AFDLIYLNGESLVREPLSRR | |
| | RQLLRENFVETEGEFVFATS | |
| | LDTKDIEQIAEFLEQSVKDS | |
| | CEGLMVKTLDVDATYEIAKR | |
| | SHNWLKLKKDYLDGVGDTLD | |
| | LVVIGAYLGRGKRAGRYGGF | |
| | LLASYDEDSEELQAICKLGT | |
| | GFSDEELEEHHQSLKALVLP | |
| | SPRPYVRIDGAVIPDHWLDP | |
| | SAVWEVKCADLSLSPIYPAA | |
| | RGLVDSDKGISLRFPRFIRV | |
| | REDKQPEQATTSAQVACLYR | |
| | KQSQIQNQQGEDSGSDPEDT | |
| | Y | |
| hLIG3 | MSLAFKIFFPQTLRALSRKE | 59 |
| isoform 1 | LCLFRKHHWRDVRQFSQWSE | |
| | TDLLHGHPLFLRRKPVLSFQ | |
| | GSHLRSRATYLVFLPGLHVG | |
| | LCSGPCEMAEQRFCVDYAKR | |
| | GTAGCKKCKEKIVKGVCRIG | |
| | KVVPNPFSESGGDMKEWYHI | |
| | KCMFEKLERARATTKKIEDL | |
| | TELEGWEELEDNEKEQITQH | |
| | IADLSSKAAGTPKKKAVVQA | |
| | KLTTTGQVTSPVKGASFVTS | |
| | TNPRKFSGFSAKPNNSGEAP | |
| | SSPTPKRSLSSSKCDPRHKD | |
| | CLLREFRKLCAMVADNPSYN | |
| | TKTQIIQDFLRKGSAGDGFH | |
| | GDVYLTVKLLLPGVIKTVYN | |
| | LNDKQIVKLFSRIFNCNPDD | |
| | MARDLEQGDVSETIRVFFEQ | |
| | SKSFPPAAKSLLTIQEVDEF | |
| | LLRLSKLTKEDEQQQALQDI | |
| | ASRCTANDLKCIIRLIKHDL | |
| | KMNSGAKHVLDALDPNAYEA | |
| | FKASRNLQDVVERVLHNAQE | |
| | VEKEPGQRRALSVQASLMTP | |
| | VQPMLAEACKSVEYAMKKCP | |
| | NGMFSEIKYDGERVQVHKNG | |
| | DHFSYFSRSLKPVLPHKVAH | |
| | FKDYIPQAFPGGHSMILDSE | |
| | VLLIDNKTGKPLPFGTLGVH | |
| | KKAAFQDANVCLFVFDCIYF | |
| | NDVSLMDRPLCERRKFLHDN | |
| | MVEIPNRIMFSEMKRVTKAL | |
| | DLADMITRVIQEGLEGLVLK | |
| | DVKGTYEPGKRHWLKVKKDY | |
| | LNEGAMADTADLVVLGAFYG | |
| | QGSKGGMMSIFLMGCYDPGS | |
| | QKWCTVTKCAGGHDDATLAR | |
| | LQNELDMVKISKDPSKIPSW | |
| | LKVNKIYYPDFIVPDPKKAA | |
| | VWEITGAEFSKSEAHTADGI | |
| | SIRFPRCTRIRDDKDWKSAT | |
| | NLPQLKELYQLSKEKADFTV | |
| | VAGDEGSSTTGGSSEENKGP | |
| | SGSAVSRKAPSKPSASTKKA | |
| | EGKLSNSNSKDGNMQTAKPS | |
| | AMKVGEKLATKSSPVKVGEK | |
| | RKAADETLCQTKVLLDIFTG | |
| | VRLYLPPSTPDFSRLRRYFV | |
| | AFDGDLVQEFDMTSATHVLG | |
| | SRDKNPAAQQVSPEWIWACI | |
| | RKRRLVAPC | |
| hLIG3 | MSLAFKIFFPQTLRALSRKE | 60 |
| isoform 2 | LCLFRKHHWRDVRQFSQWSE | |
| | TDLLHGHPLFLRRKPVLSFQ | |
| | GSHLRSRATYLVFLPGLHVG | |
| | LCSGPCEMAEQRFCVDYAKR | |
| | GTAGCKKCKEKIVKGVCRIG | |
| | KVVPNPFSESGGDMKEWYHI | |
| | KCMFEKLERARATTKKIEDL | |
| | TELEGWEELEDNEKEQITQH | |
| | IADLSSKAAGTPKKKAVVQA | |
| | KLTTTGQVTSPVKGASFVTS | |
| | TNPRKFSGFSAKPNNSGEAP | |
| | SSPTPKRSLSSSKCDPRHKD | |
| | CLLREFRKLCAMVADNPSYN | |
| | TKTQIIQDFLRKGSAGDGFH | |
| | GDVYLTVKLLLPGVIKTVYN | |
| | LNDKQIVKLFSRIFNCNPDD | |
| | MARDLEQGDVSETIRVFFEQ | |
| | SKSFPPAAKSLLTIQEVDEF | |
| | LLRLSKLTKEDEQQQALQDI | |
| | ASRCTANDLKCIIRLIKHDL | |
| | KMNSGAKHVLDALDPNAYEA | |
| | FKASRNLQDVVERVLHNAQE | |
| | VEKEPGQRRALSVQASLMTP | |
| | VQPMLAEACKSVEYAMKKCP | |
| | NGMFSEIKYDGERVQVHKNG | |
| | DHFSYFSRSLKPVLPHKVAH | |
| | FKDYIPQAFPGGHSMILDSE | |
| | VLLIDNKTGKPLPFGTLGVH | |
| | KKAAFQDANVCLFVFDCIYF | |
| | NDVSLMDRPLCERRKFLHDN | |
| | MVEIPNRIMFSEMKRVTKAL | |
| | DLADMITRVIQEGLEGLVLK | |
| | DVKGTYEPGKRHWLKVKKDY | |
| | LNEGAMADTADLVVLGAFYG | |
| | QGSKGGMMSIFLMGCYDPGS | |
| | QKWCTVTKCAGGHDDATLAR | |
| | LQNELDMVKISKDPSKIPSW | |
| | LKVNKIYYPDFIVPDPKKAA | |
| | VWEITGAEFSKSEAHTADGI | |
| | SIRFPRCTRIRDDKDWKSAT | |
| | NLPQLKELYQLSKEKADFTV | |
| | VAGDEGSSTTGGSSEENKGP | |
| | SGSAVSRKAPSKPSASTKKA | |
| | EGKLSNSNSKDGNMQTAKPS | |
| | AMKVGEKLATKSSPVKVGEK | |
| | RKAADETLCQTKRRPASEQR | |
| | GRTVPAGRR | |
| hLIG3 | MAEQRFCVDYAKRGTAGCKK | 61 |
| isoform 3 | CKEKIVKGVCRIGKVVPNPF | |
| | SESGGDMKEWYHIKCMFEKL | |
| | ERARATTKKIEDLTELEGWE | |
| | ELEDNEKEQITQHIADLSSK | |
| | AAGTPKKKAVVQAKLTTTGQ | |
| | VTSPVKGASFVTSTNPRKFS | |
| | GFSAKPNNSGEAPSSPTPKR | |
| | SLSSSKCDPRHKDCLLREFR | |
| | KLCAMVADNPSYNTKTQIIQ | |
| | DFLRKGSAGDGFHGDVYLTV | |
| | KLLLPGVIKTVYNLNDKQIV | |
| | KLFSRIFNCNPDDMARDLEQ | |
| | GDVSETIRVFFEQSKSFPPA | |
| | AKSLLTIQEVDEFLLRLSKL | |
| | TKEDEQQQALQDIASRCTAN | |
| | DLKCIIRLIKHDLKMNSGAK | |
| | HVLDALDPNAYEAFKASRNL | |
| | QDVVERVLHNAQEVEKEPGQ | |
| | RRALSVQASLMTPVQPMLAE | |
| | ACKSVEYAMKKCPNGMFSEI | |
| | KYDGERVQVHKNGDHFSYFS | |
| | RSLKPVLPHKVAHFKDYIPQ | |
| | AFPGGHSMILDSEVLLIDNK | |
| | TGKPLPFGTLGVHKKAAFQD | |
| | ANVCLFVFDCIYFNDVSLMD | |
| | RPLCERRKFLHDNMVEIPNR | |
| | IMFSEMKRVTKALDLADMIT | |
| | RVIQEGLEGLVLKDVKGTYE | |
| | PGKRHWLKVKKDYLNEGAMA | |
| | DTADLVVLGAFYGQGSKGGM | |
| | MSIFLMGCYDPGSQKWCTVT | |
| | KCAGGHDDATLARLQNELDM | |
| | VKISKDPSKIPSWLKVNKIY | |
| | YPDFIVPDPKKAAVWEITGA | |
| | EFSKSEAHTADGISIRFPRC | |
| | TRIRDDKDWKSATNLPQLKE | |
| | LYQLSKEKADFTVVAGDEGS | |
| | STTGGSSEENKGPSGSAVSR | |
| | KAPSKPSASTKKAEGKLSNS | |
| | NSKDGNMQTAKPSAMKVGEK | |
| | LATKSSPVKVGEKRKAADET | |
| | LCQTKVLLDIFTGVRLYLPP | |
| | STPDFSRLRRYFVAFDGDLV | |
| | QEFDMTSATHVLGSRDKNPA | |
| | AQQVSPEWIWACIRKRRLVA | |
| | PC | |
| hLIG3 | MAEQRFCVDYAKRGTAGCKK | 62 |
| isoform 4 | CKEKIVKGVCRIGKVVPNPF | |
| | SESGGDMKEWYHIKCMFEKL | |
| | ERARATTKKIEDLTELEGWE | |
| | ELEDNEKEQITQHIADLSSK | |
| | AAGTPKKKAVVQAKLTTTGQ | |
| | VTSPVKGASFVTSTNPRKFS | |
| | GFSAKPNNSGEAPSSPTPKR | |
| | SLSSSKCDPRHKDCLLREFR | |
| | KLCAMVADNPSYNTKTQIIQ | |
| | DFLRKGSAGDGFHGDVYLTV | |
| | KLLLPGVIKTVYNLNDKQIV | |
| | KLFSRIFNCNPDDMARDLEQ | |
| | GDVSETIRVFFEQSKSFPPA | |
| | AKSLLTIQEVDEFLLRLSKL | |
| | TKEDEQQQALQDIASRCTAN | |
| | DLKCIIRLIKHDLKMNSGAK | |
| | HVLDALDPNAYEAFKASRNL | |
| | QDVVERVLHNAQEVEKEPGQ | |
| | RRALSVQASLMTPVQPMLAE | |
| | ACKSVEYAMKKCPNGMFSEI | |
| | KYDGERVQVHKNGDHFSYFS | |
| | RSLKPVLPHKVAHFKDYIPQ | |
| | AFPGGHSMILDSEVLLIDNK | |
| | TGKPLPFGTLGVHKKAAFQD | |
| | ANVCLFVFDCIYFNDVSLMD | |
| | RPLCERRKFLHDNMVEIPNR | |
| | IMFSEMKRVTKALDLADMIT | |
| | RVIQEGLEGLVLKDVKGTYE | |
| | PGKRHWLKVKKDYLNEGAMA | |
| | DTADLVVLGAFYGQGSKGGM | |
| | MSIFLMGCYDPGSQKWCTVT | |
| | KCAGGHDDATLARLQNELDM | |
| | VKISKDPSKIPSWLKVNKIY | |
| | YPDFIVPDPKKAAVWEITGA | |
| | EFSKSEAHTADGISIRFPRC | |
| | TRIRDDKDWKSATNLPQLKE | |
| | LYQLSKEKADFTVVAGDEGS | |
| | STTGGSSEENKGPSGSAVSR | |
| | KAPSKPSASTKKAEGKLSNS | |
| | NSKDGNMQTAKPSAMKVGEK | |
| | LATKSSPVKVGEKRKAADET | |
| | LCQTKRRPASEQRGRTVPAG | |
| | RR | |
| hLIG4 | MAASQTSQTVASHVPFADLC | 63 |
| | STLERIQKSKGRAEKIRHFR | |
| | EFLDSWRKFHDALHKNHKDV | |
| | TDSFYPAMRLILPQLERERM | |
| | AYGIKETMLAKLYIELLNLP | |
| | RDGKDALKLLNYRTPTGTHG | |
| | DAGDFAMIAYFVLKPRCLQK | |
| | GSLTIQQVNDLLDSIASNNS | |
| | AKRKDLIKKSLLQLITQSSA | |
| | LEQKWLIRMIIKDLKLGVSQ | |
| | QTIFSVFHNDAAELHNVTTD | |
| | LEKVCRQLHDPSVGLSDISI | |
| | TLFSAFKPMLAAIADIEHIE | |
| | KDMKHQSFYIETKLDGERMQ | |
| | MHKDGDVYKYFSRNGYNYTD | |
| | QFGASPTEGSLTPFIHNAFK | |
| | ADIQICILDGEMMAYNPNTQ | |
| | TFMQKGTKFDIKRMVEDSDL | |
| | QTCYCVFDVLMVNNKKLGHE | |
| | TLRKRYEILSSIFTPIPGRI | |
| | EIVQKTQAHTKNEVIDALNE | |
| | AIDKREEGIMVKQPLSIYKP | |
| | DKRGEGWLKIKPEYVSGLMD | |
| | ELDILIVGGYWGKGSRGGMM | |
| | SHFLCAVAEKPPPGEKPSVF | |
| | HTLSRVGSGCTMKELYDLGL | |
| | KLAKYWKPFHRKAPPSSILC | |
| | GTEKPEVYIEPCNSVIVQIK | |
| | AAEIVPSDMYKTGCTLRFPR | |
| | IEKIRDDKEWHECMTLDDLE | |
| | QLRGKASGKLASKHLYIGGD | |
| | DEPQEKKRKAAPKMKKVIGI | |
| | IEHLKAPNLTNVNKISNIFE | |
| | DVEFCVMSGTDSQPKPDLEN | |
| | RIAEFGGYIVQNPGPDTYCV | |
| | IAGSENIRVKNIILSNKHDV | |
| | VKPAWLLECFKTKSFVPWQP | |
| | RFMIHMCPSTKEHFAREYDC | |
| | YGDSYFIDTDLNQLKEVFSG | |
| | IKNSNEQTPEEMASLIADLE | |
| | YRYSWDCSPLSMFRRHTVYL | |
| | DSYAVINDLSTKNEGTRLAI | |
| | KALELRFHGAKVVSCLAEGV | |
| | SHVIIGEDHSRVADFKAFRR | |
| | TFKRKFKILKESWVTDSIDK | |
| | CELQEENQYLI | |
| T4 Ligase | MILKILNEIASIGSTKQKQA | 64 |
| | ILEKNKDNELLKRVYRLTYS | |
| | RGLQYYIKKWPKPGIATQSF | |
| | GMLTLTDMLDFIEFTLATRK | |
| | LTGNAAIEELTGYITDGKKD | |
| | DVEVLRRVMMRDLECGASVS | |
| | IANKVWPGLIPEQPQMLASS | |
| | YDEKGINKNIKFPAFAQLKA | |
| | DGARCFAEVRGDELDDVRLL | |
| | SRAGNEYLGLDLLKEELIKM | |
| | TAEARQIHPEGVLIDGELVY | |
| | HEQVKKEPEGLDFLFDAYPE | |
| | NSKAKEFAEVAESRTASNGI | |
| | ANKSLKGTISEKEAQCMKFQ | |
| | VWDYVPLVEIYSLPAFRLKY | |
| | DVRFSKLEQMTSGYDKVILI | |
| | ENQVVNNLDEAKVIYKKYID | |
| | QGLEGIILKNIDGLWENARS | |
| | KNLYKFKEVIDVDLKIVGIY | |
| | PHRKDPTKAGGFILESECGK | |
| | IKVNAGSGLKDKAGVKSHEL | |
| | DRTRIMENQNYYIGKILECE | |
| | CNGWLKSDGRTDYVKLFLPI | |
| | AIRLREDKTKANTFEDVFGD | |
| | FHEVTGL | |
| T7 Ligase | MMNIKTNPFKAVSFVESAIK | 65 |
| | KALDNAGYLIAEIKYDGVRG | |
| | NICVDNTANSYWLSRVSKTI | |
| | PALEHLNGFDVRWKRLLNDD | |
| | RCFYKDGFMLDGELMVKGVD | |
| | FNTGSGLLRTKWTDTKNQEF | |
| | HEELFVEPIRKKDKVPFKLH | |
| | TGHLHIKLYAILPLHIVESG | |
| | EDCDVMTLLMQEHVKNMLPL | |
| | LQEYFPEIEWQAAESYEVYD | |
| | MVELQQLYEQKRAEGHEGLI | |
| | VKDPMCIYKRGKKSGWWKMK | |
| | PENEADGIIQGLVWGTKGLA | |
| | NEGKVIGFEVLLESGRLVNA | |
| | TNISRALMDEFTETVKEATL | |
| | SQWGFFSPYGIGDNDACTIN | |
| | PYDGWACQISYMEETPDGSL | |
| | RHPSFVMFRGTEDNPQEKM | |
| Taq Ligase | MTLEAARRRVNELRDLIRYH | 66 |
| | NYLYYVLDAPEISDAEYDRL | |
| | LRELKELEERFPELQSPDSP | |
| | TEQVGARPLESTFRPVRHPT | |
| | RMYSLDNAFSLDEVRAFEER | |
| | IERALGRKGPFLYTVEHKVD | |
| | GLSVNLYYEEGILVFGATRG | |
| | DGETGEEVTQNLLTIRTIPR | |
| | RLTGVPDRLEVRGEVYMPIE | |
| | AFLRLNQELEEAGERIFKNP | |
| | RNAAAGSLRQKDPRVTARRG | |
| | LRATFYALGLGLEETGLKSQ | |
| | HDLLLWLRERGFPVEHGFTR | |
| | ALGAEGVEEVYQAWLKERRK | |
| | LPFEADGVVVKLDDLALWRE | |
| | LGYTARAPRFALAYKFPAEE | |
| | KETRLLSVAFQVGRTGRITP | |
| | VGVLEPVFIEGSEVSRVTLH | |
| | NESFIEELDVRIGDWVLVHK | |
| | AGGVIPEVLRVLKERRTGEE | |
| | KPILWPENCPECGHALIKEG | |
| | KVHRCPNPLCPAKRFEAIRH | |
| | YASRKAMDIQGLGEKLIEKL | |
| | LEKGLVRDVADLYRLKKEDL | |
| | VNLERMGEKSAENLLRQIEE | |
| | SKGRGLERLLYALGLPGVGE | |
| | VLARNLALRFGHMDRLLEAG | |
| | LEDLLEVEGVGELTARAILN | |
| | TLKDPEFRDLVRRLKEAGVE | |
| | MEAKEREGEALKGLTFVITG | |
| | ELSRPREEVKALLRRLGAKV | |
| | TDSVSRKTGFLVVGENPGSK | |
| | LEKARALGVPTLSEEELYRL | |
| | IEERTGKDPRALTA | |
| T3 Ligase | MNIFNTNPFKAVSFVESAVK | 67 |
| | KALETSGYLIADCKYDGVRG | |
| | NIVVDNVAEAAWLSRVSKFI | |
| | PALEHLNGFDKRWQQLLNDD | |
| | RCIFPDGFMLDGELMVKGVD | |
| | FNTGSGLLRTKWVKRDNMGF | |
| | HLTNVPTKLTPKGREVIDGK | |
| | FEFHLDPKRLSVRLYAVMPI | |
| | HIAESGEDYDVQNLLMPYHV | |
| | EAMRSLLVEYFPEIEWLIAE | |
| | TYEVYDMDSLTELYEEKRAE | |
| | GHEGLIVKDPQGIYKRGKKS | |
| | GWWKLKPECEADGIIQGVNW | |
| | GTEGLANEGKVIGFSVLLET | |
| | GRLVDANNISRALMDEFTSN | |
| | VKAHGEDFYNGWACQVNYME | |
| | ATPDGSLRHPSFEKFRGTED | |
| | NPQEKM | |
| NAD-dependent | MESIEQQLTELRTTLRHHEY | 68 |
| E coli | LYHVMDAPEIPDAEYDRLMR | |
| DNA ligase LigA | ELRELETKHPELITPDSPTQ | |
| | RVGAAPLAAFSQIRHEVPML | |
| | SLDNVFDEESFLAFNKRVQD | |
| | RLKNNEKVTWCCELKLDGLA | |
| | VSILYENGVLVSAATRGDGT | |
| | TGEDITSNVRTIRAIPLKLH | |
| | GENIPARLEVRGEVFLPQAG | |
| | FEKINEDARRTGGKVFANPR | |
| | NAAAGSLRQLDPRITAKRPL | |
| | TFFCYGVGVLEGGELPDTHL | |
| | GRLLQFKKWGLPVSDRVTLC | |
| | ESAEEVLAFYHKVEEDRPTL | |
| | GFDIDGVVIKVNSLAQQEQL | |
| | GFVARAPRWAVAFKFPAQEQ | |
| | MTFVRDVEFQVGRTGAITPV | |
| | ARLEPVHVAGVLVSNATLHN | |
| | ADEIERLGLRIGDKVVIRRA | |
| | GDVIPQVVNVVLSERPEDTR | |
| | EVVFPTHCPVCGSDVERVEG | |
| | EAVARCTGGLICGAQRKESL | |
| | KHFVSRRAMDVDGMGDKIID | |
| | QLVEKEYVHTPADLFKLTAG | |
| | KLTGLERMGPKSAQNVVNAL | |
| | EKAKETTFARFLYALGIREV | |
| | GEATAAGLAAYFGTLEALEA | |
| | ASIEELQKVPDVGIVVASHV | |
| | HNFFAEESNRNVISELLAEG | |
| | VHWPAPIVINAEEIDSPFAG | |
| | KTVVLTGSLSQMSRDDAKAR | |
| | LVELGAKVAGSVSKKTDLVI | |
| | AGEAAGSKLAKAQELGIEVI | |
| | DEAEMLRLLGS | |
| Thermococcus | MRYSELADLYRRLEKTTLKT | 69 |
| kodakarensis | LKTKFVADFLKKTPDELLEI | |
| DNA ligase | VPYLILGKVFPDWDERELGV | |
| | GEKLLIKAVSMATGVPEKEI | |
| | EDSVRDTGDLGESVALAIKK | |
| | KKQKSFFSQPLTIKRVYDTF | |
| | VKIAEAQGEGSQDRKMKYLA | |
| | NLFMDAEPEEGKYLARTVLG | |
| | TMRTGVAEGILRDAIAEAFR | |
| | VKPELVERAYMLTSDFGYVA | |
| | KIAKLEGNEGLSKVRIQIGK | |
| | PIRPMLAQNAASVKDALIEM | |
| | GGEAAFEIKYDGARVQVHKD | |
| | GDKVIVYSRRLENVTRSIPE | |
| | VIEAIKAALKPEKAIVEGEL | |
| | VAVGENGRPRPFQYVLRRFR | |
| | RKYNIDEMIEKIPLELNLFD | |
| | VMFVDGESLIETKFIDRRNK | |
| | LEEIVKESEKIKLAEQLITK | |
| | KVEEAEAFYRRALELGHEGL | |
| | MAKRLDSIYEPGNRGKKWLK | |
| | IKPTMENLDLVIIGAEWGEG | |
| | RRAHLLGSFLVAAYDPHSGE | |
| | FLPVGKVGSGFTDEDLVEFT | |
| | KMLKPYIVRQEGKFVEIEPK | |
| | FVIEVTYQEIQKSPKYKSGF | |
| | ALRFPRYVALREDKSPEEAD | |
| | TIERVAELYELQERFKAKK | |
| African swine | MLNQFPGQYSNNIFCFPPIE | 70 |
| fever virus | SETKSGKKASWIICVQVVQH | |
| DNA ligase | NTIIPITDEMFSTDVKDAVA | |
| | EIFTKFFVEEGAVRISKMTR | |
| | VTEGKNLGKKNATTVVHQAF | |
| | KDALSKYNRHARQKRGAHTN | |
| | RGMIPPMLVKYFNIIPKTFF | |
| | EEETDPIVQRKRNGVRAVAC | |
| | QQGDGCILLYSRTEKEFLGL | |
| | DNIKKELKQLYLFIDVRVYL | |
| | DGELYLHRKPLQWIAGQANA | |
| | KTDSSELHFYVFDCFWSDQL | |
| | QMPSNKRQQLLTNIFKQKED | |
| | LTFIHQVENFSVKNVDEALR | |
| | LKAQFIKEGYEGAIVRNANG | |
| | PYEPGYNNYHSAHLAKLKPL | |
| | LDAEFILVDYTQGKKGKDLG | |
| | AILWVCELPNKKRFVVTPKH | |
| | LTYADRYALFQKLTPALFKK | |
| | HLYGKELTVEYAELSPKTGI | |
| | PLQARAVGFREPINVLEII | |
| Vaccinia | MTSLREFRKLCCDIYHASGY | 71 |
| virus DNA | KEKSKLIRDFITDRDDKYLI | |
| ligase | IKLLLPGLDDRIYNMNDKQI | |
| (strain Western | IKLYSIIFKQSQEDMLQDLG | |
| Reserve) | YGYIGDTIRTFFKENTEIRP | |
| | RDKSILTLEDVDSFLTTLSS | |
| | VTKESHQIKLLTDIASVCTC | |
| | NDLKCVVMLIDKDLKIKAGP | |
| | RYVLNAISPNAYDVFRKSNN | |
| | LKEIIENASKQNLDSISISV | |
| | MTPINPMLAESCDSVNKAFK | |
| | KFPSGMFAEVKYDGERVQVH | |
| | KNNNEFAFFSRNMKPVLSHK | |
| | VDYLKEYIPKAFKKATSIVL | |
| | DSEIVLVDEHNVPLPFGSLG | |
| | IHKKKEYKNSNMCLFVFDCL | |
| | YFDGFDMTDIPLYERRSFLK | |
| | DVMVEIPNRIVFSELTNISN | |
| | ESQLTDVLDDALTRKLEGLV | |
| | LKDINGVYEPGKRRWLKIKR | |
| | DYLNEGSMADSADLVVLGAY | |
| | YGKGAKGGIMAVFLMGCYDD | |
| | ESGKWKTVTKCSGHDDNTLR | |
| | VLQDQLTMVKINKDPKKIPE | |
| | WLVVNKIYIPDFVVEDPKQS | |
| | QIWEISGAEFTSSKSHTANG | |
| | ISIRFPRFTRIREDKTWKES | |
| | THLNDLVNLTKS | |
| Vaccinia | MTSLREFRKLCCDIYHASGY | 72 |
| virus DNA | KEKSKLIRDFITDRDDKYLI | |
| ligase | IKLLLPGLDDRIYNMNDKQI | |
| (strain Ankara) | IKLYSIIFKQSQEDMLQDLG | |
| | YGYIGDTIRTFFKENTEIRP | |
| | RDKSILTLEEVDSFLTTLSS | |
| | VTKESHQIKLLTDIASVCTC | |
| | NDLKCVVMLIDKDLKIKAGP | |
| | RYVLNAISPHAYDVFRKSNN | |
| | LKEIIENASKQNLDSISISV | |
| | MTPINPMLAESCDSVNKAFK | |
| | KFPSGMFAEVKYDGERVQVH | |
| | KNNNEFAFFSRNMKPVLSHK | |
| | VDYLKEYIPKAFKKATSIVL | |
| | DSEIVLVDEHNVPLPFGSLG | |
| | IHKKKEYKNSNMCLFVFDCL | |
| | YFDGFDMTDIPLYERRSFLK | |
| | DVMVEIPNRIVFSELTNISN | |
| | ESQLTDVLDDALTRKLEGLV | |
| | LKDINGVYEPGKRRWLKIKR | |
| | DYLNEGSMADSADLVVLGAY | |
| | YGKGAKGGIMAVFLMGCYDD | |
| | ESGKWKTVTKCSGHDDNTLR | |
| | ELQDQLKMIKINKDPKKIPE | |
| | WLVVNKIYIPDFVVEDPKQS | |
| | QIWEISGAEFTSSKSHTANG | |
| | ISIRFPRFTRIREDKTWKES | |
| | THLNDLVNLTKS | |
| Burkholderia | MARSPVEPPASQPAKRAAWL | 73 |
| pseudomallei | RAELERANYAYYVLDQPDLP | |
| DNA ligase | DAEYDRLFVELQRIEAEHPD | |
| | LVTPDSPTQRVGGEAASGFT | |
| | PVVHDKPMLSLNNGFADEDV | |
| | IAFDKRVADGLDKATDLAGT | |
| | VTEPVEYACELKFDGLAISL | |
| | RYENGRFVQASTRGDGTTGE | |
| | DVTENIRTIRAIPLTLKGKR | |
| | IPRMLDVRGEVLMFKRDFAR | |
| | LNERQRAAGQREFANPRNAA | |
| | AGSLRQLDSKITASRPLSFF | |
| | AYGIGVLDGADMPDTHSGLL | |
| | DWYETLGLPVNRERAVVRGA | |
| | AGLLAFFHSVGERRESLPYD | |
| | IDGVVYKVNRRDEQDRLGFV | |
| | SRAPRFALAHKFPAQEALTK | |
| | LIAIDVQVGRTGAITPVARL | |
| | EPVFVGGATVTNATLHNEDE | |
| | VRRKDIRIGDTVIVRRAGDV | |
| | IPEVVSAVLDRRPADAQEFV | |
| | MPTECPECGSRIERLPDEAI | |
| | ARCTGGLFCPAQRKQALWHF | |
| | AQRRALDIDGLGEKIIDQLV | |
| | EQNLVRTPADLFNLGFSTLV | |
| | GLDRFAEKSARNLIDSLEKA | |
| | KHTTLARFIYALGIRHVGES | |
| | TAKDLAKHFGSLDPIMDAPI | |
| | DALLEVNDVGPIVAESIHQF | |
| | FAEEHNRTVIEQLRARGKVT | |
| | WPEGPPAPRAPQGVLAGKTV | |
| | VLTGTLPTLTREAAKEMLEA | |
| | AGAKVAGSVSKKTDYVVAGA | |
| | DAGSKLAKAEELGIPVLDEA | |
| | GMHTLLEGHAR | |
| Alteromonas | MQFFLTVFCLLLITAVTHVN | 74 |
| mediterranea | AEDKLDIVDGLQLAKQYSHS | |
| DNA ligase | RQDINIAEYWVSEKLDGIRA | |
| | RWDGTELRTRNNNKIAAPAW | |
| | FTANWPKATIDGELWIARGQ | |
| | FERTASIVLSKLTSVAPHSV | |
| | AGSLPRTESTVGAMTATHSL | |
| | PSKRWAKIRFMAFDMPVAGQ | |
| | SFDSRLNMLNNLKEATPNPT | |
| | FAVVSQFTLSSVNALEEKLE | |
| | QVTLSGGEGLMLHHKKAFYH | |
| | SGRSDKLLKVKQFEDAEAKV | |
| | LAHLPGKGKFKGMMGSLLVE | |
| | TPAGVQFKLGTGFSEKERQA | |
| | PPAIGSWVTFKFYGVTKNGK | |
| | PRFASFLRVRPPSDLPK | |
| Yeast DNA | MRRLLTGCLLSSARPLKSRL | 75 |
| ligase I | PLLMSSSLPSSAGKKPKQAT | |
| (Cdc9p) | LARFFTSMKNKPTEGTPSPK | |
| | KSSKHMLEDRMDNVSGEEEY | |
| | ATKKLKQTAVTHTVAAPSSM | |
| | GSNFSSIPSSAPSSGVADSP | |
| | QQSQRLVGEVEDALSSNNND | |
| | HYSSNIPYSEVCEVFNKIEA | |
| | ISSRLEIIRICSDFFIKIMK | |
| | QSSKNLIPTTYLFINRLGPD | |
| | YEAGLELGLGENLLMKTISE | |
| | TCGKSMSQIKLKYKDIGDLG | |
| | EIAMGARNVQPTMFKPKPLT | |
| | VGEVFKNLRAIAKTQGKDSQ | |
| | LKKMKLIKRMLTACKGIEAK | |
| | FLIRSLESKLRIGLAEKTVL | |
| | ISLSKALLLHDENREDSPDK | |
| | DVPMDVLESAQQKIRDAFCQ | |
| | VPNYEIVINSCLEHGIMNLD | |
| | KYCTLRPGIPLKPMLAKPTK | |
| | AINEVLDRFQGETFTSEYKY | |
| | DGERAQVHLLNDGTMRIYSR | |
| | NGENMTERYPEINITDFIQD | |
| | LDTTKNLILDCEAVAWDKDQ | |
| | GKILPFQVLSTRKRKDVELN | |
| | DVKVKVCLFAFDILCYNDER | |
| | LINKSLKERREYLTKVTKVV | |
| | PGEFQYATQITTNNLDELQK | |
| | FLDESVNHSCEGLMVKMLEG | |
| | PESHYEPSKRSRNWLKLKKD | |
| | YLEGVGDSLDLCVLGAYYGR | |
| | GKRTGTYGGFLLGCYNQDTG | |
| | EFETCCKIGTGFSDEMLQLL | |
| | HDRLTPTIIDGPKATFVFDS | |
| | SAEPDVWFEPTTLFEVLTAD | |
| | LSLSPIYKAGSATFDKGVSL | |
| | RFPRFLRIREDKGVEDATSS | |
| | DQIVELYENQSHMQN | |
| Yeast DNA | MISALDSIPEPQNFAPSPDF | 76 |
| ligase IV | KWLCEELFVKIHEVQINGTA | |
| | GTGKSRSFKYYEIISNFVEM | |
| | WRKTVGNNIYPALVLALPYR | |
| | DRRIYNIKDYVLIRTICSYL | |
| | KLPKNSATEQRLKDWKQRVG | |
| | KGGNLSSLLVEEIAKRRAEP | |
| | SSKAITIDNVNHYLDSLSGD | |
| | RFASGRGFKSLVKSKPFLHC | |
| | VENMSFVELKYFFDIVLKNR | |
| | VIGGQEHKLLNCWHPDAQDY | |
| | LSVISDLKVVTSKLYDPKVR | |
| | LKDDDLSIKVGFAFAPQLAK | |
| | KVNLSYEKICRTLHDDFLVE | |
| | EKMDGERIQVHYMNYGESIK | |
| | FFSRRGIDYTYLYGASLSSG | |
| | TISQHLRFTDSVKECVLDGE | |
| | MVTFDAKRRVILPFGLVKGS | |
| | AKEALSFNSINNVDFHPLYM | |
| | VFDLLYLNGTSLTPLPLHQR | |
| | KQYLNSILSPLKNIVEIVRS | |
| | SRCYGVESIKKSLEVAISLG | |
| | SEGVVLKYYNSSYNVASRNN | |
| | NWIKVKPEYLEEFGENLDLI | |
| | VIGRDSGKKDSFMLGLLVLD | |
| | EEEYKKHQGDSSEIVDHSSQ | |
| | EKHIQNSRRRVKKILSFCSI | |
| | ANGISQEEFKEIDRKTRGHW | |
| | KRTSEVAPPASILEFGSKIP | |
| | AEWIDPSESIVLEIKSRSLD | |
| | NTETNMQKYATNCTLYGGYC | |
| | KRIRYDKEWTDCYTLNDLYE | |
| | SRTVKSNPSYQAERSQLGLI | |
| | RKKRKRVLISDSFHQNRKQL | |
| | PISNIFAGLLFYVLSDYVTE | |
| | DTGIRITRAELEKTIVEHGG | |
| | KLIYNVILKRHSIGDVRLIS | |
| | CKTTTECKALIDRGYDILHP | |
| | NWVLDCIAYKRLILIEPNYC | |
| | FNVSQKMRAVAEKRVDCLGD | |
| | SFENDISETKLSSLYKSQLS | |
| | LPPMGELEIDSEVRRFPLFL | |
| | FSNRIAYVPRRKISTEDDII | |
| | EMKIKLFGGKITDQQSLCNL | |
| | IIIPYTDPILRKDCMNEVHE | |
| | KIKEQIKASDTIPKIARVVA | |
| | PEWVDHSINENCQVPEEDFP | |
| | VVNY | |
| T6 ligase | MILKILNEIASIGSTKQKQA | 77 |
| | ILEKNKDNELLKRVYRLTYS | |
| | RGLQYYIKKWPKPGIATQSF | |
| | GMLTLTDMLDFIEFTLATRK | |
| | LTGNAAIEELTGYITDGKKD | |
| | DVEVLRRVMMRDLECGASVS | |
| | IANKVWPGLIPEQPQMLASS | |
| | YDEKGINKNIKFPAFAQLKA | |
| | DGARCFAEVRGDELDDVRLL | |
| | SRAGNEYLGLDLLKEELIKM | |
| | TAEARQIHPEGVLIDGELVY | |
| | HEQVKKEPEGLDFLFDAHPE | |
| | NSKVKDFTEVAESRTASNGI | |
| | ANKSLKGTISEKEAQCMKFQ | |
| | VWDYVPLVEVYGLPAFRLKY | |
| | DVRFSKLEQMTSGYDKVILI | |
| | ENQVVNNLDEAKVIYKKYID | |
| | QGLEGIILKNIDGLWENARS | |
| | KNLYKFKEVIDVDLKIVGIY | |
| | PHRKDPTKAGGFILESECGK | |
| | IKVNAGSGLKDKAGVKSHEL | |
| | DRTRIMENQNYYIGKILECE | |
| | CNGWLKSDGRTDYVKLFLPI | |
| | AIRLREDKTKANTFEDVFGD | |
| | FHEVTGL | |
| Mouse DNA | MQRSIMSFFQPTKEGKAKKP | 78 |
| ligase I | EKETPSSIREKEPPPKVALK | |
| | ERNQVVPESDSPVKRTGRKV | |
| | AQVLSCEGEDEDEAPGTPKV | |
| | QKPVSDSEQSSPPSPDTCPE | |
| | NSPVFNCSSPMDISPSGFPK | |
| | RRTARKQLPKRTIQDTLEEQ | |
| | NEDKTKTAKKRKKEEETPKE | |
| | SLAEAEDIKQKEEKEGDQLI | |
| | VPSEPTKSPESVTLTKTENI | |
| | PVCKAGVKLKPQEEEQSKPP | |
| | ARGAKTLSSFFTPRKPAVKT | |
| | EVKQEESGTLRKEETKGTLD | |
| | PANYNPSKNNYHPIEDACWK | |
| | HGQKVPFLAVARTFEKIEEV | |
| | SARLKMVETLSNLLRSVVAL | |
| | SPPDLLPVLYLSLNRLGPPQ | |
| | QGLELGVGDGVLLKAVAQAT | |
| | GRQLESIRAEVAEKGDVGLV | |
| | AENSRSTQRLMLPPPPLTIS | |
| | GVFTKFCDIARLTGSASMAK | |
| | KMDIIKGLFVACRHSEARYI | |
| | ARSLSGRLRLGLAEQSVLAA | |
| | LAQAVSLTPPGQEFPTVVVD | |
| | AGKGKTAEARKMWLEEQGMI | |
| | LKQTFCEVPDLDRIIPVLLE | |
| | HGLERLPEHCKLSPGVPLKP | |
| | MLAHPTRGVSEVLKRFEEVD | |
| | FTCEYKYDGQRAQIHVLEGG | |
| | EVKIFSRNQEDNTGKYPDII | |
| | SRIPKIKHPSVTSFILDTEA | |
| | VAWDREKKQIQPFQVLTTRK | |
| | RKEVDASEIQVQVCLYAFDL | |
| | IYLNGESLVRQPLSRRRQLL | |
| | RENFVETEGEFVFTTSLDTK | |
| | DTEQIAEFLEQSVKDSCEGL | |
| | MVKTLDVDATYEIAKRSHNW | |
| | LKLKKDYLDGVGDTLDLVVI | |
| | GAYLGRGKRAGRYGGFLLAA | |
| | YDEESEELQAICKLGTGFSD | |
| | EELEEHHQSLQALVLPTPRP | |
| | YVRIDGAVAPDHWLDPSIVW | |
| | EVKCADLSLSPIYPAARGLV | |
| | DKEKGISLRFPRFIRVRKDK | |
| | QPEQATTSNQVASLYRKQSQ | |
| | IQNQQSSDLDSDVEDY | |
| Mouse DNA | MASSQTSQTVAAHVPFADLC | 79 |
| ligase IV | STLERIQKGKDRAEKIRHFK | |
| | EFLDSWRKFHDALHKNRKDV | |
| | TDSFYPAMRLILPQLERERM | |
| | AYGIKETMLAKLYIELLNLP | |
| | REGKDAQKLLNYRTPSGART | |
| | DAGDFAMIAYFVLKPRCLQK | |
| | GSLTIQQVNELLDLVASNNS | |
| | GKKKDLVKKSLLQLITQSSA | |
| | LEQKWLIRMIIKDLKLGISQ | |
| | QTIFSIFHNDAVELHNVTTD | |
| | LEKVCRQLHDPSVGLSDISI | |
| | TLFSAFKPMLAAVADVERVE | |
| | KDMKQQSFYIETKLDGERMQ | |
| | MHKDGALYRYFSRNGYNYTD | |
| | QFGESPQEGSLTPFIHNAFG | |
| | TDVQACILDGEMMAYNPTTQ | |
| | TFMQKGVKFDIKRMVEDSGL | |
| | QTCYSVFDVLMVNKKKLGRE | |
| | TLRKRYEILSSTFTPIQGRI | |
| | EIVQKTQAHTKKEVVDALND | |
| | AIDKREEGIMVKHPLSIYKP | |
| | DKRGEGWLKIKPEYVSGLMD | |
| | ELDVLIVGGYWGKGSRGGMM | |
| | SHFLCAVAETPPPGDRPSVF | |
| | HTLCRVGSGYTMKELYDLGL | |
| | KLAKYWKPFHKKSPPSSILC | |
| | GTEKPEVYIEPQNSVIVQIK | |
| | AAEIVPSDMYKTGSTLRFPR | |
| | IEKIRDDKEWHECMTLGDLE | |
| | QLRGKASGKLATKHLHVGDD | |
| | DEPREKRRKPISKTKKAIRI | |
| | IEHLKAPNLSNVNKVSNVFE | |
| | DVEFCVMSGLDGYPKADLEN | |
| | RIAEFGGYIVQNPGPDTYCV | |
| | IAGSENVRVKNIISSDKNDV | |
| | VKPEWLLECFKTKTCVPWQP | |
| | RFMIHMCPSTKQHFAREYDC | |
| | YGDSYFVDTDLDQLKEVFLG | |
| | IKPSEQQTPEEMAPVIADLE | |
| | CRYSWDHSPLSMFRHYTIYL | |
| | DLYAVINDLSSRIEATRLGI | |
| | TALELRFHGAKVVSCLSEGV | |
| | SHVIIGEDQRRVTDFKIFRR | |
| | MLKKKFKILQESWVSDSVDK | |
| | GELQEENQYLL | |
| Arabidopsis | MLAIRSSNYLRCIPSLCTKT | 80 |
| DNA ligase I | QISQFSSVLISFSRQISHLR | |
| | LSSCHRAMSSSRPSAFDALM | |
| | SNARAAAKKKTPQTTNLSRS | |
| | PNKRKIGETQDANLGKTIVS | |
| | EGTLPKTEDLLEPVSDSANP | |
| | RSDTSSIAEDSKTGAKKAKT | |
| | LSKTDEMKSKIGLLKKKPND | |
| | FDPEKMSCWEKGERVPFLFV | |
| | ALAFDLISNESGRIVITDIL | |
| | CNMLRTVIATTPEDLVATVY | |
| | LSANEIAPAHEGVELGIGES | |
| | TIIKAISEAFGRTEDHVKKQ | |
| | NTELGDLGLVAKGSRSTQTM | |
| | MFKPEPLTVVKVFDTFRQIA | |
| | KESGKDSNEKKKNRMKALLV | |
| | ATTDCEPLYLTRLLQAKLRL | |
| | GFSGQTVLAALGQAAVYNEE | |
| | HSKPPPNTKSPLEEAAKIVK | |
| | QVFTVLPVYDIIVPALLSGG | |
| | VWNLPKTCNFTLGVPIGPML | |
| | AKPTKGVAEILNKFQDIVFT | |
| | CEYKYDGERAQIHFMEDGTF | |
| | EIYSRNAERNTGKYPDVALA | |
| | LSRLKKPSVKSFILDCEVVA | |
| | FDREKKKILPFQILSTRARK | |
| | NVNVNDIKVGVCIFAFDMLY | |
| | LNGQQLIQENLKIRREKLYE | |
| | SFEEDPGYFQFATAVTSNDI | |
| | DEIQKFLDASVDVGCEGLII | |
| | KTLDSDATYEPAKRSNNWLK | |
| | LKKDYMDSIGDSVDLVPIAA | |
| | FHGRGKRTGVYGAFLLACYD | |
| | VDKEEFQSICKIGTGFSDAM | |
| | LDERSSSLRSQVIATPKQYY | |
| | RVGDSLNPDVWFEPTEVWEV | |
| | KAADLTISPVHRAATGIVDP | |
| | DKGISLRFPRLLRVREDKKP | |
| | EEATSSEQIADLYQAQKHNH | |
| | PSNEVKGDDD | |
| Arabidopsis | MTEEIKFSVLVSLFNWIQKS | 81 |
| DNA ligase IV | KTSSQKRSKFRKFLDTYCKP | |
| | SDYFVAVRLIIPSLDRERGS | |
| | YGLKESVLATCLIDALGISR | |
| | DAPDAVRLLNWRKGGTAKAG | |
| | ANAGNFSLIAAEVLQRRQGM | |
| | ASGGLTIKELNDLLDRLASS | |
| | ENRAEKTLVLSTLIQKTNAQ | |
| | EMKWVIRIILKDLKLGMSEK | |
| | SIFQEFHPDAEDLFNVTCDL | |
| | KLVCEKLRDRHQRHKRQDIE | |
| | VGKAVRPQLAMRIGDVNAAW | |
| | KKLHGKDVVAECKFDGDRIQ | |
| | IHKNGTDIHYFSRNFLDHSE | |
| | YAHAMSDLIVQNILVDKCIL | |
| | DGEMLVWDTSLNRFAEFGSN | |
| | QEIAKAAREGLDSHKQLCYV | |
| | AFDVLYVGDTSVIHQSLKER | |
| | HELLKKVVKPLKGRLEVLVP | |
| | EGGLNVHRPSGEPSWSIVVH | |
| | AAADVERFFKETVENRDEGI | |
| | VLKDLESKWEPGDRSGKWMK | |
| | LKPEYIRAGADLDVLIIGGY | |
| | YGSGRRGGEVAQFLVALADR | |
| | AEANVYPRRFMSFCRVGTGL | |
| | SDDELNTVVSKLKPYFRKNE | |
| | HPKKAPPSFYQVTNHSKERP | |
| | DVWIDSPEKSIILSITSDIR | |
| | TIRSEVFVAPYSLRFPRIDK | |
| | VRYDKPWHECLDVQAFVELV | |
| | NSSNGTTQKQKESESTQDNP | |
| | KVNKSSKRGEKKNVSLVPSQ | |
| | FIQTDVSDIKGKTSIFSNMI | |
| | FYFVNVPRSHSLETFHKMVV | |
| | ENGGKFSMNLNNSVTHCIAA | |
| | ESSGIKYQAAKRQRDVIHFS | |
| | WVLDCCSRNKMLPLLPKYFL | |
| | HLTDASRTKLQDDIDEFSDS | |
| | YYWDLDLEGLKQVLSNAKQS | |
| | EDSKSIDYYKKKLCPEKRWS | |
| | CLLSCCVYFYPYSQTLSTEE | |
| | EALLGIMAKRLMLEVLMAGG | |
| | KVSNNLAHASHLVVLAMAEE | |
| | PLDFTLVSKSFSEMEKRLLL | |
| | KKRLHVVSSHWLEESLQREE | |
| | KLCEDVYTLRPKYMEESDTE | |
| | ESDKSEHDTTEVASQGSAQT | |
| | KEPASSKIAITSSRGRSNTR | |
| | AVKRGRSSTNSLQRVQRRRG | |
| | KQPSKISGDETEESDASEEK | |
| | VSTRLSDIAEETDSFGEAQR | |
| | NSSRGKCAKRGKSRVGQTQR | |
| | VQRSRRGKKAAKIGGDESDE | |
| | NDELDGNNNVSADAEEGNAA | |
| | GRSVENEETREPDIAKYTES | |
| | QQRDNTVAVEEALQDSRNAK | |
| | TEMDMKEKLQIHEDPLQAML | |
| | MKMFPIPSQKTTETSNRTTG | |
| | EYRKANVSGECESSEKRKLD | |
| | AETDNTSVNAGAESDVVPPL | |
| | VKKKKVSYRDVAGELLKDW | |
| Arabidopsis | MASDSAGATISGNFSNSDNS | 82 |
| DNA ligase 6 | ETLNLNTTKLYSSAISSISP | |
| | QFPSPKPTSSCPSIPNSKRI | |
| | PNTNFIVDLFRLPHQSSSVA | |
| | FFLSHFHSDHYSGLSSSWSK | |
| | GIIYCSHKTARLVAEILQVP | |
| | SQFVFALPMNQMVKIDGSEV | |
| | VLIEANHCPGAVQFLFKVKL | |
| | ESSGFEKYVHTGDFRFCDEM | |
| | RFDPFLNGFVGCDGVFLDTT | |
| | YCNPKFVFPSQEESVGYVVS | |
| | VIDKISEEKVLFLVATYVVG | |
| | KEKILVEIARRCKRKIVVDA | |
| | RKMSMLSVLGCGEEGMFTED | |
| | ENESDVHVVGWNVLGETWPY | |
| | FRPNFVKMNEIMVEKGYDKV | |
| | VGFVPTGWTYEVKRNKFAVR | |
| | FKDSMEIHLVPYSEHSNYDE | |
| | LREFIKFLKPKRVIPTVGVD | |
| | IEKFDCKEVNKMQKHFSGLV | |
| | DEMANKKDFLLGFYRQSYQK | |
| | NEKSDVDVVSHSAEVYEEEE | |
| | KNACEDGGENVPSSRGPILH | |
| | DTTPSSDSRLLIKLRDSLPA | |
| | WVTEEQMLDLIKKHAGNPVD | |
| | IVSNFYEYEAELYKQASLPT | |
| | PSLNNQAVLFDDDVTDLQPN | |
| | PVKGICPDVQAIQKGFDLPR | |
| | KMNLTKGTISPGKRGKSSGS | |
| | KSNKKAKKDPKSKPVGPGQP | |
| | TLFKFFNKVLDGGSNSVSVG | |
| | SETEECNTDKKMVHIDASEA | |
| | YKEVTDQFIDIVNGSESLRD | |
| | YAASIIDEAKGDISRALNIY | |
| | YSKPREIPGDHAGERGLSSK | |
| | TIQYPKCSEACSSQEDKKAS | |
| | ENSGHAVNICVQTSAEESVD | |
| | KNYVSLPPEKYQPKEHACWR | |
| | EGQPAPYIHLVRTFASVESE | |
| | KGKIKAMSMLCNMFRSLFAL | |
| | SPEDVLPAVYLCTNKIAADH | |
| | ENIELNIGGSLISSALEEAC | |
| | GISRSTVRDMYNSLGDLGDV | |
| | AQLCRQTQKLLVPPPPLLVR | |
| | DVFSTLRKISVQTGTGSTRL | |
| | KKNLIVKLMRSCREKEIKFL | |
| | VRTLARNLRIGAMLRTVLPA | |
| | LGRAIVMNSFWNDHNKELSE | |
| | SCFREKLEGVSAAVVEAYNI | |
| | LPSLDVVVPSLMDKDIEFST | |
| | STLSMVPGIPIKPMLAKIAK | |
| | GVQEFFNLSQEKAFTCEYKY | |
| | DGQRAQIHKLLDGTVCIFSR | |
| | NGDETTSRFPDLVDVIKQFS | |
| | CPAAETFMLDAEVVATDRIN | |
| | GNKLMSFQELSTRERGSKDA | |
| | LITTESIKVEVCVFVFDIMF | |
| | VNGEQLLALPLRERRRRLKE | |
| | VFPETRPGYLEYAKEITVGA | |
| | EEASLNNHDTLSRINAFLEE | |
| | AFQSSCEGIMVKSLDVNAGY | |
| | CPTKRSDSWLKVKRDYVDGL | |
| | GDTLDLVPIGAWYGNGRKAG | |
| | WYSPFLMACFNPETEEFQSV | |
| | CRVMSGFSDAFYIEMKEFYS | |
| | EDKILAKKPPYYRTGETPDM | |
| | WFSAEVVWEIRGADFTVSPV | |
| | HSASLGLVHPSRGISVRFPR | |
| | FISKVTDRNPEECSTATDIA | |
| | EMFHAQTRKMNITSQH | |
| Bacillus | MDKETAKQRAEELRRTINKY | 83 |
| subtilis DNA | SYEYYTLDEPSVPDAEYDRL | |
| ligase | MQELIAIEEEHPDLRTPDSP | |
| | TQRVGGAVLEAFQKVTHGTP | |
| | MLSLGNAFNADDLRDFDRRV | |
| | RQSVGDDVAYNVELKIDGLA | |
| | VSLRYEDGYFVRGATRGDGT | |
| | TGEDITENLKTIRNIPLKMN | |
| | RELSIEVRGEAYMPKRSFEA | |
| | LNEERIKNEEEPFANPRNAA | |
| | AGSLRQLDPKIAAKRNLDIF | |
| | VYSIAELDEMGVETQSQGLD | |
| | FLDELGFKTNQERKKCGSIE | |
| | EVITLIDELQAKRADLPYEI | |
| | DGIVIKVDSLDQQEELGFTA | |
| | KSPRWAIAYKFPAEEVVTKL | |
| | LDIELNVGRTGVITPTAILE | |
| | PVKVAGTTVSRASLHNEDLI | |
| | KEKDIRILDKVVVKKAGDII | |
| | PEVVNVLVDQRTGEEKEFSM | |
| | PTECPECGSELVRIEGEVAL | |
| | RCINPECPAQIREGLIHFVS | |
| | RNAMNIDGLGERVITQLFEE | |
| | NLVRNVADLYKLTKERVIQL | |
| | ERMGEKSTENLISSIQKSKE | |
| | NSLERLLFGLGIRFIGSKAA | |
| | KTLAMHFESLENLKKASKEE | |
| | LLAVDEIGEKMADAVITYFH | |
| | KEEMLELLNELQELGVNTLY | |
| | KGPKKVKAEDSDSYFAGKTI | |
| | VLTGKLEELSRNEAKAQIEA | |
| | LGGKLTGSVSKNTDLVIAGE | |
| | AAGSKLTKAQELNIEVWNEE | |
| | QLMGELKK | |
| Bacillus | MDRQQAERRAAELRELLNRY | 84 |
| stearo- | GYEYYVLDRPSVPDAEYDRL | |
| thermophilus | MQELIAIEEQYPELKTSDSP | |
| | TQRIGGPPLEAFRKVAHRVP | |
| | MMSLANAFGEGDLRDFDRRV | |
| | RQEVGEAAYVCELKIDGLAV | |
| | SVRYEDGYFVQGATRGDGTT | |
| | GEDITENLKTIRSLPLRLKE | |
| | PVSLEARGEAFMPKASFLRL | |
| | NEERKARGEELFANPRNAAA | |
| | GSLRQLDPKVAASRQLDLFV | |
| | YGLADAEALGIASHSEALDY | |
| | LQALGFKVNPERRRCANIDE | |
| | VIAFVSEWHDKRPQLPYEID | |
| | GIVIKVDSFAQQRALGATAK | |
| | SPRWAIAYKFPAEEVVTTLI | |
| | GIEVNVGRTGVVTPTAILEP | |
| | VRVAGTTVQRATLHNEDFIR | |
| | EKDIRIGDAVIIKKAGDIIP | |
| | EVVGVVVDRRDGDETPFAMP | |
| | THCPECESELVRLEGEVALR | |
| | CLNPNCPAQLRERLIHFASR | |
| | AAMNIEGLGEKVVTQLFNAG | |
| | LVRDVADLYCLTKEQLVGLE | |
| | RMGEKSAANLLAAIEASKQN | |
| | SLERLLFGLGIRYVGAKAAQ | |
| | LLAEHFETMERLERATKEEL | |
| | MAVPEIGEKMADAITAFFAQ | |
| | PEATELLQELRAYGVNMAYK | |
| | GPKRSAEAPADSAFAGKTVV | |
| | LTGKLASMSRNEAKEQIERL | |
| | GGRVTGSVSRSTDLVIAGED | |
| | AGSKLEKAQQLGIEIWDESR | |
| | FLQEINRGKR | |
| Haemophilus | MKFYRTLLLFFASSFAFANS | 85 |
| influenzae Rd | DLMLLHTYNNQPIEGWVMSE | |
| | KLDGVRGYWNGKQLLTRQGQ | |
| | RLSPPAYFIKDFPPFAIDGE | |
| | LFSERNHFEEISTITKSFKG | |
| | DGWEKLKLYVFDVPDAEGNL | |
| | FERLAKLKAHLLEHPTTYIE | |
| | IIEQIPVKDKTHLYQFLAQV | |
| | ENLQGEGVVVRNPNAPYERK | |
| | RSSQILKLKTARGEECTVIA | |
| | HHKGKGQFENVMGALTCKNH | |
| | RGEFKIGSGFNLNERENPPP | |
| | IGSVITYKYRGITNSGKPRF | |
| | ATYWREKK | |
| Pseudo- | MSSSISEQVNHLRIILEQHN | 86 |
| alteromonas | YNYYVLDTPSIPDSEYDRLL | |
| haloplanktis | RELSALETEHPEFLTADSPT | |
| | QKVGGAALSKFEQVAHQVPM | |
| | LSLDNAFSEEEFTAFNRRIK | |
| | ERLMSTDELTFCCEPKLDGL | |
| | AVSIIYRDGVLVQAATRGDG | |
| | FTGENITQNVKTIRNVPLKL | |
| | RGDYPKELEVRGEVFMDSAG | |
| | FDKLNTEAEKRGEKVFVNPR | |
| | NAAAGSLRQLDSKITAKRPL | |
| | MFYAYSTGLVADGNIPEDHY | |
| | QQLEKLTDWGLPLCPETKLV | |
| | EGPKAALAYYRDILTRRSEL | |
| | KYEIDGVVIKINQKTLQERL | |
| | GFVARAPRWAIAYKFPAQEE | |
| | ITQLLDVDFQVGRTGAITPV | |
| | ARLKPVFVGGVTVSNATLHN | |
| | SDEVARLGVKVGDTVIIRRA | |
| | GDVIPQITQVVLERRPDDAR | |
| | DIEFPTTCPICDSHVEKVEG | |
| | EAVARCTGGLVCPAQRKQAI | |
| | KHFASRKALDIDGLGDKIVD | |
| | QLVDRELIKTPADLFILKQG | |
| | HFESLERMGPKSAKNLVTAL | |
| | EEAKGTTLAKFLYSLGIREA | |
| | GEATAQNLANHFLTLENVIN | |
| | ASIDSLTQVSDVGEIVAAHV | |
| | RGFFDEEHNLAVVNALIDQG | |
| | VNWPALSAPSEEEQPLAGLT | |
| | YVLTGTLNTLNRNDAKARLQ | |
| | QLGAKVSGSVSAKTDALVAG | |
| | EKAGSKLTKAQDLGIDILTE | |
| | DELIELLIKHNG | |
| Rhodothermus | METHTAPQTAEARLLEATHT | 87 |
| marinus | LLQTVRQRDLEAIDRKEAEA | |
| | LAARLREVLNQHAYRYYVLD | |
| | NPLIPDADYDLLMQALRKLE | |
| | ARFPELVTPDSPTQRVGGPP | |
| | LGRFEKVRHPEPLLSLNNAF | |
| | GEEDVRVWYERCCRMLAERL | |
| | GQPVQPAVTAELKIDGLAMA | |
| | LTYENGVLSVGATRGDGIEG | |
| | ENVTQNVRTIPAIPLRIPVD | |
| | PAVGPPPTRLEVRGEVYMRK | |
| | RDFERLNEQLQARGERPFAN | |
| | PRNAAAGSVRQLNPQVTALR | |
| | PLSFFAYGIGPVEGAEVPDS | |
| | QYEVLQWLGRLGFPVNEHAR | |
| | RFEHLDDVLEYCRYWTEHRD | |
| | ELDYEIDGVVLKIDHRPWQA | |
| | LLGAISNAPRWAVAYKFPAR | |
| | EAITRLLDIMVSVGRTGVVK | |
| | PVAVLEPVEVGGVTVSQATL | |
| | HNEDYVRSRDIRIGDLVVVI | |
| | RAGDVIPQVVRPVVEARTGN | |
| | ERPWRMPERCPSCGSQLVRL | |
| | PGEADYYCVASDCPAQFVRL | |
| | LEHFAGRDAMDIEGMGSQVA | |
| | RQLAESGLVRPLSDLYRLKL | |
| | EDLLKLEGFAETRARNLLRA | |
| | IEASKQRPLSRLLFGLGIRH | |
| | VGKTTAELLVQRFASIDELA | |
| | AATIDELAALEGVGPITAES | |
| | IANWFRVEDNRRLIEELKEL | |
| | GVNTQRLPEEAPAAESPVRG | |
| | KTFVLTGALPHLTRKEAEEL | |
| | IKRAGGRVASSVSRNTDYVV | |
| | VGENPGSKYDRARQLGIPML | |
| | DEDGLLRLLGMK | |
| Thermus | MTREEARRRINELRDLIRYH | 88 |
| filiformis | NYRYYVLADPEISDAEYDRL | |
| | LRELKELEERFPEFKSPDSP | |
| | TEQVGARPLEPTFRPVRHPT | |
| | RMYSLDNAFTYEEVLAFEER | |
| | LERALGRKRPFLYTVEHKVD | |
| | GLSVNLYYEEGVLVFGATRG | |
| | DGEVGEEVTQNLLTIPTIPR | |
| | RLKGVPDRLEVRGEVYMPIE | |
| | AFLRLNEELEERGEKVFKNP | |
| | RNAAAGSLRQKDPRVTAKRG | |
| | LRATFYALGLGLEESGLKSQ | |
| | YELLLWLKEKGFPVEHGYEK | |
| | ALGAEGVEEVYRRFLAQRHA | |
| | LPFEADGVVVKLDDLALWRE | |
| | LGYTARAPRFALAYKFPAEE | |
| | KETRLLDVVFQVGRTGRVTP | |
| | VGVLEPVFIEGSEVSRVTLH | |
| | NESYIEELDIRIGDWVLVHK | |
| | AGGVIPEVLRVLKERRTGEE | |
| | RPIRWPETCPECGHRLVKEG | |
| | KVHRCPNPLCPAKRFEAIRH | |
| | YASRKAMDIEGLGEKLIERL | |
| | LEKGLVRDVADLYHLRKEDL | |
| | LGLERMGEKSAQNLLRQIEE | |
| | SKHRGLERLLYALGLPGVGE | |
| | VLARNLARRFGTMDRLLEAS | |
| | LEELLEVEEVGELTARAILE | |
| | TLKDPAFRDLVRRLKEAGVS | |
| | MESKEEVSDLLSGLTFVLTG | |
| | ELSRPREEVKALLQRLGAKV | |
| | TDSVSRKTSYLVVGENPGSK | |
| | LEKARALGVAVLTEEEFWRF | |
| | LKEKGAPVPA | |
| Thermus | MTLEEARKRVNELRDLIRYH | 89 |
| scotoductus | NYRYYVLADPEISDAEYDRL | |
| | LRELKELEERFPELKSPDSP | |
| | TEQVGAKPLEATFRPIRHPT | |
| | RMYSLDNAFNFDELKAFEER | |
| | IGRALGREGPFAYTVEHKVD | |
| | GLSVNLYYEDGVLVWGATRG | |
| | DGEVGEEVTQNLLTIPTIPR | |
| | RVKGVPERLEVRGEVYMPIE | |
| | AFLRLNEELEEKGEKIFKNP | |
| | RNAAAGSLRQKDPRITARRG | |
| | LRATFYALGLGLEESGLKTQ | |
| | LDLLHWLREKGFPVEHGFAR | |
| | AEGAEGVERIYQGWLKERRS | |
| | LPFEADGVVVKLDELSLWRE | |
| | LGYTARAPRFAIAYKFPAEE | |
| | KETRLLQVVFQVGRTGRVTP | |
| | VGILEPVFIEGSVVSRVTLH | |
| | NESYIEELDVRIGDWVLVHK | |
| | AGGVIPEVLRVLKEKRTGEE | |
| | RPIRWPETCPECGHRLVKEG | |
| | KVHRCPNPLCPAKRFEAIRH | |
| | YASRKAMDIGGLGEKLIEKL | |
| | LEKGLVKDVADLYRLKKEDL | |
| | LGLERMGEKSAQNLLRQIEE | |
| | SKGRGLERLLYALGLPGVGE | |
| | VLARNLAAHFGTMDRLLEAS | |
| | LEELLQVEEVGELTARGIYE | |
| | TLQDPAFRDLVRRLKEAGVV | |
| | MEAKERGEEALKGLTFVITG | |
| | ELSRPREEVKALLRRLGAKV | |
| | TDSVSRKTSYLVVGENPGSK | |
| | LEKARALGVPTLTEEELYRL | |
| | IEERTGKPVETLAS | |
| Thermus | MTLEEARRRVNELRDLIRYH | 90 |
| species AK16D | NYLYYVLDAPEISDAEYDRL | |
| | LRELKELEERFPELKSPDSP | |
| | TEQVGARPLEATFRPVRHPT | |
| | RMYSLDNAFSLDEVRAFEER | |
| | IERALGRKGPFLYTVERKVD | |
| | GLSVNLYYEEGILVFGATRG | |
| | DGETGEEVTQNLLTIPTIPR | |
| | RLTGVPDRLEVRGEVYMPIE | |
| | AFLRLNQELEEAGERIFKNP | |
| | RNAAAGSLRQKDPRVTARRG | |
| | LRATFYALGLGLEETGLKSQ | |
| | HDLLLWLRERGFPVEHGFTR | |
| | ALGAEGVEEVYQAWLKERRK | |
| | LPFEADGVVVKLDDLALWRE | |
| | LGYTARTPRFALAYKFPAEE | |
| | KETRLLSVAFQVGRTGRITP | |
| | VGVLEPVFIEGSEVSRVTLH | |
| | NESFIEELDVRIGDWVLVHK | |
| | AGGVIPEVLRVLKERRTGEE | |
| | KPIIWPENCPECGHALIKEG | |
| | KVHRCPNPLCPAKRFEAIRH | |
| | YASRKAMDIQGLGEKLIEKL | |
| | LEKGLVRDVADLYRLKKEDL | |
| | VNLERMGEKSAENLLRQIEE | |
| | SKGRGLERLLYALGLPGVGE | |
| | VLARNLALRFGHMDRLLEAG | |
| | LEDLLEVEGVGELTARAILN | |
| | TLKDPEFRDLVRRLKEAGVE | |
| | MEAKEREGEALKGLTFVITG | |
| | ELSRPREEVKALLRRLGAKV | |
| | TDSVSRKTSFLVVGENPGSK | |
| | LEKARALGVPTLSEEELYRL | |
| | IEERTGKDPRALTA | |
| Thermus | MTLEEARKRVNELRDLIRYH | 91 |
| thermophilus | NYRYYVLADPEISDAEYDRL | |
| HB8 | LRELKELEERFPELKSPDSP | |
| | TLQVGARPLEATFRPVRHPT | |
| | RMYSLDNAFNLDELKAFEER | |
| | IERALGRKGPFAYTVEHKVD | |
| | GLSVNLYYEEGVLVYGATRG | |
| | DGEVGEEVTQNLLTIPTIPR | |
| | RLKGVPERLEVRGEVYMPIE | |
| | AFLRLNEELEERGERIFKNP | |
| | RNAAAGSLRQKDPRITAKRG | |
| | LRATFYALGLGLEEVEREGV | |
| | ATQFALLHWLKEKGFPVEHG | |
| | YARAVGAEGVEAVYQDWLKK | |
| | RRALPFEADGVVVKLDELAL | |
| | WRELGYTARAPRFAIAYKFP | |
| | AEEKETRLLDVVFQVGRTGR | |
| | VTPVGILEPVFLEGSEVSRV | |
| | TLHNESYIEELDIRIGDWVL | |
| | VHKAGGVIPEVLRVLKERRT | |
| | GEERPIRWPETCPECGHRLL | |
| | KEGKVHRCPNPLCPAKRFEA | |
| | IRHFASRKAMDIQGLGEKLI | |
| | ERLLEKGLVKDVADLYRLRK | |
| | EDLVGLERMGEKSAQNLLRQ | |
| | IEESKKRGLERLLYALGLPG | |
| | VGEVLARNLAARFGNMDRLL | |
| | EASLEELLEVEEVGELTARA | |
| | ILETLKDPAFRDLVRRLKEA | |
| | GVEMEAKEKGGEALKGLTFV | |
| | ITGELSRPREEVKALLRRLG | |
| | AKVTDSVSRKTSYLVVGENP | |
| | GSKLEKARALGVPTLTEEEL | |
| | YRLLEARTGKKAEELV | |
| Zymomonas | MNADIDLFSYLNPEKQDLSA | 92 |
| mobilis | LAPKDLSREQAVIELERLAK | |
| | LISHYDHLYHDKDNPAVPDS | |
| | EYDALVLRNRRIEQFFPDLI | |
| | RPDSPSKKVGSRPNSRLPKI | |
| | AHRAAMLSLDNGFLDQDVED | |
| | FLGRVRRFFNLKENQAVICT | |
| | VEPKIDGLSCSLRYEKGILT | |
| | QAVTRGDGVIGEDVTPNVRV | |
| | IDDIPKTLKGDNWPEIIEIR | |
| | GEVYMAKSDFAALNARQTEE | |
| | NKKLFANPRNAAAGSLRQLD | |
| | PNITARRSLRFLAHGWGEAT | |
| | SLPADTQYGMMKVIESYGLS | |
| | VSNLLARADDIGQMLDFYQK | |
| | IEAERADLDFDIDGVVYKLD | |
| | QLDWQQRFGFSARAPRFALA | |
| | HKFPAEKAQTTLLDIEIQVG | |
| | RTGVLTPVAKLEPVTVGGVV | |
| | VSSATLHNSDEIERLGVRPG | |
| | DRVLVQRAGDVIPQIVENLT | |
| | PDVDRPIWRFPHRCPVCDSV | |
| | ARREEGEVAWRCTGGLICPA | |
| | QRVERLCHFVSRTAFEIEGL | |
| | GKSHIESFFADKLIETPADI | |
| | FRLFQKRQLLIEREGWGELS | |
| | VDNLISAIDKRRKVPFDRFL | |
| | FALGIRHVGAVTARDLAKSY | |
| | QTWDNFKAAIDEAAHLRTIL | |
| | QPSSEESEEKYQKRVDKELI | |
| | SFFHIPNMGGKIIRSLLDFF | |
| | AETHNSDVVSDLLQEVQIEP | |
| | LYFELASSPLSGKIIVFTGS | |
| | LQKITRDEAKRQAENLGAKV | |
| | ASSVSKKTNLVVAGEAAGSK | |
| | LSKAKELDISIIDEDRWHRI | |
| | VENDGQDSIKI | |
| Campylobacter | MKKEEYLEKVALANLWMRAY | 93 |
| jejuni | YEKDEPLASDEEYDVLIREL | |
| | RVFEEQNKDEISKDSPTQKI | |
| | APTIQSEFKKIAHLKRMWSM | |
| | EDVFDESELRAWAKRAKCEK | |
| | NFFIEPKFDGASLNLLYENG | |
| | KLVSGATRGDGEVGEDITLN | |
| | VFEIENIPKNIAYKERIEIR | |
| | GEVVILKDDFEKINEKRALL | |
| | NQSLFANPRNAASGSLRQLD | |
| | TSITKERNLKFYPWGVGENT | |
| | LNFTKHSEVMQFIRELGFLK | |
| | DDFIKLCANLDEVLKAYDEL | |
| | LALREKKPMMMDGMVVRIDD | |
| | LALCEELGYTVKFPKFMAAF | |
| | KFPALEKTTRLIGVNLQVGR | |
| | SGVITPVAVLEPVNLDGVVV | |
| | KSATLHNFDEIARLDVKIND | |
| | FVSVIRSGDVIPKITKVFKD | |
| | RREGLEMEISRPKLCPTCQS | |
| | ELLDEGTLIKCQNIDCEDRL | |
| | VNSIIHFVSKKCLNIDGLGE | |
| | NIVELLYKHKKITTLESIFH | |
| | LKFSDFEGLEGFKEKKINNL | |
| | LNAIEQARECELFRFITALG | |
| | IEHIGEVAAKKLSLSFGKEW | |
| | HKQSFEAYANLEGFGEQMAL | |
| | SLCEFTRVNHVRIDEFYKLL | |
| | NLKIEKLEIKSDGVIFGKTF | |
| | VITGTLSRPRDEFKALIEKL | |
| | GGKVSSSVSKKTDYVLFGEE | |
| | AGSKLIKAKELEVKCIDESA | |
| | FNELVKE | |
| Mycobacterium | MSSPDADQTAPEVLRQWQAL | 94 |
| tuberculosis | AEEVREHQFRYYVRDAPIIS | |
| ligA | DAEFDELLRRLEALEEQHPE | |
| | LRTPDSPTQLVGGAGFATDF | |
| | EPVDHLERMLSLDNAFTADE | |
| | LAAWAGRIHAEVGDAAHYLC | |
| | ELKIDGVALSLVYREGRLTR | |
| | ASTRGDGRTGEDVTLNARTI | |
| | ADVPERLTPGDDYPVPEVLE | |
| | VRGEVFFRLDDFQALNASLV | |
| | EEGKAPFANPRNSAAGSLRQ | |
| | KDPAVTARRRLRMICHGLGH | |
| | VEGFRPATLHQAYLALRAWG | |
| | LPVSEHTTLATDLAGVRERI | |
| | DYWGEHRHEVDHEIDGVVVK | |
| | VDEVALQRRLGSTSRAPRWA | |
| | IAYKYPPEEAQTKLLDIRVN | |
| | VGRTGRITPFAFMTPVKVAG | |
| | STVGQATLHNASEIKRKGVL | |
| | IGDTVVIRKAGDVIPEVLGP | |
| | VVELRDGSEREFIMPTTCPE | |
| | CGSPLAPEKEGDADIRCPNA | |
| | RGCPGQLRERVFHVASRNGL | |
| | DIEVLGYEAGVALLQAKVIA | |
| | DEGELFALTERDLLRTDLFR | |
| | TKAGELSANGKRLLVNLDKA | |
| | KAAPLWRVLVALSIRHVGPT | |
| | AARALATEFGSLDAIAAAST | |
| | DQLAAVEGVGPTIAAAVTEW | |
| | FAVDWHREIVDKWRAAGVRM | |
| | VDERDESVPRTLAGLTIVVT | |
| | GSLTGFSRDDAKEAIVARGG | |
| | KAAGSVSKKTNYVVAGDSPG | |
| | SKYDKAVELGVPILDEDGFR | |
| | RLLADGPASRT | |
| Emiliania | MEAMCTECEDRDARLDVIDI | 95 |
| huxleyi virus | QLFHALNPKSCNRTTWEQVP | |
| DNA ligase | KIMGKQGDFVAEGKLDGERD | |
| | ASHLYGESMEDVLCECVRED | |
| | VTSLLLDGEMMVVDLETGRY | |
| | LPFGENRSLKDFGTSMRHCF | |
| | VAFDLLLYNGRSMTGATLAE | |
| | RSELLRKAVRTKQHALTLIE | |
| | RFEVGERGAGATTAVMRQLD | |
| | VMMSRGLEGVVFKSLSSKYD | |
| | PGSRDKSWIKLKPDFVDGMG | |
| | DTLDLLILGGYYGEGRRRSG | |
| | AVSTFLMGVRAPPEAAKRVG | |
| | GAAHPLFYPFCKVGTGYSLP | |
| | QLRELRERLMPASLTRRRGN | |
| | ALGHGASLTAVSCEQVSHEW | |
| | KNSRRPAHLCHWEPSKRDDI | |
| | PDYWFEPEASVVLELTAFEI | |
| | ITPRESFLPANYTLRFPRVK | |
| | RVRYDKGWEGAETFERVVEL | |
| | FKECDGRLSANKRRAEEIAA | |
| | SRASAGPAAKKRAAGVAPTV | |
| | GVPWHLKLSADLANTAVECY | |
| | ALDGVVAVVKGTLSRRPGVE | |
| | TQIKRLGGKVHKNMTSLTTH | |
| | LVDAPGAEVLAEVERARRGG | |
| | GSFEVVTAAWVDECSRVHAR | |
| | VTLEPRYVRHVSEATREQIE | |
| | AIMDEWGDNYTIAADPESLV | |
| | DSMRLVREQRSAGGNCGDSP | |
| | LAREAHVADALRDLDDETAV | |
| | ALRTRYAMLRGVVAYVPRGS | |
| | VALRLRLRLLGAQTVDEPSA | |
| | DSTHAVLSASTSADERQRLR | |
| | DKFTEDRVRDGRPSCGRHIV | |
| | SDRWLAECERRGQREPEAQE | |
| | DAWFGDRVGIRDRAL | |
| Lymantria | MENHDSFYKFCQLCQSLYDA | 96 |
| dispar | DDHQEKRDALERHFADFRGS | |
| multicapsid | AFMWRELLAPAESDAAADRE | |
| nuclear | LTLIFETILSIERTEQENVT | |
| polyhedrosis | RNLKCTIDGAAVPLSRESRI | |
| virus | TVPQVYEFINDLRGSGSRQE | |
| | RLRLIGQFAAGCTDEDLLTV | |
| | FRVVSDHAHAGLSAEDVMEL | |
| | VEPWERFQKPVPPALAQPCR | |
| | RLASVLVKHPEGALAEVKYD | |
| | GERVQVHKAGSRFKFFSRTL | |
| | KPVPEHKVAGCREHLTRAFP | |
| | RARNFILDAEIVMVDGSGEA | |
| | LPFGTLGRLKQMEHADGHVC | |
| | MYIFDCLRYNGVSYLNATPL | |
| | DFRRRVLQDEIVPIEGRVVL | |
| | SAMERTNTLSELRRFVHRTL | |
| | ATGAEGVVLKGRLSSYAPNK | |
| | RDWFKMKKEHLCDGALVDTL | |
| | DLVVLGAYYGTGRNCRKMSV | |
| | FLMGCLDRESNVWTTVTKVH | |
| | SGLADAALTALSKELRPLMA | |
| | APRDDLPEWFDCNESMVPHL | |
| | LAADPEKMPVWEIACSEMKA | |
| | NIGAHTAGVTMRFPRVKRFR | |
| | PDKDWSTATDLQEAEQLIRN | |
| | SQENTKKTFARLATTYDGPS | |
| | PNKKLKLN | |

Some aspects include a DNA ligase that ligates DNA strands base paired to a DNA splint. In some embodiments, the DNA ligase ligates DNA strands base paired to an RNA splint. In some embodiments, the DNA ligase comprises an amino acid sequence at least 80% identical to the amino acid sequence of any one of SEQ ID NOS: 55-96, or a functional fragment thereof.

In some aspects, the ligases comprises at least one NLS (e.g., any one of the NLS in Table 2). In some aspects, the ligase comprises at least one additional domain. In some aspects, the at least one additional domain is a dimerization domain (e.g., any one of the dimerization domain in Table 3). In some aspects, the ligase comprising a dimerization domain can be dimerized with an endonuclease to form a heterodimer. In some aspects, the at least one additional domain is a functional domain. For example, the functional domain can comprises a chromatin modifying domain (e.g., any one of the chromatin modifying domain in Table 4) or a cell penetrating peptide (e.g., any one of the cell penetrating peptide in Table 5). In some aspects, the ligase comprises a linker, where the linker can covalently connect the ligase with another polypeptide (e.g., the endonuclease). In some aspects, the linker covalently connects the ligase to the at least one additional domain. In some aspects, the ligase comprises a tag (e.g., any one of the tag in Table 6), where the tag can be used for increasing expression, identifying, or purifying the ligase. A linker may separate the ligase from a nuclear localization signal, a chromatin modifying domain, a cell penetrating peptide, or a tag polypeptide. Any linker described herein may be included.

The ligase may comprise a binding motif for binding to a nucleic acid motif (e.g., a hairpin motif). In some aspects, the ligase (e.g. DNA ligase) comprises an MS2 coat protein (MCP) peptide. The ligase may include a hairpin binding motif such as an MCP peptide. The MCP peptide may be useful for recruiting the ligase to a guide nucleic acid comprising an MS2 hairpin. A benefit of using a MCP peptide and MS2 hairpin is to separate the ligase and endonuclease such as a Cas nickase (or a portion of them), and allow fitting within separate vectors such as AAV vectors. In some aspects, the ligase comprises a loop region. In some aspects, the loop region is a 2a loop or a 3a loop. The loop region may comprise a 2a loop. The loop region may comprise a 3a loop.

### Fusion Proteins

Disclosed herein are fusion proteins. Some aspects include a nucleic acid (e.g. an expression vector) encoding a fusion protein. The fusion protein may include an endonuclease. The fusion protein may include a ligase. The fusion protein may include a linker. The endonuclease and ligase may be connected through a linker. The fusion protein may be an example of a covalently coupled endonuclease and DNA ligase. The fusion protein may comprise an endonuclease such as an RNA-guided endonuclease fused to a DNA ligase.

The fusion protein may be non-naturally occurring. The fusion protein may be engineered. The fusion protein may be synthetic. The fusion protein may be pre-synthetized. The fusion protein may be added to a subject or a cell. The fusion protein may be encoded by a nucleic acid. The encoding nucleic acid may be engineered, synthetic, or added to a subject or a cell.

The fusion protein may include one of various orientations. For example, the fusion protein may include an RNA-guided endonuclease upstream (e.g. N-terminal or in the N-direction) or downstream (e.g. C-terminal or in the C-direction) relative to the DNA ligase. The fusion protein may include an RNA-guided endonuclease amino (N)-terminal to the DNA ligase. The fusion protein may include an RNA-guided endonuclease carboxy (C)-terminal to the DNA ligase. The endonuclease may be in the amino direction within the fusion polypeptide relative to the ligase. The endonuclease may be in the carboxy direction within the fusion polypeptide relative to the ligase. The endonuclease may be N-terminal. The endonuclease may be C-terminal. The ligase may be N-terminal. The ligase may be C-terminal.

The fusion protein may include a nuclear localization signal, chromatin modifying domain, cell penetrating peptide, tag polypeptide, or exonuclease. The fusion protein may include a nuclear localization signal. The fusion protein may include a chromatin modifying domain. The fusion protein may include a cell penetrating peptide. The fusion protein may include a tag polypeptide. The fusion protein may include an exonuclease. Any of the nuclear localization signal, chromatin modifying domain, cell penetrating peptide, tag polypeptide, or exonuclease, endonuclease, or ligase may be directly connected to another or to the endonuclease or ligase. Any of the nuclear localization signal, chromatin modifying domain, cell penetrating peptide, tag polypeptide, or exonuclease, endonuclease, or ligase may be connected by a linker to another or to the endonuclease or ligase. Multiple linkers may be included in the fusion protein. The fusion protein may exclude a polymerase.

A linker may include an amino acid linker. The amino acid linker may include a length of residues. The length may include 1, 2, 3, 4, 5, 6, 7, 8, 9, 10, 15, 20, 25, 30, 40, 50, 60, 70, 80, 90, or 100 residues, or a range of residues defined by any two of the aforementioned integers. The length may include at least 1 residue, at least 2 residues, at least 3 residues, at least 4 residues, at least 5 residues, at least 6 residues, at least 7 residues, at least 8 residues, at least 9 residues, at least 10 residues, at least 15 residues, at least 20 residues, at least 25 residues, at least 30 residues, at least 40 residues, at least 50 residues, at least 60 residues, at least 70 residues, at least 80 residues, at least 90 residues, or at least 100 residues. In some aspects, the length may include less than 2 residues, less than 3 residues, less than 4 residues, less than 5 residues, less than 6 residues, less than 7 residues, less than 8 residues, less than 9 residues, less than 10 residues, less than 15 residues, less than 20 residues, less than 25 residues, less than 30 residues, less than 40 residues, less than 50 residues, less than 60 residues, less than 70 residues, less than 80 residues, less than 90 residues, or less than 100 residues. Examples of residues may include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine, or any combination thereof. The linker may be non-enzymatic, or may lack any enzymatic activity.

A connection may be covalent. A covalent connection may include a peptide bond. The peptide bond may include amide bond. A connection may be between an N-terminus and another N-terminus. A connection may be between a C-terminus and another C-terminus. A connection may be between an N-terminus and a C-terminus. A connection may be between a C-terminus and an N-terminus.

The fusion protein may include connections in various orientations. The endonuclease may be connected at its C-terminus. The endonuclease may be connected at its N-terminus. The ligase may be connected at its C-terminus. The ligase may be connected at its N-terminus.

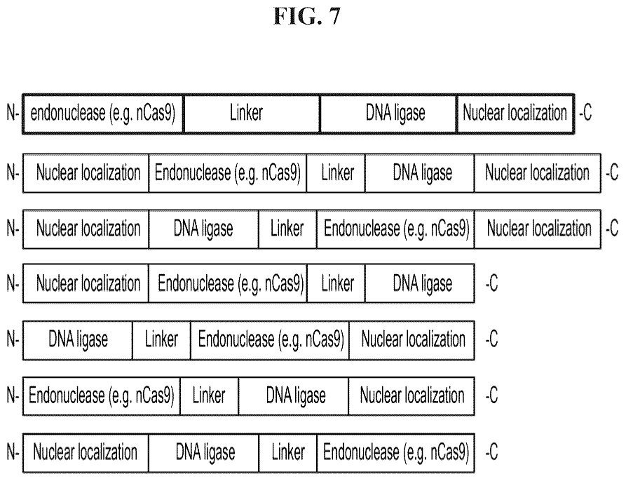

FIG. 7 illustrates some examples of fusion protein. The figure includes examples of arrangements and orientations of the endonuclease, linker, ligase, or nuclear localization signal. Other aspects may be incorporated into the examples shown.

### Non-Covalently Coupled Proteins

Disclosed herein are non-covalently coupled proteins. Some aspects relate to a nucleic acid (e.g. an expression vector) encoding a protein, or encoding at least part of a protein. The proteins may include an endonuclease such as an RNA-guided endonuclease. A protein of the non-covalently coupled proteins may include a portion of an endonuclease. A protein of the non-covalently coupled proteins may include a portion of a ligase. The proteins may include a ligase such as a DNA ligase. A protein of the non-covalently coupled proteins may include a fusion protein.

The non-covalently coupled proteins may be bound together through heterodimerization domains. Examples of heterodimerization domains may include a leucine zipper, PDZ domain, streptavidin, streptavidin binding protein, foldon domain, hydrophobic moiety, or a functional binding fragment thereof. A heterodimerization domain may include a leucine zipper. A heterodimerization domain may include a PDZ domain. A heterodimerization domain may include a streptavidin. A heterodimerization domain may include a streptavidin binding protein. A heterodimerization domain may include a foldon domain. A heterodimerization domain may include a hydrophobic moiety. A heterodimerization domain may include an antibody or antibody fragment. The non-covalently coupled proteins may be bound together through inteins.

The endonuclease and ligase may be coupled together by a separate molecule. The separate molecule may comprise a nucleic acid (e.g. a guide nucleic acid). The ligase may include a hairpin binding motif, where the RNA-guided endonuclease and the DNA ligase are coupled with the nucleic acid. The nucleic acid may include a scaffold that binds the RNA-guided endonuclease and a hairpin that binds to the hairpin binding motif. The hairpin binding motif may include an MS2 coat protein (MCP) peptide. The hairpin may include an MS2 hairpin.

The endonuclease and ligase may be coupled together by a heterobifunctional molecule. The heterobifunctional molecule may include an endonuclease binding domain and a DNA ligase binding domain. The heterobifunctional molecule may include an endonuclease binding domain. The endonuclease binding domain may include a heterodimerization domain. The endonuclease binding domain may include an antibody or antibody binding fragment. The heterobifunctional molecule may include a ligase binding domain such as a DNA ligase binding domain. The DNA ligase binding domain may include a heterodimerization domain. The DNA ligase binding domain may include an antibody or antibody binding fragment. The heterobifunctional molecule may include a small molecule. The small molecule may comprise a proteolysis targeting chimera (PROTAC), or a related heterobifunctional molecule.

Some aspects include a protein complex, comprising: an RNA-guided endonuclease bound to a DNA ligase. The endonuclease and the DNA ligase may be bound together through heterodimerization domains. The protein complex of embodiment 75, wherein the heterodimerization domains may comprise leucine zippers, PDZ domains, streptavidin and streptavidin binding protein, foldon domains, hydrophobic polypeptides, an antibody that binds the Cas nickase, or an antibody that binds the DNA ligase, or one or more binding fragments thereof. The protein complex may be included in a cell. The cell may further include a heterologous RNA-guided endonuclease and a DNA ligase that that was introduced into the cell. The cell may further include a nuclease that is different from the RNA-guided endonuclease.

### Guide Nucleic Acids

Disclosed herein are guide nucleic acids. The guide nucleic acid may be included in a composition, system or method disclosed herein. Some aspects relate to a nucleic acid (e.g. DNA or an expression vector) that encodes a guide nucleic acid such as a guide RNA. Provided herein are guide nucleic acids (e.g., gRNAs) that direct a programmable endonuclease (e.g., a nCas9) to a target nucleic acid (e.g. a genomic locus). The guide nucleic acid may guide an RNA-guided endonuclease to a target nucleic acid locus for nucleic acid replacement or gene editing at the locus. A guide nucleic acid of the present disclosure may facilitate a donor strand to be inserted into a target site of the target nucleic acid. A guide nucleic acid of the present disclosure may facilitate editing of a nucleic acid sequence at a target site of the target nucleic acid. The guide nucleic acid may, in some instances, also act as a splint for a DNA ligase described herein, such as for ligating two nucleic acid strands base paired to a portion of the guide nucleic acid. The guide nucleic acid may be single stranded. The guide nucleic acid may include RNA. The guide nucleic acid may be RNA. The guide nucleic acid may include a guide RNA (gRNA). In some cases, a guide nucleic acid may include DNA.

The guide nucleic acid may be non-naturally occurring. The guide nucleic acid may be engineered. The guide nucleic acid may be synthetic. The guide nucleic acid may be pre-synthetized. The guide nucleic acid may be added to a subject or a cell. In some aspects, the guide nucleic acid does not include a template for a polymerase.

The guide nucleic acid may include an integrating nucleic acid binding site. The integrating nucleic acid binding site may be referred to as a “donor binding site.”

Disclosed herein are guide nucleic acids, comprising: a spacer reverse complementary to a first region of a target nucleic acid; a scaffold configured to bind to an endonuclease; and an integrating nucleic acid binding site and optionally a flap binding site reverse complementary to a nucleic acid flap.

In some aspects, the guide nucleic acid comprises a spacer complementary to a genomic locus in a cell; a scaffold for complexing with the at least one endonuclease; a donor binding site that is at least partially complementary to a donor strand; a flap binding site that is at least partially identical or complementary to a genomic flap at or adjacent to the genomic locus; or a combination thereof. In some aspects, the guide nucleic acid can direct the at least one endonuclease to cleave at least one strand of the genomic locus. In some aspects, the guide nucleic acid can be at least partially complementary to the donor strand or at least partially complementary to a genomic flap (e.g., a genomic nucleic acid sequence that is displaced and become single-stranded when the guide nucleic acid recruits the endonuclease to the genomic locus). In some aspects, the guide nucleic acid, being at least partially complementary to the donor strand or at least partially complementary to a genomic flap, brings the donor strand to close proximity of the cleaving of the genomic locus.

Disclosed herein, in some embodiments, are guide nucleic acids comprising a scaffold. The scaffold may bind a nuclease. The scaffold may bind a Cas nuclease. The scaffold may bind a nickase. The scaffold may bind a Cas nickase. The scaffold may bind an S. Pyogenes Cas9 nuclease. The scaffold may bind an S. Pyogenes Cas9 nickase. The scaffold may include a scaffold nucleic acid sequence. A system described herein may include a first guide nucleic acid. The system can include a second guide nucleic acid. The first guide nucleic acid may bind to a first Cas nickase. The second guide nucleic acid may bind to a second Cas nickase.

A guide nucleic acid may include any aspect of (i)-(iv): (i) a spacer complementary to a region of a genomic locus of a genomic strand, (ii) a scaffold for complexing with an RNA-guided endonuclease, (iii) a donor binding site that is at least partially complementary to an integrating nucleic acid, or (iv) a flap binding site that is at least partially identical or complementary to a genomic flap at or adjacent to the genomic locus. A guide nucleic acid may include any aspect of (i)-(iii): (i) a spacer complementary to a region of a genomic locus of a genomic strand, (ii) a scaffold for complexing with an RNA-guided endonuclease, or (iii) a donor binding site that is at least partially complementary to a splinting nucleic acid. A component of (i), (ii), or (iii) may be included in a single guide nucleic acid, or may be split between or collectively included among multiple guide nucleic acids.

In some aspects, the guide nucleic acid comprises a modified internucleoside linkage. In some aspects, the modified internucleoside linkage comprises a phosphorothioate linkage. In some aspects, the modified internucleoside linkage is between any of the 4 terminal nucleosides at a 5′ end or at a 3′ end of the guide nucleic acid. The guide nucleic acid may include multiple modified internucleoside linkages. For example, the guide nucleic acid may include modified internucleoside linkages at nucleic acids of the 5′ and 3′ ends of the guide nucleic acid, such as between the last 4 nucleic acids at the 5′ end and between the last 4 nucleic acids at the 3′ end. In some aspects, the guide nucleic acid comprises a modified nucleoside. In some aspects, the modified nucleoside comprises a locked nucleic acid (LNA), a 2′ fluoro, a 2′ O-alkyl, or a combination thereof. The modified nucleoside may include an LNA, a 2′fluoro, a 2′ O-alkyl, a methylated cytosine, an inverted thymidine, or a combination thereof. The modified nucleoside may include an LNA. The modified nucleoside may include a 2′fluoro. The modified nucleoside may include a 2′ O-alkyl. The modified nucleoside may include a methylated cytosine. In some aspects, the modified nucleoside is any of the 3 terminal nucleosides at a 5′ end or at a 3′ end of the guide nucleic acid. The guide nucleic acid may include multiple modified nucleosides. For example, the guide nucleic acid may include modified nucleosides at nucleic acids of the 5′ and 3′ ends of the guide nucleic acid, such as the last 3 nucleic acids at the 5′ end and the last 3 nucleic acids at the 3′ end.

In some aspects, the guide nucleic acid comprises at least one nucleic acid modification. In some aspect, the at least nucleic acid modification comprises modifying a backbone, a sugar, a base, or a combination thereof of the guide nucleic acid. In some aspects, the at least one nucleic acid modification can increase resistance of the guide nucleic acid to degradation (e.g., against nuclease degradation or hydrolysis). In some aspects, the at least one nucleic acid modification can increase the complexing of the guide nucleic acid to the at least one endonuclease. In some aspects, the at least one nucleic acid modification can increase the complexing of the guide nucleic acid to the donor strand. In some aspects, the at least one nucleic acid modification can increase the complexing of the guide nucleic acid to the genomic locus via by being complementary to the genomic flap.

In some aspects, the guide nucleic acid comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleic acid modifications. In some aspects, nucleic acid modification can occur at 3′OH group, 5′OH group, at the backbone, at the sugar component, or at the nucleotide base. Nucleic acid modification can include non-naturally occurring linker molecules of interstrand or intrastrand cross links. In one aspect, the modified nucleic acid comprises modification of one or more of the 3′OH or 5′OH group, the backbone, the sugar component, or the nucleotide base, or addition of non-naturally occurring linker molecules. In some aspects, modified backbone comprises a backbone other than a phosphodiester backbone. In some aspects, a modified sugar comprises a sugar other than deoxyribose (in modified DNA) or other than ribose (modified RNA). In some aspects, a modified base comprises a base other than adenine, guanine, cytosine, thymine or uracil. In some aspects, the guide nucleic acid comprises at least one modified base. In some instances, the guide nucleic acid comprises at least one, two, three, four, five, six, seven, eight, nine, 10, 15, 20, or more modified bases. In some cases, the nucleic acid modifications to the base moiety include natural and synthetic modifications of adenine, guanine, cytosine, thymine, or uracil, and purine or pyrimidine bases.

In some aspects, the at least one nucleic acid modification of the guide nucleic acid comprises a modification of any one of or any combination of: 2′ modified nucleotide comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA); modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Non limiting examples of nucleic acid modification to the guide nucleic acid can include: modification of one or both of non-linking or linking phosphate oxygens in the phosphodiester backbone linkage (e.g., sulfur (S), selenium (Se), BR3 (wherein R can be, e.g., hydrogen, alkyl, or aryl), C (e.g., an alkyl group, an aryl group, and the like), H, NR2, wherein R can be, e.g., hydrogen, alkyl, or aryl, or wherein R can be, e.g., alkyl or aryl); replacement of the phosphate moiety with “dephospho” linkers (e.g., replacement with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino); modification or replacement of a naturally occurring nucleobase with nucleic acid analog; modification of deoxyribose-phosphate or ribose-phosphate backbone (e.g., modifying the ribose-phosphate backbone to incorporate phosphorothioate, phosphonothioacetate, phosphoroselenates, boranophosphates, borano phosphate esters, hydrogen phosphonates, phosphonocarboxylate, phosphoroamidates, alkyl or aryl phosphonates, phosphonoacetate, or phosphotriesters; modification of 5′ end (e.g., 5′ cap or modification of 5′ cap —OH) or 3′ end of the nucleic acid sequence (3′ tail or modification of 3′ end —OH); substitution of the phosphate group with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino; modification of the ribophosphate backbone to incorporate morpholino (phosphorodiamidate morpholino oligomer PMO), cyclobutyl, pyrrolidine, or peptide nucleic acid (PNA) nucleoside surrogates; modifications to the sugar of a nucleotide to incorporate locked nucleic acid (LNA), unlocked nucleic acid (UNA), ethylene nucleic acid (ENA), constrained ethyl (cEt) sugar, or bridged nucleic acid (BNA); modification of a constituent of the ribose sugar (e.g., 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE)); modification to the base of a nucleotide (of A, T, C, G, or U); and stereopure of nucleotide (e.g., S conformation of phosphorothioate or R conformation of phosphorothioate).

In some aspects, the nucleic acid modification comprises at least one substitution of one or both of non-linking phosphate oxygen atoms in a phosphodiester backbone linkage of the guide nucleic acid. In some aspects, the at least one nucleic acid modification of the guide nucleic acid comprises a substitution of one or more of linking phosphate oxygen atoms in a phosphodiester backbone linkage of the guide nucleic acid. A non-limiting example of a nucleic acid modification of a phosphate oxygen atom is a sulfur atom. In some aspects, the nucleic acid modification comprises at least one modification to a sugar. In some aspects, the nucleic acid modification comprises at least one nucleic acid modification to the sugar comprising a modification of a constituent of the sugar, where the sugar is a ribose sugar. In some aspects, the nucleic acid modification of the guide nucleic acid comprises at least one modification to the constituent of the ribose sugar of the nucleotide of the guide nucleic acid comprising a 2′-O-Methyl group. In some aspects, the nucleic acid modification comprises at least one modification comprising replacement of a phosphate moiety of the guide nucleic acid with a dephospho linker. In some aspects, the nucleic acid modification of comprises at least one modification of a phosphate backbone. In some aspects, the modification comprises a phosphorothioate group. In some aspects, the nucleic acid modifications comprises at least one modification comprising a modification to a base of a nucleotide of the guide nucleic acid. In some aspects, the nucleic acid modifications comprises at least one modification comprising an unnatural base of a nucleotide. In some aspects, the nucleic acid modifications comprises at least one modification comprising at least one stereopure nucleic acid. In some aspects, the at least one nucleic acid modification can be positioned proximal to a 5′ end of the guide nucleic acid. In some aspects, the at least one nucleic acid modification can be positioned proximal to a 3′ end of the guide nucleic acid. In some aspects, the at least one nucleic acid modification can be positioned proximal to both 5′ and 3′ ends of the guide nucleic acid.

In some aspects, the guide nucleic acid described herein comprises a backbone comprising a plurality of sugar and phosphate moieties covalently linked together. In some cases, a backbone of the guide nucleic acid comprises a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA. In some aspects, a backbone of the guide nucleic acid can lack a 5′ reducing hydroxyl, a 3′ reducing hydroxyl, or both, capable of being exposed to a solvent. In some aspects, a backbone of the guide nucleic acid can lack a 5′ reducing hydroxyl, a 3′ reducing hydroxyl, or both, capable of being exposed to nucleases. In some aspects, a backbone of the guide nucleic acid can lack a 5′ reducing hydroxyl, a 3′ reducing hydroxyl, or both, capable of being exposed to hydrolytic enzymes. In some instances, a backbone of the guide nucleic acid can be represented as a polynucleotide sequence in a circular 2-dimensional format with one nucleotide after the other. In some instances, a backbone of the guide nucleic acid can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety. In some aspects, the guide nucleic acid comprises at least one nucleic acid modification comprising any one of: 5′ adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, LNA, cEt, pseudouridine-5′-triphosphate, 5-methylcytidine-5′-triphosphate, 2-O-methyl-phosphorothioate or any combinations thereof.

A nucleic acid modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases. In some aspects, the guide nucleic acid comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 50, 100, or more internucleotide linkage comprising PS bond. In some aspects, the guide nucleic acid comprises only PS bond as the internucleotide linkage modification. In some aspects, all internucleotide linkages of the guide nucleic acid herein are fully PS-modified or include phosphorothioate internucleotide linkages.

The guide nucleic acid may include a hairpin. The hairpin may bind to a hairpin binding motif such as a hairpin binding motif on a DNA ligase. The hairpin may include an MS2 hairpin A hairpin such as an MS2 hairpin may be useful for recruiting a DNA ligase that includes an MCP peptide.

The guide nucleic acid may include any aspect included in FIG. 1A-6C. Table 8 illustrates non-limiting examples of some of the guide nucleic acids described herein. Some of the guide nucleic acids in the table include nucleic acid modifications.

TABLE 8
Examples of nucleic acid sequences

| | Nucleic acid sequence | SEQ ID |
|---|---|---|
| Name | (5′ to 3′) | NO: |
| BFP | atggtgagcaagggcgagga | 97 |
| | gctgttcaccggggtggtgc | |
| | ccatcctggtcgagctggac | |
| | ggcgacgtaaacggccacaa | |
| | gttcagcgtgtccggcgagg | |
| | gcgagggcgatgccacctac | |
| | ggcaagctgaccctgaagtt | |
| | catctgcaccaccggcaagc | |
| | tgccGgtgccctggcccacc | |
| | CTCgtgaccaccctgaccCA | |
| | Tggcgtgcagtgcttcagcc | |
| | gctaccccgaccacatgaag | |
| | cagcacgacttcttcaagtc | |
| | cgccatgcccgaaggctacg | |
| | tccaggagcgcaccatcttc | |
| | ttcaaggacgacggcaacta | |
| | caagacccgcgccgaggtga | |
| | agttcgagggcgacaccctg | |
| | gtgaaccgcatcgagctgaa | |
| | gggcatcgacttcaaggagg | |
| | acggcaacatcctggggcac | |
| | aagctggagtacaactacaa | |
| | cagccacaacgtctatatca | |
| | tggccgacaagcagaagaac | |
| | ggcatcaaggtgaacttcaa | |
| | gatccgccacaacatcgagg | |
| | acggcagcgtgcagctcgcc | |
| | gaccactaccagcagaacac | |
| | ccccatcggcgacggccccg | |
| | tgctgctgcccgacaaccac | |
| | tacctgagcacccagtccaa | |
| | gctgagcaaagaccccaacg | |
| | agaagcgcgatcacatggtc | |
| | ctgctggagttcgtgaccgc | |
| | cgccgggatcactctcggca | |
| | tggacgagctgtacaagTAA | |
| Rep1. | mC*mU*mG*AAGUUCAUCUG | 98 |
| BFP. | CACCACGUUUAAGAGCUAUG | |
| FwdGuide | CUGGAAACAGCAUAGCAAGU | |
| | UUAAAUAAGGCUAGUCCGUU | |
| | AUCAACUUGAAAAAGUGGCA | |
| | CCGAGUCGGUGCGUGGGCCA | |
| | GGGCACCGGCAGCUUGCCGG | |
| | UGGUGCAGAUGmA*mA*mC*U | |
| Rep1. | mG*mA*mC*GUAGCCUUCGG | 99 |
| BFP. | GCAUGGGUUUAAGAGCUAUG | |
| RevGuide | CUGGAAACAGCAUAGCAAGU | |
| | UUAAAUAAGGCUAGUCCGUU | |
| | AUCAACUUGAAAAAGUGGCA | |
| | CCGAGUCGGUGCUGAAGCAG | |
| | CACGACUUCUUCAAGUCCGC | |
| | CAUGCCCGAAGmG*mC*mU*A | |
| Rep1. | mC*mU*mG*AAGUUCAUCUG | 100 |
| BFP. | CACCACGUUUAAGAGCUAUG | |
| FwdGuide. | CUGGAAACAGCAUAGCAAGU | |
| SpPAMmut | UUAAAUAAGGCUAGUCCGUU | |
| | AUCAACUUGAAAAAGUGGCA | |
| | CCGAGUCGGUGCGUGGGCCA | |
| | GGGCACCGGCAGCUUGCCGG | |
| | UUGUGCAGAUGmA*mA*mC*U | |
| Rep1. | mG*mA*mC*GUAGCCUUCGG | 101 |
| BFP. | GCAUGGGUUUAAGAGCUAUG | |
| RevGuide. | CUGGAAACAGCAUAGCAAGU | |
| SpPAMmut | UUAAAUAAGGCUAGUCCGUU | |
| | AUCAACUUGAAAAAGUGGCA | |
| | CCGAGUCGGUGCUGAAGCAG | |
| | CACGACUUCUUCAAGUCAGC | |
| | CAUGCCCGAAGmG*mC*mU*A | |
| Rep1. | /5Phos/caccggcaagctg | 102 |
| BFP2GFP. | ccGgtgccctggcccaccCT | |
| TopDonor. | CgtgaccaccctgaccTACg | |
| 5P | gcgtgcagtgcttcagccgc | |
| | taccccgaccaca | |
| Rep1. | /5Phos/tggcggacttgaa | 103 |
| BFP2GFP. | gaagtcgtgctgcttcatgt | |
| BotDonor. | ggtcggggtagcggctgaag | |
| 5P | cactgcacgccGTAggtcag | |
| | ggtggtcacGAGg | |
| Rep1. | /5Phos/caccggcaagctg | 104 |
| BFP2GFP. | ccGgtgccctggcccacTCT | |
| TopDonor. | TGTGACCACCTTGACCtACG | |
| Recoded. | GTGTCCAGTGTTTTAGCAGG | |
| 5P | TATCCGGATCACA | |
| Rep1. | /5Phos/tggcggacttgaa | 105 |
| BFP2GFP. | gaagtcgtgctgcttcaTGT | |
| BotDonor. | GATCCGGATACCTGCTAAAA | |
| Recoded. | CACTGGACACCGTaGGTCAA | |
| 5P | GGTGGTCACAAGA | |
| Rep1. | /5Phos/Aaccggcaagctg | 106 |
| BFP2GFP. | ccGgtgccctggcccacTCT | |
| TopDonor. | TGTGACCACCTTGACCtACG | |
| SpPAMmut. | GTGTCCAGTGTTTTAGCAGG | |
| Recoded.5P | TATCCGGATCACA | |
| Rep1. | /5Phos/tggcTgacttgaa | 107 |
| BFP2GFP. | gaagtcgtgctgcttcaTGT | |
| BotDonor. | GATCCGGATACCTGCTAAAA | |
| SpPAMmut. | CACTGGACACCGTaGGTCAA | |
| Recoded.5P | GGTGGTCACAAGA | |
| Rep1. | /5Phos/c*a*c*cggcaag | 108 |
| BFP2GFP. | ctgccGgtgccctggcccac | |
| TopDonor. | TCTTGTGACCACCTTGACCt | |
| Recoded.5P. | ACGGTGTCCAGTGTTTTAGC | |
| endPhos | AGGTATCCGGATC*A*C*A | |
| Rep1. | /5Phos/t*g*g*cggactt | 109 |
| BFP2GFP. | gaagaagtcgtgctgcttca | |
| BotDonor. | TGTGATCCGGATACCTGCTA | |
| Recoded.5P. | AAACACTGGACACCGTaGGT | |
| endPhos | CAAGGTGGTCACA*A*G*A | |
| Rep2. | mC*mU*mG*AAGUUCAUCUG | 110 |
| BFP. | CACCACGUUUAAGAGCUAUG | |
| FwdGuide | CUGGAAACAGCAUAGCAAGU | |
| | UUAAAUAAGGCUAGUCCGUU | |
| | AUCAACUUGAAAAAGUGGCA | |
| | CCGAGUCGGUGCCUACGGCA | |
| | AGCUGACCmC*mU*mG*A | |
| Rep2. | mG*mA*mC*GUAGCCUUCGG | 111 |
| BFP. | GCAUGGGUUUAAGAGCUAUG | |
| RevGuide | CUGGAAACAGCAUAGCAAGU | |
| | UUAAAUAAGGCUAGUCCGUU | |
| | AUCAACUUGAAAAAGUGGCA | |
| | CCGAGUCGGUGCAGAUGGUG | |
| | CGCUCCUGmG*mA*mC*G | |
| Rep2. | /5Phos/Aaccggcaagctg | 112 |
| BFP2GFP. | ccGgtgccctggcccaccCT | |
| TopDonor. | CgtgaccaccctgaccTACg | |
| SpPAMmut. | gcgtgcagtgcttcagccgc | |
| 5P | taccccgaccacatgaagca | |
| | gcacgacttcttcaagtcAg | |
| | ccatgcccgaaggctacgtc | |
| | caggagcgcaccatct | |
| Rep2. | /5Phos/tggcTgacttgaa | 113 |
| BFP2GFP. | gaagtcgtgctgcttcatgt | |
| BotDonor. | ggtcggggtagcggctgaag | |
| SpPAMmut. | cactgcacgccGTAggtcag | |
| 5P | ggtggtcacGAGggtgggcc | |
| | agggcacCggcagcttgccg | |
| | gtTgtgcagatgaacttcag | |
| | ggtcagcttgccgtag | |
| Rep2. | /5Phos/AACCGGTAAGTTG | 114 |
| BFP2GFP. | CCAGTCCCGTGGCCTACTCT | |
| TopDonor. | TGTGACCACCTTGACCtACG | |
| SpPAMmut. | GTGTCCAGTGTTTTAGCAGG | |
| Recoded. | TATCCGGATCACATGAAACA | |
| 5P | GCATGACTTCTTTAAATCAG | |
| | CTATGcccgaaggctacgtc | |
| | caggagcgcaccatct | |
| Rep2. | /5Phos/TAGCTGATTTAAA | 115 |
| BFP2GFP. | GAAGTCATGCTGTTTCATGT | |
| BotDonor. | GATCCGGATACCTGCTAAAA | |
| SpPAMmut. | CACTGGACACCGTaGGTCAA | |
| Recoded. | GGTGGTCACAAGAGTAGGCC | |
| 5P | ACGGGACTGGCAACTTACCG | |
| | GTTgtgcagatgaacttcag | |
| | ggtcagcttgccgtag | |
| Rep2. | /5Phos/caccggcaagctg | 116 |
| BFP2GFP. | ccGgtgccctggcccaccCT | |
| TopDonor. | CgtgaccaccctgaccTACg | |
| 5P | gcgtgcagtgcttcagccgc | |
| | taccccgaccacatgaagca | |
| | gcacgacttcttcaagtccg | |
| | ccatgcccgaaggctacgtc | |
| | caggagcgcaccatct | |
| Rep2. | /5Phos/tggcggacttgaa | 117 |
| BFP2GFP. | gaagtcgtgctgcttcatgt | |
| BotDonor. | ggtcggggtagcggctgaag | |
| 5P | cactgcacgccGTAggtcag | |
| | ggtggtcacGAGggtgggcc | |
| | agggcacCggcagcttgccg | |
| | gtggtgcagatgaacttcag | |
| | ggtcagcttgccgtag | |
| Rep2. | /5Phos/A*A*C*CGGTAAG | 118 |
| BFP2GFP. | TTGCCAGTCCCGTGGCCTAC | |
| TopDonor. | TCTTGTGACCACCTTGACCt | |
| SpPAMmut. | ACGGTGTCCAGTGTTTTAGC | |
| Recoded. | AGGTATCCGGATCACATGAA | |
| 5P. | ACAGCATGACTTCTTTAAAT | |
| endPhos | CAGCTATGcccgaaggctac | |
| | gtccaggagcgcacca*t*c*t | |
| Rep2. | /5Phos/T*A*G*CTGATTT | 119 |
| BFP2GFP. | AAAGAAGTCATGCTGTTTCA | |
| BotDonor. | TGTGATCCGGATACCTGCTA | |
| SpPAMmut. | AAACACTGGACACCGTaGGT | |
| Recoded. | CAAGGTGGTCACAAGAGTAG | |
| 5P. | GCCACGGGACTGGCAACTTA | |
| endPhos | CCGGTTgtgcagatgaactt | |
| | cagggtcagcttgccg*t*a*g | |
| Rep2. | mG*mA*mA*AGCUGGCGGGC | 120 |
| CBXl. | ACUAUGGUUUAAGAGCUAUG | |
| FwdGuide | CUGGAAACAGCAUAGCAAGU | |
| | UUAAAUAAGGCUAGUCCGUU | |
| | AUCAACUUGAAAAAGUGGCA | |
| | CCGAGUCGGUGCGUCACCCU | |
| | UUACACCAmG*mA*mA*A | |
| Rep2. | mC*mU*mU*UGCCCUUUACC | 121 |
| CBXl. | ACUCGAGUUUAAGAGCUAUG | |
| RevGuide | CUGGAAACAGCAUAGCAAGU | |
| | UUAAAUAAGGCUAGUCCGUU | |
| | AUCAACUUGAAAAAGUGGCA | |
| | CCGAGUCGGUGCUUUAGGAG | |
| | GUACUCCAmC*mU*mU*U | |
| Rep2. | ATGgtgagcaagggcgagga | 122 |
| mGL-CBX1. | gctgttcaccggggtggtgc | |
| TopDonor. | ccatcctggtcgagctggac | |
| SpPAMmut. | ggcgacgtaaacggccacaa | |
| 5P | gttcagcgtccgcggcgagg | |
| | gcgagggcgatgccaccaac | |
| | ggcaagctgaccctgaagtt | |
| | catctgcaccaccggcaagc | |
| | tgcccgtgccctggcccacc | |
| | ctcgtgaccaccttaggcta | |
| | cggcgtggcctgcttcgccc | |
| | gctaccccgaccacatgaag | |
| | cagcacgacttcttcaagtc | |
| | cgccatgcccgaaggctacg | |
| | tccaggagcgcaccatctct | |
| | ttcaaggacgacggtaccta | |
| | caagacccgcgccgaggtga | |
| | agttcgagggcgacaccctg | |
| | gtgaaccgcatcgtgctgaa | |
| | gggcatcgacttcaaggagg | |
| | acggcaacatcctggggcac | |
| | aagctggagtacaacttcaa | |
| | cagccacaaggtctatatca | |
| | cggccgacaagcagaagaac | |
| | ggcatcaaggctaacttcaa | |
| | gacccgccacaacgttgagg | |
| | acggcggcgtgcagctcgcc | |
| | gaccactaccagcagaacac | |
| | ccccatcggcgacggccccg | |
| | tgctgctgcccgacaaccac | |
| | tacctgagccatcagtccaa | |
| | actgagcaaagaccccaacg | |
| | agaagcgcgatcacatggtc | |
| | ctgaaggagagggtgaccgc | |
| | cgccgggattacacatgaca | |
| | tggacgagctgtacaagtct | |
| | ggaggatctagcggaggatc | |
| | cGGGAAGAAACAAAACAAGA | |
| | AGAAAGTGGAGGAGGTGCTA | |
| | GAAGAGGAGGAAGAGGAATA | |
| | TGTGGTGGAAAAAGTTCTCG | |
| | AtCGTCGAGTGGTAAAGGGC | |
| | AAAGTGGAGTACCTCCTAAA | |
| Rep2. | CGACGaTCGAGAACTTTTTC | 123 |
| mGL-CBX1. | CACCACATATTCCTCTTCCT | |
| BotDonor. | CCTCTTCTAGCACCTCCTCC | |
| SpPAMmut. | ACTTTCTTCTTGTTTTGTTT | |
| 5P | CTTCCCggatcctccgctag | |
| | atcctccagacttgtacagc | |
| | tcgtccatgtcatgtgtaat | |
| | cccggcggcggtcaccctct | |
| | ccttcaggaccatgtgatcg | |
| | cgcttctcgttggggtcttt | |
| | gctcagtttggactgatggc | |
| | tcaggtagtggttgtcgggc | |
| | agcagcacggggccgtcgcc | |
| | gatgggggtgttctgctggt | |
| | agtggtcggcgagctgcacg | |
| | ccgccgtcctcaacgttgtg | |
| | gcgggtcttgaagttagcct | |
| | tgatgccgttcttctgcttg | |
| | tcggccgtgatatagacctt | |
| | gtggctgttgaagttgtact | |
| | ccagcttgtgccccaggatg | |
| | ttgccgtcctccttgaagtc | |
| | gatgcccttcagcacgatgc | |
| | ggttcaccagggtgtcgccc | |
| | tcgaacttcacctcggcgcg | |
| | ggtcttgtaggtaccgtcgt | |
| | ccttgaaagagatggtgcgc | |
| | tcctggacgtagccttcggg | |
| | catggcggacttgaagaagt | |
| | cgtgctgcttcatgtggtcg | |
| | gggtagcgggcgaagcaggc | |
| | cacgccgtagcctaaggtgg | |
| | tcacgagggtgggccagggc | |
| | acgggcagcttgccggtggt | |
| | gcagatgaacttcagggtca | |
| | gcttgccgttggtggcatcg | |
| | ccctcgccctcgccgcggac | |
| | gctgaacttgtggccgttta | |
| | cgtcgccgtccagctcgacc | |
| | aggatgggcaccaccccggt | |
| | gaacagctcctcgcccttgc | |
| | tcacCATAGTGCCCGCCAGC | |
| | TTTCTGGTGTAAAGGGTGAC | |
| CBX1-001 | CAGCGTCACCCTTTACACCA | 124 |
| Exon 2 | GAAAGCTGGCGGGCACTATG | |
| (includes | GGGAAAAAACAAAACAAGAA | |
| beginning | GAAAGTGGAGGAGGTGCTAG | |
| of | AAGAGGAGGAAGAGGAATAT | |
| ORF) | GTGGTGGAAAAAGTTCTCGA | |
| | CCGTCGAGTGGTAAAGGGCA | |
| | AAGTGGAGTACCTCCTAAAG | |
| | TGGAAGGGATTCTCAGA | |

The guide nucleic acid may include a sequence of linking nucleic acids (e.g. linking RNA or DNA nucleotides) between components of the guide nucleic acid. For example, the guide nucleic acid may include a sequence of linking nucleic acids between any of the following components: a spacer, a scaffold, a donor binding site, or a flap binding site. The guide nucleic acid may include a sequence of linking nucleic acids between a spacer, a scaffold, or a donor binding site. The guide nucleic acid include a sequence of linking nucleic acids between the scaffold and the donor binding site The guide nucleic acid may include a sequence of linking nucleic acids between a spacer and a scaffold. The guide nucleic acid may include multiple sequences of linking nucleic acids between components.

The sequence of linking nucleic acids may include any base, such as A, U, T, G, or C, or a combination thereof. The sequence of linking nucleic acids may include A, T, G, or C, or a combination thereof. The sequence of linking nucleic acids may include A, U, G, or C, or a combination thereof. The sequence of linking nucleic acids may include a series of As. The sequence of linking nucleic acids may include a series of Ts. The sequence of linking nucleic acids may include a series of Us. The sequence of linking nucleic acids may include a series of Cs. The sequence of linking nucleic acids may include a series of Gs.

The sequence of linking nucleic acids may include a length, such as a number of nucleotides. The length may include 1, 2, 3, 4, 5, 6, 7, 8, 9 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 nucleotides, or a range defined by any two of the aforementioned numbers of nucleotides. The length may include at least 1, at least 2, at least 3, at least 4, at least 5, at least 6, at least 7, at least 8, at least 9 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, or at least 100 nucleotides. In some aspects, the length may be less than 2, less than 3, less than 4, less than 5, less than 6, less than 7, less than 8, less than 9 10, less than 11, less than 12, less than 13, less than 14, less than 15, less than 16, less than 17, less than 18, less than 19, less than 20, less than 21, less than 22, less than 23, less than 24, less than 25, less than 30, less than 35, less than 40, less than 45, less than 50, less than 55, less than 60, less than 65, less than 70, less than 75, less than 80, less than 85, less than 90, less than 95, or less than 100 nucleotides.

Some aspects relate to a guide nucleic acid comprising: a spacer that is at least partially complementary to a genomic locus in a cell; a scaffold for complexing with a RNA-guided endonuclease; and a donor binding site that is at least partially complementary to an integrating nucleic acid. The guide nucleic acid may further comprise a flap binding site that is at least partially complementary to a genomic sequence of the genomic locus. The guide nucleic acid may further comprise at least one nucleic acid modification. The at least one nucleic acid modification may comprise a modification to a backbone, a sugar, a base, or a combination thereof. The guide nucleic acid may comprise RNA.

Some aspects include a guide nucleic acid, comprising: a spacer at least partially reverse complementary to a first region of a target nucleic acid; a scaffold configured to bind to an endonuclease; and a flap binding site at least partially reverse complementary to a nucleic acid flap, and an integrating nucleic acid binding site.

### Integrating Nucleic Acids

Disclosed herein are integrating nucleic acids. The integrating nucleic acid may be included in a composition, system, or method disclosed herein. Some aspects relate to a nucleic acid that encodes an integrating nucleic acid. Provided herein are integrating nucleic acids that are inserted into a target nucleic acid such as a host genome at a genetic locus. For example, the integrating nucleic acid may replace a nucleic acid in the target nucleic acid. The integrating nucleic acid may be referred to as a “donor nucleic acid,” “donor” or “donor strand.” Where a genomic locus is described, a genetic locus may be included, or vice versa. For example, the locus may be part of a host genome or may be a part of a non-genome nucleic acid. The donor may include DNA. Likewise, the target nucleic acid may include DNA. In some cases, the donor may include RNA, for example when a target nucleic acid includes RNA. The integrating nucleic acid may include any insert, such as a gene or a regulatory element, to be inserted at a genomic locus of a target nucleic acid. The donor strand may include a sequence that is at least partially homologous to the genomic locus. The integrating nucleic acid may, in some instances, also act as a splint for a DNA ligase described herein, such as for ligating two nucleic acid strands base paired to a portion of the splinting integrating nucleic acid. In some cases, the splint includes one strand of the integrating nucleic acid, and the portion being ligated may be another strand of the integrating nucleic acid. In some cases, the splint includes a strand of the integrating nucleic acid, and the portion being ligated may be an upstream or downstream portion of the same strand of the integrating nucleic acid. The integrating nucleic acid may be single stranded. The integrating nucleic acid may be double stranded. The integrating nucleic acid may be delivered as two strands. The integrating nucleic acid may be delivered as multiple strands, e.g. 2 strands.

The integrating nucleic acid may be non-naturally occurring. The integrating nucleic acid may be engineered. The integrating nucleic acid may be synthetic. The integrating nucleic acid may be pre-synthetized. The integrating nucleic acid may be added to a subject or a cell. In some aspects, the integrating nucleic acid does not include a template for a polymerase.

Disclosed herein are integrating nucleic acids, comprising: a double-stranded DNA region to be inserted into a target nucleic acid, wherein the double-stranded DNA region is flanked by at least one overhang comprising a flap binding site and/or guide binding site.

The integrating nucleic acid may be ligated into a target nucleic acid such as a genomic strand. The integrating nucleic acid may include a 5′ end that may be ligated to a 3′ terminus of a genomic strand generated by an RNA-guided endonuclease.

The donor may include any aspect included in FIG. 1A-6C. For example, the donor may include an aspect such as a guide binding site, a flap binding site, or an overhang. The donor may include a guide binding site. The donor may include 2 guide binding sites. The donor may include a flap binding site. The donor may include 2 flap binding sites. The donor may include an overhang. The donor may include 2 overhangs. The aspects may be included at a 5′ end or a 3′ end of the donor, or at both ends. A guide binding site or a flap binding site may be in an internal region of the donor.

Some aspects include an integrating nucleic acid, comprising: a double-stranded DNA region to be inserted into a target nucleic acid, wherein the double-stranded DNA region is flanked by at least one overhang comprising a flap binding site or guide binding site.

In some aspects, the integrating nucleic acid comprises a modified internucleoside linkage. In some aspects, the modified internucleoside linkage comprises a phosphorothioate linkage. In some aspects, the modified internucleoside linkage is between any of the 4 terminal nucleosides at a 5′ end or at a 3′ end of the integrating nucleic acid. The integrating nucleic acid may include multiple modified internucleoside linkages. For example, the integrating nucleic acid may include modified internucleoside linkages at nucleic acids of the 5′ and 3′ ends of the integrating nucleic acid, such as between the last 4 nucleic acids at the 5′ end and between the last 4 nucleic acids at the 3′ end. In some aspects, the integrating nucleic acid comprises a modified nucleoside. In some aspects, the modified nucleoside comprises a locked nucleic acid (LNA), a 2′ fluoro, a 2′ O-alkyl, a 5′ O-methyl, a 2′-O-methyl, or a combination thereof. The modified nucleoside may include an LNA, a 2′fluoro, a 2′ O-alkyl, a methylated cytosine, an inverted thymidine, or a combination thereof. The modified nucleoside may include an LNA. The modified nucleoside may include a 2′fluoro. The modified nucleoside may include a 2′ O-alkyl. The modified nucleoside may include a methylated cytosine. In some aspects, the modified nucleoside is any of the 3 terminal nucleosides at a 5′ end or at a 3′ end of the integrating nucleic acid. The integrating nucleic acid may include multiple modified nucleosides. For example, the integrating nucleic acid may include modified nucleosides at nucleic acids of the 5′ and 3′ ends of the integrating nucleic acid, such as the last 3 nucleic acids at the 5′ end and the last 3 nucleic acids at the 3′ end. The integrating nucleic acid may include any modification such as a modified nucleoside or modified internucleoside linkage described in relation to guide nucleic acids, insofar as it does not interfere with the function of the integrating nucleic acid after it is ligated into a target nucleic acid such as a host genome. The integrating nucleic acid may include any number or combination of modifications such as a number or combination described in relation to guide nucleic acids, insofar as it does not interfere with a function of the integrating nucleic acid. Table 8 includes some examples of integrating nucleic acid sequences.

The integrating nucleic acid may include a methylated nucleotide. The integrating nucleic acid may include an unmethylated nucleotide. An example of a methylated nucleotide may include a nucleotide including methylated cytosine. The cytosine may be methylated at a C-5 position of the cytosine ring. An example of an unmethylated nucleotide may include an unmethylated cytosine. The unmethylated nucleotide may include a cytosine that is not methylated at a C-5 position of the cytosine ring.

### Target Nucleic Acids

Disclosed herein are target nucleic acids. The target nucleic acid may include DNA. The target nucleic acid may be DNA. The target nucleic acid may include RNA. The target nucleic acid may be in a cell. The target nucleic acid may be methylated. The target nucleic acid may be unmethylated. The target nucleic acid may comprise a genome. The target nucleic acid may comprise genomic DNA. The target nucleic acid may comprise a chromosome. The target nucleic acid may comprise a gene.

The target nucleic acid may be in a subject. The target nucleic acid may be in a cell. The target nucleic acid may be in a test tube.

The target nucleic acid may be edited. The target nucleic acid may be edited in vitro. The target nucleic acid may be edited in vivo.

### Systems

Described herein are systems for nucleic acid editing (also known as gene editing). The editing system may include an endonuclease such as an RNA-guided endonuclease, a guide nucleic acid, and an integrating nucleic acid. Where gene editing is described, it is contemplated that the editing may be of a gene, regulatory element, or any sequence of a nucleic acid. Also, where genome editing is described, such as genome editing at a genetic locus, it is contemplated that nucleic acid editing not comprising a genome may also be performed. For example, genome editing may refer to editing of a genome of an organism, or may include editing of a nucleic acid that is not part of a genome. The systems described herein may be used in gene editing methods.

Described herein, in some aspects, is a system comprising at least one endonuclease; at least one guide nucleic acid; at least one ligase; at least one donor strand; or a combination thereof. In some aspects, the guide nucleic acid directs the endonuclease to the genomic locus for cleaving at least one strand of the genomic locus, where, after cleavage, the donor strand is ligated and thus incorporated into the genomic locus by the ligase. In some aspects, the system comprises: a first endonuclease to be complexed with a first guide nucleic acid, where the first endonuclease can be operatively coupled to a first ligase; and a second endonuclease to be complexed with a second guide nucleic acid, where the second endonuclease can be operatively coupled to a second ligase. In such system each of the first endonuclease and the second endonuclease can each cleave at least one strand of the genomic locus for incorporation of the donor strand.

In some aspects, the system comprises one, two, three, or more endonucleases. In some aspects, the system comprises one endonucleases. In some aspects, the two endonucleases can each be complexed with a different guide nucleic acid. In some aspects, the two endonucleases can each be operatively coupled to a ligase. In some aspects, the endonuclease is a programmable endonuclease. In some aspects, the endonuclease comprises a RNA-guided endonuclease, where the guide nucleic acid comprises a guide RNA. In some aspects, the endonuclease comprises a nickase, where the endonuclease only cleaves one strand (as opposed to making a double-stranded break). In some aspects, the endonuclease comprises a localization signal sequence to increase the accumulation of the endonuclease in the proximity of the genomic locus (e.g., in the nucleus). In some aspects, the endonuclease comprises at least one additional domain. In some aspects, the at least one additional domain is a dimerization domain. In some aspects, the endonuclease comprising a dimerization domain can be dimerized with a ligase to form a heterodimer. In some aspects, the at least one additional domain is a functional domain. For example, the functional domain can comprises a chromatin modifying domain or a cell penetrating peptide. In some aspects, the endonuclease comprises a linker, where the linker can covalently connect the endonuclease with another polypeptide (e.g., the ligase). In some aspects, the linker covalently connects the endonuclease to the at least one additional domain. In some aspects, the endonuclease comprises a tag, where the tag can be used for increasing expression, identifying, or purifying the endonuclease.

In some aspects, the system comprises one, two, three, or more guide nucleic acids. In some aspects, the system comprises one guide nucleic acid, where the one guide nucleic acid can be complexed with at least one endonuclease. In some aspects, the system comprises two guide nucleic acids, where the two guide nucleic acids can each be complexed with the at least one endonuclease. In some aspects, the guide nucleic acid comprises a spacer complementary to a genomic locus in a cell; a scaffold for complexing with the at least one endonuclease; a donor binding site that is at least partially complementary to a donor strand; a flap binding site that is at least partially identical or complementary to a genomic flap at or adjacent to the genomic locus; or a combination thereof. In some aspects, the guide nucleic acid can direct the at least one endonuclease to cleave at least one strand of the genomic locus. In some aspects, the guide nucleic acid can be at least partially complementary to the donor strand or at least partially complementary to a genomic flap (e.g., a genomic nucleic acid sequence that is displaced and become single-stranded when the guide nucleic acid recruits the endonuclease to the genomic locus). In some aspects, the guide nucleic acid, being at least partially complementary to the donor strand or at least partially complementary to a genomic flap, brings the donor strand to close proximity of the cleaving of the genomic locus. In some aspects, the guide nucleic acid comprises at least one nucleic acid modification. In some aspect, the at least nucleic acid modification comprises modifying a backbone, a sugar, a base, or a combination thereof of the guide nucleic acid. In some aspects, the at least one nucleic acid modification can increase resistance of the guide nucleic acid to degradation (e.g., against nuclease degradation or hydrolysis). In some aspects, the at least one nucleic acid modification can increase the complexing of the guide nucleic acid to the at least one endonuclease. In some aspects, the at least one nucleic acid modification can increase the complexing of the guide nucleic acid to the donor strand. In some aspects, the at least one nucleic acid modification can increase the complexing of the guide nucleic acid to the genomic locus via by being complementary to the genomic flap.

In some aspects, the system comprises one, two, three, or more ligase. In some aspects, the system comprises one ligase. In some aspects, the one ligase is operatively coupled with at least one endonuclease, where the ligase can ligate at least one end of the donor strand to the cleaved genomic locus, thus incorporating the donor strand into the genomic locus. In some aspects, the system comprises two ligases. In some aspects, the two ligases can each be operatively coupled to a different endonuclease, where the genomic locus is cleaved at two or more locations. In such scenario, the two ligases can each ligate one end of the donor strand to the cleaved genomic locus, thus incorporating the donor strand into the genomic locus. In some aspects, the ligase comprises a ligase that can ligate a substrate comprising DNA. In some aspects, the ligase comprises a ligase that can ligate a substrate comprising a DNA splint. In some aspects, the ligase comprises a ligase that can ligate a substrate comprising a DNA/RNA. In some aspects, the ligase comprises a ligase that can ligate a substrate comprising a RNA splint. In some aspects, the ligase comprises at least one additional domain. In some aspects, the at least one additional domain is a dimerization domain. In some aspects, the ligase comprising a dimerization domain can be dimerized with a endonuclease to form a heterodimer. In some aspects, the at least one additional domain is a functional domain. For example, the functional domain can comprises a chromatin modifying domain or a cell penetrating peptide. In some aspects, the ligase comprises a linker, where the linker can covalently connect the ligase with another polypeptide (e.g., the endonuclease). In some aspects, the linker covalently connects the ligase to the at least one additional domain. In some aspects, the ligase comprises a tag, where the tag can be used for increasing expression, identifying, or purifying the ligase.

Disclosed herein are fusion proteins comprising: an RNA-guided endonuclease fused to a ligase. Table 9 illustrates non-limiting examples of polypeptide and nucleic acid sequences encoding a fusion polypeptide comprising components (e.g., a endonuclease fused to a ligase) of a system described herein. SEQ ID NO: 125 illustrates a nucleic acid sequence encoding the polypeptide sequence of SEQ ID NO: 126, where SEQ ID NO: 126 illustrates a fusion protein (NLS-nCas9-linker-hLIG1(119-919)-bpNLS) comprising a N-terminus NLS followed by a endonuclease (nCas9) covalently connected to a ligase (hLIG1, 119-919 fragment) via a linker followed by a C-terminus NLS. SEQ ID NO: 127 illustrates a nucleic acid sequence encoding the polypeptide sequence of SEQ ID NO: 128, where SEQ ID NO: 128 illustrates a fusion protein (NLS-nCas9-linker-hLIG1(233-919)-bpNLS) comprising a N-terminus NLS followed by a endonuclease (nCas9) covalently connected to a ligase (hLIG1, 233-919 fragment) via a linker followed by a C-terminus NLS. SEQ ID NO: 129 illustrates a nucleic acid sequence encoding the polypeptide sequence of SEQ ID NO: 130, where SEQ ID NO: 130 illustrates a fusion protein (NLS-nCas9-linker-SplintR-bpNLS) comprising a N-terminus NLS followed by a endonuclease (nCas9) covalently connected to a ligase (SplintR) via a linker followed by a C-terminus NLS. SEQ ID NO: 131 illustrates a nucleic acid sequence encoding the polypeptide sequence of SEQ ID NO: 132, where SEQ ID NO: 132 illustrates a fusion protein (NLS-nCas9-linker-T4LIG-bpNLS) comprising a N-terminus NLS followed by a endonuclease (nCas9) covalently connected to a ligase (T4LIG) via a linker followed by a C-terminus NLS. SEQ ID NO: 133 illustrates a nucleic acid sequence encoding a endonuclease (nCas9) comprising a N-terminus NLS and a leucine zipper (LZ) dimerization domain. SEQ ID NO: 134 illustrates a fusion protein (NLS1-hFEN1-linker1-nCas9-linker2-T4LIG-NLS2) comprising first NLS (NLS1) at N-terminus followed by a exonuclease (hFEN1) covalently connected to a endonuclease (nCas9) via linker1 and further covalently connected to a ligase (T4LIG) via linker 2 followed by a second NLS (NLS2) at C-terminus. SEQ ID NO: 135 illustrates a fusion protein (NLS1-hFEN1-linker1-T4LIG-linker2-nCas9-NLS2) comprising a N-terminus NLS1 followed by a exonuclease (hFEN1) covalently connected to a ligase (T4LIG) via linker 1 and further covalently connected to a endonuclease (nCas9) via linker 2 followed by a C-terminus NLS2. SEQ ID NO: 136 illustrates a fusion protein (NLS1-nCas9-linker1-hFEN1-linker2-T4LIG-NLS2) comprising a N-terminus NLS1 followed by a endonuclease (nCas9) covalently connected to a exonuclease (hFEN1) via linker 1 and further covalently connected to a ligase (T4LIG) via linker 2 followed by a C-terminus NLS2. SEQ ID NO: 137 illustrates a fusion protein (NLS1-T4LIG-linker1-nCas9-linker2-hFEN1-NLS2) comprising a N-terminus NLS1 followed by a ligase (T4LIG) covalently connected to a endonuclease (nCas9) via linker 1 and further covalently connected to a exonuclease (hFEN1) via linker 2 followed by a C-terminus NLS2. SEQ ID NO: 138 illustrates a fusion protein (NLS1-nCas9-linker1-T4LIG-linker2-hFEN1-NLS2) comprising a N-terminus NLS1 followed by a endonuclease (nCas9) covalently connected to a ligase (T4LIG) via linker 1 and further covalently connected to a exonuclease (hFEN1) via linker 2 followed by a C-terminus NLS2. SEQ ID NO: 139 illustrates a fusion protein (NLS1-T4LIG-linker1-hFEN1-linker2-nCas9-NLS2) comprising a N-terminus NLS1 followed by a ligase (T4LIG) covalently connected to a exonuclease (hFEN1) via linker 1 and further covalently connected to a endonuclease (nCas9) via linker 2 followed by a C-terminus NLS2. SEQ ID NO: 140 illustrates a fusion protein (NLS1-T5 EXO-linker1-nCas9-linker2-T4LIG-NLS2) comprising a N-terminus NLS1 followed by a exonuclease (EXO) covalently connected to a endonuclease (nCas9) via linker 1 and further covalently connected to a ligase (T4LIG) via linker 2 followed by a C-terminus NLS2. SEQ ID NO: 141 illustrates a nucleic acid sequence encoding a fusion protein (LZ-SplintR-bpNLS) comprising a ligase (SplintR) fused to a dimerization domain (LZ) and a NLS. SEQ ID NO: 142 illustrates a nucleic acid sequence encoding a fusion protein (LZ-T4LIG-bpNLS) comprising a ligase (T4LIG) fused to a dimerization domain (LZ) and a NLS. SEQ ID NO: 143 illustrates a nucleic acid sequence encoding a fusion protein (LZ-hLIG 233-919 polypeptide fragment-bpNLS) comprising a ligase (hLIG) fused to a dimerization domain (LZ) and a NLS. SEQ ID NO: 144 illustrates a nucleic acid sequence encoding a fusion protein (LZ-hLIG1 119-919 polypeptide fragment-bpNLS) comprising a ligase (hLIG) fused to a dimerization domain (LZ) and a NLS. SEQ ID NO: 145 illustrates a nucleic acid sequence encoding a fusion protein (T4-LZ) comprising a ligase (T4) fused to a dimerization domain (LZ) and a NLS. SEQ ID NO: 146 illustrates a nucleic acid sequence encoding a fusion protein (LZ-hLIG4(1-620)) comprising a ligase polypeptide fragment (hLIG4(1-620)) fused to a dimerization domain (LZ) and a NLS. SEQ ID NO: 147 illustrates a nucleic acid sequence encoding a fusion protein (LZ-nCas9) comprising an endonuclease (nCas9) fused to a dimerization domain (LZ) and a NLS. SEQ ID NO: 148 illustrates a nucleic acid sequence encoding a fusion protein (SplintR-LZ) comprising a ligase (SplintR) fused to a dimerization domain (LZ) and a NLS. SEQ ID NO: 149 illustrates a nucleic acid sequence encoding a fusion protein (hLIG4(1-620)-LZ) comprising a ligase polypeptide fragment (hLIG4(1-620)) fused to a dimerization domain (LZ) and a NLS. SEQ ID NO: 150 illustrates a nucleic acid sequence encoding a fusion protein (nCas9-hLG4(1-620)) comprising a ligase polypeptide fragment (hLIG4(1-620)) fused to an endonuclease (nCas9) and a NLS. SEQ ID NO: 151 illustrates a nucleic acid sequence encoding a fusion protein (T4-nCas9) comprising a ligase (T4) fused to an endonuclease (nCas9) and a NLS. SEQ ID NO: 152 illustrates a nucleic acid sequence encoding a fusion protein (SplintR-nCas9) comprising a ligase (SplintR) fused to an endonuclease (nCas9) and a NLS. SEQ ID NO: 153 illustrates a nucleic acid sequence encoding a fusion protein (hLIG4(1-620)-nCas9) comprising a ligase polypeptide fragment (hLIG4(1-620)) fused to an endonuclease (nCas9) and a NLS.

TABLE 9
Non-limiting examples of fusion protein polypeptide sequence or
nucleic acid sequence encoding the fusion protein

| | Fusion protein polypeptide sequence or | SEQ |
|---|---|---|
| Name | nucleic acid sequence | ID NO: |
| NLS- | atgaaacggacagccgacggaagcgagttcgagtcaccaaagaagaagcg | 125 |
| nCas9- | gaaagtcgacaagaagtacagcatcggcctggacatcggcaccaactctg | |
| linker- | tgggctgggccgtgatcaccgacgagtacaaggtgcccagcaagaaattc | |
| hLIG1 | aaggtgctgggcaacaccgaccggcacagcatcaagaagaacctgatcgg | |
| (119- | agccctgctgttcgacagcggcgaaacagccgaggccacccggctgaaga | |
| 919)- | gaaccgccagaagaagatacaccagacggaagaaccggatctgctatctg | |
| bpNLS | caagagatcttcagcaacgagatggccaaggtggacgacagcttcttcca | |
| | cagactggaagagtccttcctggtggaagaggataagaagcacgagcggc | |
| | accccatcttcggcaacatcgtggacgaggtggcctaccacgagaagtac | |
| | cccaccatctaccacctgagaaagaaactggtggacagcaccgacaaggc | |
| | cgacctgcggctgatctatctggccctggcccacatgatcaagttccggg | |
| | gccacttcctgatcgagggcgacctgaaccccgacaacagcgacgtggac | |
| | aagctgttcatccagctggtgcagacctacaaccagctgttcgaggaaaa | |
| | ccccatcaacgccagcggcgtggacgccaaggccatcctgtctgccagac | |
| | tgagcaagagcagacggctggaaaatctgatcgcccagctgcccggcgag | |
| | aagaagaatggcctgttcggaaacctgattgccctgagcctgggcctgac | |
| | ccccaacttcaagagcaacttcgacctggccgaggatgccaaactgcagc | |
| | tgagcaaggacacctacgacgacgacctggacaacctgctggcccagatc | |
| | ggcgaccagtacgccgacctgtttctggccgccaagaacctgtccgacgc | |
| | catcctgctgagcgacatcctgagagtgaacaccgagatcaccaaggccc | |
| | ccctgagcgcctctatgatcaagagatacgacgagcaccaccaggacctg | |
| | accctgctgaaagctctcgtgcggcagcagctgcctgagaagtacaaaga | |
| | gattttcttcgaccagagcaagaacggctacgccggctacattgacggcg | |
| | gagccagccaggaagagttctacaagttcatcaagcccatcctggaaaag | |
| | atggacggcaccgaggaactgctcgtgaagctgaacagagaggacctgct | |
| | gcggaagcagcggaccttcgacaacggcagcatcccccaccagatccacc | |
| | tgggagagctgcacgccattctgcggcggcaggaagatttttacccattc | |
| | ctgaaggacaaccgggaaaagatcgagaagatcctgaccttccgcatccc | |
| | ctactacgtgggccctctggccaggggaaacagcagattcgcctggatga | |
| | ccagaaagagcgaggaaaccatcaccccctggaacttcgaggaagtggtg | |
| | gacaagggcgcttccgcccagagcttcatcgagcggatgaccaacttcga | |
| | taagaacctgcccaacgagaaggtgctgcccaagcacagcctgctgtacg | |
| | agtacttcaccgtgtataacgagctgaccaaagtgaaatacgtgaccgag | |
| | ggaatgagaaagcccgccttcctgagcggcgagcagaaaaaggccatcgt | |
| | ggacctgctgttcaagaccaaccggaaagtgaccgtgaagcagctgaaag | |
| | aggactacttcaagaaaatcgagtgcttcgactccgtggaaatctccggc | |
| | gtggaagatcggttcaacgcctccctgggcacataccacgatctgctgaa | |
| | aattatcaaggacaaggacttcctggacaatgaggaaaacgaggacattc | |
| | tggaagatatcgtgctgaccctgacactgtttgaggacagagagatgatc | |
| | gaggaacggctgaaaacctatgcccacctgttcgacgacaaagtgatgaa | |
| | gcagctgaagcggcggagatacaccggctggggcaggctgagccggaagc | |
| | tgatcaacggcatccgggacaagcagtccggcaagacaatcctggatttc | |
| | ctgaagtccgacggcttcgccaacagaaacttcatgcagctgatccacga | |
| | cgacagcctgacctttaaagaggacatccagaaagcccaggtgtccggcc | |
| | agggcgatagcctgcacgagcacattgccaatctggccggcagccccgcc | |
| | attaagaagggcatcctgcagacagtgaaggtggtggacgagctcgtgaa | |
| | agtgatgggccggcacaagcccgagaacatcgtgatcgaaatggccagag | |
| | agaaccagaccacccagaagggacagaagaacagccgcgagagaatgaag | |
| | cggatcgaagagggcatcaaagagctgggcagccagatcctgaaagaaca | |
| | ccccgtggaaaacacccagctgcagaacgagaagctgtacctgtactacc | |
| | tgcagaatgggcgggatatgtacgtggaccaggaactggacatcaaccgg | |
| | ctgtccgactacgatgtggacgctatcgtgcctcagagctttctgaagga | |
| | cgactccatcgacaacaaggtgctgaccagaagcgacaagaaccggggca | |
| | agagcgacaacgtgccctccgaagaggtcgtgaagaagatgaagaactac | |
| | tggcggcagctgctgaacgccaagctgattacccagagaaagttcgacaa | |
| | tctgaccaaggccgagagaggcggcctgagcgaactggataaggccggct | |
| | tcatcaagagacagctggtggaaacccggcagatcacaaagcacgtggca | |
| | cagatcctggactcccggatgaacactaagtacgacgagaatgacaagct | |
| | gatccgggaagtgaaagtgatcaccctgaagtccaagctggtgtccgatt | |
| | tccggaaggatttccagttttacaaagtgcgcgagatcaacaactaccac | |
| | cacgcccacgacgcctacctgaacgccgtcgtgggaaccgccctgatcaa | |
| | aaagtaccctaagctggaaagcgagttcgtgtacggcgactacaaggtgt | |
| | acgacgtgcggaagatgatcgccaagagcgagcaggaaatcggcaaggct | |
| | accgccaagtacttcttctacagcaacatcatgaactttttcaagaccga | |
| | gattaccctggccaacggcgagatccggaagcggcctctgatcgagacaa | |
| | acggcgaaaccggggagatcgtgtgggataagggccgggattttgccacc | |
| | gtgcggaaagtgctgagcatgccccaagtgaatatcgtgaaaaagaccga | |
| | ggtgcagacaggcggcttcagcaaagagtctatcctgcccaagaggaaca | |
| | gcgataagctgatcgccagaaagaaggactgggaccctaagaagtacggc | |
| | ggcttcgacagccccaccgtggcctattctgtgctggtggtggccaaagt | |
| | ggaaaagggcaagtccaagaaactgaagagtgtgaaagagctgctgggga | |
| | tcaccatcatggaaagaagcagcttcgagaagaatcccatcgactttctg | |
| | gaagccaagggctacaaagaagtgaaaaaggacctgatcatcaagctgcc | |
| | taagtactccctgttcgagctggaaaacggccggaagagaatgctggcct | |
| | ctgccggcgaactgcagaagggaaacgaactggccctgccctccaaatat | |
| | gtgaacttcctgtacctggccagccactatgagaagctgaagggctcccc | |
| | cgaggataatgagcagaaacagctgtttgtggaacagcacaagcactacc | |
| | tggacgagatcatcgagcagatcagcgagttctccaagagagtgatcctg | |
| | gccgacgctaatctggacaaagtgctgtccgcctacaacaagcaccggga | |
| | taagcccatcagagagcaggccgagaatatcatccacctgtttaccctga | |
| | ccaatctgggagcccctgccgccttcaagtactttgacaccaccatcgac | |
| | cggaagaggtacaccagcaccaaagaggtgctggacgccaccctgatcca | |
| | ccagagcatcaccggcctgtacgagacacggatcgacctgtctcagctgg | |
| | gaggtgacTCCGGCGGAAGCTCTGGTGGCAGCAAGCGGACCGCCGACGGC | |
| | TCTGAATTCGAGAGCCCTAAGAAGAAAAGAAAGGTGAGCGGAGGCTCTAG | |
| | CGGCGGAAGCCCGAAGCGCCGGACTGCACGAAAGCAACTGCCAAAACGGA | |
| | CTATACAAGAAGTCCTGGAAGAACAAAGCGAAGATGAGGATCGCGAAGCC | |
| | AAGCGCAAGAAAGAGGAAGAGGAAGAAGAGACTCCAAAGGAGTCCTTGAC | |
| | CGAAGCAGAAGTCGCAACGGAGAAGGAAGGTGAGGATGGGGATCAGCCAA | |
| | CAACCCCGCCTAAACCTCTGAAAACCTCTAAGGCGGAGACACCAACTGAG | |
| | AGTGTCAGCGAACCGGAGGTAGCCACGAAACAAGAGCTTCAGGAGGAAGA | |
| | AGAACAGACAAAGCCACCTCGGCGGGCTCCCAAAACCCTTAGCTCCTTCT | |
| | TCACGCCTCGAAAGCCAGCAGTGAAGAAAGAAGTGAAGGAGGAGGAACCT | |
| | GGCGCCCCTGGAAAGGAGGGCGCAGCCGAGGGCCCGCTGGACCCTTCAGG | |
| | GTATAACCCGGCAAAAAATAATTACCACCCGGTCGAGGACGCTTGTTGGA | |
| | AACCAGGCCAAAAGGTACCTTACCTCGCCGTCGCTAGGACCTTTGAGAAG | |
| | ATAGAGGAAGTTAGTGCTAGGTTGAGAATGGTCGAAACCCTTAGTAACCT | |
| | TCTCAGGTCCGTAGTCGCCCTTAGTCCCCCAGACCTGCTTCCGGTGCTGT | |
| | ACCTGTCCCTGAACCATCTCGGTCCCCCCCAACAGGGACTGGAGTTGGGC | |
| | GTCGGTGACGGCGTTCTCCTGAAAGCGGTTGCACAAGCTACAGGAAGGCA | |
| | ACTGGAATCTGTCCGGGCTGAGGCTGCAGAGAAAGGTGACGTGGGGCTTG | |
| | TGGCAGAGAATAGTCGGTCAACACAGCGGCTGATGCTGCCACCGCCCCCG | |
| | CTTACGGCTAGTGGGGTATTCTCCAAATTTAGAGATATAGCACGGCTGAC | |
| | GGGATCAGCTTCCACTGCGAAGAAGATCGATATCATTAAGGGTTTGTTCG | |
| | TGGCTTGCAGGCATTCCGAAGCACGCTTCATTGCACGCTCCCTTTCAGGG | |
| | AGACTCAGACTTGGGCTGGCCGAGCAATCTGTACTGGCGGCCCTGTCTCA | |
| | GGCGGTGAGCCTTACGCCGCCCGGGCAAGAGTTCCCTCCTGCGATGGTCG | |
| | ATGCTGGGAAGGGAAAAACCGCCGAAGCTCGAAAAACATGGCTGGAGGAG | |
| | CAAGGAATGATTTTGAAGCAGACGTTCTGTGAAGTACCGGACTTGGATCG | |
| | CATCATACCTGTGCTTCTCGAACATGGTTTGGAGCGGCTCCCCGAGCATT | |
| | GCAAACTCTCTCCGGGCATCCCCCTCAAGCCAATGCTCGCCCACCCCACG | |
| | CGCGGAATCAGTGAGGTACTGAAACGCTTTGAAGAGGCAGCGTTTACTTG | |
| | TGAATACAAGTACGATGGCCAAAGGGCACAAATTCATGCACTTGAAGGCG | |
| | GGGAAGTTAAGATATTCAGCAGGAATCAGGAGGACAACACGGGAAAATAT | |
| | CCTGACATAATATCTCGAATCCCTAAAATTAAGTTGCCTAGCGTAACCAG | |
| | CTTCATCCTGGATACCGAAGCCGTGGCGTGGGATAGGGAGAAAAAGCAAA | |
| | TACAGCCATTTCAGGTGCTTACAACTAGAAAACGAAAAGAGGTGGACGCT | |
| | AGTGAAATCCAAGTCCAGGTATGTCTTTATGCCTTCGATTTGATATACCT | |
| | TAATGGTGAGTCCCTTGTACGGGAACCGCTTAGTAGGAGGCGGCAGTTGC | |
| | TGAGGGAAAATTTTGTCGAAACTGAGGGAGAGTTTGTATTTGCAACGTCA | |
| | TTGGATACAAAGGACATAGAACAAATAGCAGAATTTCTGGAGCAGTCAGT | |
| | AAAAGACTCCTGCGAGGGCCTGATGGTGAAAACTCTTGATGTGGACGCCA | |
| | CTTATGAAATCGCAAAAAGGTCACACAATTGGCTGAAACTTAAAAAGGAT | |
| | TACTTGGACGGGGTCGGGGATACCCTCGATCTCGTCGTAATCGGAGCTTA | |
| | TCTCGGTAGGGGGAAGCGAGCCGGGCGATACGGAGGCTTTCTCTTGGCTA | |
| | GTTATGACGAAGATTCCGAAGAGCTGCAGGCCATATGCAAGCTTGGAACG | |
| | GGTTTCAGCGATGAGGAATTGGAGGAGCATCATCAGAGCTTGAAGGCACT | |
| | GGTGCTCCCCTCTCCTAGGCCGTACGTTAGAATAGACGGAGCAGTGATAC | |
| | CCGATCATTGGCTCGATCCGTCAGCTGTTTGGGAGGTGAAGTGTGCAGAC | |
| | CTGTCCCTCTCTCCTATTTACCCTGCAGCACGCGGTCTGGTTGACTCTGA | |
| | CAAAGGGATTAGCTTGAGGTTCCCTAGATTTATTCGGGTGCGCGAAGATA | |
| | AACAGCCTGAACAGGCGACAACGTCCGCGCAGGTCGCATGCCTTTATCGA | |
| | AAACAGAGTCAGATCCAGAATCAACAAGGAGAAGATTCAGGGAGTGACCC | |
| | GGAGGACACTTATAGTGGCGGCTCAAAACGAACCGCCGATAGTCAGCATT | |
| | CAACACCTCCAAAAACTAAAAGGAAAGTCGAGTTTGAGCCAAAGAAGAAG | |
| | CGCAAAGTCTAA | |
| NLS- | MKRTADGSEFESPKKKRKVDKKYSIGLDIGTNSVGWAVITDEYKVPSKKF | 126 |
| nCas9- | KVLGNTDRHSIKKNLIGALLFDSGETAEATRLKRTARRRYTRRKNRICYL | |
| linker- | QEIFSNEMAKVDDSFFHRLEESFLVEEDKKHERHPIFGNIVDEVAYHEKY | |
| hLIG1 | PTIYHLRKKLVDSTDKADLRLIYLALAHMIKFRGHFLIEGDLNPDNSDVD | |
| (119- | KLFIQLVQTYNQLFEENPINASGVDAKAILSARLSKSRRLENLIAQLPGE | |
| 919)- | KKNGLFGNLIALSLGLTPNFKSNFDLAEDAKLQLSKDTYDDDLDNLLAQI | |
| bpNLS | GDQYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMK | |
| | RIEEGIKELGSQILKEHPVENTQLQNEKLYLYYLQNGRDMYVDQELDINR | |
| | LSDYDVDAIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYH | |
| | HAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYG | |
| | GFDSPTVAYSVLVVAKVEKGKSKKLKSVKELLGITIMERSSFEKNPIDFL | |
| | EAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVIL | |
| | ADANLDKVLSAYNKHRDKPIREQAENIIHLFTLTNLGAPAAFKYFDTTID | |
| | RKRYTSTKEVLDATLIHQSITGLYETRIDLSQLGGDSGGSSGGSKRTADG | |
| | SEFESPKKKRKVSGGSSGGSPKRRTARKQLPKRTIQEVLEEQSEDEDREA | |
| | KRKKEEEEEETPKESLTEAEVATEKEGEDGDQPTTPPKPLKTSKAETPTE | |
| | SVSEPEVATKQELQEEEEQTKPPRRAPKTLSSFFTPRKPAVKKEVKEEEP | |
| | GAPGKEGAAEGPLDPSGYNPAKNNYHPVEDACWKPGQKVPYLAVARTFEK | |
| | IEEVSARLRMVETLSNLLRSVVALSPPDLLPVLYLSLNHLGPPQQGLELG | |
| | VGDGVLLKAVAQATGRQLESVRAEAAEKGDVGLVAENSRSTQRLMLPPPP | |
| | LTASGVFSKFRDIARLTGSASTAKKIDIIKGLFVACRHSEARFIARSLSG | |
| | RLRLGLAEQSVLAALSQAVSLTPPGQEFPPAMVDAGKGKTAEARKTWLEE | |
| | QGMILKQTFCEVPDLDRIIPVLLEHGLERLPEHCKLSPGIPLKPMLAHPT | |
| | RGISEVLKRFEEAAFTCEYKYDGQRAQIHALEGGEVKIFSRNQEDNTGKY | |
| | PDIISRIPKIKLPSVTSFILDTEAVAWDREKKQIQPFQVLTTRKRKEVDA | |
| | SEIQVQVCLYAFDLIYLNGESLVREPLSRRRQLLRENFVETEGEFVFATS | |
| | LDTKDIEQIAEFLEQSVKDSCEGLMVKTLDVDATYEIAKRSHNWLKLKKD | |
| | YLDGVGDTLDLVVIGAYLGRGKRAGRYGGFLLASYDEDSEELQAICKLGT | |
| | GFSDEELEEHHQSLKALVLPSPRPYVRIDGAVIPDHWLDPSAVWEVKCAD | |
| | LSLSPIYPAARGLVDSDKGISLRFPRFIRVREDKQPEQATTSAQVACLYR | |
| | KQSQIQNQQGEDSGSDPEDTYSGGSKRTADSQHSTPPKTKRKVEFEPKKK | |
| | RKV* | |
| NLS- | atgaaacggacagccgacggaagcgagttcgagtcaccaaagaagaagcg | 127 |
| nCas9- | gaaagtcgacaagaagtacagcatcggcctggacatcggcaccaactctg | |
| linker- | tgggctgggccgtgatcaccgacgagtacaaggtgcccagcaagaaattc | |
| hLIG1 | aaggtgctgggcaacaccgaccggcacagcatcaagaagaacctgatcgg | |
| (233- | agccctgctgttcgacagcggcgaaacagccgaggccacccggctgaaga | |
| 919)- | gaaccgccagaagaagatacaccagacggaagaaccggatctgctatctg | |
| bpNLS | caagagatcttcagcaacgagatggccaaggtggacgacagcttcttcca | |
| | cagactggaagagtccttcctggtggaagaggataagaagcacgagcggc | |
| | accccatcttcggcaacatcgtggacgaggtggcctaccacgagaagtac | |
| | cccaccatctaccacctgagaaagaaactggtggacagcaccgacaaggc | |
| | cgacctgcggctgatctatctggccctggcccacatgatcaagttccggg | |
| | gccacttcctgatcgagggcgacctgaaccccgacaacagcgacgtggac | |
| | aagctgttcatccagctggtgcagacctacaaccagctgttcgaggaaaa | |
| | ccccatcaacgccagcggcgtggacgccaaggccatcctgtctgccagac | |
| | tgagcaagagcagacggctggaaaatctgatcgcccagctgcccggcgag | |
| | aagaagaatggcctgttcggaaacctgattgccctgagcctgggcctgac | |
| | ccccaacttcaagagcaacttcgacctggccgaggatgccaaactgcagc | |
| | tgagcaaggacacctacgacgacgacctggacaacctgctggcccagatc | |
| | ggcgaccagtacgccgacctgtttctggccgccaagaacctgtccgacgc | |
| | catcctgctgagcgacatcctgagagtgaacaccgagatcaccaaggccc | |
| | ccctgagcgcctctatgatcaagagatacgacgagcaccaccaggacctg | |
| | accctgctgaaagctctcgtgcggcagcagctgcctgagaagtacaaaga | |
| | gattttcttcgaccagagcaagaacggctacgccggctacattgacggcg | |
| | gagccagccaggaagagttctacaagttcatcaagcccatcctggaaaag | |
| | atggacggcaccgaggaactgctcgtgaagctgaacagagaggacctgct | |
| | gcggaagcagcggaccttcgacaacggcagcatcccccaccagatccacc | |
| | tgggagagctgcacgccattctgcggcggcaggaagatttttacccattc | |
| | ctgaaggacaaccgggaaaagatcgagaagatcctgaccttccgcatccc | |
| | ctactacgtgggccctctggccaggggaaacagcagattcgcctggatga | |
| | ccagaaagagcgaggaaaccatcaccccctggaacttcgaggaagtggtg | |
| | gacaagggcgcttccgcccagagcttcatcgagcggatgaccaacttcga | |
| | taagaacctgcccaacgagaaggtgctgcccaagcacagcctgctgtacg | |
| | agtacttcaccgtgtataacgagctgaccaaagtgaaatacgtgaccgag | |
| | ggaatgagaaagcccgccttcctgagcggcgagcagaaaaaggccatcgt | |
| | ggacctgctgttcaagaccaaccggaaagtgaccgtgaagcagctgaaag | |
| | aggactacttcaagaaaatcgagtgcttcgactccgtggaaatctccggc | |
| | gtggaagatcggttcaacgcctccctgggcacataccacgatctgctgaa | |
| | aattatcaaggacaaggacttcctggacaatgaggaaaacgaggacattc | |
| | tggaagatatcgtgctgaccctgacactgtttgaggacagagagatgatc | |
| | gaggaacggctgaaaacctatgcccacctgttcgacgacaaagtgatgaa | |
| | gcagctgaagcggcggagatacaccggctggggcaggctgagccggaagc | |
| | tgatcaacggcatccgggacaagcagtccggcaagacaatcctggatttc | |
| | ctgaagtccgacggcttcgccaacagaaacttcatgcagctgatccacga | |
| | cgacagcctgacctttaaagaggacatccagaaagcccaggtgtccggcc | |
| | agggcgatagcctgcacgagcacattgccaatctggccggcagccccgcc | |
| | attaagaagggcatcctgcagacagtgaaggtggtggacgagctcgtgaa | |
| | agtgatgggccggcacaagcccgagaacatcgtgatcgaaatggccagag | |
| | agaaccagaccacccagaagggacagaagaacagccgcgagagaatgaag | |
| | cggatcgaagagggcatcaaagagctgggcagccagatcctgaaagaaca | |
| | ccccgtggaaaacacccagctgcagaacgagaagctgtacctgtactacc | |
| | tgcagaatgggcgggatatgtacgtggaccaggaactggacatcaaccgg | |
| | ctgtccgactacgatgtggacgctatcgtgcctcagagctttctgaagga | |
| | cgactccatcgacaacaaggtgctgaccagaagcgacaagaaccggggca | |
| | agagcgacaacgtgccctccgaagaggtcgtgaagaagatgaagaactac | |
| | tggcggcagctgctgaacgccaagctgattacccagagaaagttcgacaa | |
| | tctgaccaaggccgagagaggcggcctgagcgaactggataaggccggct | |
| | tcatcaagagacagctggtggaaacccggcagatcacaaagcacgtggca | |
| | cagatcctggactcccggatgaacactaagtacgacgagaatgacaagct | |
| | gatccgggaagtgaaagtgatcaccctgaagtccaagctggtgtccgatt | |
| | tccggaaggatttccagttttacaaagtgcgcgagatcaacaactaccac | |
| | cacgcccacgacgcctacctgaacgccgtcgtgggaaccgccctgatcaa | |
| | aaagtaccctaagctggaaagcgagttcgtgtacggcgactacaaggtgt | |
| | acgacgtgcggaagatgatcgccaagagcgagcaggaaatcggcaaggct | |
| | accgccaagtacttcttctacagcaacatcatgaactttttcaagaccga | |
| | gattaccctggccaacggcgagatccggaagcggcctctgatcgagacaa | |
| | acggcgaaaccggggagatcgtgtgggataagggccgggattttgccacc | |
| | gtgcggaaagtgctgagcatgccccaagtgaatatcgtgaaaaagaccga | |
| | ggtgcagacaggcggcttcagcaaagagtctatcctgcccaagaggaaca | |
| | gcgataagctgatcgccagaaagaaggactgggaccctaagaagtacggc | |
| | ggcttcgacagccccaccgtggcctattctgtgctggtggtggccaaagt | |
| | ggaaaagggcaagtccaagaaactgaagagtgtgaaagagctgctgggga | |
| | tcaccatcatggaaagaagcagcttcgagaagaatcccatcgactttctg | |
| | gaagccaagggctacaaagaagtgaaaaaggacctgatcatcaagctgcc | |
| | taagtactccctgttcgagctggaaaacggccggaagagaatgctggcct | |
| | ctgccggcgaactgcagaagggaaacgaactggccctgccctccaaatat | |
| | gtgaacttcctgtacctggccagccactatgagaagctgaagggctcccc | |
| | cgaggataatgagcagaaacagctgtttgtggaacagcacaagcactacc | |
| | tggacgagatcatcgagcagatcagcgagttctccaagagagtgatcctg | |
| | gccgacgctaatctggacaaagtgctgtccgcctacaacaagcaccggga | |
| | taagcccatcagagagcaggccgagaatatcatccacctgtttaccctga | |
| | ccaatctgggagcccctgccgccttcaagtactttgacaccaccatcgac | |
| | cggaagaggtacaccagcaccaaagaggtgctggacgccaccctgatcca | |
| | ccagagcatcaccggcctgtacgagacacggatcgacctgtctcagctgg | |
| | gaggtgacTCCGGCGGAAGCTCTGGTGGCAGCAAGCGGACCGCCGACGGC | |
| | TCTGAATTCGAGAGCCCTAAGAAGAAAAGAAAGGTGAGCGGAGGCTCTAG | |
| | CGGCGGAAGCACACCCAGGAAACCAGCCGTGAAAAAAGAGGTTAAAGAAG | |
| | AGGAACCTGGGGCTCCGGGAAAGGAGGGAGCAGCGGAAGGTCCGCTCGAC | |
| | CCTTCAGGATACAACCCAGCCAAAAACAACTACCACCCCGTAGAGGATGC | |
| | TTGCTGGAAGCCAGGCCAAAAGGTGCCCTATTTGGCCGTTGCTAGGACTT | |
| | TCGAAAAAATTGAGGAGGTGAGCGCGCGACTCAGAATGGTAGAGACTCTG | |
| | TCTAACCTCCTTCGCTCCGTAGTGGCTCTTTCACCTCCAGATCTTCTTCC | |
| | AGTGCTGTACCTGAGCCTGAACCACTTGGGCCCTCCCCAGCAGGGACTGG | |
| | AACTGGGCGTAGGGGACGGAGTATTGCTGAAGGCTGTTGCTCAGGCAACC | |
| | GGACGACAGCTCGAGTCTGTGCGAGCAGAAGCTGCAGAAAAGGGGGACGT | |
| | CGGGTTGGTTGCCGAAAATTCAAGATCTACCCAACGATTGATGTTGCCAC | |
| | CGCCGCCTCTGACTGCGTCAGGTGTATTCTCCAAGTTCCGGGATATTGCC | |
| | AGGCTTACGGGTAGCGCTTCCACTGCTAAAAAGATCGACATAATAAAAGG | |
| | TCTGTTCGTCGCTTGTCGCCATTCAGAGGCGAGGTTTATAGCCAGATCCC | |
| | TTTCCGGACGACTTCGACTCGGCTTGGCTGAGCAGTCAGTACTGGCAGCT | |
| | TTGTCTCAAGCTGTATCACTCACGCCCCCCGGACAAGAATTTCCACCCGC | |
| | CATGGTTGACGCAGGCAAGGGTAAGACTGCTGAGGCAAGAAAGACGTGGC | |
| | TGGAGGAACAAGGTATGATACTTAAACAAACGTTTTGCGAAGTTCCGGAC | |
| | TTGGACCGGATCATACCTGTGTTGCTGGAGCACGGCCTCGAGCGCTTGCC | |
| | CGAACACTGTAAACTGTCTCCAGGAATACCTCTCAAACCCATGTTGGCTC | |
| | ATCCTACGAGGGGAATCTCAGAGGTACTTAAACGGTTTGAAGAAGCCGCT | |
| | TTCACGTGCGAATACAAGTATGATGGTCAGAGAGCGCAAATCCACGCATT | |
| | GGAAGGGGGTGAGGTAAAGATTTTTTCAAGGAATCAGGAGGACAATACAG | |
| | GGAAGTACCCCGATATCATCAGTCGGATTCCTAAAATTAAGCTTCCATCA | |
| | GTCACGTCCTTCATACTGGACACTGAGGCAGTGGCTTGGGACCGAGAGAA | |
| | GAAGCAGATACAACCCTTTCAGGTACTTACAACCAGAAAGCGCAAGGAAG | |
| | TCGACGCTTCTGAGATTCAAGTACAAGTCTGCCTTTATGCGTTTGACCTG | |
| | ATCTATCTTAATGGAGAGAGTTTGGTGAGAGAACCCTTGAGCAGACGACG | |
| | GCAGCTCTTGAGAGAAAATTTCGTAGAAACTGAGGGGGAGTTCGTCTTTG | |
| | CGACTAGTCTCGACACCAAAGACATTGAGCAAATCGCGGAATTCCTCGAA | |
| | CAGTCAGTTAAAGACTCCTGCGAAGGTCTGATGGTTAAGACTCTTGACGT | |
| | GGATGCTACCTACGAGATAGCTAAGCGGTCACACAATTGGCTGAAACTGA | |
| | AAAAGGACTATCTGGATGGAGTTGGGGACACGCTGGATTTGGTCGTTATC | |
| | GGGGCCTATCTGGGACGCGGTAAGCGGGCAGGGAGATATGGTGGATTCCT | |
| | CCTCGCTTCATACGATGAGGACTCTGAAGAGCTGCAGGCTATATGCAAAC | |
| | TTGGGACGGGTTTTTCCGATGAAGAATTGGAGGAACATCATCAGTCACTG | |
| | AAGGCCCTTGTATTGCCAAGTCCACGCCCATACGTACGAATCGATGGAGC | |
| | AGTAATCCCTGACCACTGGCTTGACCCGTCCGCCGTCTGGGAAGTAAAGT | |
| | GCGCGGATCTCTCTCTCAGTCCGATCTACCCAGCCGCACGGGGGCTGGTT | |
| | GACAGTGACAAGGGTATCAGCCTGCGATTTCCTCGATTCATACGCGTCCG | |
| | GGAAGACAAGCAACCGGAACAGGCTACGACCTCTGCACAGGTCGCATGTT | |
| | TGTATAGAAAACAGAGCCAAATTCAGAATCAACAAGGCGAAGACAGTGGG | |
| | TCCGATCCTGAAGATACCTACTCAGGCGGCAGTAAACGGACAGCTGATAG | |
| | CCAACACTCAACTCCTCCGAAGACTAAAAGGAAGGTAGAGTTCGAACCAA | |
| | AAAAGAAAAGGAAAGTGTAA | |
| NLS- | MKRTADGSEFESPKKKRKVDKKYSIGLDIGTNSVGWAVITDEYKVPSKKF | 128 |
| nCas9- | KVLGNTDRHSIKKNLIGALLFDSGETAEATRLKRTARRRYTRRKNRICYL | |
| linker- | QEIFSNEMAKVDDSFFHRLEESFLVEEDKKHERHPIFGNIVDEVAYHEKY | |
| hLIG1 | PTIYHLRKKLVDSTDKADLRLIYLALAHMIKFRGHFLIEGDLNPDNSDVD | |
| (233- | KLFIQLVQTYNQLFEENPINASGVDAKAILSARLSKSRRLENLIAQLPGE | |
| 919)- | KKNGLFGNLIALSLGLTPNFKSNFDLAEDAKLQLSKDTYDDDLDNLLAQI | |
| bpNLS | GDQYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMK | |
| | RIEEGIKELGSQILKEHPVENTQLQNEKLYLYYLQNGRDMYVDQELDINR | |
| | LSDYDVDAIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYH | |
| | HAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYG | |
| | GFDSPTVAYSVLVVAKVEKGKSKKLKSVKELLGITIMERSSFEKNPIDFL | |
| | EAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVIL | |
| | ADANLDKVLSAYNKHRDKPIREQAENIIHLFTLTNLGAPAAFKYFDTTID | |
| | RKRYTSTKEVLDATLIHQSITGLYETRIDLSQLGGDSGGSSGGSKRTADG | |
| | SEFESPKKKRKVSGGSSGGSTPRKPAVKKEVKEEEPGAPGKEGAAEGPLD | |
| | PSGYNPAKNNYHPVEDACWKPGQKVPYLAVARTFEKIEEVSARLRMVETL | |
| | SNLLRSVVALSPPDLLPVLYLSLNHLGPPQQGLELGVGDGVLLKAVAQAT | |
| | GRQLESVRAEAAEKGDVGLVAENSRSTQRLMLPPPPLTASGVFSKFRDIA | |
| | RLTGSASTAKKIDIIKGLFVACRHSEARFIARSLSGRLRLGLAEQSVLAA | |
| | LSQAVSLTPPGQEFPPAMVDAGKGKTAEARKTWLEEQGMILKQTFCEVPD | |
| | LDRIIPVLLEHGLERLPEHCKLSPGIPLKPMLAHPTRGISEVLKRFEEAA | |
| | FTCEYKYDGQRAQIHALEGGEVKIFSRNQEDNTGKYPDIISRIPKIKLPS | |
| | VTSFILDTEAVAWDREKKQIQPFQVLTTRKRKEVDASEIQVQVCLYAFDL | |
| | IYLNGESLVREPLSRRRQLLRENFVETEGEFVFATSLDTKDIEQIAEFLE | |
| | QSVKDSCEGLMVKTLDVDATYEIAKRSHNWLKLKKDYLDGVGDTLDLVVI | |
| | GAYLGRGKRAGRYGGFLLASYDEDSEELQAICKLGTGFSDEELEEHHQSL | |
| | KALVLPSPRPYVRIDGAVIPDHWLDPSAVWEVKCADLSLSPIYPAARGLV | |
| | DSDKGISLRFPRFIRVREDKQPEQATTSAQVACLYRKQSQIQNQQGEDSG | |
| | SDPEDTYSGGSKRTADSQHSTPPKTKRKVEFEPKKKRKV* | |
| NLS- | atgaaacggacagccgacggaagcgagttcgagtcaccaaagaagaagcg | 129 |
| nCas9- | gaaagtcgacaagaagtacagcatcggcctggacatcggcaccaactctg | |
| linker- | tgggctgggccgtgatcaccgacgagtacaaggtgcccagcaagaaattc | |
| SplintR- | aaggtgctgggcaacaccgaccggcacagcatcaagaagaacctgatcgg | |
| bpNLS | agccctgctgttcgacagcggcgaaacagccgaggccacccggctgaaga | |
| | gaaccgccagaagaagatacaccagacggaagaaccggatctgctatctg | |
| | caagagatcttcagcaacgagatggccaaggtggacgacagcttcttcca | |
| | cagactggaagagtccttcctggtggaagaggataagaagcacgagcggc | |
| | accccatcttcggcaacatcgtggacgaggtggcctaccacgagaagtac | |
| | cccaccatctaccacctgagaaagaaactggtggacagcaccgacaaggc | |
| | cgacctgcggctgatctatctggccctggcccacatgatcaagttccggg | |
| | gccacttcctgatcgagggcgacctgaaccccgacaacagcgacgtggac | |
| | aagctgttcatccagctggtgcagacctacaaccagctgttcgaggaaaa | |
| | ccccatcaacgccagcggcgtggacgccaaggccatcctgtctgccagac | |
| | tgagcaagagcagacggctggaaaatctgatcgcccagctgcccggcgag | |
| | aagaagaatggcctgttcggaaacctgattgccctgagcctgggcctgac | |
| | ccccaacttcaagagcaacttcgacctggccgaggatgccaaactgcagc | |
| | tgagcaaggacacctacgacgacgacctggacaacctgctggcccagatc | |
| | ggcgaccagtacgccgacctgtttctggccgccaagaacctgtccgacgc | |
| | catcctgctgagcgacatcctgagagtgaacaccgagatcaccaaggccc | |
| | ccctgagcgcctctatgatcaagagatacgacgagcaccaccaggacctg | |
| | accctgctgaaagctctcgtgcggcagcagctgcctgagaagtacaaaga | |
| | gattttcttcgaccagagcaagaacggctacgccggctacattgacggcg | |
| | gagccagccaggaagagttctacaagttcatcaagcccatcctggaaaag | |
| | atggacggcaccgaggaactgctcgtgaagctgaacagagaggacctgct | |
| | gcggaagcagcggaccttcgacaacggcagcatcccccaccagatccacc | |
| | tgggagagctgcacgccattctgcggcggcaggaagatttttacccattc | |
| | ctgaaggacaaccgggaaaagatcgagaagatcctgaccttccgcatccc | |
| | ctactacgtgggccctctggccaggggaaacagcagattcgcctggatga | |
| | ccagaaagagcgaggaaaccatcaccccctggaacttcgaggaagtggtg | |
| | gacaagggcgcttccgcccagagcttcatcgagcggatgaccaacttcga | |
| | taagaacctgcccaacgagaaggtgctgcccaagcacagcctgctgtacg | |
| | agtacttcaccgtgtataacgagctgaccaaagtgaaatacgtgaccgag | |
| | ggaatgagaaagcccgccttcctgagcggcgagcagaaaaaggccatcgt | |
| | ggacctgctgttcaagaccaaccggaaagtgaccgtgaagcagctgaaag | |
| | aggactacttcaagaaaatcgagtgcttcgactccgtggaaatctccggc | |
| | gtggaagatcggttcaacgcctccctgggcacataccacgatctgctgaa | |
| | aattatcaaggacaaggacttcctggacaatgaggaaaacgaggacattc | |
| | tggaagatatcgtgctgaccctgacactgtttgaggacagagagatgatc | |
| | gaggaacggctgaaaacctatgcccacctgttcgacgacaaagtgatgaa | |
| | gcagctgaagcggcggagatacaccggctggggcaggctgagccggaagc | |
| | tgatcaacggcatccgggacaagcagtccggcaagacaatcctggatttc | |
| | ctgaagtccgacggcttcgccaacagaaacttcatgcagctgatccacga | |
| | cgacagcctgacctttaaagaggacatccagaaagcccaggtgtccggcc | |
| | agggcgatagcctgcacgagcacattgccaatctggccggcagccccgcc | |
| | attaagaagggcatcctgcagacagtgaaggtggtggacgagctcgtgaa | |
| | agtgatgggccggcacaagcccgagaacatcgtgatcgaaatggccagag | |
| | agaaccagaccacccagaagggacagaagaacagccgcgagagaatgaag | |
| | cggatcgaagagggcatcaaagagctgggcagccagatcctgaaagaaca | |
| | ccccgtggaaaacacccagctgcagaacgagaagctgtacctgtactacc | |
| | tgcagaatgggcgggatatgtacgtggaccaggaactggacatcaaccgg | |
| | ctgtccgactacgatgtggacgctatcgtgcctcagagctttctgaagga | |
| | cgactccatcgacaacaaggtgctgaccagaagcgacaagaaccggggca | |
| | agagcgacaacgtgccctccgaagaggtcgtgaagaagatgaagaactac | |
| | tggcggcagctgctgaacgccaagctgattacccagagaaagttcgacaa | |
| | tctgaccaaggccgagagaggcggcctgagcgaactggataaggccggct | |
| | tcatcaagagacagctggtggaaacccggcagatcacaaagcacgtggca | |
| | cagatcctggactcccggatgaacactaagtacgacgagaatgacaagct | |
| | gatccgggaagtgaaagtgatcaccctgaagtccaagctggtgtccgatt | |
| | tccggaaggatttccagttttacaaagtgcgcgagatcaacaactaccac | |
| | cacgcccacgacgcctacctgaacgccgtcgtgggaaccgccctgatcaa | |
| | aaagtaccctaagctggaaagcgagttcgtgtacggcgactacaaggtgt | |
| | acgacgtgcggaagatgatcgccaagagcgagcaggaaatcggcaaggct | |
| | accgccaagtacttcttctacagcaacatcatgaactttttcaagaccga | |
| | gattaccctggccaacggcgagatccggaagcggcctctgatcgagacaa | |
| | acggcgaaaccggggagatcgtgtgggataagggccgggattttgccacc | |
| | gtgcggaaagtgctgagcatgccccaagtgaatatcgtgaaaaagaccga | |
| | ggtgcagacaggcggcttcagcaaagagtctatcctgcccaagaggaaca | |
| | gcgataagctgatcgccagaaagaaggactgggaccctaagaagtacggc | |
| | ggcttcgacagccccaccgtggcctattctgtgctggtggtggccaaagt | |
| | ggaaaagggcaagtccaagaaactgaagagtgtgaaagagctgctgggga | |
| | tcaccatcatggaaagaagcagcttcgagaagaatcccatcgactttctg | |
| | gaagccaagggctacaaagaagtgaaaaaggacctgatcatcaagctgcc | |
| | taagtactccctgttcgagctggaaaacggccggaagagaatgctggcct | |
| | ctgccggcgaactgcagaagggaaacgaactggccctgccctccaaatat | |
| | gtgaacttcctgtacctggccagccactatgagaagctgaagggctcccc | |
| | cgaggataatgagcagaaacagctgtttgtggaacagcacaagcactacc | |
| | tggacgagatcatcgagcagatcagcgagttctccaagagagtgatcctg | |
| | gccgacgctaatctggacaaagtgctgtccgcctacaacaagcaccggga | |
| | taagcccatcagagagcaggccgagaatatcatccacctgtttaccctga | |
| | ccaatctgggagcccctgccgccttcaagtactttgacaccaccatcgac | |
| | cggaagaggtacaccagcaccaaagaggtgctggacgccaccctgatcca | |
| | ccagagcatcaccggcctgtacgagacacggatcgacctgtctcagctgg | |
| | gaggtgacTCCGGCGGAAGCTCTGGTGGCAGCAAGCGGACCGCCGACGGC | |
| | TCTGAATTCGAGAGCCCTAAGAAGAAAAGAAAGGTGAGCGGAGGCTCTAG | |
| | CGGCGGAAGCATGGCAATCACTAAGCCCCTCTTGGCGGCGACTTTGGAAA | |
| | ACATCGAGGATGTGCAATTCCCGTGCCTTGCCACACCAAAGATAGACGGG | |
| | ATCCGATCAGTGAAGCAAACGCAGATGCTCTCTAGAACGTTCAAGCCTAT | |
| | TAGAAACTCAGTGATGAATCGGCTCTTGACTGAGCTGTTGCCGGAAGGCA | |
| | GCGATGGGGAAATATCTATCGAGGGAGCCACATTTCAAGACACTACGAGC | |
| | GCCGTAATGACTGGACATAAGATGTATAATGCTAAATTCTCCTACTATTG | |
| | GTTTGACTATGTCACTGATGACCCTCTTAAGAAATATATAGACCGAGTGG | |
| | AGGATATGAAAAATTATATTACTGTACACCCGCATATTCTGGAACATGCC | |
| | CAAGTTAAGATTATTCCTCTCATTCCCGTCGAGATTAATAATATCACAGA | |
| | ACTGCTTCAGTATGAGCGCGACGTATTGTCCAAAGGCTTTGAAGGGGTTA | |
| | TGATACGCAAACCGGACGGCAAGTACAAGTTCGGAAGAAGCACATTGAAA | |
| | GAGGGTATATTGCTGAAGATGAAGCAGTTTAAGGATGCTGAGGCAACAAT | |
| | AATCAGCATGACAGCACTTTTTAAAAATACCAACACGAAAACTAAGGACA | |
| | ATTTTGGTTATAGTAAGCGGTCAACGCACAAAAGTGGGAAGGTAGAAGAA | |
| | GACGTAATGGGTAGCATTGAGGTGGATTATGACGGGGTGGTTTTCAGCAT | |
| | AGGGACTGGGTTTGATGCAGATCAACGGAGGGACTTTTGGCAGAACAAAG | |
| | AATCATATATAGGCAAAATGGTAAAGTTCAAATACTTCGAAATGGGAAGT | |
| | AAAGACTGCCCCAGATTCCCTGTATTCATTGGCATCAGGCACGAGGAGGA | |
| | CAGGAGTGGGGGATCAAAGCGGACTGCTGATAGTCAGCATAGTACTCCAC | |
| | CCAAGACCAAGCGGAAAGTTGAGTTTGAGCCGAAGAAAAAGCGAAAAGTG | |
| | TAA | |
| NLS- | MKRTADGSEFESPKKKRKVDKKYSIGLDIGTNSVGWAVITDEYKVPSKKF | 130 |
| nCas9- | KVLGNTDRHSIKKNLIGALLFDSGETAEATRLKRTARRRYTRRKNRICYL | |
| linker- | QEIFSNEMAKVDDSFFHRLEESFLVEEDKKHERHPIFGNIVDEVAYHEKY | |
| SplintR- | PTIYHLRKKLVDSTDKADLRLIYLALAHMIKFRGHFLIEGDLNPDNSDVD | |
| bpNLS | KLFIQLVQTYNQLFEENPINASGVDAKAILSARLSKSRRLENLIAQLPGE | |
| | KKNGLFGNLIALSLGLTPNFKSNFDLAEDAKLQLSKDTYDDDLDNLLAQI | |
| | GDQYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMK | |
| | RIEEGIKELGSQILKEHPVENTQLQNEKLYLYYLQNGRDMYVDQELDINR | |
| | LSDYDVDAIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYH | |
| | HAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYG | |
| | GFDSPTVAYSVLVVAKVEKGKSKKLKSVKELLGITIMERSSFEKNPIDFL | |
| | EAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVIL | |
| | ADANLDKVLSAYNKHRDKPIREQAENIIHLFTLTNLGAPAAFKYFDTTID | |
| | RKRYTSTKEVLDATLIHQSITGLYETRIDLSQLGGDSGGSSGGSKRTADG | |
| | SEFESPKKKRKVSGGSSGGSMAITKPLLAATLENIEDVQFPCLATPKIDG | |
| | IRSVKQTQMLSRTFKPIRNSVMNRLLTELLPEGSDGEISIEGATFQDTTS | |
| | AVMTGHKMYNAKFSYYWFDYVTDDPLKKYIDRVEDMKNYITVHPHILEHA | |
| | QVKIIPLIPVEINNITELLQYERDVLSKGFEGVMIRKPDGKYKFGRSTLK | |
| | EGILLKMKQFKDAEATIISMTALFKNTNTKTKDNFGYSKRSTHKSGKVEE | |
| | DVMGSIEVDYDGVVFSIGTGFDADQRRDFWQNKESYIGKMVKFKYFEMGS | |
| | KDCPRFPVFIGIRHEEDRSGGSKRTADSQHSTPPKTKRKVEFEPKKKRKV | |
| | * | |
| NLS- | atgaaacggacagccgacggaagcgagttcgagtcaccaaagaagaagcg | 131 |
| nCas9- | gaaagtcgacaagaagtacagcatcggcctggacatcggcaccaactctg | |
| linker- | tgggctgggccgtgatcaccgacgagtacaaggtgcccagcaagaaattc | |
| T4LIG- | aaggtgctgggcaacaccgaccggcacagcatcaagaagaacctgatcgg | |
| bpNLS | agccctgctgttcgacagcggcgaaacagccgaggccacccggctgaaga | |
| | gaaccgccagaagaagatacaccagacggaagaaccggatctgctatctg | |
| | caagagatcttcagcaacgagatggccaaggtggacgacagcttcttcca | |
| | cagactggaagagtccttcctggtggaagaggataagaagcacgagcggc | |
| | accccatcttcggcaacatcgtggacgaggtggcctaccacgagaagtac | |
| | cccaccatctaccacctgagaaagaaactggtggacagcaccgacaaggc | |
| | cgacctgcggctgatctatctggccctggcccacatgatcaagttccggg | |
| | gccacttcctgatcgagggcgacctgaaccccgacaacagcgacgtggac | |
| | aagctgttcatccagctggtgcagacctacaaccagctgttcgaggaaaa | |
| | ccccatcaacgccagcggcgtggacgccaaggccatcctgtctgccagac | |
| | tgagcaagagcagacggctggaaaatctgatcgcccagctgcccggcgag | |
| | aagaagaatggcctgttcggaaacctgattgccctgagcctgggcctgac | |
| | ccccaacttcaagagcaacttcgacctggccgaggatgccaaactgcagc | |
| | tgagcaaggacacctacgacgacgacctggacaacctgctggcccagatc | |
| | ggcgaccagtacgccgacctgtttctggccgccaagaacctgtccgacgc | |
| | catcctgctgagcgacatcctgagagtgaacaccgagatcaccaaggccc | |
| | ccctgagcgcctctatgatcaagagatacgacgagcaccaccaggacctg | |
| | accctgctgaaagctctcgtgcggcagcagctgcctgagaagtacaaaga | |
| | gattttcttcgaccagagcaagaacggctacgccggctacattgacggcg | |
| | gagccagccaggaagagttctacaagttcatcaagcccatcctggaaaag | |
| | atggacggcaccgaggaactgctcgtgaagctgaacagagaggacctgct | |
| | gcggaagcagcggaccttcgacaacggcagcatcccccaccagatccacc | |
| | tgggagagctgcacgccattctgcggcggcaggaagatttttacccattc | |
| | ctgaaggacaaccgggaaaagatcgagaagatcctgaccttccgcatccc | |
| | ctactacgtgggccctctggccaggggaaacagcagattcgcctggatga | |
| | ccagaaagagcgaggaaaccatcaccccctggaacttcgaggaagtggtg | |
| | gacaagggcgcttccgcccagagcttcatcgagcggatgaccaacttcga | |
| | taagaacctgcccaacgagaaggtgctgcccaagcacagcctgctgtacg | |
| | agtacttcaccgtgtataacgagctgaccaaagtgaaatacgtgaccgag | |
| | ggaatgagaaagcccgccttcctgagcggcgagcagaaaaaggccatcgt | |
| | ggacctgctgttcaagaccaaccggaaagtgaccgtgaagcagctgaaag | |
| | aggactacttcaagaaaatcgagtgcttcgactccgtggaaatctccggc | |
| | gtggaagatcggttcaacgcctccctgggcacataccacgatctgctgaa | |
| | aattatcaaggacaaggacttcctggacaatgaggaaaacgaggacattc | |
| | tggaagatatcgtgctgaccctgacactgtttgaggacagagagatgatc | |
| | gaggaacggctgaaaacctatgcccacctgttcgacgacaaagtgatgaa | |
| | gcagctgaagcggcggagatacaccggctggggcaggctgagccggaagc | |
| | tgatcaacggcatccgggacaagcagtccggcaagacaatcctggatttc | |
| | ctgaagtccgacggcttcgccaacagaaacttcatgcagctgatccacga | |
| | cgacagcctgacctttaaagaggacatccagaaagcccaggtgtccggcc | |
| | agggcgatagcctgcacgagcacattgccaatctggccggcagccccgcc | |
| | attaagaagggcatcctgcagacagtgaaggtggtggacgagctcgtgaa | |
| | agtgatgggccggcacaagcccgagaacatcgtgatcgaaatggccagag | |
| | agaaccagaccacccagaagggacagaagaacagccgcgagagaatgaag | |
| | cggatcgaagagggcatcaaagagctgggcagccagatcctgaaagaaca | |
| | ccccgtggaaaacacccagctgcagaacgagaagctgtacctgtactacc | |
| | tgcagaatgggcgggatatgtacgtggaccaggaactggacatcaaccgg | |
| | ctgtccgactacgatgtggacgctatcgtgcctcagagctttctgaagga | |
| | cgactccatcgacaacaaggtgctgaccagaagcgacaagaaccggggca | |
| | agagcgacaacgtgccctccgaagaggtcgtgaagaagatgaagaactac | |
| | tggcggcagctgctgaacgccaagctgattacccagagaaagttcgacaa | |
| | tctgaccaaggccgagagaggcggcctgagcgaactggataaggccggct | |
| | tcatcaagagacagctggtggaaacccggcagatcacaaagcacgtggca | |
| | cagatcctggactcccggatgaacactaagtacgacgagaatgacaagct | |
| | gatccgggaagtgaaagtgatcaccctgaagtccaagctggtgtccgatt | |
| | tccggaaggatttccagttttacaaagtgcgcgagatcaacaactaccac | |
| | cacgcccacgacgcctacctgaacgccgtcgtgggaaccgccctgatcaa | |
| | aaagtaccctaagctggaaagcgagttcgtgtacggcgactacaaggtgt | |
| | acgacgtgcggaagatgatcgccaagagcgagcaggaaatcggcaaggct | |
| | accgccaagtacttcttctacagcaacatcatgaactttttcaagaccga | |
| | gattaccctggccaacggcgagatccggaagcggcctctgatcgagacaa | |
| | acggcgaaaccggggagatcgtgtgggataagggccgggattttgccacc | |
| | gtgcggaaagtgctgagcatgccccaagtgaatatcgtgaaaaagaccga | |
| | ggtgcagacaggcggcttcagcaaagagtctatcctgcccaagaggaaca | |
| | gcgataagctgatcgccagaaagaaggactgggaccctaagaagtacggc | |
| | ggcttcgacagccccaccgtggcctattctgtgctggtggtggccaaagt | |
| | ggaaaagggcaagtccaagaaactgaagagtgtgaaagagctgctgggga | |
| | tcaccatcatggaaagaagcagcttcgagaagaatcccatcgactttctg | |
| | gaagccaagggctacaaagaagtgaaaaaggacctgatcatcaagctgcc | |
| | taagtactccctgttcgagctggaaaacggccggaagagaatgctggcct | |
| | ctgccggcgaactgcagaagggaaacgaactggccctgccctccaaatat | |
| | gtgaacttcctgtacctggccagccactatgagaagctgaagggctcccc | |
| | cgaggataatgagcagaaacagctgtttgtggaacagcacaagcactacc | |
| | tggacgagatcatcgagcagatcagcgagttctccaagagagtgatcctg | |
| | gccgacgctaatctggacaaagtgctgtccgcctacaacaagcaccggga | |
| | taagcccatcagagagcaggccgagaatatcatccacctgtttaccctga | |
| | ccaatctgggagcccctgccgccttcaagtactttgacaccaccatcgac | |
| | cggaagaggtacaccagcaccaaagaggtgctggacgccaccctgatcca | |
| | ccagagcatcaccggcctgtacgagacacggatcgacctgtctcagctgg | |
| | gaggtgacTCCGGCGGAAGCTCTGGTGGCAGCAAGCGGACCGCCGACGGC | |
| | TCTGAATTCGAGAGCCCTAAGAAGAAAAGAAAGGTGAGCGGAGGCTCTAG | |
| | CGGCGGAAGCATGATTCTTAAAATTCTTAACGAGATTGCGAGTATTGGCA | |
| | GCACGAAACAAAAGCAGGCCATATTGGAAAAGAATAAGGACAATGAGTTG | |
| | CTTAAACGCGTGTATAGGCTCACTTACTCTCGCGGACTGCAATACTATAT | |
| | TAAAAAATGGCCTAAGCCCGGCATCGCTACTCAAAGCTTCGGAATGCTTA | |
| | CGCTGACAGATATGCTCGACTTCATCGAGTTTACTCTCGCAACAAGGAAG | |
| | TTGACTGGCAACGCCGCGATTGAAGAATTGACGGGTTATATCACGGACGG | |
| | GAAGAAGGATGATGTTGAGGTGCTGAGGCGCGTTATGATGCGCGACCTCG | |
| | AATGTGGTGCCTCAGTTTCCATAGCCAATAAAGTTTGGCCAGGCTTGATC | |
| | CCGGAGCAGCCACAGATGCTGGCCAGTAGCTACGACGAGAAGGGTATTAA | |
| | CAAAAATATCAAGTTTCCAGCGTTTGCACAACTTAAAGCGGATGGGGCGC | |
| | GGTGTTTCGCCGAAGTCCGGGGTGACGAATTGGACGATGTGCGCCTTCTG | |
| | AGTCGCGCAGGAAATGAATATCTGGGGCTTGACCTCTTGAAGGAGGAGCT | |
| | GATTAAGATGACAGCAGAAGCCAGGCAGATCCATCCAGAGGGGGTACTTA | |
| | TTGATGGTGAACTCGTATACCATGAGCAGGTTAAGAAGGAGCCAGAGGGT | |
| | TTGGATTTCCTCTTTGACGCCTATCCCGAGAATTCAAAGGCAAAGGAGTT | |
| | CGCCGAGGTTGCAGAATCAAGAACGGCTTCCAACGGCATAGCGAATAAAT | |
| | CACTCAAAGGAACTATATCTGAAAAGGAGGCACAGTGTATGAAATTCCAA | |
| | GTGTGGGACTATGTGCCGCTTGTCGAGATTTACAGCTTGCCTGCTTTCCG | |
| | ATTGAAGTACGATGTACGGTTTAGTAAGCTCGAGCAAATGACTTCAGGTT | |
| | ACGATAAAGTCATCTTGATTGAGAACCAGGTCGTTAATAATCTTGACGAG | |
| | GCGAAGGTCATATATAAGAAATATATAGATCAAGGGCTCGAGGGTATCAT | |
| | TCTGAAGAATATAGATGGCTTGTGGGAAAACGCCAGGTCCAAAAACCTGT | |
| | ATAAGTTTAAGGAAGTAATAGATGTAGATTTGAAAATAGTTGGAATTTAC | |
| | CCCCATCGGAAGGACCCCACGAAAGCGGGTGGGTTTATCCTCGAGAGCGA | |
| | GTGTGGGAAGATAAAAGTGAATGCCGGCTCCGGATTGAAGGACAAGGCAG | |
| | GTGTGAAAAGTCATGAGCTCGATCGGACGAGAATAATGGAAAACCAGAAT | |
| | TACTACATTGGAAAGATTTTGGAATGCGAGTGTAACGGCTGGTTGAAGAG | |
| | CGACGGACGCACCGATTACGTGAAACTCTTTCTGCCAATTGCAATCAGGT | |
| | TGAGAGAGGATAAGACTAAGGCCAATACTTTCGAGGACGTCTTCGGAGAC | |
| | TTTCACGAAGTCACTGGGCTTTCTGGGGGTAGTAAGAGAACTGCAGATAG | |
| | CCAGCATTCAACGCCGCCAAAAACAAAGCGAAAGGTAGAATTCGAACCAA | |
| | AGAAAAAGCGGAAAGTATAA | |
| NLS- | MKRTADGSEFESPKKKRKVDKKYSIGLDIGTNSVGWAVITDEYKVPSKKF | 132 |
| nCas9- | KVLGNTDRHSIKKNLIGALLFDSGETAEATRLKRTARRRYTRRKNRICYL | |
| linker- | QEIFSNEMAKVDDSFFHRLEESFLVEEDKKHERHPIFGNIVDEVAYHEKY | |
| T4LIG- | PTIYHLRKKLVDSTDKADLRLIYLALAHMIKFRGHFLIEGDLNPDNSDVD | |
| bpNLS | KLFIQLVQTYNQLFEENPINASGVDAKAILSARLSKSRRLENLIAQLPGE | |
| | KKNGLFGNLIALSLGLTPNFKSNFDLAEDAKLQLSKDTYDDDLDNLLAQI | |
| | GDQYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMK | |
| | RIEEGIKELGSQILKEHPVENTQLQNEKLYLYYLQNGRDMYVDQELDINR | |
| | LSDYDVDAIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYH | |
| | HAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYG | |
| | GFDSPTVAYSVLVVAKVEKGKSKKLKSVKELLGITIMERSSFEKNPIDFL | |
| | EAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVIL | |
| | ADANLDKVLSAYNKHRDKPIREQAENIIHLFTLTNLGAPAAFKYFDTTID | |
| | RKRYTSTKEVLDATLIHQSITGLYETRIDLSQLGGDSGGSSGGSKRTADG | |
| | SEFESPKKKRKVSGGSSGGSMILKILNEIASIGSTKQKQAILEKNKDNEL | |
| | LKRVYRLTYSRGLQYYIKKWPKPGIATQSFGMLTLTDMLDFIEFTLATRK | |
| | LTGNAAIEELTGYITDGKKDDVEVLRRVMMRDLECGASVSIANKVWPGLI | |
| | PEQPQMLASSYDEKGINKNIKFPAFAQLKADGARCFAEVRGDELDDVRLL | |
| | SRAGNEYLGLDLLKEELIKMTAEARQIHPEGVLIDGELVYHEQVKKEPEG | |
| | LDFLFDAYPENSKAKEFAEVAESRTASNGIANKSLKGTISEKEAQCMKFQ | |
| | VWDYVPLVEIYSLPAFRLKYDVRFSKLEQMTSGYDKVILIENQVVNNLDE | |
| | AKVIYKKYIDQGLEGIILKNIDGLWENARSKNLYKFKEVIDVDLKIVGIY | |
| | PHRKDPTKAGGFILESECGKIKVNAGSGLKDKAGVKSHELDRTRIMENQN | |
| | YYIGKILECECNGWLKSDGRTDYVKLFLPIAIRLREDKTKANTFEDVFGD | |
| | FHEVTGLSGGSKRTADSQHSTPPKTKRKVEFEPKKKRKV* | |
| NLS- | atgaaacggacagccgacggaagcgagttcgagtcaccaaagaagaagcg | 133 |
| nCas9-LZ | gaaagtcgacaagaagtacagcatcggcctggacatcggcaccaactctg | |
| | tgggctgggccgtgatcaccgacgagtacaaggtgcccagcaagaaattc | |
| | aaggtgctgggcaacaccgaccggcacagcatcaagaagaacctgatcgg | |
| | agccctgctgttcgacagcggcgaaacagccgaggccacccggctgaaga | |
| | gaaccgccagaagaagatacaccagacggaagaaccggatctgctatctg | |
| | caagagatcttcagcaacgagatggccaaggtggacgacagcttcttcca | |
| | cagactggaagagtccttcctggtggaagaggataagaagcacgagcggc | |
| | accccatcttcggcaacatcgtggacgaggtggcctaccacgagaagtac | |
| | cccaccatctaccacctgagaaagaaactggtggacagcaccgacaaggc | |
| | cgacctgcggctgatctatctggccctggcccacatgatcaagttccggg | |
| | gccacttcctgatcgagggcgacctgaaccccgacaacagcgacgtggac | |
| | aagctgttcatccagctggtgcagacctacaaccagctgttcgaggaaaa | |
| | ccccatcaacgccagcggcgtggacgccaaggccatcctgtctgccagac | |
| | tgagcaagagcagacggctggaaaatctgatcgcccagctgcccggcgag | |
| | aagaagaatggcctgttcggaaacctgattgccctgagcctgggcctgac | |
| | ccccaacttcaagagcaacttcgacctggccgaggatgccaaactgcagc | |
| | tgagcaaggacacctacgacgacgacctggacaacctgctggcccagatc | |
| | ggcgaccagtacgccgacctgtttctggccgccaagaacctgtccgacgc | |
| | catcctgctgagcgacatcctgagagtgaacaccgagatcaccaaggccc | |
| | ccctgagcgcctctatgatcaagagatacgacgagcaccaccaggacctg | |
| | accctgctgaaagctctcgtgcggcagcagctgcctgagaagtacaaaga | |
| | gattttcttcgaccagagcaagaacggctacgccggctacattgacggcg | |
| | gagccagccaggaagagttctacaagttcatcaagcccatcctggaaaag | |
| | atggacggcaccgaggaactgctcgtgaagctgaacagagaggacctgct | |
| | gcggaagcagcggaccttcgacaacggcagcatcccccaccagatccacc | |
| | tgggagagctgcacgccattctgcggcggcaggaagatttttacccattc | |
| | ctgaaggacaaccgggaaaagatcgagaagatcctgaccttccgcatccc | |
| | ctactacgtgggccctctggccaggggaaacagcagattcgcctggatga | |
| | ccagaaagagcgaggaaaccatcaccccctggaacttcgaggaagtggtg | |
| | gacaagggcgcttccgcccagagcttcatcgagcggatgaccaacttcga | |
| | taagaacctgcccaacgagaaggtgctgcccaagcacagcctgctgtacg | |
| | agtacttcaccgtgtataacgagctgaccaaagtgaaatacgtgaccgag | |
| | ggaatgagaaagcccgccttcctgagcggcgagcagaaaaaggccatcgt | |
| | ggacctgctgttcaagaccaaccggaaagtgaccgtgaagcagctgaaag | |
| | aggactacttcaagaaaatcgagtgcttcgactccgtggaaatctccggc | |
| | gtggaagatcggttcaacgcctccctgggcacataccacgatctgctgaa | |
| | aattatcaaggacaaggacttcctggacaatgaggaaaacgaggacattc | |
| | tggaagatatcgtgctgaccctgacactgtttgaggacagagagatgatc | |
| | gaggaacggctgaaaacctatgcccacctgttcgacgacaaagtgatgaa | |
| | gcagctgaagcggcggagatacaccggctggggcaggctgagccggaagc | |
| | tgatcaacggcatccgggacaagcagtccggcaagacaatcctggatttc | |
| | ctgaagtccgacggcttcgccaacagaaacttcatgcagctgatccacga | |
| | cgacagcctgacctttaaagaggacatccagaaagcccaggtgtccggcc | |
| | agggcgatagcctgcacgagcacattgccaatctggccggcagccccgcc | |
| | attaagaagggcatcctgcagacagtgaaggtggtggacgagctcgtgaa | |
| | agtgatgggccggcacaagcccgagaacatcgtgatcgaaatggccagag | |
| | agaaccagaccacccagaagggacagaagaacagccgcgagagaatgaag | |
| | cggatcgaagagggcatcaaagagctgggcagccagatcctgaaagaaca | |
| | ccccgtggaaaacacccagctgcagaacgagaagctgtacctgtactacc | |
| | tgcagaatgggcgggatatgtacgtggaccaggaactggacatcaaccgg | |
| | ctgtccgactacgatgtggacgctatcgtgcctcagagctttctgaagga | |
| | cgactccatcgacaacaaggtgctgaccagaagcgacaagaaccggggca | |
| | agagcgacaacgtgccctccgaagaggtcgtgaagaagatgaagaactac | |
| | tggcggcagctgctgaacgccaagctgattacccagagaaagttcgacaa | |
| | tctgaccaaggccgagagaggcggcctgagcgaactggataaggccggct | |
| | tcatcaagagacagctggtggaaacccggcagatcacaaagcacgtggca | |
| | cagatcctggactcccggatgaacactaagtacgacgagaatgacaagct | |
| | gatccgggaagtgaaagtgatcaccctgaagtccaagctggtgtccgatt | |
| | tccggaaggatttccagttttacaaagtgcgcgagatcaacaactaccac | |
| | cacgcccacgacgcctacctgaacgccgtcgtgggaaccgccctgatcaa | |
| | aaagtaccctaagctggaaagcgagttcgtgtacggcgactacaaggtgt | |
| | acgacgtgcggaagatgatcgccaagagcgagcaggaaatcggcaaggct | |
| | accgccaagtacttcttctacagcaacatcatgaactttttcaagaccga | |
| | gattaccctggccaacggcgagatccggaagcggcctctgatcgagacaa | |
| | acggcgaaaccggggagatcgtgtgggataagggccgggattttgccacc | |
| | gtgcggaaagtgctgagcatgccccaagtgaatatcgtgaaaaagaccga | |
| | ggtgcagacaggcggcttcagcaaagagtctatcctgcccaagaggaaca | |
| | gcgataagctgatcgccagaaagaaggactgggaccctaagaagtacggc | |
| | ggcttcgacagccccaccgtggcctattctgtgctggtggtggccaaagt | |
| | ggaaaagggcaagtccaagaaactgaagagtgtgaaagagctgctgggga | |
| | tcaccatcatggaaagaagcagcttcgagaagaatcccatcgactttctg | |
| | gaagccaagggctacaaagaagtgaaaaaggacctgatcatcaagctgcc | |
| | taagtactccctgttcgagctggaaaacggccggaagagaatgctggcct | |
| | ctgccggcgaactgcagaagggaaacgaactggccctgccctccaaatat | |
| | gtgaacttcctgtacctggccagccactatgagaagctgaagggctcccc | |
| | cgaggataatgagcagaaacagctgtttgtggaacagcacaagcactacc | |
| | tggacgagatcatcgagcagatcagcgagttctccaagagagtgatcctg | |
| | gccgacgctaatctggacaaagtgctgtccgcctacaacaagcaccggga | |
| | taagcccatcagagagcaggccgagaatatcatccacctgtttaccctga | |
| | ccaatctgggagcccctgccgccttcaagtactttgacaccaccatcgac | |
| | cggaagaggtacaccagcaccaaagaggtgctggacgccaccctgatcca | |
| | ccagagcatcaccggcctgtacgagacacggatcgacctgtctcagctgg | |
| | gaggtgacggctcaaaaagaaccgccgacggcagcgaattcgagcccaag | |
| | aagaagaggaaagtcGGAGGAGGAGGCAGTGGTGGGCGACTTGAAATTAG | |
| | AGCCGCGTTCCTGCGCCAGAGGAATACGGCTCTCCGCACGGAGGTAGCCG | |
| | AACTTGAGCAAGAAGTACAGAGATTGGAGAACGAGGTTTCACAGTATGAG | |
| | ACACGATATGGCCCCCTTGGCGGCGGAAAGtaa | |
| NLS1- | KRTADGSEFESPKKKRKVMGIQGLAKLIADVAPSAIRENDIKSYFGRKVA | 134 |
| hFEN1- | IDASMSIYQFLIAVRQGGDVLQNEEGETTSHLMGMFYRTIRMMENGIKPV | |
| linker1- | YVFDGKPPQLKSGELAKRSERRAEAEKQLQQAQAAGAEQEVEKFTKRLVK | |
| nCas9- | VTKQHNDECKHLLSLMGIPYLDAPSEAEASCAALVKAGKVYAAATEDMDC | |
| linker2- | LTFGSPVLMRHLTASEAKKLPIQEFHLSRILQELGLNQEQFVDLCILLGS | |
| T4LIG- | DYCESIRGIGPKRAVDLIQKHKSIEEIVRRLDPNKYPVPENWLHKEAHQL | |
| NLS2 | FLEPEVLDPESVELKWSEPNEEELIKFMCGEKQFSEERIRSGVKRLSKSR | |
| | QGSTQGRLDDFFKVTGSLSSAKRKEPEPKGSTKKKAKTGAAGKFKRGKSG | |
| | GSSGGSSGSETPGTSESATPESSGGSSGGSSMDKKYSIGLDIGTNSVGWA | |
| | VITDEYKVPSKKFKVLGNTDRHSIKKNLIGALLFDSGETAEATRLKRTAR | |
| | RRYTRRKNRICYLQEIFSNEMAKVDDSFFHRLEESFLVEEDKKHERHPIF | |
| | GNIVDEVAYHEKYPTIYHLRKKLVDSTDKADLRLIYLALAHMIKFRGHFL | |
| | IEGDLNPDNSDVDKLFIQLVQTYNQLFEENPINASGVDAKAILSARLSKS | |
| | RRLENLIAQLPGEKKNGLFGNLIALSLGLTPNFKSNFDLAEDAKLQLSKD | |
| | TYDDDLDNLLAQIGDQYADLFLAAKNLSDAILLSDILRVNTEITKAPLSA | |
| | SMIKRYDEHHQDLTLLKALVRQQLPEKYKEIFFDQSKNGYAGYIDGGASQ | |
| | EEFYKFIKPILEKMDGTEELLVKLNREDLLRKQRTFDNGSIPHQIHLGEL | |
| | HAILRRQEDFYPFLKDNREKIEKILTFRIPYYVGPLARGNSRFAWMTRKS | |
| | EETITPWNFEEVVDKGASAQSFIERMTNFDKNLPNEKVLPKHSLLYEYFT | |
| | VYNELTKVKYVTEGMRKPAFLSGEQKKAIVDLLFKTNRKVTVKQLKEDYF | |
| | KKIECFDSVEISGVEDRFNASLGTYHDLLKIIKDKDFLDNEENEDILEDI | |
| | VLTLTLFEDREMIEERLKTYAHLFDDKVMKQLKRRRYTGWGRLSRKLING | |
| | IRDKQSGKTILDFLKSDGFANRNFMQLIHDDSLTFKEDIQKAQVSGQGDS | |
| | LHEHIANLAGSPAIKKGILQTVKVVDELVKVMGRHKPENIVIEMARENQT | |
| | TQKGQKNSRERMKRIEEGIKELGSQILKEHPVENTQLQNEKLYLYYLQNG | |
| | RDMYVDQELDINRLSDYDVDAIVPQSFLKDDSIDNKVLTRSDKNRGKSDN | |
| | VPSEEVVKKMKNYWRQLLNAKLITQRKFDNLTKAERGGLSELDKAGFIKR | |
| | QLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVR | |
| | KMIAKSEQEIGKATAKYFFYSNIMNFFKTEITLANGEIRKRPLIETNGET | |
| | GEIVWDKGRDFATVRKVLSMPQVNIVKKTEVQTGGFSKESILPKRNSDKL | |
| | IARKKDWDPKKYGGFDSPTVAYSVLVVAKVEKGKSKKLKSVKELLGITIM | |
| | ERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEI | |
| | IEQISEFSKRVILADANLDKVLSAYNKHRDKPIREQAENIIHLFTLTNLG | |
| | APAAFKYFDTTIDRKRYTSTKEVLDATLIHQSITGLYETRIDLSQLGGDS | |
| | GGSSGGSKRTADGSEFESPKKKRKVSGGSSGGSMILKILNEIASIGSTKQ | |
| | KQAILEKNKDNELLKRVYRLTYSRGLQYYIKKWPKPGIATQSFGMLTLTD | |
| | MLDFIEFTLATRKLTGNAAIEELTGYITDGKKDDVEVLRRVMMRDLECGA | |
| | SVSIANKVWPGLIPEQPQMLASSYDEKGINKNIKFPAFAQLKADGARCFA | |
| | EVRGDELDDVRLLSRAGNEYLGLDLLKEELIKMTAEARQIHPEGVLIDGE | |
| | LVYHEQVKKEPEGLDFLFDAYPENSKAKEFAEVAESRTASNGIANKSLKG | |
| | TISEKEAQCMKFQVWDYVPLVEIYSLPAFRLKYDVRFSKLEQMTSGYDKV | |
| | ILIENQVVNNLDEAKVIYKKYIDQGLEGIILKNIDGLWENARSKNLYKFK | |
| | EVIDVDLKIVGIYPHRKDPTKAGGFILESECGKIKVNAGSGLKDKAGVKS | |
| | HELDRTRIMENQNYYIGKILECECNGWLKSDGRTDYVKLFLPIAIRLRED | |
| | KTKANTFEDVFGDFHEVTGLSGGSKRTADSQHSTPPKTKRKVEFEPKKKR | |
| | KV | |
| NLS1- | KRTADGSEFESPKKKRKVMILKILNEIASIGSTKQKQAILEKNKDNELLK | 135 |
| hFEN1- | RVYRLTYSRGLQYYIKKWPKPGIATQSFGMLTLTDMLDFIEFTLATRKLT | |
| linker1- | GNAAIEELTGYITDGKKDDVEVLRRVMMRDLECGASVSIANKVWPGLIPE | |
| T4LIG- | QPQMLASSYDEKGINKNIKFPAFAQLKADGARCFAEVRGDELDDVRLLSR | |
| linker2- | AGNEYLGLDLLKEELIKMTAEARQIHPEGVLIDGELVYHEQVKKEPEGLD | |
| nCas9- | FLFDAYPENSKAKEFAEVAESRTASNGIANKSLKGTISEKEAQCMKFQVW | |
| NLS2 | DYVPLVEIYSLPAFRLKYDVRFSKLEQMTSGYDKVILIENQVVNNLDEAK | |
| | VIYKKYIDQGLEGIILKNIDGLWENARSKNLYKFKEVIDVDLKIVGIYPH | |
| | RKDPTKAGGFILESECGKIKVNAGSGLKDKAGVKSHELDRTRIMENQNYY | |
| | IGKILECECNGWLKSDGRTDYVKLFLPIAIRLREDKTKANTFEDVFGDFH | |
| | EVTGLSGGSSGGSSGSETPGTSESATPESSGGSSGGSSMGIQGLAKLIAD | |
| | VAPSAIRENDIKSYFGRKVAIDASMSIYQFLIAVRQGGDVLQNEEGETTS | |
| | HLMGMFYRTIRMMENGIKPVYVFDGKPPQLKSGELAKRSERRAEAEKQLQ | |
| | QAQAAGAEQEVEKFTKRLVKVTKQHNDECKHLLSLMGIPYLDAPSEAEAS | |
| | CAALVKAGKVYAAATEDMDCLTFGSPVLMRHLTASEAKKLPIQEFHLSRI | |
| | LQELGLNQEQFVDLCILLGSDYCESIRGIGPKRAVDLIQKHKSIEEIVRR | |
| | LDPNKYPVPENWLHKEAHQLFLEPEVLDPESVELKWSEPNEEELIKFMCG | |
| | EKQFSEERIRSGVKRLSKSRQGSTQGRLDDFFKVTGSLSSAKRKEPEPKG | |
| | STKKKAKTGAAGKFKRGKSGGSSGGSKRTADGSEFESPKKKRKVSGGSSG | |
| | GSMDKKYSIGLDIGTNSVGWAVITDEYKVPSKKFKVLGNTDRHSIKKNLI | |
| | GALLFDSGETAEATRLKRTARRRYTRRKNRICYLQEIFSNEMAKVDDSFF | |
| | HRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGL | |
| | TPNFKSNFDLAEDAKLQLSKDTYDDDLDNLLAQIGDQYADLFLAAKNLSD | |
| | AILLSDILRVNTEITKAPLSASMIKRYDEHHQDLTLLKALVRQQLPEKYK | |
| | EIFFDQSKNGYAGYIDGGASQEEFYKFIKPILEKMDGTEELLVKLNREDL | |
| | LRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNF | |
| | DKNLPNEKVLPKHSLLYEYFTVYNELTKVKYVTEGMRKPAFLSGEQKKAI | |
| | VDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVM | |
| | KQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKE | |
| | HPVENTQLQNEKLYLYYLQNGRDMYVDQELDINRLSDYDVDAIVPQSFLK | |
| | DDSIDNKVLTRSDKNRGKSDNVPSEEVVKKMKNYWRQLLNAKLITQRKFD | |
| | NLTKAERGGLSELDKAGFIKRQLVETRQITKHVAQILDSRMNTKYDENDK | |
| | LIREVKVITLKSKLVSDFRKDFQFYKVREINNYHHAHDAYLNAVVGTALI | |
| | KKYPKLESEFVYGDYKVYDVRKMIAKSEQEIGKATAKYFFYSNIMNFFKT | |
| | EITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAK | |
| | VEKGKSKKLKSVKELLGITIMERSSFEKNPIDFLEAKGYKEVKKDLIIKL | |
| | PKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLI | |
| | HQSITGLYETRIDLSQLGGDSGGSKRTADSQHSTPPKTKRKVEFEPKKKR | |
| | KV | |
| NLS1- | KRTADGSEFESPKKKRKVMDKKYSIGLDIGTNSVGWAVITDEYKVPSKKF | 136 |
| nCas9- | KVLGNTDRHSIKKNLIGALLFDSGETAEATRLKRTARRRYTRRKNRICYL | |
| linker1- | QEIFSNEMAKVDDSFFHRLEESFLVEEDKKHERHPIFGNIVDEVAYHEKY | |
| hFENI- | PTIYHLRKKLVDSTDKADLRLIYLALAHMIKFRGHFLIEGDLNPDNSDVD | |
| linker2- | KLFIQLVQTYNQLFEENPINASGVDAKAILSARLSKSRRLENLIAQLPGE | |
| T4LIG- | KKNGLFGNLIALSLGLTPNFKSNFDLAEDAKLQLSKDTYDDDLDNLLAQI | |
| NLS2 | GDQYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMK | |
| | RIEEGIKELGSQILKEHPVENTQLQNEKLYLYYLQNGRDMYVDQELDINR | |
| | LSDYDVDAIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYH | |
| | HAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYG | |
| | GFDSPTVAYSVLVVAKVEKGKSKKLKSVKELLGITIMERSSFEKNPIDFL | |
| | EAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVIL | |
| | ADANLDKVLSAYNKHRDKPIREQAENIIHLFTLTNLGAPAAFKYFDTTID | |
| | RKRYTSTKEVLDATLIHQSITGLYETRIDLSQLGGDSGGSSGGSSGSETP | |
| | GTSESATPESSGGSSGGSSMGIQGLAKLIADVAPSAIRENDIKSYFGRKV | |
| | AIDASMSIYQFLIAVRQGGDVLQNEEGETTSHLMGMFYRTIRMMENGIKP | |
| | VYVFDGKPPQLKSGELAKRSERRAEAEKQLQQAQAAGAEQEVEKFTKRLV | |
| | KVTKQHNDECKHLLSLMGIPYLDAPSEAEASCAALVKAGKVYAAATEDMD | |
| | CLTFGSPVLMRHLTASEAKKLPIQEFHLSRILQELGLNQEQFVDLCILLG | |
| | SDYCESIRGIGPKRAVDLIQKHKSIEEIVRRLDPNKYPVPENWLHKEAHQ | |
| | LFLEPEVLDPESVELKWSEPNEEELIKFMCGEKQFSEERIRSGVKRLSKS | |
| | RQGSTQGRLDDFFKVTGSLSSAKRKEPEPKGSTKKKAKTGAAGKFKRGKS | |
| | GGSSGGSKRTADGSEFESPKKKRKVSGGSSGGSMILKILNEIASIGSTKQ | |
| | KQAILEKNKDNELLKRVYRLTYSRGLQYYIKKWPKPGIATQSFGMLTLTD | |
| | MLDFIEFTLATRKLTGNAAIEELTGYITDGKKDDVEVLRRVMMRDLECGA | |
| | SVSIANKVWPGLIPEQPQMLASSYDEKGINKNIKFPAFAQLKADGARCFA | |
| | EVRGDELDDVRLLSRAGNEYLGLDLLKEELIKMTAEARQIHPEGVLIDGE | |
| | LVYHEQVKKEPEGLDFLFDAYPENSKAKEFAEVAESRTASNGIANKSLKG | |
| | TISEKEAQCMKFQVWDYVPLVEIYSLPAFRLKYDVRFSKLEQMTSGYDKV | |
| | ILIENQVVNNLDEAKVIYKKYIDQGLEGIILKNIDGLWENARSKNLYKFK | |
| | EVIDVDLKIVGIYPHRKDPTKAGGFILESECGKIKVNAGSGLKDKAGVKS | |
| | HELDRTRIMENQNYYIGKILECECNGWLKSDGRTDYVKLFLPIAIRLRED | |
| | KTKANTFEDVFGDFHEVTGLSGGSKRTADSQHSTPPKTKRKVEFEPKKKR | |
| | KV | |
| NLS1- | KRTADGSEFESPKKKRKVMILKILNEIASIGSTKQKQAILEKNKDNELLK | 137 |
| T4LIG- | RVYRLTYSRGLQYYIKKWPKPGIATQSFGMLTLTDMLDFIEFTLATRKLT | |
| linker1- | GNAAIEELTGYITDGKKDDVEVLRRVMMRDLECGASVSIANKVWPGLIPE | |
| nCas9- | QPQMLASSYDEKGINKNIKFPAFAQLKADGARCFAEVRGDELDDVRLLSR | |
| linker2- | AGNEYLGLDLLKEELIKMTAEARQIHPEGVLIDGELVYHEQVKKEPEGLD | |
| hFEN1- | FLFDAYPENSKAKEFAEVAESRTASNGIANKSLKGTISEKEAQCMKFQVW | |
| NLS2 | DYVPLVEIYSLPAFRLKYDVRFSKLEQMTSGYDKVILIENQVVNNLDEAK | |
| | VIYKKYIDQGLEGIILKNIDGLWENARSKNLYKFKEVIDVDLKIVGIYPH | |
| | RKDPTKAGGFILESECGKIKVNAGSGLKDKAGVKSHELDRTRIMENQNYY | |
| | IGKILECECNGWLKSDGRTDYVKLFLPIAIRLREDKTKANTFEDVFGDFH | |
| | EVTGLSGGSSGGSSGSETPGTSESATPESSGGSSGGSSMDKKYSIGLDIG | |
| | TNSVGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEEDKK | |
| | HERHPIFGNIVDEVAYHEKYPTIYHLRKKLVDSTDKADLRLIYLALAHMI | |
| | KFRGHFLIEGDLNPDNSDVDKLFIQLVQTYNQLFEENPINASGVDAKAIL | |
| | SARLSKSRRLENLIAQLPGEKKNGLFGNLIALSLGLTPNFKSNFDLAEDA | |
| | KLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGY | |
| | IDGGASQEEFYKFIKPILEKMDGTEELLVKLNREDLLRKQRTFDNGSIPH | |
| | QIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHS | |
| | LLYEYFTVYNELTKVKYVTEGMRKPAFLSGEQKKAIVDLLFKTNRKVTVK | |
| | QLKEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQKAQ | |
| | VSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLY | |
| | LYYLQNGRDMYVDQELDINRLSDYDVDAIVPQSFLKDDSIDNKVLTRSDK | |
| | NRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKL | |
| | VSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILP | |
| | KRNSDKLIARKKDWDPKKYGGFDSPTVAYSVLVVAKVEKGKSKKLKSVKE | |
| | LLGITIMERSSFEKNPIDFLEAKGYKEVKKDLIIKLPKYSLFELENGRKR | |
| | MLASAGELQKGNELALPSKYVNFLYLASHYEKLKGSPEDNEQKQLFVEQH | |
| | KHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDL | |
| | SQLGGDSGGSSGGSKRTADGSEFESPKKKRKVSGGSSGGSMGIQGLAKLI | |
| | ADVAPSAIRENDIKSYFGRKVAIDASMSIYQFLIAVRQGGDVLQNEEGET | |
| | TSHLMGMFYRTIRMMENGIKPVYVFDGKPPQLKSGELAKRSERRAEAEKQ | |
| | LQQAQAAGAEQEVEKFTKRLVKVTKQHNDECKHLLSLMGIPYLDAPSEAE | |
| | ASCAALVKAGKVYAAATEDMDCLTFGSPVLMRHLTASEAKKLPIQEFHLS | |
| | RILQELGLNQEQFVDLCILLGSDYCESIRGIGPKRAVDLIQKHKSIEEIV | |
| | RRLDPNKYPVPENWLHKEAHQLFLEPEVLDPESVELKWSEPNEEELIKFM | |
| | CGEKQFSEERIRSGVKRLSKSRQGSTQGRLDDFFKVTGSLSSAKRKEPEP | |
| | KGSTKKKAKTGAAGKFKRGKSGGSKRTADSQHSTPPKTKRKVEFEPKKKR | |
| | KV | |
| NLS1- | KRTADGSEFESPKKKRKVMDKKYSIGLDIGTNSVGWAVITDEYKVPSKKF | 138 |
| nCas9- | KVLGNTDRHSIKKNLIGALLFDSGETAEATRLKRTARRRYTRRKNRICYL | |
| linker1- | QEIFSNEMAKVDDSFFHRLEESFLVEEDKKHERHPIFGNIVDEVAYHEKY | |
| T4LIG- | PTIYHLRKKLVDSTDKADLRLIYLALAHMIKFRGHFLIEGDLNPDNSDVD | |
| linker2- | KLFIQLVQTYNQLFEENPINASGVDAKAILSARLSKSRRLENLIAQLPGE | |
| hFEN1- | KKNGLFGNLIALSLGLTPNFKSNFDLAEDAKLQLSKDTYDDDLDNLLAQI | |
| NLS2 | GDQYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPILEK | |
| | MDGTEELLVKLNREDLLRKQRTFDNGSIPHQIHLGELHAILRRQEDFYPF | |
| | LKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISG | |
| | VEDRFNASLGTYHDLLKIIKDKDFLDNEENEDILEDIVLTLTLFEDREMI | |
| | EERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPA | |
| | IKKGILQTVKVVDELVKVMGRHKPENIVIEMARENQTTQKGQKNSRERMK | |
| | RIEEGIKELGSQILKEHPVENTQLQNEKLYLYYLQNGRDMYVDQELDINR | |
| | LSDYDVDAIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYH | |
| | HAHDAYLNAVVGTALIKKYPKLESEFVYGDYKVYDVRKMIAKSEQEIGKA | |
| | TAKYFFYSNIMNFFKTEITLANGEIRKRPLIETNGETGEIVWDKGRDFAT | |
| | VRKVLSMPQVNIVKKTEVQTGGFSKESILPKRNSDKLIARKKDWDPKKYG | |
| | GFDSPTVAYSVLVVAKVEKGKSKKLKSVKELLGITIMERSSFEKNPIDFL | |
| | EAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVIL | |
| | ADANLDKVLSAYNKHRDKPIREQAENIIHLFTLTNLGAPAAFKYFDTTID | |
| | RKRYTSTKEVLDATLIHQSITGLYETRIDLSQLGGDSGGSSGGSSGSETP | |
| | GTSESATPESSGGSSGGSSMILKILNEIASIGSTKQKQAILEKNKDNELL | |
| | KRVYRLTYSRGLQYYIKKWPKPGIATQSFGMLTLTDMLDFIEFTLATRKL | |
| | TGNAAIEELTGYITDGKKDDVEVLRRVMMRDLECGASVSIANKVWPGLIP | |
| | EQPQMLASSYDEKGINKNIKFPAFAQLKADGARCFAEVRGDELDDVRLLS | |
| | RAGNEYLGLDLLKEELIKMTAEARQIHPEGVLIDGELVYHEQVKKEPEGL | |
| | DFLFDAYPENSKAKEFAEVAESRTASNGIANKSLKGTISEKEAQCMKFQV | |
| | WDYVPLVEIYSLPAFRLKYDVRFSKLEQMTSGYDKVILIENQVVNNLDEA | |
| | KVIYKKYIDQGLEGIILKNIDGLWENARSKNLYKFKEVIDVDLKIVGIYP | |
| | HRKDPTKAGGFILESECGKIKVNAGSGLKDKAGVKSHELDRTRIMENQNY | |
| | YIGKILECECNGWLKSDGRTDYVKLFLPIAIRLREDKTKANTFEDVFGDF | |
| | HEVTGLSGGSSGGSKRTADGSEFESPKKKRKVSGGSSGGSMGIQGLAKLI | |
| | ADVAPSAIRENDIKSYFGRKVAIDASMSIYQFLIAVRQGGDVLQNEEGET | |
| | TSHLMGMFYRTIRMMENGIKPVYVFDGKPPQLKSGELAKRSERRAEAEKQ | |
| | LQQAQAAGAEQEVEKFTKRLVKVTKQHNDECKHLLSLMGIPYLDAPSEAE | |
| | ASCAALVKAGKVYAAATEDMDCLTFGSPVLMRHLTASEAKKLPIQEFHLS | |
| | RILQELGLNQEQFVDLCILLGSDYCESIRGIGPKRAVDLIQKHKSIEEIV | |
| | RRLDPNKYPVPENWLHKEAHQLFLEPEVLDPESVELKWSEPNEEELIKFM | |
| | CGEKQFSEERIRSGVKRLSKSRQGSTQGRLDDFFKVTGSLSSAKRKEPEP | |
| | KGSTKKKAKTGAAGKFKRGKSGGSKRTADSQHSTPPKTKRKVEFEPKKKR | |
| | KV | |
| NLS1- | KRTADGSEFESPKKKRKVMILKILNEIASIGSTKQKQAILEKNKDNELLK | 139 |
| T4LIG- | RVYRLTYSRGLQYYIKKWPKPGIATQSFGMLTLTDMLDFIEFTLATRKLT | |
| linker1- | GNAAIEELTGYITDGKKDDVEVLRRVMMRDLECGASVSIANKVWPGLIPE | |
| hFENl- | QPQMLASSYDEKGINKNIKFPAFAQLKADGARCFAEVRGDELDDVRLLSR | |
| linker2- | AGNEYLGLDLLKEELIKMTAEARQIHPEGVLIDGELVYHEQVKKEPEGLD | |
| nCas9- | FLFDAYPENSKAKEFAEVAESRTASNGIANKSLKGTISEKEAQCMKFQVW | |
| NLS2 | DYVPLVEIYSLPAFRLKYDVRFSKLEQMTSGYDKVILIENQVVNNLDEAK | |
| | VIYKKYIDQGLEGIILKNIDGLWENARSKNLYKFKEVIDVDLKIVGIYPH | |
| | RKDPTKAGGFILESECGKIKVNAGSGLKDKAGVKSHELDRTRIMENQNYY | |
| | IGKILECECNGWLKSDGRTDYVKLFLPIAIRLREDKTKANTFEDVFGDFH | |
| | EVTGLSGGSSGGSSGSETPGTSESATPESSGGSSGGSSMGIQGLAKLIAD | |
| | VAPSAIRENDIKSYFGRKVAIDASMSIYQFLIAVRQGGDVLQNEEGETTS | |
| | HLMGMFYRTIRMMENGIKPVYVFDGKPPQLKSGELAKRSERRAEAEKQLQ | |
| | QAQAAGAEQEVEKFTKRLVKVTKQHNDECKHLLSLMGIPYLDAPSEAEAS | |
| | CAALVKAGKVYAAATEDMDCLTFGSPVLMRHLTASEAKKLPIQEFHLSRI | |
| | LQELGLNQEQFVDLCILLGSDYCESIRGIGPKRAVDLIQKHKSIEEIVRR | |
| | LDPNKYPVPENWLHKEAHQLFLEPEVLDPESVELKWSEPNEEELIKFMCG | |
| | EKQFSEERIRSGVKRLSKSRQGSTQGRLDDFFKVTGSLSSAKRKEPEPKG | |
| | STKKKAKTGAAGKFKRGKSGGSSGGSKRTADGSEFESPKKKRKVSGGSSG | |
| | GSMDKKYSIGLDIGTNSVGWAVITDEYKVPSKKFKVLGNTDRHSIKKNLI | |
| | GALLFDSGETAEATRLKRTARRRYTRRKNRICYLQEIFSNEMAKVDDSFF | |
| | HRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEE | |
| | NPINASGVDAKAILSARLSKSRRLENLIAQLPGEKKNGLFGNLIALSLGL | |
| | TPNFKSNFDLAEDAKLQLSKDTYDDDLDNLLAQIGDQYADLFLAAKNLSD | |
| | AILLSDILRVNTEITKAPLSASMIKRYDEHHQDLTLLKALVRQQLPEKYK | |
| | EIFFDQSKNGYAGYIDGGASQEEFYKFIKPILEKMDGTEELLVKLNREDL | |
| | LRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNF | |
| | DKNLPNEKVLPKHSLLYEYFTVYNELTKVKYVTEGMRKPAFLSGEQKKAI | |
| | VDLLFKTNRKVTVKQLKEDYFKKIECFDSVEISGVEDRFNASLGTYHDLL | |
| | KIIKDKDFLDNEENEDILEDIVLTLTLFEDREMIEERLKTYAHLFDDKVM | |
| | KQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELV | |
| | KVMGRHKPENIVIEMARENQTTQKGQKNSRERMKRIEEGIKELGSQILKE | |
| | HPVENTQLQNEKLYLYYLQNGRDMYVDQELDINRLSDYDVDAIVPQSFLK | |
| | DDSIDNKVLTRSDKNRGKSDNVPSEEVVKKMKNYWRQLLNAKLITQRKFD | |
| | NLTKAERGGLSELDKAGFIKRQLVETRQITKHVAQILDSRMNTKYDENDK | |
| | LIREVKVITLKSKLVSDFRKDFQFYKVREINNYHHAHDAYLNAVVGTALI | |
| | KKYPKLESEFVYGDYKVYDVRKMIAKSEQEIGKATAKYFFYSNIMNFFKT | |
| | EITLANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLVVAK | |
| | VEKGKSKKLKSVKELLGITIMERSSFEKNPIDFLEAKGYKEVKKDLIIKL | |
| | PKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLI | |
| | HQSITGLYETRIDLSQLGGDSGGSKRTADSQHSTPPKTKRKVEFEPKKKR | |
| | KV | |
| NLS1-T5 | KRTADGSEFESPKKKRKVMSKSWGKFIEEEEAEMASRRNLMIVDGTNLGF | 140 |
| EXO- | RFKHNNSKKPFASSYVSTIQSLAKSYSARTTIVLGDKGKSVFRLEHLPEY | |
| linker1- | KGNRDEKYAQRTEEEKALDEQFFEYLKDAFELCKTTFPTFTIRGVEADDM | |
| nCas9- | AAYIVKLIGHLYDHVWLISTDGDWDTLLTDKVSRFSFTTRREYHLRDMYE | |
| linker2- | HHNVDDVEQFISLKAIMGDLGDNIRGVEGIGAKRGYNIIREFGNVLDIID | |
| T4LIG- | QLPLPGKQKYIQNLNASEELLFRNLILVDLPTYCVDAIAAVGQDVLDKFT | |
| NLS2 | KDILEIAEQSGGSSGGSSGSETPGTSESATPESSGGSSGGSSMDKKYSIG | |
| | LDIGTNSVGWAVITDEYKVPSKKFKVLGNTDRHSIKKNLIGALLFDSGET | |
| | AEATRLKRTARRRYTRRKNRICYLQEIFSNEMAKVDDSFFHRLEESFLVE | |
| | EDKKHERHPIFGNIVDEVAYHEKYPTIYHLRKKLVDSTDKADLRLIYLAL | |
| | AHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDL | |
| | AEDAKLQLSKDTYDDDLDNLLAQIGDQYADLFLAAKNLSDAILLSDILRV | |
| | NTEITKAPLSASMIKRYDEHHQDLTLLKALVRQQLPEKYKEIFFDQSKNG | |
| | YAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARG | |
| | NSRFAWMTRKSEETITPWNFEEVVDKGASAQSFIERMTNFDKNLPNEKVL | |
| | PKHSLLYEYFTVYNELTKVKYVTEGMRKPAFLSGEQKKAIVDLLFKTNRK | |
| | VTVKQLKEDYFKKIECFDSVEISGVEDRFNASLGTYHDLLKIIKDKDFLD | |
| | NEENEDILEDIVLTLTLFEDREMIEERLKTYAHLFDDKVMKQLKRRRYTG | |
| | WGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPEN | |
| | IVIEMARENQTTQKGQKNSRERMKRIEEGIKELGSQILKEHPVENTQLQN | |
| | EKLYLYYLQNGRDMYVDQELDINRLSDYDVDAIVPQSFLKDDSIDNKVLT | |
| | RSDKNRGKSDNVPSEEVVKKMKNYWRQLLNAKLITQRKFDNLTKAERGGL | |
| | SELDKAGFIKRQLVETRQITKHVAQILDSRMNTKYDENDKLIREVKVITL | |
| | KSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIR | |
| | KRPLIETNGETGEIVWDKGRDFATVRKVLSMPQVNIVKKTEVQTGGFSKE | |
| | SILPKRNSDKLIARKKDWDPKKYGGFDSPTVAYSVLVVAKVEKGKSKKLK | |
| | SVKELLGITIMERSSFEKNPIDFLEAKGYKEVKKDLIIKLPKYSLFELEN | |
| | GRKRMLASAGELQKGNELALPSKYVNFLYLASHYEKLKGSPEDNEQKQLF | |
| | VEQHKHYLDEIIEQISEFSKRVILADANLDKVLSAYNKHRDKPIREQAEN | |
| | IIHLFTLTNLGAPAAFKYFDTTIDRKRYTSTKEVLDATLIHQSITGLYET | |
| | RIDLSQLGGDSGGSSGGSKRTADGSEFESPKKKRKVSGGSSGGSMILKIL | |
| | NEIASIGSTKQKQAILEKNKDNELLKRVYRLTYSRGLQYYIKKWPKPGIA | |
| | TQSFGMLTLTDMLDFIEFTLATRKLTGNAAIEELTGYITDGKKDDVEVLR | |
| | RVMMRDLECGASVSIANKVWPGLIPEQPQMLASSYDEKGINKNIKFPAFA | |
| | QLKADGARCFAEVRGDELDDVRLLSRAGNEYLGLDLLKEELIKMTAEARQ | |
| | IHPEGVLIDGELVYHEQVKKEPEGLDFLFDAYPENSKAKEFAEVAESRTA | |
| | SNGIANKSLKGTISEKEAQCMKFQVWDYVPLVEIYSLPAFRLKYDVRFSK | |
| | LEQMTSGYDKVILIENQVVNNLDEAKVIYKKYIDQGLEGIILKNIDGLWE | |
| | NARSKNLYKFKEVIDVDLKIVGIYPHRKDPTKAGGFILESECGKIKVNAG | |
| | SGLKDKAGVKSHELDRTRIMENQNYYIGKILECECNGWLKSDGRTDYVKL | |
| | FLPIAIRLREDKTKANTFEDVFGDFHEVTGLSGGSKRTADSQHSTPPKTK | |
| | RKVEFEPKKKRKV | |
| LZ- | ATGTTGGAGATAGAGGCTGCTTTCCTTGAACGGGAGAATACGGCCCTCGA | 141 |
| SplintR- | GACTAGGGTTGCTGAGCTTAGACAGCGAGTCCAAAGACTGCGAAACCGGG | |
| bpNLS | TGTCCCAATATAGGACCAGATACGGACCTCTGGGTGGAGGGAAATCCGGT | |
| | GGGAGTAGCGGCGGGTCTAGTGGCTCAGAGACACCTGGCACGAGCGAGAG | |
| | TGCGACTCCTGAAAGCTCCGGCGGCAGCAGTGGGGGAAGTTCCATGGCAA | |
| | TCACTAAGCCCCTCTTGGCGGCGACTTTGGAAAACATCGAGGATGTGCAA | |
| | TTCCCGTGCCTTGCCACACCAAAGATAGACGGGATCCGATCAGTGAAGCA | |
| | AACGCAGATGCTCTCTAGAACGTTCAAGCCTATTAGAAACTCAGTGATGA | |
| | ATCGGCTCTTGACTGAGCTGTTGCCGGAAGGCAGCGATGGGGAAATATCT | |
| | ATCGAGGGAGCCACATTTCAAGACACTACGAGCGCCGTAATGACTGGACA | |
| | TAAGATGTATAATGCTAAATTCTCCTACTATTGGTTTGACTATGTCACTG | |
| | ATGACCCTCTTAAGAAATATATAGACCGAGTGGAGGATATGAAAAATTAT | |
| | ATTACTGTACACCCGCATATTCTGGAACATGCCCAAGTTAAGATTATTCC | |
| | TCTCATTCCCGTCGAGATTAATAATATCACAGAACTGCTTCAGTATGAGC | |
| | GCGACGTATTGTCCAAAGGCTTTGAAGGGGTTATGATACGCAAACCGGAC | |
| | GGCAAGTACAAGTTCGGAAGAAGCACATTGAAAGAGGGTATATTGCTGAA | |
| | GATGAAGCAGTTTAAGGATGCTGAGGCAACAATAATCAGCATGACAGCAC | |
| | TTTTTAAAAATACCAACACGAAAACTAAGGACAATTTTGGTTATAGTAAG | |
| | CGGTCAACGCACAAAAGTGGGAAGGTAGAAGAAGACGTAATGGGTAGCAT | |
| | TGAGGTGGATTATGACGGGGTGGTTTTCAGCATAGGGACTGGGTTTGATG | |
| | CAGATCAACGGAGGGACTTTTGGCAGAACAAAGAATCATATATAGGCAAA | |
| | ATGGTAAAGTTCAAATACTTCGAAATGGGAAGTAAAGACTGCCCCAGATT | |
| | CCCTGTATTCATTGGCATCAGGCACGAGGAGGACAGGAGTGGGGGATCAA | |
| | AGCGGACTGCTGATAGTCAGCATAGTACTCCACCCAAGACCAAGCGGAAA | |
| | GTTGAGTTTGAGCCGAAGAAAAAGCGAAAAGTGTAA | |
| LZ-T4LIG- | ATGCTTGAGATCGAGGCGGCGTTCCTCGAAAGAGAGAACACCGCACTTGA | 142 |
| bpNLS | AACTCGCGTGGCAGAATTGAGGCAGCGGGTGCAAAGACTTAGAAATAGAG | |
| | TCTCTCAGTATCGGACCCGATATGGTCCTCTGGGAGGCGGGAAGTCTGGA | |
| | GGTTCAAGCGGAGGCAGTTCCGGGAGTGAGACACCGGGAACTTCTGAGAG | |
| | TGCAACTCCTGAGAGCTCTGGTGGATCATCCGGAGGCTCCAGTATGATTC | |
| | TTAAAATTCTTAACGAGATTGCGAGTATTGGCAGCACGAAACAAAAGCAG | |
| | GCCATATTGGAAAAGAATAAGGACAATGAGTTGCTTAAACGCGTGTATAG | |
| | GCTCACTTACTCTCGCGGACTGCAATACTATATTAAAAAATGGCCTAAGC | |
| | CCGGCATCGCTACTCAAAGCTTCGGAATGCTTACGCTGACAGATATGCTC | |
| | GACTTCATCGAGTTTACTCTCGCAACAAGGAAGTTGACTGGCAACGCCGC | |
| | GATTGAAGAATTGACGGGTTATATCACGGACGGGAAGAAGGATGATGTTG | |
| | AGGTGCTGAGGCGCGTTATGATGCGCGACCTCGAATGTGGTGCCTCAGTT | |
| | TCCATAGCCAATAAAGTTTGGCCAGGCTTGATCCCGGAGCAGCCACAGAT | |
| | GCTGGCCAGTAGCTACGACGAGAAGGGTATTAACAAAAATATCAAGTTTC | |
| | CAGCGTTTGCACAACTTAAAGCGGATGGGGCGCGGTGTTTCGCCGAAGTC | |
| | CGGGGTGACGAATTGGACGATGTGCGCCTTCTGAGTCGCGCAGGAAATGA | |
| | ATATCTGGGGCTTGACCTCTTGAAGGAGGAGCTGATTAAGATGACAGCAG | |
| | AAGCCAGGCAGATCCATCCAGAGGGGGTACTTATTGATGGTGAACTCGTA | |
| | TACCATGAGCAGGTTAAGAAGGAGCCAGAGGGTTTGGATTTCCTCTTTGA | |
| | CGCCTATCCCGAGAATTCAAAGGCAAAGGAGTTCGCCGAGGTTGCAGAAT | |
| | CAAGAACGGCTTCCAACGGCATAGCGAATAAATCACTCAAAGGAACTATA | |
| | TCTGAAAAGGAGGCACAGTGTATGAAATTCCAAGTGTGGGACTATGTGCC | |
| | GCTTGTCGAGATTTACAGCTTGCCTGCTTTCCGATTGAAGTACGATGTAC | |
| | GGTTTAGTAAGCTCGAGCAAATGACTTCAGGTTACGATAAAGTCATCTTG | |
| | ATTGAGAACCAGGTCGTTAATAATCTTGACGAGGCGAAGGTCATATATAA | |
| | GAAATATATAGATCAAGGGCTCGAGGGTATCATTCTGAAGAATATAGATG | |
| | GCTTGTGGGAAAACGCCAGGTCCAAAAACCTGTATAAGTTTAAGGAAGTA | |
| | ATAGATGTAGATTTGAAAATAGTTGGAATTTACCCCCATCGGAAGGACCC | |
| | CACGAAAGCGGGTGGGTTTATCCTCGAGAGCGAGTGTGGGAAGATAAAAG | |
| | TGAATGCCGGCTCCGGATTGAAGGACAAGGCAGGTGTGAAAAGTCATGAG | |
| | CTCGATCGGACGAGAATAATGGAAAACCAGAATTACTACATTGGAAAGAT | |
| | TTTGGAATGCGAGTGTAACGGCTGGTTGAAGAGCGACGGACGCACCGATT | |
| | ACGTGAAACTCTTTCTGCCAATTGCAATCAGGTTGAGAGAGGATAAGACT | |
| | AAGGCCAATACTTTCGAGGACGTCTTCGGAGACTTTCACGAAGTCACTGG | |
| | GCTTTCTGGGGGTAGTAAGAGAACTGCAGATAGCCAGCATTCAACGCCGC | |
| | CAAAAACAAAGCGAAAGGTAGAATTCGAACCAAAGAAAAAGCGGAAAGTA | |
| | TAA | |
| LZ- | ATGCTCGAGATCGAAGCTGCATTTCTGGAGAGGGAGAATACCGCCCTCGA | 143 |
| hLIG1 | AACCCGGGTGGCTGAATTGCGACAGAGAGTGCAACGGCTCCGGAATAGAG | |
| (233- | TATCTCAATATCGAACCCGCTATGGGCCTCTCGGAGGGGGTAAATCTGGC | |
| 919)- | GGAAGTTCTGGCGGTAGTTCAGGAAGTGAGACACCGGGAACTAGTGAATC | |
| bpNLS | CGCGACTCCCGAATCAAGTGGGGGATCATCTGGAGGGTCAAGCACACCCA | |
| | GGAAACCAGCCGTGAAAAAAGAGGTTAAAGAAGAGGAACCTGGGGCTCCG | |
| | GGAAAGGAGGGAGCAGCGGAAGGTCCGCTCGACCCTTCAGGATACAACCC | |
| | AGCCAAAAACAACTACCACCCCGTAGAGGATGCTTGCTGGAAGCCAGGCC | |
| | AAAAGGTGCCCTATTTGGCCGTTGCTAGGACTTTCGAAAAAATTGAGGAG | |
| | GTGAGCGCGCGACTCAGAATGGTAGAGACTCTGTCTAACCTCCTTCGCTC | |
| | CGTAGTGGCTCTTTCACCTCCAGATCTTCTTCCAGTGCTGTACCTGAGCC | |
| | TGAACCACTTGGGCCCTCCCCAGCAGGGACTGGAACTGGGCGTAGGGGAC | |
| | GGAGTATTGCTGAAGGCTGTTGCTCAGGCAACCGGACGACAGCTCGAGTC | |
| | TGTGCGAGCAGAAGCTGCAGAAAAGGGGGACGTCGGGTTGGTTGCCGAAA | |
| | ATTCAAGATCTACCCAACGATTGATGTTGCCACCGCCGCCTCTGACTGCG | |
| | TCAGGTGTATTCTCCAAGTTCCGGGATATTGCCAGGCTTACGGGTAGCGC | |
| | TTCCACTGCTAAAAAGATCGACATAATAAAAGGTCTGTTCGTCGCTTGTC | |
| | GCCATTCAGAGGCGAGGTTTATAGCCAGATCCCTTTCCGGACGACTTCGA | |
| | CTCGGCTTGGCTGAGCAGTCAGTACTGGCAGCTTTGTCTCAAGCTGTATC | |
| | ACTCACGCCCCCCGGACAAGAATTTCCACCCGCCATGGTTGACGCAGGCA | |
| | AGGGTAAGACTGCTGAGGCAAGAAAGACGTGGCTGGAGGAACAAGGTATG | |
| | ATACTTAAACAAACGTTTTGCGAAGTTCCGGACTTGGACCGGATCATACC | |
| | TGTGTTGCTGGAGCACGGCCTCGAGCGCTTGCCCGAACACTGTAAACTGT | |
| | CTCCAGGAATACCTCTCAAACCCATGTTGGCTCATCCTACGAGGGGAATC | |
| | TCAGAGGTACTTAAACGGTTTGAAGAAGCCGCTTTCACGTGCGAATACAA | |
| | GTATGATGGTCAGAGAGCGCAAATCCACGCATTGGAAGGGGGTGAGGTAA | |
| | AGATTTTTTCAAGGAATCAGGAGGACAATACAGGGAAGTACCCCGATATC | |
| | ATCAGTCGGATTCCTAAAATTAAGCTTCCATCAGTCACGTCCTTCATACT | |
| | GGACACTGAGGCAGTGGCTTGGGACCGAGAGAAGAAGCAGATACAACCCT | |
| | TTCAGGTACTTACAACCAGAAAGCGCAAGGAAGTCGACGCTTCTGAGATT | |
| | CAAGTACAAGTCTGCCTTTATGCGTTTGACCTGATCTATCTTAATGGAGA | |
| | GAGTTTGGTGAGAGAACCCTTGAGCAGACGACGGCAGCTCTTGAGAGAAA | |
| | ATTTCGTAGAAACTGAGGGGGAGTTCGTCTTTGCGACTAGTCTCGACACC | |
| | AAAGACATTGAGCAAATCGCGGAATTCCTCGAACAGTCAGTTAAAGACTC | |
| | CTGCGAAGGTCTGATGGTTAAGACTCTTGACGTGGATGCTACCTACGAGA | |
| | TAGCTAAGCGGTCACACAATTGGCTGAAACTGAAAAAGGACTATCTGGAT | |
| | GGAGTTGGGGACACGCTGGATTTGGTCGTTATCGGGGCCTATCTGGGACG | |
| | CGGTAAGCGGGCAGGGAGATATGGTGGATTCCTCCTCGCTTCATACGATG | |
| | AGGACTCTGAAGAGCTGCAGGCTATATGCAAACTTGGGACGGGTTTTTCC | |
| | GATGAAGAATTGGAGGAACATCATCAGTCACTGAAGGCCCTTGTATTGCC | |
| | AAGTCCACGCCCATACGTACGAATCGATGGAGCAGTAATCCCTGACCACT | |
| | GGCTTGACCCGTCCGCCGTCTGGGAAGTAAAGTGCGCGGATCTCTCTCTC | |
| | AGTCCGATCTACCCAGCCGCACGGGGGCTGGTTGACAGTGACAAGGGTAT | |
| | CAGCCTGCGATTTCCTCGATTCATACGCGTCCGGGAAGACAAGCAACCGG | |
| | AACAGGCTACGACCTCTGCACAGGTCGCATGTTTGTATAGAAAACAGAGC | |
| | CAAATTCAGAATCAACAAGGCGAAGACAGTGGGTCCGATCCTGAAGATAC | |
| | CTACTCAGGCGGCAGTAAACGGACAGCTGATAGCCAACACTCAACTCCTC | |
| | CGAAGACTAAAAGGAAGGTAGAGTTCGAACCAAAAAAGAAAAGGAAAGTG | |
| | TAA | |
| LZ- | ATGCTCGAGATCGAGGCGGCGTTCCTTGAACGCGAGAACACTGCGCTGGA | 144 |
| hLIG1 | AACGAGGGTCGCGGAACTCCGCCAGAGGGTTCAACGGTTGAGGAATCGAG | |
| (119- | TGAGTCAGTACCGAACCCGATATGGACCACTGGGTGGCGGGAAATCAGGG | |
| 919)- | GGCTCATCCGGCGGCTCCAGCGGGAGCGAAACCCCGGGTACCTCAGAATC | |
| bpNLS | TGCGACGCCAGAAAGCTCAGGCGGATCTAGCGGCGGTAGTTCACCGAAGC | |
| | GCCGGACTGCACGAAAGCAACTGCCAAAACGGACTATACAAGAAGTCCTG | |
| | GAAGAACAAAGCGAAGATGAGGATCGCGAAGCCAAGCGCAAGAAAGAGGA | |
| | AGAGGAAGAAGAGACTCCAAAGGAGTCCTTGACCGAAGCAGAAGTCGCAA | |
| | CGGAGAAGGAAGGTGAGGATGGGGATCAGCCAACAACCCCGCCTAAACCT | |
| | CTGAAAACCTCTAAGGCGGAGACACCAACTGAGAGTGTCAGCGAACCGGA | |
| | GGTAGCCACGAAACAAGAGCTTCAGGAGGAAGAAGAACAGACAAAGCCAC | |
| | CTCGGCGGGCTCCCAAAACCCTTAGCTCCTTCTTCACGCCTCGAAAGCCA | |
| | GCAGTGAAGAAAGAAGTGAAGGAGGAGGAACCTGGCGCCCCTGGAAAGGA | |
| | GGGCGCAGCCGAGGGCCCGCTGGACCCTTCAGGGTATAACCCGGCAAAAA | |
| | ATAATTACCACCCGGTCGAGGACGCTTGTTGGAAACCAGGCCAAAAGGTA | |
| | CCTTACCTCGCCGTCGCTAGGACCTTTGAGAAGATAGAGGAAGTTAGTGC | |
| | TAGGTTGAGAATGGTCGAAACCCTTAGTAACCTTCTCAGGTCCGTAGTCG | |
| | CCCTTAGTCCCCCAGACCTGCTTCCGGTGCTGTACCTGTCCCTGAACCAT | |
| | CTCGGTCCCCCCCAACAGGGACTGGAGTTGGGCGTCGGTGACGGCGTTCT | |
| | CCTGAAAGCGGTTGCACAAGCTACAGGAAGGCAACTGGAATCTGTCCGGG | |
| | CTGAGGCTGCAGAGAAAGGTGACGTGGGGCTTGTGGCAGAGAATAGTCGG | |
| | TCAACACAGCGGCTGATGCTGCCACCGCCCCCGCTTACGGCTAGTGGGGT | |
| | ATTCTCCAAATTTAGAGATATAGCACGGCTGACGGGATCAGCTTCCACTG | |
| | CGAAGAAGATCGATATCATTAAGGGTTTGTTCGTGGCTTGCAGGCATTCC | |
| | GAAGCACGCTTCATTGCACGCTCCCTTTCAGGGAGACTCAGACTTGGGCT | |
| | GGCCGAGCAATCTGTACTGGCGGCCCTGTCTCAGGCGGTGAGCCTTACGC | |
| | CGCCCGGGCAAGAGTTCCCTCCTGCGATGGTCGATGCTGGGAAGGGAAAA | |
| | ACCGCCGAAGCTCGAAAAACATGGCTGGAGGAGCAAGGAATGATTTTGAA | |
| | GCAGACGTTCTGTGAAGTACCGGACTTGGATCGCATCATACCTGTGCTTC | |
| | TCGAACATGGTTTGGAGCGGCTCCCCGAGCATTGCAAACTCTCTCCGGGC | |
| | ATCCCCCTCAAGCCAATGCTCGCCCACCCCACGCGCGGAATCAGTGAGGT | |
| | ACTGAAACGCTTTGAAGAGGCAGCGTTTACTTGTGAATACAAGTACGATG | |
| | GCCAAAGGGCACAAATTCATGCACTTGAAGGCGGGGAAGTTAAGATATTC | |
| | AGCAGGAATCAGGAGGACAACACGGGAAAATATCCTGACATAATATCTCG | |
| | AATCCCTAAAATTAAGTTGCCTAGCGTAACCAGCTTCATCCTGGATACCG | |
| | AAGCCGTGGCGTGGGATAGGGAGAAAAAGCAAATACAGCCATTTCAGGTG | |
| | CTTACAACTAGAAAACGAAAAGAGGTGGACGCTAGTGAAATCCAAGTCCA | |
| | GGTATGTCTTTATGCCTTCGATTTGATATACCTTAATGGTGAGTCCCTTG | |
| | TACGGGAACCGCTTAGTAGGAGGCGGCAGTTGCTGAGGGAAAATTTTGTC | |
| | GAAACTGAGGGAGAGTTTGTATTTGCAACGTCATTGGATACAAAGGACAT | |
| | AGAACAAATAGCAGAATTTCTGGAGCAGTCAGTAAAAGACTCCTGCGAGG | |
| | GCCTGATGGTGAAAACTCTTGATGTGGACGCCACTTATGAAATCGCAAAA | |
| | AGGTCACACAATTGGCTGAAACTTAAAAAGGATTACTTGGACGGGGTCGG | |
| | GGATACCCTCGATCTCGTCGTAATCGGAGCTTATCTCGGTAGGGGGAAGC | |
| | GAGCCGGGCGATACGGAGGCTTTCTCTTGGCTAGTTATGACGAAGATTCC | |
| | GAAGAGCTGCAGGCCATATGCAAGCTTGGAACGGGTTTCAGCGATGAGGA | |
| | ATTGGAGGAGCATCATCAGAGCTTGAAGGCACTGGTGCTCCCCTCTCCTA | |
| | GGCCGTACGTTAGAATAGACGGAGCAGTGATACCCGATCATTGGCTCGAT | |
| | CCGTCAGCTGTTTGGGAGGTGAAGTGTGCAGACCTGTCCCTCTCTCCTAT | |
| | TTACCCTGCAGCACGCGGTCTGGTTGACTCTGACAAAGGGATTAGCTTGA | |
| | GGTTCCCTAGATTTATTCGGGTGCGCGAAGACAAACAGCCTGAACAGGCG | |
| | ACAACGTCCGCGCAGGTCGCATGCCTTTATCGAAAACAGAGTCAGATCCA | |
| | GAATCAACAAGGAGAAGATTCAGGGAGTGACCCGGAGGACACTTATAGTG | |
| | GCGGCTCAAAACGAACCGCCGATAGTCAGCATTCAACACCTCCAAAAACT | |
| | AAAAGGAAAGTCGAGTTTGAGCCAAAGAAGAAGCGCAAAGTCTAA | |
| T4-LZ | ATGATCCTTAAGATTCTCAACGAAATCGCTAGTATAGGGTCCACTAAGCA | 145 |
| | GAAGCAGGCCATATTGGAAAAAAATAAGGACAATGAACTTTTGAAGAGAG | |
| | TCTATAGACTGACGTACTCTAGGGGGCTCCAGTACTACATCAAGAAATGG | |
| | CCTAAACCTGGCATTGCGACGCAGTCATTCGGTATGCTGACATTGACGGA | |
| | TATGTTGGATTTCATTGAGTTTACGCTGGCCACCAGAAAACTTACGGGTA | |
| | ATGCTGCGATAGAAGAACTTACAGGGTACATAACAGACGGGAAGAAAGAT | |
| | GACGTGGAAGTGCTCAGACGAGTTATGATGCGCGATCTCGAGTGCGGCGC | |
| | TAGCGTGTCAATCGCGAACAAAGTCTGGCCCGGCCTCATACCAGAGCAGC | |
| | CACAGATGCTGGCATCTTCCTATGACGAAAAAGGCATAAACAAGAATATT | |
| | AAATTCCCGGCCTTCGCTCAACTCAAAGCAGATGGTGCCAGGTGTTTTGC | |
| | CGAAGTTCGGGGTGATGAACTTGATGACGTGCGGCTCTTGTCTAGGGCAG | |
| | GTAACGAGTACCTCGGCCTGGACTTGCTTAAAGAGGAACTGATTAAAATG | |
| | ACAGCTGAGGCGCGGCAGATACACCCCGAGGGCGTCCTTATCGACGGGGA | |
| | GCTGGTGTATCACGAACAAGTTAAAAAGGAACCGGAGGGTCTTGATTTTC | |
| | TTTTCGACGCGTATCCTGAGAACAGCAAGGCGAAAGAATTTGCAGAAGTT | |
| | GCAGAAAGCAGGACCGCAAGTAATGGAATCGCTAATAAAAGCCTCAAGGG | |
| | TACCATCAGCGAAAAAGAAGCCCAGTGCATGAAATTTCAAGTTTGGGACT | |
| | ATGTCCCCTTGGTCGAAATTTACTCCCTGCCCGCATTCCGGCTGAAGTAT | |
| | GATGTTCGCTTCAGTAAACTGGAGCAAATGACGAGCGGTTATGATAAGGT | |
| | TATACTTATTGAGAATCAGGTCGTAAATAATTTGGACGAGGCGAAAGTTA | |
| | TATACAAAAAGTATATAGACCAAGGGTTGGAGGGGATCATTTTGAAGAAC | |
| | ATAGACGGACTTTGGGAGAACGCCCGGTCCAAGAATTTGTATAAATTCAA | |
| | AGAAGTCATAGATGTTGACCTCAAGATAGTAGGTATATATCCCCACAGAA | |
| | AGGACCCAACCAAAGCAGGCGGATTCATTTTGGAGTCCGAGTGTGGGAAG | |
| | ATAAAGGTCAATGCTGGATCTGGACTCAAGGACAAAGCTGGTGTGAAGTC | |
| | ACATGAACTGGACCGAACCAGGATTATGGAGAATCAGAACTATTACATCG | |
| | GGAAGATATTGGAGTGTGAATGCAACGGCTGGCTTAAATCAGATGGAAGA | |
| | ACTGATTACGTTAAATTGTTCCTGCCCATAGCCATACGACTCCGCGAGGA | |
| | CAAAACGAAGGCTAACACGTTTGAAGACGTATTCGGAGATTTCCATGAGG | |
| | TGACTGGCCTTAGTGgaggctccaaacggacagcagactcccaacattca | |
| | acacccccaaaaacaaagcggaaggtagagtttgagccaaaaaagaaaag | |
| | aaaggtcGGAGGAGGAGGCAGTGGTGGGCGACTTGAAATTAGAGCCGCGT | |
| | TCCTGCGCCAGAGGAATACGGCTCTCCGCACGGAGGTAGCCGAACTTGAG | |
| | CAAGAAGTACAGAGATTGGAGAACGAGGTTTCACAGTATGAGACACGATA | |
| | TGGCCCCCTTGGCGGCGGAAAGTAA | |
| LZ- | ATGCTTGAGATCGAGGCGGCGTTCCTCGAAAGAGAGAACACCGCACTTGA | 146 |
| hLIG4(1- | AACTCGCGTGGCAGAATTGAGGCAGCGGGTGCAAAGACTTAGAAATAGAG | |
| 620) | TCTCTCAGTATCGGACCCGATATGGTCCTCTGGGAGGCGGGAAGTCTGGA | |
| | GGTTCAAGCGGAGGCAGTTCCGGGAGTGAGACACCGGGAACTTCTGAGAG | |
| | TGCAACTCCTGAGAGCTCTGGTGGATCATCCGGAGGCTCCAGTGCAGCTT | |
| | CTCAGACCTCTCAAACAGTAGCCTCTCATGTACCGTTCGCTGACTTGTGT | |
| | TCTACGCTCGAACGCATCCAGAAATCAAAGGGGCGCGCCGAGAAAATCCG | |
| | GCACTTCAGAGAATTCTTGGATTCCTGGAGGAAGTTTCATGATGCTCTCC | |
| | ACAAAAATCACAAAGATGTAACGGATAGTTTCTACCCTGCTATGAGACTT | |
| | ATACTGCCGCAGCTTGAGAGGGAACGCATGGCGTATGGTATAAAGGAGAC | |
| | AATGTTGGCGAAATTGTATATTGAGCTGCTGAACTTGCCAAGAGATGGAA | |
| | AGGACGCGCTCAAACTGCTGAACTATAGAACACCCACGGGTACCCATGGT | |
| | GACGCCGGTGACTTTGCCATGATCGCCTATTTCGTACTGAAACCTCGATG | |
| | TCTTCAAAAAGGTTCTCTTACAATTCAGCAAGTCAACGACCTGCTGGATT | |
| | CAATTGCGAGTAACAACAGCGCTAAGCGAAAGGATCTCATTAAGAAAAGC | |
| | CTCCTGCAGCTGATAACTCAGTCCTCTGCACTCGAACAAAAATGGCTGAT | |
| | TCGGATGATTATCAAGGATTTGAAGTTGGGGGTATCTCAGCAAACTATTT | |
| | TCAGCGTGTTTCACAATGATGCAGCAGAATTGCATAATGTCACAACAGAT | |
| | CTTGAGAAAGTCTGCCGACAGTTGCACGACCCCTCTGTAGGCTTGAGTGA | |
| | CATATCTATAACACTTTTTTCTGCGTTCAAACCCATGTTGGCTGCTATTG | |
| | CGGACATAGAACACATCGAGAAAGACATGAAACATCAGTCATTCTATATA | |
| | GAGACTAAATTGGACGGCGAGAGGATGCAAATGCACAAAGATGGTGATGT | |
| | GTATAAATATTTTTCCCGCAACGGCTACAACTACACTGATCAATTCGGAG | |
| | CGTCCCCAACTGAAGGGTCCCTCACTCCTTTCATACACAATGCGTTTAAG | |
| | GCCGATATTCAGATATGTATCCTCGACGGCGAAATGATGGCGTACAATCC | |
| | CAATACCCAGACCTTCATGCAAAAGGGAACGAAGTTCGATATTAAACGGA | |
| | TGGTTGAAGATTCCGACCTCCAAACATGTTACTGTGTGTTTGATGTCCTG | |
| | ATGGTGAATAACAAAAAACTCGGCCATGAAACCCTTCGAAAGCGATACGA | |
| | AATACTCAGCAGTATATTTACTCCAATACCAGGCCGAATCGAGATCGTAC | |
| | AGAAAACACAAGCCCATACTAAGAATGAAGTTATTGATGCACTGAACGAA | |
| | GCCATAGACAAGAGGGAAGAAGGCATAATGGTCAAGCAGCCTCTGAGTAT | |
| | ATATAAACCTGACAAAAGGGGGGAAGGATGGCTGAAGATAAAGCCAGAAT | |
| | ACGTGTCTGGTCTTATGGACGAATTGGACATTCTCATCGTCGGAGGATAT | |
| | TGGGGTAAGGGTTCCAGGGGGGGGATGATGTCCCACTTTCTGTGTGCGGT | |
| | TGCCGAGAAACCGCCCCCAGGGGAAAAACCATCAGTGTTCCATACGTTGT | |
| | CACGCGTCGGCTCAGGTTGTACGATGAAGGAACTTTACGATCTGGGGTTG | |
| | AAACTCGCCAAATATTGGAAGCCATTCCATCGGAAAGCACCGCCCTCTAG | |
| | TATCTTGTGTGGGACGGAGAAGCCAGAAGTTTATATAGAGCCATGTAACT | |
| | CAGTAATTGTTCAAATCAAAGCCGCAGAGATCGTCCCGTCAGACATGTAC | |
| | AAGACTGGATGCACCCTTAGATTTCCTCGCATCGAAAAAATAAGAGATGA | |
| | TAAAGAGTGGCATGAGTGCATGACTCTTGACGACCTTGAACAGCTCCGCG | |
| | GGAAGGCCAGCGGTAAACTGGCTAGTAAGCACCTCTACATCGGGGGTGAC | |
| | AGTGgaggctccaaacggacagcagactcccaacattcaacacccccaaa | |
| | aacaaagcggaaggtagagtttgagccaaaaaagaaaagaaaggtctaa | |
| LZ-nCas9 | ATGCTTGAGATCGAGGCGGCGTTCCTCGAAAGAGAGAACACCGCACTTGA | 147 |
| | AACTCGCGTGGCAGAATTGAGGCAGCGGGTGCAAAGACTTAGAAATAGAG | |
| | TCTCTCAGTATCGGACCCGATATGGTCCTCTGGGAGGCGGGAAGTCTGGA | |
| | GGTTCAAGCGGAGGCAGTTCCGGGAGTGAGACACCGGGAACTTCTGAGAG | |
| | TGCAACTCCTGAGAGCTCTGGTGGATCATCCGGAGGCTCCAGTaaacgga | |
| | cagccgacggaagcgagttcgagtcaccaaagaagaagcggaaagtcgac | |
| | aagaagtacagcatcggcctggacatcggcaccaactctgtgggctgggc | |
| | cgtgatcaccgacgagtacaaggtgcccagcaagaaattcaaggtgctgg | |
| | gcaacaccgaccggcacagcatcaagaagaacctgatcggagccctgctg | |
| | ttcgacagcggcgaaacagccgaggccacccggctgaagagaaccgccag | |
| | aagaagatacaccagacggaagaaccggatctgctatctgcaagagatct | |
| | tcagcaacgagatggccaaggtggacgacagcttcttccacagactggaa | |
| | gagtccttcctggtggaagaggataagaagcacgagcggcaccccatctt | |
| | cggcaacatcgtggacgaggtggcctaccacgagaagtaccccaccatct | |
| | accacctgagaaagaaactggtggacagcaccgacaaggccgacctgcgg | |
| | ctgatctatctggccctggcccacatgatcaagttccggggccacttcct | |
| | gatcgagggcgacctgaaccccgacaacagcgacgtggacaagctgttca | |
| | tccagctggtgcagacctacaaccagctgttcgaggaaaaccccatcaac | |
| | gccagcggcgtggacgccaaggccatcctgtctgccagactgagcaagag | |
| | cagacggctggaaaatctgatcgcccagctgcccggcgagaagaagaatg | |
| | gcctgttcggaaacctgattgccctgagcctgggcctgacccccaacttc | |
| | aagagcaacttcgacctggccgaggatgccaaactgcagctgagcaagga | |
| | cacctacgacgacgacctggacaacctgctggcccagatcggcgaccagt | |
| | acgccgacctgtttctggccgccaagaacctgtccgacgccatcctgctg | |
| | agcgacatcctgagagtgaacaccgagatcaccaaggcccccctgagcgc | |
| | ctctatgatcaagagatacgacgagcaccaccaggacctgaccctgctga | |
| | aagctctcgtgcggcagcagctgcctgagaagtacaaagagattttcttc | |
| | gaccagagcaagaacggctacgccggctacattgacggcggagccagcca | |
| | ggaagagttctacaagttcatcaagcccatcctggaaaagatggacggca | |
| | ccgaggaactgctcgtgaagctgaacagagaggacctgctgcggaagcag | |
| | cggaccttcgacaacggcagcatcccccaccagatccacctgggagagct | |
| | gcacgccattctgcggcggcaggaagatttttacccattcctgaaggaca | |
| | accgggaaaagatcgagaagatcctgaccttccgcatcccctactacgtg | |
| | ggccctctggccaggggaaacagcagattcgcctggatgaccagaaagag | |
| | cgaggaaaccatcaccccctggaacttcgaggaagtggtggacaagggcg | |
| | cttccgcccagagcttcatcgagcggatgaccaacttcgataagaacctg | |
| | cccaacgagaaggtgctgcccaagcacagcctgctgtacgagtacttcac | |
| | cgtgtataacgagctgaccaaagtgaaatacgtgaccgagggaatgagaa | |
| | agcccgccttcctgagcggcgagcagaaaaaggccatcgtggacctgctg | |
| | ttcaagaccaaccggaaagtgaccgtgaagcagctgaaagaggactactt | |
| | caagaaaatcgagtgcttcgactccgtggaaatctccggcgtggaagatc | |
| | ggttcaacgcctccctgggcacataccacgatctgctgaaaattatcaag | |
| | gacaaggacttcctggacaatgaggaaaacgaggacattctggaagatat | |
| | cgtgctgaccctgacactgtttgaggacagagagatgatcgaggaacggc | |
| | tgaaaacctatgcccacctgttcgacgacaaagtgatgaagcagctgaag | |
| | cggcggagatacaccggctggggcaggctgagccggaagctgatcaacgg | |
| | catccgggacaagcagtccggcaagacaatcctggatttcctgaagtccg | |
| | acggcttcgccaacagaaacttcatgcagctgatccacgacgacagcctg | |
| | acctttaaagaggacatccagaaagcccaggtgtccggccagggcgatag | |
| | cctgcacgagcacattgccaatctggccggcagccccgccattaagaagg | |
| | gcatcctgcagacagtgaaggtggtggacgagctcgtgaaagtgatgggc | |
| | cggcacaagcccgagaacatcgtgatcgaaatggccagagagaaccagac | |
| | cacccagaagggacagaagaacagccgcgagagaatgaagcggatcgaag | |
| | agggcatcaaagagctgggcagccagatcctgaaagaacaccccgtggaa | |
| | aacacccagctgcagaacgagaagctgtacctgtactacctgcagaatgg | |
| | gcgggatatgtacgtggaccaggaactggacatcaaccggctgtccgact | |
| | acgatgtggacgctatcgtgcctcagagctttctgaaggacgactccatc | |
| | gacaacaaggtgctgaccagaagcgacaagaaccggggcaagagcgacaa | |
| | cgtgccctccgaagaggtcgtgaagaagatgaagaactactggcggcagc | |
| | tgctgaacgccaagctgattacccagagaaagttcgacaatctgaccaag | |
| | gccgagagaggcggcctgagcgaactggataaggccggcttcatcaagag | |
| | acagctggtggaaacccggcagatcacaaagcacgtggcacagatcctgg | |
| | actcccggatgaacactaagtacgacgagaatgacaagctgatccgggaa | |
| | gtgaaagtgatcaccctgaagtccaagctggtgtccgatttccggaagga | |
| | tttccagttttacaaagtgcgcgagatcaacaactaccaccacgcccacg | |
| | acgcctacctgaacgccgtcgtgggaaccgccctgatcaaaaagtaccct | |
| | aagctggaaagcgagttcgtgtacggcgactacaaggtgtacgacgtgcg | |
| | gaagatgatcgccaagagcgagcaggaaatcggcaaggctaccgccaagt | |
| | acttcttctacagcaacatcatgaactttttcaagaccgagattaccctg | |
| | gccaacggcgagatccggaagcggcctctgatcgagacaaacggcgaaac | |
| | cggggagatcgtgtgggataagggccgggattttgccaccgtgcggaaag | |
| | tgctgagcatgccccaagtgaatatcgtgaaaaagaccgaggtgcagaca | |
| | ggcggcttcagcaaagagtctatcctgcccaagaggaacagcgataagct | |
| | gatcgccagaaagaaggactgggaccctaagaagtacggcggcttcgaca | |
| | gccccaccgtggcctattctgtgctggtggtggccaaagtggaaaagggc | |
| | aagtccaagaaactgaagagtgtgaaagagctgctggggatcaccatcat | |
| | ggaaagaagcagcttcgagaagaatcccatcgactttctggaagccaagg | |
| | gctacaaagaagtgaaaaaggacctgatcatcaagctgcctaagtactcc | |
| | ctgttcgagctggaaaacggccggaagagaatgctggcctctgccggcga | |
| | actgcagaagggaaacgaactggccctgccctccaaatatgtgaacttcc | |
| | tgtacctggccagccactatgagaagctgaagggctcccccgaggataat | |
| | gagcagaaacagctgtttgtggaacagcacaagcactacctggacgagat | |
| | catcgagcagatcagcgagttctccaagagagtgatcctggccgacgcta | |
| | atctggacaaagtgctgtccgcctacaacaagcaccgggataagcccatc | |
| | agagagcaggccgagaatatcatccacctgtttaccctgaccaatctggg | |
| | agcccctgccgccttcaagtactttgacaccaccatcgaccggaagaggt | |
| | acaccagcaccaaagaggtgctggacgccaccctgatccaccagagcatc | |
| | accggcctgtacgagacacggatcgacctgtctcagctgggaggtgacgg | |
| | ctcaaaaagaaccgccgacggcagcgaattcgagtcacccaagaagaaga | |
| | ggaaagtctaa | |
| SplintR- | atggcgataacgaagcccttgttggcagctacgttggaaaatattgagga | 148 |
| LZ | cgtacagttcccatgccttgccactccgaagatcgatggaatccgatccg | |
| | tgaaacagacacaaatgcttagcagaacattcaaacccatcaggaacagc | |
| | gtaatgaatagattgcttacggaactcttgcccgaagggtctgacggcga | |
| | gattagcatcgaaggagcgactttccaagatactacctcagcagttatga | |
| | cgggacacaagatgtataatgctaaattctcatattactggtttgactat | |
| | gttactgacgatcctttgaagaaatacatagacagggttgaagatatgaa | |
| | aaattacataactgtccaccctcatatcctggagcatgcacaggtaaaga | |
| | ttatcccgctcataccagtagaaattaacaatataaccgaattgttgcag | |
| | tatgaacgcgatgtgctctctaaaggcttcgagggcgtgatgataaggaa | |
| | gcctgatggcaaatataagttcggtaggtccacattgaaagagggaattc | |
| | tcttgaagatgaaacagtttaaggatgcggaagctactatcattagtatg | |
| | acggcactgtttaaaaacactaacactaaaaccaaggacaactttggcta | |
| | tagtaaaaggagcacacacaaatcaggaaaagttgaggaggacgtaatgg | |
| | gcagtatagaggtagattacgatggtgtggtgtttagcattggaacgggc | |
| | ttcgacgctgaccagcggagggacttttggcagaataaggaaagttacat | |
| | tggcaagatggttaaattcaaatacttcgagatgggctcaaaagactgtc | |
| | cgagatttcctgtgtttattggaatcagacacgaagaggataggAGTGga | |
| | ggctccaaacggacagcagactcccaacattcaacacccccaaaaacaaa | |
| | gcggaaggtagagtttgagccaaaaaagaaaagaaaggtcGGAGGAGGAG | |
| | GCAGTGGTGGGCGACTTGAAATTAGAGCCGCGTTCCTGCGCCAGAGGAAT | |
| | ACGGCTCTCCGCACGGAGGTAGCCGAACTTGAGCAAGAAGTACAGAGATT | |
| | GGAGAACGAGGTTTCACAGTATGAGACACGATATGGCCCCCTTGGCGGCG | |
| | GAAAGtaa | |
| hLIG4(1- | ATGGCAGCTTCTCAGACCTCTCAAACAGTAGCCTCTCATGTACCGTTCGC | 149 |
| 620)-LZ | TGACTTGTGTTCTACGCTCGAACGCATCCAGAAATCAAAGGGGCGCGCCG | |
| | AGAAAATCCGGCACTTCAGAGAATTCTTGGATTCCTGGAGGAAGTTTCAT | |
| | GATGCTCTCCACAAAAATCACAAAGATGTAACGGATAGTTTCTACCCTGC | |
| | TATGAGACTTATACTGCCGCAGCTTGAGAGGGAACGCATGGCGTATGGTA | |
| | TAAAGGAGACAATGTTGGCGAAATTGTATATTGAGCTGCTGAACTTGCCA | |
| | AGAGATGGAAAGGACGCGCTCAAACTGCTGAACTATAGAACACCCACGGG | |
| | TACCCATGGTGACGCCGGTGACTTTGCCATGATCGCCTATTTCGTACTGA | |
| | AACCTCGATGTCTTCAAAAAGGTTCTCTTACAATTCAGCAAGTCAACGAC | |
| | CTGCTGGATTCAATTGCGAGTAACAACAGCGCTAAGCGAAAGGATCTCAT | |
| | TAAGAAAAGCCTCCTGCAGCTGATAACTCAGTCCTCTGCACTCGAACAAA | |
| | AATGGCTGATTCGGATGATTATCAAGGATTTGAAGTTGGGGGTATCTCAG | |
| | CAAACTATTTTCAGCGTGTTTCACAATGATGCAGCAGAATTGCATAATGT | |
| | CACAACAGATCTTGAGAAAGTCTGCCGACAGTTGCACGACCCCTCTGTAG | |
| | GCTTGAGTGACATATCTATAACACTTTTTTCTGCGTTCAAACCCATGTTG | |
| | GCTGCTATTGCGGACATAGAACACATCGAGAAAGACATGAAACATCAGTC | |
| | ATTCTATATAGAGACTAAATTGGACGGCGAGAGGATGCAAATGCACAAAG | |
| | ATGGTGATGTGTATAAATATTTTTCCCGCAACGGCTACAACTACACTGAT | |
| | CAATTCGGAGCGTCCCCAACTGAAGGGTCCCTCACTCCTTTCATACACAA | |
| | TGCGTTTAAGGCCGATATTCAGATATGTATCCTCGACGGCGAAATGATGG | |
| | CGTACAATCCCAATACCCAGACCTTCATGCAAAAGGGAACGAAGTTCGAT | |
| | ATTAAACGGATGGTTGAAGATTCCGACCTCCAAACATGTTACTGTGTGTT | |
| | TGATGTCCTGATGGTGAATAACAAAAAACTCGGCCATGAAACCCTTCGAA | |
| | AGCGATACGAAATACTCAGCAGTATATTTACTCCAATACCAGGCCGAATC | |
| | GAGATCGTACAGAAAACACAAGCCCATACTAAGAATGAAGTTATTGATGC | |
| | ACTGAACGAAGCCATAGACAAGAGGGAAGAAGGCATAATGGTCAAGCAGC | |
| | CTCTGAGTATATATAAACCTGACAAAAGGGGGGAAGGATGGCTGAAGATA | |
| | AAGCCAGAATACGTGTCTGGTCTTATGGACGAATTGGACATTCTCATCGT | |
| | CGGAGGATATTGGGGTAAGGGTTCCAGGGGGGGGATGATGTCCCACTTTC | |
| | TGTGTGCGGTTGCCGAGAAACCGCCCCCAGGGGAAAAACCATCAGTGTTC | |
| | CATACGTTGTCACGCGTCGGCTCAGGTTGTACGATGAAGGAACTTTACGA | |
| | TCTGGGGTTGAAACTCGCCAAATATTGGAAGCCATTCCATCGGAAAGCAC | |
| | CGCCCTCTAGTATCTTGTGTGGGACGGAGAAGCCAGAAGTTTATATAGAG | |
| | CCATGTAACTCAGTAATTGTTCAAATCAAAGCCGCAGAGATCGTCCCGTC | |
| | AGACATGTACAAGACTGGATGCACCCTTAGATTTCCTCGCATCGAAAAAA | |
| | TAAGAGATGATAAAGAGTGGCATGAGTGCATGACTCTTGACGACCTTGAA | |
| | CAGCTCCGCGGGAAGGCCAGCGGTAAACTGGCTAGTAAGCACCTCTACAT | |
| | CGGGGGTGACAGTGgaggctccaaacggacagcagactcccaacattcaa | |
| | cacccccaaaaacaaagcggaaggtagagtttgagccaaaaaagaaaaga | |
| | aaggtcGGAGGAGGAGGCAGTGGTGGGCGACTTGAAATTAGAGCCGCGTT | |
| | CCTGCGCCAGAGGAATACGGCTCTCCGCACGGAGGTAGCCGAACTTGAGC | |
| | AAGAAGTACAGAGATTGGAGAACGAGGTTTCACAGTATGAGACACGATAT | |
| | GGCCCCCTTGGCGGCGGAAAGtaa | |
| nCas9- | atgaaacggacagccgacggaagcgagttcgagtcaccaaagaagaagcg | 150 |
| hLIG4(1- | gaaagtcgacaagaagtacagcatcggcctggacatcggcaccaactctg | |
| 620) | tgggctgggccgtgatcaccgacgagtacaaggtgcccagcaagaaattc | |
| | aaggtgctgggcaacaccgaccggcacagcatcaagaagaacctgatcgg | |
| | agccctgctgttcgacagcggcgaaacagccgaggccacccggctgaaga | |
| | gaaccgccagaagaagatacaccagacggaagaaccggatctgctatctg | |
| | caagagatcttcagcaacgagatggccaaggtggacgacagcttcttcca | |
| | cagactggaagagtccttcctggtggaagaggataagaagcacgagcggc | |
| | accccatcttcggcaacatcgtggacgaggtggcctaccacgagaagtac | |
| | cccaccatctaccacctgagaaagaaactggtggacagcaccgacaaggc | |
| | cgacctgcggctgatctatctggccctggcccacatgatcaagttccggg | |
| | gccacttcctgatcgagggcgacctgaaccccgacaacagcgacgtggac | |
| | aagctgttcatccagctggtgcagacctacaaccagctgttcgaggaaaa | |
| | ccccatcaacgccagcggcgtggacgccaaggccatcctgtctgccagac | |
| | tgagcaagagcagacggctggaaaatctgatcgcccagctgcccggcgag | |
| | aagaagaatggcctgttcggaaacctgattgccctgagcctgggcctgac | |
| | ccccaacttcaagagcaacttcgacctggccgaggatgccaaactgcagc | |
| | tgagcaaggacacctacgacgacgacctggacaacctgctggcccagatc | |
| | ggcgaccagtacgccgacctgtttctggccgccaagaacctgtccgacgc | |
| | catcctgctgagcgacatcctgagagtgaacaccgagatcaccaaggccc | |
| | ccctgagcgcctctatgatcaagagatacgacgagcaccaccaggacctg | |
| | accctgctgaaagctctcgtgcggcagcagctgcctgagaagtacaaaga | |
| | gattttcttcgaccagagcaagaacggctacgccggctacattgacggcg | |
| | gagccagccaggaagagttctacaagttcatcaagcccatcctggaaaag | |
| | atggacggcaccgaggaactgctcgtgaagctgaacagagaggacctgct | |
| | gcggaagcagcggaccttcgacaacggcagcatcccccaccagatccacc | |
| | tgggagagctgcacgccattctgcggcggcaggaagatttttacccattc | |
| | ctgaaggacaaccgggaaaagatcgagaagatcctgaccttccgcatccc | |
| | ctactacgtgggccctctggccaggggaaacagcagattcgcctggatga | |
| | ccagaaagagcgaggaaaccatcaccccctggaacttcgaggaagtggtg | |
| | gacaagggcgcttccgcccagagcttcatcgagcggatgaccaacttcga | |
| | taagaacctgcccaacgagaaggtgctgcccaagcacagcctgctgtacg | |
| | agtacttcaccgtgtataacgagctgaccaaagtgaaatacgtgaccgag | |
| | ggaatgagaaagcccgccttcctgagcggcgagcagaaaaaggccatcgt | |
| | ggacctgctgttcaagaccaaccggaaagtgaccgtgaagcagctgaaag | |
| | aggactacttcaagaaaatcgagtgcttcgactccgtggaaatctccggc | |
| | gtggaagatcggttcaacgcctccctgggcacataccacgatctgctgaa | |
| | aattatcaaggacaaggacttcctggacaatgaggaaaacgaggacattc | |
| | tggaagatatcgtgctgaccctgacactgtttgaggacagagagatgatc | |
| | gaggaacggctgaaaacctatgcccacctgttcgacgacaaagtgatgaa | |
| | gcagctgaagcggcggagatacaccggctggggcaggctgagccggaagc | |
| | tgatcaacggcatccgggacaagcagtccggcaagacaatcctggatttc | |
| | ctgaagtccgacggcttcgccaacagaaacttcatgcagctgatccacga | |
| | cgacagcctgacctttaaagaggacatccagaaagcccaggtgtccggcc | |
| | agggcgatagcctgcacgagcacattgccaatctggccggcagccccgcc | |
| | attaagaagggcatcctgcagacagtgaaggtggtggacgagctcgtgaa | |
| | agtgatgggccggcacaagcccgagaacatcgtgatcgaaatggccagag | |
| | agaaccagaccacccagaagggacagaagaacagccgcgagagaatgaag | |
| | cggatcgaagagggcatcaaagagctgggcagccagatcctgaaagaaca | |
| | ccccgtggaaaacacccagctgcagaacgagaagctgtacctgtactacc | |
| | tgcagaatgggcgggatatgtacgtggaccaggaactggacatcaaccgg | |
| | ctgtccgactacgatgtggacgctatcgtgcctcagagctttctgaagga | |
| | cgactccatcgacaacaaggtgctgaccagaagcgacaagaaccggggca | |
| | agagcgacaacgtgccctccgaagaggtcgtgaagaagatgaagaactac | |
| | tggcggcagctgctgaacgccaagctgattacccagagaaagttcgacaa | |
| | tctgaccaaggccgagagaggcggcctgagcgaactggataaggccggct | |
| | tcatcaagagacagctggtggaaacccggcagatcacaaagcacgtggca | |
| | cagatcctggactcccggatgaacactaagtacgacgagaatgacaagct | |
| | gatccgggaagtgaaagtgatcaccctgaagtccaagctggtgtccgatt | |
| | tccggaaggatttccagttttacaaagtgcgcgagatcaacaactaccac | |
| | cacgcccacgacgcctacctgaacgccgtcgtgggaaccgccctgatcaa | |
| | aaagtaccctaagctggaaagcgagttcgtgtacggcgactacaaggtgt | |
| | acgacgtgcggaagatgatcgccaagagcgagcaggaaatcggcaaggct | |
| | accgccaagtacttcttctacagcaacatcatgaactttttcaagaccga | |
| | gattaccctggccaacggcgagatccggaagcggcctctgatcgagacaa | |
| | acggcgaaaccggggagatcgtgtgggataagggccgggattttgccacc | |
| | gtgcggaaagtgctgagcatgccccaagtgaatatcgtgaaaaagaccga | |
| | ggtgcagacaggcggcttcagcaaagagtctatcctgcccaagaggaaca | |
| | gcgataagctgatcgccagaaagaaggactgggaccctaagaagtacggc | |
| | ggcttcgacagccccaccgtggcctattctgtgctggtggtggccaaagt | |
| | ggaaaagggcaagtccaagaaactgaagagtgtgaaagagctgctgggga | |
| | tcaccatcatggaaagaagcagcttcgagaagaatcccatcgactttctg | |
| | gaagccaagggctacaaagaagtgaaaaaggacctgatcatcaagctgcc | |
| | taagtactccctgttcgagctggaaaacggccggaagagaatgctggcct | |
| | ctgccggcgaactgcagaagggaaacgaactggccctgccctccaaatat | |
| | gtgaacttcctgtacctggccagccactatgagaagctgaagggctcccc | |
| | cgaggataatgagcagaaacagctgtttgtggaacagcacaagcactacc | |
| | tggacgagatcatcgagcagatcagcgagttctccaagagagtgatcctg | |
| | gccgacgctaatctggacaaagtgctgtccgcctacaacaagcaccggga | |
| | taagcccatcagagagcaggccgagaatatcatccacctgtttaccctga | |
| | ccaatctgggagcccctgccgccttcaagtactttgacaccaccatcgac | |
| | cggaagaggtacaccagcaccaaagaggtgctggacgccaccctgatcca | |
| | ccagagcatcaccggcctgtacgagacacggatcgacctgtctcagctgg | |
| | gaggtgacTCCGGTGGCTCCTCAGGGGGATCTAAACGCACGGCCGATGGG | |
| | TCCGAGTTTGAGTCTCCCAAGAAGAAAAGGAAAGTGAGTGGTGGAAGTAG | |
| | CGGCGGTAGCGCAGCTTCTCAGACCTCTCAAACAGTAGCCTCTCATGTAC | |
| | CGTTCGCTGACTTGTGTTCTACGCTCGAACGCATCCAGAAATCAAAGGGG | |
| | CGCGCCGAGAAAATCCGGCACTTCAGAGAATTCTTGGATTCCTGGAGGAA | |
| | GTTTCATGATGCTCTCCACAAAAATCACAAAGATGTAACGGATAGTTTCT | |
| | ACCCTGCTATGAGACTTATACTGCCGCAGCTTGAGAGGGAACGCATGGCG | |
| | TATGGTATAAAGGAGACAATGTTGGCGAAATTGTATATTGAGCTGCTGAA | |
| | CTTGCCAAGAGATGGAAAGGACGCGCTCAAACTGCTGAACTATAGAACAC | |
| | CCACGGGTACCCATGGTGACGCCGGTGACTTTGCCATGATCGCCTATTTC | |
| | GTACTGAAACCTCGATGTCTTCAAAAAGGTTCTCTTACAATTCAGCAAGT | |
| | CAACGACCTGCTGGATTCAATTGCGAGTAACAACAGCGCTAAGCGAAAGG | |
| | ATCTCATTAAGAAAAGCCTCCTGCAGCTGATAACTCAGTCCTCTGCACTC | |
| | GAACAAAAATGGCTGATTCGGATGATTATCAAGGATTTGAAGTTGGGGGT | |
| | ATCTCAGCAAACTATTTTCAGCGTGTTTCACAATGATGCAGCAGAATTGC | |
| | ATAATGTCACAACAGATCTTGAGAAAGTCTGCCGACAGTTGCACGACCCC | |
| | TCTGTAGGCTTGAGTGACATATCTATAACACTTTTTTCTGCGTTCAAACC | |
| | CATGTTGGCTGCTATTGCGGACATAGAACACATCGAGAAAGACATGAAAC | |
| | ATCAGTCATTCTATATAGAGACTAAATTGGACGGCGAGAGGATGCAAATG | |
| | CACAAAGATGGTGATGTGTATAAATATTTTTCCCGCAACGGCTACAACTA | |
| | CACTGATCAATTCGGAGCGTCCCCAACTGAAGGGTCCCTCACTCCTTTCA | |
| | TACACAATGCGTTTAAGGCCGATATTCAGATATGTATCCTCGACGGCGAA | |
| | ATGATGGCGTACAATCCCAATACCCAGACCTTCATGCAAAAGGGAACGAA | |
| | GTTCGATATTAAACGGATGGTTGAAGATTCCGACCTCCAAACATGTTACT | |
| | GTGTGTTTGATGTCCTGATGGTGAATAACAAAAAACTCGGCCATGAAACC | |
| | CTTCGAAAGCGATACGAAATACTCAGCAGTATATTTACTCCAATACCAGG | |
| | CCGAATCGAGATCGTACAGAAAACACAAGCCCATACTAAGAATGAAGTTA | |
| | TTGATGCACTGAACGAAGCCATAGACAAGAGGGAAGAAGGCATAATGGTC | |
| | AAGCAGCCTCTGAGTATATATAAACCTGACAAAAGGGGGGAAGGATGGCT | |
| | GAAGATAAAGCCAGAATACGTGTCTGGTCTTATGGACGAATTGGACATTC | |
| | TCATCGTCGGAGGATATTGGGGTAAGGGTTCCAGGGGGGGGATGATGTCC | |
| | CACTTTCTGTGTGCGGTTGCCGAGAAACCGCCCCCAGGGGAAAAACCATC | |
| | AGTGTTCCATACGTTGTCACGCGTCGGCTCAGGTTGTACGATGAAGGAAC | |
| | TTTACGATCTGGGGTTGAAACTCGCCAAATATTGGAAGCCATTCCATCGG | |
| | AAAGCACCGCCCTCTAGTATCTTGTGTGGGACGGAGAAGCCAGAAGTTTA | |
| | TATAGAGCCATGTAACTCAGTAATTGTTCAAATCAAAGCCGCAGAGATCG | |
| | TCCCGTCAGACATGTACAAGACTGGATGCACCCTTAGATTTCCTCGCATC | |
| | GAAAAAATAAGAGATGATAAAGAGTGGCATGAGTGCATGACTCTTGACGA | |
| | CCTTGAACAGCTCCGCGGGAAGGCCAGCGGTAAACTGGCTAGTAAGCACC | |
| | TCTACATCGGGGGTGACtaa | |
| T4-nCas9 | atgatgatccttaagattctcaacgaaatcgctagtatagggtccactaa | 151 |
| | gcagaagcaggccatattggaaaaaaataaggacaatgaacttttgaaga | |
| | gagtctatagactgacgtactctagggggctccagtactacatcaagaaa | |
| | tggcctaaacctggcattgcgacgcagtcattcggtatgctgacattgac | |
| | ggatatgttggatttcattgagtttacgctggccaccagaaaacttacgg | |
| | gtaatgctgcgatagaagaacttacagggtacataacagacgggaagaaa | |
| | gatgacgtggaagtgctcagacgagttatgatgcgcgatctcgagtgcgg | |
| | cgctagcgtgtcaatcgcgaacaaagtctggcccggcctcataccagagc | |
| | agccacagatgctggcatcttcctatgacgaaaaaggcataaacaagaat | |
| | attaaattcccggccttcgctcaactcaaagcagatggtgccaggtgttt | |
| | tgccgaagttcggggtgatgaacttgatgacgtgcggctcttgtctaggg | |
| | caggtaacgagtacctcggcctggacttgcttaaagaggaactgattaaa | |
| | atgacagctgaggcgcggcagatacaccccgagggcgtccttatcgacgg | |
| | ggagctggtgtatcacgaacaagttaaaaaggaaccggagggtcttgatt | |
| | ttcttttcgacgcgtatcctgagaacagcaaggcgaaagaatttgcagaa | |
| | gttgcagaaagcaggaccgcaagtaatggaatcgctaataaaagcctcaa | |
| | gggtaccatcagcgaaaaagaagcccagtgcatgaaatttcaagtttggg | |
| | actatgtccccttggtcgaaatttactccctgcccgcattccggctgaag | |
| | tatgatgttcgcttcagtaaactggagcaaatgacgagcggttatgataa | |
| | ggttatacttattgagaatcaggtcgtaaataatttggacgaggcgaaag | |
| | ttatatacaaaaagtatatagaccaagggttggaggggatcattttgaag | |
| | aacatagacggactttgggagaacgcccggtccaagaatttgtataaatt | |
| | caaagaagtcatagatgttgacctcaagatagtaggtatatatccccaca | |
| | gaaaggacccaaccaaagcaggcggattcattttggagtccgagtgtggg | |
| | aagataaaggtcaatgctggatctggactcaaggacaaagctggtgtgaa | |
| | gtcacatgaactggaccgaaccaggattatggagaatcagaactattaca | |
| | tcgggaagatattggagtgtgaatgcaacggctggcttaaatcagatgga | |
| | agaactgattacgttaaattgttcctgcccatagccatacgactccgcga | |
| | ggacaaaacgaaggctaacacgtttgaagacgtattcggagatttccatg | |
| | aggtgactggcctttccggtggctcctcagggggatctaaacgcacggcc | |
| | gatgggtccgagtttgagtctcccaagaagaaaaggaaagtgagtggtgg | |
| | aagtagcggcggtagcgacaagaagtacagcatcggcctggacatcggca | |
| | ccaactctgtgggctgggccgtgatcaccgacgagtacaaggtgcccagc | |
| | aagaaattcaaggtgctgggcaacaccgaccggcacagcatcaagaagaa | |
| | cctgatcggagccctgctgttcgacagcggcgaaacagccgaggccaccc | |
| | ggctgaagagaaccgccagaagaagatacaccagacggaagaaccggatc | |
| | tgctatctgcaagagatcttcagcaacgagatggccaaggtggacgacag | |
| | cttcttccacagactggaagagtccttcctggtggaagaggataagaagc | |
| | acgagcggcaccccatcttcggcaacatcgtggacgaggtggcctaccac | |
| | gagaagtaccccaccatctaccacctgagaaagaaactggtggacagcac | |
| | cgacaaggccgacctgcggctgatctatctggccctggcccacatgatca | |
| | agttccggggccacttcctgatcgagggcgacctgaaccccgacaacagc | |
| | gacgtggacaagctgttcatccagctggtgcagacctacaaccagctgtt | |
| | cgaggaaaaccccatcaacgccagcggcgtggacgccaaggccatcctgt | |
| | ctgccagactgagcaagagcagacggctggaaaatctgatcgcccagctg | |
| | cccggcgagaagaagaatggcctgttcggaaacctgattgccctgagcct | |
| | gggcctgacccccaacttcaagagcaacttcgacctggccgaggatgcca | |
| | aactgcagctgagcaaggacacctacgacgacgacctggacaacctgctg | |
| | gcccagatcggcgaccagtacgccgacctgtttctggccgccaagaacct | |
| | gtccgacgccatcctgctgagcgacatcctgagagtgaacaccgagatca | |
| | ccaaggcccccctgagcgcctctatgatcaagagatacgacgagcaccac | |
| | caggacctgaccctgctgaaagctctcgtgcggcagcagctgcctgagaa | |
| | gtacaaagagattttcttcgaccagagcaagaacggctacgccggctaca | |
| | ttgacggcggagccagccaggaagagttctacaagttcatcaagcccatc | |
| | ctggaaaagatggacggcaccgaggaactgctcgtgaagctgaacagaga | |
| | ggacctgctgcggaagcagcggaccttcgacaacggcagcatcccccacc | |
| | agatccacctgggagagctgcacgccattctgcggcggcaggaagatttt | |
| | tacccattcctgaaggacaaccgggaaaagatcgagaagatcctgacctt | |
| | ccgcatcccctactacgtgggccctctggccaggggaaacagcagattcg | |
| | cctggatgaccagaaagagcgaggaaaccatcaccccctggaacttcgag | |
| | gaagtggtggacaagggcgcttccgcccagagcttcatcgagcggatgac | |
| | caacttcgataagaacctgcccaacgagaaggtgctgcccaagcacagcc | |
| | tgctgtacgagtacttcaccgtgtataacgagctgaccaaagtgaaatac | |
| | gtgaccgagggaatgagaaagcccgccttcctgagcggcgagcagaaaaa | |
| | ggccatcgtggacctgctgttcaagaccaaccggaaagtgaccgtgaagc | |
| | agctgaaagaggactacttcaagaaaatcgagtgcttcgactccgtggaa | |
| | atctccggcgtggaagatcggttcaacgcctccctgggcacataccacga | |
| | tctgctgaaaattatcaaggacaaggacttcctggacaatgaggaaaacg | |
| | aggacattctggaagatatcgtgctgaccctgacactgtttgaggacaga | |
| | gagatgatcgaggaacggctgaaaacctatgcccacctgttcgacgacaa | |
| | agtgatgaagcagctgaagcggcggagatacaccggctggggcaggctga | |
| | gccggaagctgatcaacggcatccgggacaagcagtccggcaagacaatc | |
| | ctggatttcctgaagtccgacggcttcgccaacagaaacttcatgcagct | |
| | gatccacgacgacagcctgacctttaaagaggacatccagaaagcccagg | |
| | tgtccggccagggcgatagcctgcacgagcacattgccaatctggccggc | |
| | agccccgccattaagaagggcatcctgcagacagtgaaggtggtggacga | |
| | gctcgtgaaagtgatgggccggcacaagcccgagaacatcgtgatcgaaa | |
| | tggccagagagaaccagaccacccagaagggacagaagaacagccgcgag | |
| | agaatgaagcggatcgaagagggcatcaaagagctgggcagccagatcct | |
| | gaaagaacaccccgtggaaaacacccagctgcagaacgagaagctgtacc | |
| | tgtactacctgcagaatgggcgggatatgtacgtggaccaggaactggac | |
| | atcaaccggctgtccgactacgatgtggacgctatcgtgcctcagagctt | |
| | tctgaaggacgactccatcgacaacaaggtgctgaccagaagcgacaaga | |
| | accggggcaagagcgacaacgtgccctccgaagaggtcgtgaagaagatg | |
| | aagaactactggcggcagctgctgaacgccaagctgattacccagagaaa | |
| | gttcgacaatctgaccaaggccgagagaggcggcctgagcgaactggata | |
| | aggccggcttcatcaagagacagctggtggaaacccggcagatcacaaag | |
| | cacgtggcacagatcctggactcccggatgaacactaagtacgacgagaa | |
| | tgacaagctgatccgggaagtgaaagtgatcaccctgaagtccaagctgg | |
| | tgtccgatttccggaaggatttccagttttacaaagtgcgcgagatcaac | |
| | aactaccaccacgcccacgacgcctacctgaacgccgtcgtgggaaccgc | |
| | cctgatcaaaaagtaccctaagctggaaagcgagttcgtgtacggcgact | |
| | acaaggtgtacgacgtgcggaagatgatcgccaagagcgagcaggaaatc | |
| | ggcaaggctaccgccaagtacttcttctacagcaacatcatgaacttttt | |
| | caagaccgagattaccctggccaacggcgagatccggaagcggcctctga | |
| | tcgagacaaacggcgaaaccggggagatcgtgtgggataagggccgggat | |
| | tttgccaccgtgcggaaagtgctgagcatgccccaagtgaatatcgtgaa | |
| | aaagaccgaggtgcagacaggcggcttcagcaaagagtctatcctgccca | |
| | agaggaacagcgataagctgatcgccagaaagaaggactgggaccctaag | |
| | aagtacggcggcttcgacagccccaccgtggcctattctgtgctggtggt | |
| | ggccaaagtggaaaagggcaagtccaagaaactgaagagtgtgaaagagc | |
| | tgctggggatcaccatcatggaaagaagcagcttcgagaagaatcccatc | |
| | gactttctggaagccaagggctacaaagaagtgaaaaaggacctgatcat | |
| | caagctgcctaagtactccctgttcgagctggaaaacggccggaagagaa | |
| | tgctggcctctgccggcgaactgcagaagggaaacgaactggccctgccc | |
| | tccaaatatgtgaacttcctgtacctggccagccactatgagaagctgaa | |
| | gggctcccccgaggataatgagcagaaacagctgtttgtggaacagcaca | |
| | agcactacctggacgagatcatcgagcagatcagcgagttctccaagaga | |
| | gtgatcctggccgacgctaatctggacaaagtgctgtccgcctacaacaa | |
| | gcaccgggataagcccatcagagagcaggccgagaatatcatccacctgt | |
| | ttaccctgaccaatctgggagcccctgccgccttcaagtactttgacacc | |
| | accatcgaccggaagaggtacaccagcaccaaagaggtgctggacgccac | |
| | cctgatccaccagagcatcaccggcctgtacgagacacggatcgacctgt | |
| | ctcagctgggaggtgacaaacggacagccgacggaagcgagttcgagtca | |
| | ccaaagaagaagcggaaagtctaa | |
| SplintR- | atgatggcgataacgaagcccttgttggcagctacgttggaaaatattga | 152 |
| nCas9 | ggacgtacagttcccatgccttgccactccgaagatcgatggaatccgat | |
| | ccgtgaaacagacacaaatgcttagcagaacattcaaacccatcaggaac | |
| | agcgtaatgaatagattgcttacggaactcttgcccgaagggtctgacgg | |
| | cgagattagcatcgaaggagcgactttccaagatactacctcagcagtta | |
| | tgacgggacacaagatgtataatgctaaattctcatattactggtttgac | |
| | tatgttactgacgatcctttgaagaaatacatagacagggttgaagatat | |
| | gaaaaattacataactgtccaccctcatatcctggagcatgcacaggtaa | |
| | agattatcccgctcataccagtagaaattaacaatataaccgaattgttg | |
| | cagtatgaacgcgatgtgctctctaaaggcttcgagggcgtgatgataag | |
| | gaagcctgatggcaaatataagttcggtaggtccacattgaaagagggaa | |
| | ttctcttgaagatgaaacagtttaaggatgcggaagctactatcattagt | |
| | atgacggcactgtttaaaaacactaacactaaaaccaaggacaactttgg | |
| | ctatagtaaaaggagcacacacaaatcaggaaaagttgaggaggacgtaa | |
| | tgggcagtatagaggtagattacgatggtgtggtgtttagcattggaacg | |
| | ggcttcgacgctgaccagcggagggacttttggcagaataaggaaagtta | |
| | cattggcaagatggttaaattcaaatacttcgagatgggctcaaaagact | |
| | gtccgagatttcctgtgtttattggaatcagacacgaagaggataggtCC | |
| | GGTGGCTCCTCAgggggatctaaacgcacggccgatgggtccgagtttga | |
| | gtctcccaagaagaaaaggaaagtgagtggtggaagtagcggcggtagcg | |
| | acaagaagtacagcatcggcctggacatcggcaccaactctgtgggctgg | |
| | gccgtgatcaccgacgagtacaaggtgcccagcaagaaattcaaggtgct | |
| | gggcaacaccgaccggcacagcatcaagaagaacctgatcggagccctgc | |
| | tgttcgacagcggcgaaacagccgaggccacccggctgaagagaaccgcc | |
| | agaagaagatacaccagacggaagaaccggatctgctatctgcaagagat | |
| | cttcagcaacgagatggccaaggtggacgacagcttcttccacagactgg | |
| | aagagtccttcctggtggaagaggataagaagcacgagcggcaccccatc | |
| | ttcggcaacatcgtggacgaggtggcctaccacgagaagtaccccaccat | |
| | ctaccacctgagaaagaaactggtggacagcaccgacaaggccgacctgc | |
| | ggctgatctatctggccctggcccacatgatcaagttccggggccacttc | |
| | ctgatcgagggcgacctgaaccccgacaacagcgacgtggacaagctgtt | |
| | catccagctggtgcagacctacaaccagctgttcgaggaaaaccccatca | |
| | acgccagcggcgtggacgccaaggccatcctgtctgccagactgagcaag | |
| | agcagacggctggaaaatctgatcgcccagctgcccggcgagaagaagaa | |
| | tggcctgttcggaaacctgattgccctgagcctgggcctgacccccaact | |
| | tcaagagcaacttcgacctggccgaggatgccaaactgcagctgagcaag | |
| | gacacctacgacgacgacctggacaacctgctggcccagatcggcgacca | |
| | gtacgccgacctgtttctggccgccaagaacctgtccgacgccatcctgc | |
| | tgagcgacatcctgagagtgaacaccgagatcaccaaggcccccctgagc | |
| | gcctctatgatcaagagatacgacgagcaccaccaggacctgaccctgct | |
| | gaaagctctcgtgcggcagcagctgcctgagaagtacaaagagattttct | |
| | tcgaccagagcaagaacggctacgccggctacattgacggcggagccagc | |
| | caggaagagttctacaagttcatcaagcccatcctggaaaagatggacgg | |
| | caccgaggaactgctcgtgaagctgaacagagaggacctgctgcggaagc | |
| | agcggaccttcgacaacggcagcatcccccaccagatccacctgggagag | |
| | ctgcacgccattctgcggcggcaggaagatttttacccattcctgaagga | |
| | caaccgggaaaagatcgagaagatcctgaccttccgcatcccctactacg | |
| | tgggccctctggccaggggaaacagcagattcgcctggatgaccagaaag | |
| | agcgaggaaaccatcaccccctggaacttcgaggaagtggtggacaaggg | |
| | cgcttccgcccagagcttcatcgagcggatgaccaacttcgataagaacc | |
| | tgcccaacgagaaggtgctgcccaagcacagcctgctgtacgagtacttc | |
| | accgtgtataacgagctgaccaaagtgaaatacgtgaccgagggaatgag | |
| | aaagcccgccttcctgagcggcgagcagaaaaaggccatcgtggacctgc | |
| | tgttcaagaccaaccggaaagtgaccgtgaagcagctgaaagaggactac | |
| | ttcaagaaaatcgagtgcttcgactccgtggaaatctccggcgtggaaga | |
| | tcggttcaacgcctccctgggcacataccacgatctgctgaaaattatca | |
| | aggacaaggacttcctggacaatgaggaaaacgaggacattctggaagat | |
| | atcgtgctgaccctgacactgtttgaggacagagagatgatcgaggaacg | |
| | gctgaaaacctatgcccacctgttcgacgacaaagtgatgaagcagctga | |
| | agcggcggagatacaccggctggggcaggctgagccggaagctgatcaac | |
| | ggcatccgggacaagcagtccggcaagacaatcctggatttcctgaagtc | |
| | cgacggcttcgccaacagaaacttcatgcagctgatccacgacgacagcc | |
| | tgacctttaaagaggacatccagaaagcccaggtgtccggccagggcgat | |
| | agcctgcacgagcacattgccaatctggccggcagccccgccattaagaa | |
| | gggcatcctgcagacagtgaaggtggtggacgagctcgtgaaagtgatgg | |
| | gccggcacaagcccgagaacatcgtgatcgaaatggccagagagaaccag | |
| | accacccagaagggacagaagaacagccgcgagagaatgaagcggatcga | |
| | agagggcatcaaagagctgggcagccagatcctgaaagaacaccccgtgg | |
| | aaaacacccagctgcagaacgagaagctgtacctgtactacctgcagaat | |
| | gggcgggatatgtacgtggaccaggaactggacatcaaccggctgtccga | |
| | ctacgatgtggacgctatcgtgcctcagagctttctgaaggacgactcca | |
| | tcgacaacaaggtgctgaccagaagcgacaagaaccggggcaagagcgac | |
| | aacgtgccctccgaagaggtcgtgaagaagatgaagaactactggcggca | |
| | gctgctgaacgccaagctgattacccagagaaagttcgacaatctgacca | |
| | aggccgagagaggcggcctgagcgaactggataaggccggcttcatcaag | |
| | agacagctggtggaaacccggcagatcacaaagcacgtggcacagatcct | |
| | ggactcccggatgaacactaagtacgacgagaatgacaagctgatccggg | |
| | aagtgaaagtgatcaccctgaagtccaagctggtgtccgatttccggaag | |
| | gatttccagttttacaaagtgcgcgagatcaacaactaccaccacgccca | |
| | cgacgcctacctgaacgccgtcgtgggaaccgccctgatcaaaaagtacc | |
| | ctaagctggaaagcgagttcgtgtacggcgactacaaggtgtacgacgtg | |
| | cggaagatgatcgccaagagcgagcaggaaatcggcaaggctaccgccaa | |
| | gtacttcttctacagcaacatcatgaactttttcaagaccgagattaccc | |
| | tggccaacggcgagatccggaagcggcctctgatcgagacaaacggcgaa | |
| | accggggagatcgtgtgggataagggccgggattttgccaccgtgcggaa | |
| | agtgctgagcatgccccaagtgaatatcgtgaaaaagaccgaggtgcaga | |
| | caggcggcttcagcaaagagtctatcctgcccaagaggaacagcgataag | |
| | ctgatcgccagaaagaaggactgggaccctaagaagtacggcggcttcga | |
| | cagccccaccgtggcctattctgtgctggtggtggccaaagtggaaaagg | |
| | gcaagtccaagaaactgaagagtgtgaaagagctgctggggatcaccatc | |
| | atggaaagaagcagcttcgagaagaatcccatcgactttctggaagccaa | |
| | gggctacaaagaagtgaaaaaggacctgatcatcaagctgcctaagtact | |
| | ccctgttcgagctggaaaacggccggaagagaatgctggcctctgccggc | |
| | gaactgcagaagggaaacgaactggccctgccctccaaatatgtgaactt | |
| | cctgtacctggccagccactatgagaagctgaagggctcccccgaggata | |
| | atgagcagaaacagctgtttgtggaacagcacaagcactacctggacgag | |
| | atcatcgagcagatcagcgagttctccaagagagtgatcctggccgacgc | |
| | taatctggacaaagtgctgtccgcctacaacaagcaccgggataagccca | |
| | tcagagagcaggccgagaatatcatccacctgtttaccctgaccaatctg | |
| | ggagcccctgccgccttcaagtactttgacaccaccatcgaccggaagag | |
| | gtacaccagcaccaaagaggtgctggacgccaccctgatccaccagagca | |
| | tcaccggcctgtacgagacacggatcgacctgtctcagctgggaggtgac | |
| | aaacggacagccgacggaagcgagttcgagtcaccaaagaagaagcggaa | |
| | agtctaa | |
| hLIG4(1- | ATGGCAGCTTCTCAGACCTCTCAAACAGTAGCCTCTCATGTACCGTTCGC | 153 |
| 620)- | TGACTTGTGTTCTACGCTCGAACGCATCCAGAAATCAAAGGGGCGCGCCG | |
| nCas9 | AGAAAATCCGGCACTTCAGAGAATTCTTGGATTCCTGGAGGAAGTTTCAT | |
| | GATGCTCTCCACAAAAATCACAAAGATGTAACGGATAGTTTCTACCCTGC | |
| | TATGAGACTTATACTGCCGCAGCTTGAGAGGGAACGCATGGCGTATGGTA | |
| | TAAAGGAGACAATGTTGGCGAAATTGTATATTGAGCTGCTGAACTTGCCA | |
| | AGAGATGGAAAGGACGCGCTCAAACTGCTGAACTATAGAACACCCACGGG | |
| | TACCCATGGTGACGCCGGTGACTTTGCCATGATCGCCTATTTCGTACTGA | |
| | AACCTCGATGTCTTCAAAAAGGTTCTCTTACAATTCAGCAAGTCAACGAC | |
| | CTGCTGGATTCAATTGCGAGTAACAACAGCGCTAAGCGAAAGGATCTCAT | |
| | TAAGAAAAGCCTCCTGCAGCTGATAACTCAGTCCTCTGCACTCGAACAAA | |
| | AATGGCTGATTCGGATGATTATCAAGGATTTGAAGTTGGGGGTATCTCAG | |
| | CAAACTATTTTCAGCGTGTTTCACAATGATGCAGCAGAATTGCATAATGT | |
| | CACAACAGATCTTGAGAAAGTCTGCCGACAGTTGCACGACCCCTCTGTAG | |
| | GCTTGAGTGACATATCTATAACACTTTTTTCTGCGTTCAAACCCATGTTG | |
| | GCTGCTATTGCGGACATAGAACACATCGAGAAAGACATGAAACATCAGTC | |
| | ATTCTATATAGAGACTAAATTGGACGGCGAGAGGATGCAAATGCACAAAG | |
| | ATGGTGATGTGTATAAATATTTTTCCCGCAACGGCTACAACTACACTGAT | |
| | CAATTCGGAGCGTCCCCAACTGAAGGGTCCCTCACTCCTTTCATACACAA | |
| | TGCGTTTAAGGCCGATATTCAGATATGTATCCTCGACGGCGAAATGATGG | |
| | CGTACAATCCCAATACCCAGACCTTCATGCAAAAGGGAACGAAGTTCGAT | |
| | ATTAAACGGATGGTTGAAGATTCCGACCTCCAAACATGTTACTGTGTGTT | |
| | TGATGTCCTGATGGTGAATAACAAAAAACTCGGCCATGAAACCCTTCGAA | |
| | AGCGATACGAAATACTCAGCAGTATATTTACTCCAATACCAGGCCGAATC | |
| | GAGATCGTACAGAAAACACAAGCCCATACTAAGAATGAAGTTATTGATGC | |
| | ACTGAACGAAGCCATAGACAAGAGGGAAGAAGGCATAATGGTCAAGCAGC | |
| | CTCTGAGTATATATAAACCTGACAAAAGGGGGGAAGGATGGCTGAAGATA | |
| | AAGCCAGAATACGTGTCTGGTCTTATGGACGAATTGGACATTCTCATCGT | |
| | CGGAGGATATTGGGGTAAGGGTTCCAGGGGGGGGATGATGTCCCACTTTC | |
| | TGTGTGCGGTTGCCGAGAAACCGCCCCCAGGGGAAAAACCATCAGTGTTC | |
| | CATACGTTGTCACGCGTCGGCTCAGGTTGTACGATGAAGGAACTTTACGA | |
| | TCTGGGGTTGAAACTCGCCAAATATTGGAAGCCATTCCATCGGAAAGCAC | |
| | CGCCCTCTAGTATCTTGTGTGGGACGGAGAAGCCAGAAGTTTATATAGAG | |
| | CCATGTAACTCAGTAATTGTTCAAATCAAAGCCGCAGAGATCGTCCCGTC | |
| | AGACATGTACAAGACTGGATGCACCCTTAGATTTCCTCGCATCGAAAAAA | |
| | TAAGAGATGATAAAGAGTGGCATGAGTGCATGACTCTTGACGACCTTGAA | |
| | CAGCTCCGCGGGAAGGCCAGCGGTAAACTGGCTAGTAAGCACCTCTACAT | |
| | CGGGGGTGACTCCGGTGGCTCCTCAGGGGGATCTAAACGCACGGCCGATG | |
| | GGTCCGAGTTTGAGTCTCCCAAGAAGAAAAGGAAAGTGAGTGGTGGAAGT | |
| | AGCGGCGGTAGCgacaagaagtacagcatcggcctggacatcggcaccaa | |
| | ctctgtgggctgggccgtgatcaccgacgagtacaaggtgcccagcaaga | |
| | aattcaaggtgctgggcaacaccgaccggcacagcatcaagaagaacctg | |
| | atcggagccctgctgttcgacagcggcgaaacagccgaggccacccggct | |
| | gaagagaaccgccagaagaagatacaccagacggaagaaccggatctgct | |
| | atctgcaagagatcttcagcaacgagatggccaaggtggacgacagcttc | |
| | ttccacagactggaagagtccttcctggtggaagaggataagaagcacga | |
| | gcggcaccccatcttcggcaacatcgtggacgaggtggcctaccacgaga | |
| | agtaccccaccatctaccacctgagaaagaaactggtggacagcaccgac | |
| | aaggccgacctgcggctgatctatctggccctggcccacatgatcaagtt | |
| | ccggggccacttcctgatcgagggcgacctgaaccccgacaacagcgacg | |
| | tggacaagctgttcatccagctggtgcagacctacaaccagctgttcgag | |
| | gaaaaccccatcaacgccagcggcgtggacgccaaggccatcctgtctgc | |
| | cagactgagcaagagcagacggctggaaaatctgatcgcccagctgcccg | |
| | gcgagaagaagaatggcctgttcggaaacctgattgccctgagcctgggc | |
| | ctgacccccaacttcaagagcaacttcgacctggccgaggatgccaaact | |
| | gcagctgagcaaggacacctacgacgacgacctggacaacctgctggccc | |
| | agatcggcgaccagtacgccgacctgtttctggccgccaagaacctgtcc | |
| | gacgccatcctgctgagcgacatcctgagagtgaacaccgagatcaccaa | |
| | ggcccccctgagcgcctctatgatcaagagatacgacgagcaccaccagg | |
| | acctgaccctgctgaaagctctcgtgcggcagcagctgcctgagaagtac | |
| | aaagagattttcttcgaccagagcaagaacggctacgccggctacattga | |
| | cggcggagccagccaggaagagttctacaagttcatcaagcccatcctgg | |
| | aaaagatggacggcaccgaggaactgctcgtgaagctgaacagagaggac | |
| | ctgctgcggaagcagcggaccttcgacaacggcagcatcccccaccagat | |
| | ccacctgggagagctgcacgccattctgcggcggcaggaagatttttacc | |
| | cattcctgaaggacaaccgggaaaagatcgagaagatcctgaccttccgc | |
| | atcccctactacgtgggccctctggccaggggaaacagcagattcgcctg | |
| | gatgaccagaaagagcgaggaaaccatcaccccctggaacttcgaggaag | |
| | tggtggacaagggcgcttccgcccagagcttcatcgagcggatgaccaac | |
| | ttcgataagaacctgcccaacgagaaggtgctgcccaagcacagcctgct | |
| | gtacgagtacttcaccgtgtataacgagctgaccaaagtgaaatacgtga | |
| | ccgagggaatgagaaagcccgccttcctgagcggcgagcagaaaaaggcc | |
| | atcgtggacctgctgttcaagaccaaccggaaagtgaccgtgaagcagct | |
| | gaaagaggactacttcaagaaaatcgagtgcttcgactccgtggaaatct | |
| | ccggcgtggaagatcggttcaacgcctccctgggcacataccacgatctg | |
| | ctgaaaattatcaaggacaaggacttcctggacaatgaggaaaacgagga | |
| | cattctggaagatatcgtgctgaccctgacactgtttgaggacagagaga | |
| | tgatcgaggaacggctgaaaacctatgcccacctgttcgacgacaaagtg | |
| | atgaagcagctgaagcggcggagatacaccggctggggcaggctgagccg | |
| | gaagctgatcaacggcatccgggacaagcagtccggcaagacaatcctgg | |
| | atttcctgaagtccgacggcttcgccaacagaaacttcatgcagctgatc | |
| | cacgacgacagcctgacctttaaagaggacatccagaaagcccaggtgtc | |
| | cggccagggcgatagcctgcacgagcacattgccaatctggccggcagcc | |
| | ccgccattaagaagggcatcctgcagacagtgaaggtggtggacgagctc | |
| | gtgaaagtgatgggccggcacaagcccgagaacatcgtgatcgaaatggc | |
| | cagagagaaccagaccacccagaagggacagaagaacagccgcgagagaa | |
| | tgaagcggatcgaagagggcatcaaagagctgggcagccagatcctgaaa | |
| | gaacaccccgtggaaaacacccagctgcagaacgagaagctgtacctgta | |
| | ctacctgcagaatgggcgggatatgtacgtggaccaggaactggacatca | |
| | accggctgtccgactacgatgtggacgctatcgtgcctcagagctttctg | |
| | aaggacgactccatcgacaacaaggtgctgaccagaagcgacaagaaccg | |
| | gggcaagagcgacaacgtgccctccgaagaggtcgtgaagaagatgaaga | |
| | actactggcggcagctgctgaacgccaagctgattacccagagaaagttc | |
| | gacaatctgaccaaggccgagagaggcggcctgagcgaactggataaggc | |
| | cggcttcatcaagagacagctggtggaaacccggcagatcacaaagcacg | |
| | tggcacagatcctggactcccggatgaacactaagtacgacgagaatgac | |
| | aagctgatccgggaagtgaaagtgatcaccctgaagtccaagctggtgtc | |
| | cgatttccggaaggatttccagttttacaaagtgcgcgagatcaacaact | |
| | accaccacgcccacgacgcctacctgaacgccgtcgtgggaaccgccctg | |
| | atcaaaaagtaccctaagctggaaagcgagttcgtgtacggcgactacaa | |
| | ggtgtacgacgtgcggaagatgatcgccaagagcgagcaggaaatcggca | |
| | aggctaccgccaagtacttcttctacagcaacatcatgaactttttcaag | |
| | accgagattaccctggccaacggcgagatccggaagcggcctctgatcga | |
| | gacaaacggcgaaaccggggagatcgtgtgggataagggccgggattttg | |
| | ccaccgtgcggaaagtgctgagcatgccccaagtgaatatcgtgaaaaag | |
| | accgaggtgcagacaggcggcttcagcaaagagtctatcctgcccaagag | |
| | gaacagcgataagctgatcgccagaaagaaggactgggaccctaagaagt | |
| | acggcggcttcgacagccccaccgtggcctattctgtgctggtggtggcc | |
| | aaagtggaaaagggcaagtccaagaaactgaagagtgtgaaagagctgct | |
| | ggggatcaccatcatggaaagaagcagcttcgagaagaatcccatcgact | |
| | ttctggaagccaagggctacaaagaagtgaaaaaggacctgatcatcaag | |
| | ctgcctaagtactccctgttcgagctggaaaacggccggaagagaatgct | |
| | ggcctctgccggcgaactgcagaagggaaacgaactggccctgccctcca | |
| | aatatgtgaacttcctgtacctggccagccactatgagaagctgaagggc | |
| | tcccccgaggataatgagcagaaacagctgtttgtggaacagcacaagca | |
| | ctacctggacgagatcatcgagcagatcagcgagttctccaagagagtga | |
| | tcctggccgacgctaatctggacaaagtgctgtccgcctacaacaagcac | |
| | cgggataagcccatcagagagcaggccgagaatatcatccacctgtttac | |
| | cctgaccaatctgggagcccctgccgccttcaagtactttgacaccacca | |
| | tcgaccggaagaggtacaccagcaccaaagaggtgctggacgccaccctg | |
| | atccaccagagcatcaccggcctgtacgagacacggatcgacctgtctca | |
| | gctgggaggtgacaaacggacagccgacggaagcgagttcgagtcaccaa | |
| | agaagaagcggaaagtctaa | |

Disclosed herein are protein complexes comprising: an RNA-guided endonuclease bound to a ligase. The endonuclease and the ligase may be bound together through heterodimerization domains. The heterodimerization domains may include one or more of leucine zippers, PDZ domains, streptavidin and streptavidin binding protein, foldon domains, hydrophobic polypeptides, an antibody that binds the Cas nickase, or an antibody that binds the ligase, or one or more binding fragments thereof.

In some aspects, the system comprises at least one donor strand. In some aspects, the donor strand comprises a nucleic acid sequence that is at least partially homologous to the genomic locus targeted by the at least one guide nucleic acid. In some aspects, the donor strand comprises a nucleic acid sequence that is not homologous to the genomic locus targeted by the at least one guide nucleic acid. In some aspects, the donor strand is a single-stranded or a double-stranded nucleic acid. In some aspects, the donor strand comprising double-stranded nucleic acid comprises at least one overhang. In some aspects, the overhang comprises a guide binding site that is at least partially complementary to a guide nucleic acid. In some aspects, the overhang comprises a genomic flap binding site that is at least partially identical or complementary to a genomic flap at or adjacent to the genomic locus. In some aspects, the donor strand comprises two overhangs, where the first overhang: comprises a first guide binding site that is at least partially complementary to a first guide nucleic acid; or a first genomic flap binding site that is at least partially identical or complementary to a first genomic flap at or adjacent to the genomic locus; and the second overhang: comprises a second guide binding site that is at least partially complementary to a second guide nucleic acid; or a second genomic flap binding site that is at least partially identical or complementary to a second genomic flap at or adjacent to the genomic locus. In some aspects, the donor strand corrects at least one genetic mutation in the at least one genomic locus. In some aspects, the donor strand comprises a coding sequence. In some aspects, the coding sequence encodes a full length protein or a fragment thereof. In some aspects, the donor strand comprises a non-coding sequence. In some aspects, the non-coding sequence knocks out an endogenous gene. In some aspects, the non-coding sequence comprises a regulatory element.

In some aspects, the system comprises a nuclease. The nuclease may be heterologous. In some aspects, the nuclease comprises an exonuclease for digesting the genomic flap. In some aspects, the exonuclease is a 5′ exonuclease. Non-limiting example of the exonuclease can include a human flap endonuclease 1 (hFEN1), a human exonuclease 5 (hEXO5), a T5 exonuclease, a T7 exonuclease, an exonuclease VIII, a flap endonuclease domain of E. coli Poll, a RecJF, a Lambda exonuclease, a Xni (ExoIXI), a SaFEN (Staphylococcus aureus FEN), a nuclease BAL-31, or a fragment thereof. In some aspects, the exonuclease comprises an exonuclease in Table 10. In some aspects, the exonuclease comprises a polypeptide sequence at least 70%, at least 75%, at least 80%, at least 85%, at least 90%, at least 95%, at least 99%, or more identical to the polypeptide sequence of any one of the exonuclease in Table 10.

TABLE 10
Non-limiting examples of exonuclease polypeptide sequence

| | | SEQ |
|---|---|---|
| | | ID |
| Name | Exonuclease polypeptide sequence | NO: |
| hFEN1 | MGIQGLAKLIADVAPSAIRENDIKSYFGRKVAIDASMSIYQFLIAVRQGG | 212 |
| | DVLQNEEGETTSHLMGMFYRTIRMMENGIKPVYVFDGKPPQLKSGELA | |
| | KRSERRAEAEKQLQQAQAAGAEQEVEKFTKRLVKVTKQHNDECKHLLS | |
| | LMGIPYLDAPSEAEASCAALVKAGKVYAAATEDMDCLTFGSPVLMRHL | |
| | TASEAKKLPIQEFHLSRILQELGLNQEQFVDLCILLGSDYCESIRGIGPKRA | |
| | VDLIQKHKSIEEIVRRLDPNKYPVPENWLHKEAHQLFLEPEVLDPESVEL | |
| | KWSEPNEEELIKFMCGEKQFSEERIRSGVKRLSKSRQGSTQGRLDDFFKV | |
| | TGSLSSAKRKEPEPKGSTKKKAKTGAAGKFKRGK | |
| hFen1 | MGIQGLAKLIADVAPSAIRENDIKSYFGRKVAIDASMSIYQFLIAVRQGG | 213 |
| (1-333) | DVLQNEEGETTSHLMGMFYRTIRMMENGIKPVYVFDGKPPQLKSGELA | |
| | KRSERRAEAEKQLQQAQAAGAEQEVEKFTKRLVKVTKQHNDECKHLLS | |
| | LMGIPYLDAPSEAEASCAALVKAGKVYAAATEDMDCLTFGSPVLMRHL | |
| | TASEAKKLPIQEFHLSRILQELGLNQEQFVDLCILLGSDYCESIRGIGPKRA | |
| | VDLIQKHKSIEEIVRRLDPNKYPVPENWLHKEAHQLFLEPEVLDPESVEL | |
| | KWSEPNEEELIKFMCGEKQFSEERIRSGVKRLSKSRQ | |
| hEXO5 | MAETREEETVSAEASGFSDLSDSEFLEFLDLEDAQESKALVNMPGPSSES | 214 |
| | LGKDDKPISLQNWKRGLDILSPMERFHLKYLYVTDLATQNWCELQTAY | |
| | GKELPGFLAPEKAAVLDTGASIHLARELELHDLVTVPVTTKEDAWAIKF | |
| | LNILLLIPTLQSEGHIREFPVFGEGEGVLLVGVIDELHYTAKGELELAELK | |
| | TRRRPMLPLEAQKKKDCFQVSLYKYIFDAMVQGKVTPASLIHHTKLCLE | |
| | KPLGPSVLRHAQQGGFSVKSLGDLMELVFLSLTLSDLPVIDILKIEYIHQE | |
| | TATVLGTEIVAFKEKEVRAKVQHYMAYWMGHREPQGVDVEEAWKCR | |
| | TCTYADICEWRKGSGVLSSTLAPQVKKAK | |
| T5 EXO | MSKSWGKFIEEEEAEMASRRNLMIVDGTNLGFRFKHNNSKKPFASSYVS | 215 |
| | TIQSLAKSYSARTTIVLGDKGKSVFRLEHLPEYKGNRDEKYAQRTEEEK | |
| | ALDEQFFEYLKDAFELCKTTFPTFTIRGVEADDMAAYIVKLIGHLYDHV | |
| | WLISTDGDWDTLLTDKVSRFSFTTRREYHLRDMYEHHNVDDVEQFISLK | |
| | AIMGDLGDNIRGVEGIGAKRGYNIIREFGNVLDIIDQLPLPGKQKYIQNLN | |
| | ASEELLFRNLILVDLPTYCVDAIAAVGQDVLDKFTKDILEIAEQ | |
| T7 EXO | MALLDLKQFYELREGCDDKGILVMDGDWLVFQAMSAAEFDASWEEEI | 216 |
| | WHRCCDHAKARQILEDSIKSYETRKKAWAGAPIVLAFTDSVNWRKELV | |
| | DPNYKANRKAVKKPVGYFEFLDALFEREEFYCIREPMLEGDDVMGVIAS | |
| | NPSAFGARKAVIISCDKDFKTIPNCDFLWCTTGNILTQTEESADWWHLFQ | |
| | TIKGDITDGYSGIAGWGDTAEDFLNNPFITEPKTSVLKSGKNKGQEVTK | |
| | WVKRDPEPHETLWDCIKSIGAKAGMTEEDIIKQGQMARILRFNEYNFIDK | |
| | EIYLWRP | |
| EXO VIII | MSTKPLFLLRKAKKSSGEPDVVLWASNDFESTCATLDYLIVKSGKKLSS | 217 |
| (RecE) | YFKAVATNFPVVNDLPAEGEIDFTWSERYQLSKDSMTWELKPGAAPDN | |
| | AHYQGNTNVNGEDMTEIEENMLLPISGQELPIRWLAQHGSEKPVTHVSR | |
| | DGLQALHIARAEELPAVTALAVSHKTSLLDPLEIRELHKLVRDTDKVFPN | |
| | PGNSNLGLITAFFEAYLNADYTDRGLLTKEWMKGNRVSHITRTASGANA | |
| | GGGNLTDRGEGFVHDLTSLARDVATGVLARSMDLDIYNLHPAHAKRIE | |
| | EIIAENKPPFSVFRDKFITMPGGLDYSRAIVVASVKEAPIGIEVIPAHVTEY | |
| | LNKVLTETDHANPDPEIVDIACGRSSAPMPQRVTEEGKQDDEEKPQPSGT | |
| | TAVEQGEAETMEPDATEHHQDTQPLDAQSQVNSVDAKYQELRAELHEA | |
| | RKNIPSKNPVDDDKLLAASRGEFVDGISDPNDPKWVKGIQTRDCVYQNQ | |
| | PETEKTSPDMNQPEPVVQQEPEIACNACGQTGGDNCPDCGAVMGDATY | |
| | QETFDEESQVEAKENDPEEMEGAEHPHNENAGSDPHRDCSDETGEVAD | |
| | PVIVEDIEPGIYYGISNENYHAGPGISKSQLDDIADTPALYLWRKNAPVDT | |
| | TKTKTLDLGTAFHCRVLEPEEFSNRFIVAPEFNRRTNAGKEEEKAFLMEC | |
| | ASTGKTVITAEEGRKIELMYQSVMALPLGQWLVESAGHAESSIYWEDPE | |
| | TGILCRCRPDKIIPEFHWIMDVKTTADIQRFKTAYYDYRYHVQDAFYSD | |
| | GYEAQFGVQPTFVFLVASTTIECGRYPVEIFMMGEEAKLAGQQEYHRNL | |
| | RTLSDCLNTDEWPAIKTLSLPRWAKEYAND | |
| EXO VIII, | EHPHNENAGSDPHRDCSDETGEVADPVIVEDIEPGIYYGISNENYHAGPG | 218 |
| truncated | ISKSQLDDIADTPALYLWRKNAPVDTTKTKTLDLGTAFHCRVLEPEEFSN | |
| | RFIVAPEFNRRTNAGKEEEKAFLMECASTGKTVITAEEGRKIELMYQSV | |
| | MALPLGQWLVESAGHAESSIYWEDPETGILCRCRPDKIIPEFHWIMDVKT | |
| | TADIQRFKTAYYDYRYHVQDAFYSDGYEAQFGVQPTFVFLVASTTIECG | |
| | RYPVEIFMMGEEAKLAGQQEYHRNLRTLSDCLNTDEWPAIKTLSLPRW | |
| | AKEYAND | |
| Flap endo | VQIPQNPLILVDGSSYLYRAYHAFPPLTNSAGEPTGAMYGVLNMLRSLI | 219 |
| domain of | MQYKPTHAAVVFDAKGKTFRDELFEHYKSHRPPMPDDLRAQIEPLHAM | |
| E coli | VKAMGLPLLAVSGVEADDVIGTLAREAEKAGRPVLISTGDKDMAQLVT | |
| PolI | PNITLINTMTNTILGPEEVVNKYGVPPELIIDFLALMGDSSDNIPGVPGVG | |
| | EKTAQALLQGLGGLDTLYAEPEKIAGLSFRGAKTMAAKLEQNKEVAYL | |
| | SYQLATIKTDVELELTCEQLEVQQPAAEELLGLFKKYEFKRWTADVEAG | |
| | KWLQAKGAKPAAKPQETSVADEAPEVTATVI | |
| RecJ | MKQQIQLRRREVDETADLPAELPPLLRRLYASRGVRSAQELERSVKGML | 220 |
| | PWQQLSGVEKAVEILYNAFREGTRIIVVGDFDADGATSTALSVLAMRSL | |
| | GCSNIDYLVPNRFEDGYGLSPEVVDQAHARGAQLIVTVDNGISSHAGVE | |
| | HARSLGIPVIVTDHHLPGDTLPAAEAIINPNLRDCNFPSKSLAGVGVAFYL | |
| | MLALRTFLRDQGWFDERNIAIPNLAELLDLVALGTVADVVPLDANNRIL | |
| | TWQGMSRIRAGKCRPGIKALLEVANRDAQKLAASDLGFALGPRLNAAG | |
| | RLDDMSVGVALLLCDNIGEARVLANELDALNQTRKEIEQGMQIEALTLC | |
| | EKLERSRDTLPGGLAMYHPEWHQGVVGILASRIKERFHRPVIAFAPAGD | |
| | GTLKGSGRSIQGLHMRDALERLDTLYPGMMLKFGGHAMAAGLSLEEDK | |
| | FKLFQQRFGELVTEWLDPSLLQGEVVSDGPLSPAEMTMEVAQLLRDAGP | |
| | WGQMFPEPLFDGHFRLLQQRLVGERHLKVMVEPVGGGPLLDGIAFNVD | |
| | TALWPDNGVREVQLAYKLDINEFRGNRSLQIIIDNIWPI | |
| Lambda exo | MTPDIILQRTGIDVRAVEQGDDAWHKLRLGVITASEVHNVIAKPRSGKK | 154 |
| | WPDMKMSYFHTLLAEVCTGVAPEVNAKALAWGKQYENDARTLFEFTS | |
| | GVNVTESPIIYRDESMRTACSPDGLCSDGNGLELKCPFTSRDFMKFRLGG | |
| | FEAIKSAYMAQVQYSMWVTRKNAWYFANYDPRMKREGLHYVVIERDE | |
| | KYMASFDEIVPEFIEKMDEALAEIGFVFGEQWR | |
| Xni | MAVHLLIVDALNLIRRIHAVQGSPCVETCQHALDQLIMHSQPTHAVAVF | 155 |
| (ExoIXI) | DDENRSSGWRHQRLPDYKAGRPPMPEELHDEMPALRAAFEQRGVPCWS | |
| from | TSGNEADDLAATLAVKVTQAGHQATIVSTDKGYCQLLSPTLRIRDYFQK | |
| E coli | RWLDAPFIDKEFGVQPQQLPDYWGLAGISSSKVPGVAGIGPKSATQLLV | |
| | EFQSLEGIYENLDAVAEKWRKKLETHKEMAFLCRDIARLQTDLHIDGNL | |
| | QQLRLVR | |
| SaFEN | MPNKILLVDGMALLFRHFYATSLHKQFMYNSQGVPTNGIQGFVRHIFSAI | 156 |
| (Staphaureus) | HEIRPTHVAVCWDMGQSTFRNDMFDGYKQNRSAPPEELIPQFDYVKEIS | |
| | EQFGFVNIGVKNYEADDVIGTLAQQYSTDNDVYIITGDKDLLQCINDNV | |
| | EVWLIKKGFNIYNRYTLHRFNEEYALEPQQLIDIKAFMGDTADGYAGVK | |
| | GIGEKTAIKLIQQYQSVENVVENIDALSAGQRNKINDNLDELYLSKRLAE | |
| | IHTQVPIDSEALFEKMSFATTLNHILSICNEHELHVSGKYISSHF | |

In some aspects, the system comprises at least one additional endonuclease that is different from the at least one programmable endonuclease described herein. In some aspects, the at least one additional endonuclease can digest the genomic flap.

In some aspects, the system comprises a dominant negative MMR peptide to improve genomic editing capability, particularly in cells which overexpress the MMR pathway. In some aspects, the dominant negative MMR peptide can be delivered as a fusion (e.g., fused with any component of the system described herein), recruited, or as separate peptide. Table 11 lists non-limiting examples of the MMR peptide sequences.

TABLE 11
Non-limiting examples of MMR polypeptide sequence

| | | SEQ |
|---|---|---|
| | | ID |
| Name | MMR peptide sequence | NO: |
| MLH1 | MSFVAGVIRRLDETVVNRIAAGEVIQRPANAIKEMIENCLDAKSTSIQVIVKEGGLKLIQ | 157 |
| | IQDNGTGIRKEDLDIVCERFTTSKLQSFEDLASISTYGFRGEALASISHVAHVTITTKTAD | |
| | GKCAYRASYSDGKLKAPPKPCAGNQGTQITVEDLFYNIATRRKALKNPSEEYGKILEV | |
| | VGRYSVHNAGISFSVKKQGETVADVRTLPNASTVDNIRSIFGNAVSRELIEIGCEDKTLA | |
| | FKMNGYISNANYSVKKCIFLLFINHRLVESTSLRKAIETVYAAYLPKNTHPFLYLSLEISP | |
| | QNVDVNVHPTKHEVHFLHEESILERVQQHIESKLLGSNSSRMYFTQTLLPGLAGPSGEM | |
| | VKSTTSLTSSSTSGSSDKVYAHQMVRTDSREQKLDAFLQPLSKPLSSQPQAIVTEDKTDI | |
| | SSGRARQQDEEMLELPAPAEVAAKNQSLEGDTTKGTSEMSEKRGPTSSNPRKRHREDS | |
| | DVEMVEDDSRKEMTAACTPRRRIINLTSVLSLQEEINEQGHEVLREMLHNHSFVGCVN | |
| | PQWALAQHQTKLYLLNTTKLSEELFYQILIYDFANFGVLRLSEPAPLFDLAMLALDSPE | |
| | SGWTEEDGPKEGLAEYIVEFLKKKAEMLADYFSLEIDEEGNLIGLPLLIDNYVPPLEGLP | |
| | IFILRLATEVNWDEEKECFESLSKECAMFYSIRKQYISEESTLSGQQSEVPGSIPNSWKW | |
| | TVEHIVYKALRSHILPPKHFTEDGNILQLANLPDLYKVFERC | |
| MLH1 | MSFVAGVIRRLDETVVNRIAAGEVIQRPANAIKAMIENCLDAKSTSIQVIVKEGGLKLIQ | 158 |
| E34A | IQDNGTGIRKEDLDIVCERFTTSKLQSFEDLASISTYGFRGEALASISHVAHVTITTKTAD | |
| | GKCAYRASYSDGKLKAPPKPCAGNQGTQITVEDLFYNIATRRKALKNPSEEYGKILEV | |
| | VGRYSVHNAGISFSVKKQGETVADVRTLPNASTVDNIRSIFGNAVSRELIEIGCEDKTLA | |
| | FKMNGYISNANYSVKKCIFLLFINHRLVESTSLRKAIETVYAAYLPKNTHPFLYLSLEISP | |
| | QNVDVNVHPTKHEVHFLHEESILERVQQHIESKLLGSNSSRMYFTQTLLPGLAGPSGEM | |
| | VKSTTSLTSSSTSGSSDKVYAHQMVRTDSREQKLDAFLQPLSKPLSSQPQAIVTEDKTDI | |
| | SSGRARQQDEEMLELPAPAEVAAKNQSLEGDTTKGTSEMSEKRGPTSSNPRKRHREDS | |
| | DVEMVEDDSRKEMTAACTPRRRIINLTSVLSLQEEINEQGHEVLREMLHNHSFVGCVN | |
| | PQWALAQHQTKLYLLNTTKLSEELFYQILIYDFANFGVLRLSEPAPLFDLAMLALDSPE | |
| | SGWTEEDGPKEGLAEYIVEFLKKKAEMLADYFSLEIDEEGNLIGLPLLIDNYVPPLEGLP | |
| | IFILRLATEVNWDEEKECFESLSKECAMFYSIRKQYISEESTLSGQQSEVPGSIPNSWKW | |
| | TVEHIVYKALRSHILPPKHFTEDGNILQLANLPDLYKVFERC | |
| MLH1 | MSFVAGVIRRLDETVVNRIAAGEVIQRPANAIKEMIENCLDAKSTSIQVIVKEGGLKLIQ | 159 |
| del | IQDNGTGIRKEDLDIVCERFTTSKLQSFEDLASISTYGFRGEALASISHVAHVTITTKTAD | |
| 754- | GKCAYRASYSDGKLKAPPKPCAGNQGTQITVEDLFYNIATRRKALKNPSEEYGKILEV | |
| 756 | VGRYSVHNAGISFSVKKQGETVADVRTLPNASTVDNIRSIFGNAVSRELIEIGCEDKTLA | |
| | FKMNGYISNANYSVKKCIFLLFINHRLVESTSLRKAIETVYAAYLPKNTHPFLYLSLEISP | |
| | QNVDVNVHPTKHEVHFLHEESILERVQQHIESKLLGSNSSRMYFTQTLLPGLAGPSGEM | |
| | VKSTTSLTSSSTSGSSDKVYAHQMVRTDSREQKLDAFLQPLSKPLSSQPQAIVTEDKTDI | |
| | SSGRARQQDEEMLELPAPAEVAAKNQSLEGDTTKGTSEMSEKRGPTSSNPRKRHREDS | |
| | DVEMVEDDSRKEMTAACTPRRRIINLTSVLSLQEEINEQGHEVLREMLHNHSFVGCVN | |
| | PQWALAQHQTKLYLLNTTKLSEELFYQILIYDFANFGVLRLSEPAPLFDLAMLALDSPE | |
| | SGWTEEDGPKEGLAEYIVEFLKKKAEMLADYFSLEIDEEGNLIGLPLLIDNYVPPLEGLP | |
| | IFILRLATEVNWDEEKECFESLSKECAMFYSIRKQYISEESTLSGQQSEVPGSIPNSWKW | |
| | TVEHIVYKALRSHILPPKHFTEDGNILQLANLPDLYKVF | |
| MLH1 | MSFVAGVIRRLDETVVNRIAAGEVIQRPANAIKAMIENCLDAKSTSIQVIVKEGGLKLIQ | 160 |
| E34A | IQDNGTGIRKEDLDIVCERFTTSKLQSFEDLASISTYGFRGEALASISHVAHVTITTKTAD | |
| del | GKCAYRASYSDGKLKAPPKPCAGNQGTQITVEDLFYNIATRRKALKNPSEEYGKILEV | |
| 754- | VGRYSVHNAGISFSVKKQGETVADVRTLPNASTVDNIRSIFGNAVSRELIEIGCEDKTLA | |
| 756 | FKMNGYISNANYSVKKCIFLLFINHRLVESTSLRKAIETVYAAYLPKNTHPFLYLSLEISP | |
| | QNVDVNVHPTKHEVHFLHEESILERVQQHIESKLLGSNSSRMYFTQTLLPGLAGPSGEM | |
| | VKSTTSLTSSSTSGSSDKVYAHQMVRTDSREQKLDAFLQPLSKPLSSQPQAIVTEDKTDI | |
| | SSGRARQQDEEMLELPAPAEVAAKNQSLEGDTTKGTSEMSEKRGPTSSNPRKRHREDS | |
| | DVEMVEDDSRKEMTAACTPRRRIINLTSVLSLQEEINEQGHEVLREMLHNHSFVGCVN | |
| | PQWALAQHQTKLYLLNTTKLSEELFYQILIYDFANFGVLRLSEPAPLFDLAMLALDSPE | |
| | SGWTEEDGPKEGLAEYIVEFLKKKAEMLADYFSLEIDEEGNLIGLPLLIDNYVPPLEGLP | |
| | IFILRLATEVNWDEEKECFESLSKECAMFYSIRKQYISEESTLSGQQSEVPGSIPNSWKW | |
| | TVEHIVYKALRSHILPPKHFTEDGNILQLANLPDLYKVF | |
| MLH1 | MSFVAGVIRRLDETVVNRIAAGEVIQRPANAIKEMIENCLDAKSTSIQVIVKEGGLKLIQ | 161 |
| 1-335 | IQDNGTGIRKEDLDIVCERFTTSKLQSFEDLASISTYGFRGEALASISHVAHVTITTKTAD | |
| | GKCAYRASYSDGKLKAPPKPCAGNQGTQITVEDLFYNIATRRKALKNPSEEYGKILEV | |
| | VGRYSVHNAGISFSVKKQGETVADVRTLPNASTVDNIRSIFGNAVSRELIEIGCEDKTLA | |
| | FKMNGYISNANYSVKKCIFLLFINHRLVESTSLRKAIETVYAAYLPKNTHPFLYLSLEISP | |
| | QNVDVNVHPTKHEVHFLHEESILERVQQHIESKLL | |
| MLH1 | MSFVAGVIRRLDETVVNRIAAGEVIQRPANAIKAMIENCLDAKSTSIQVIVKEGGLKLIQ | 162 |
| 1-335 | IQDNGTGIRKEDLDIVCERFTTSKLQSFEDLASISTYGFRGEALASISHVAHVTITTKTAD | |
| E34A | GKCAYRASYSDGKLKAPPKPCAGNQGTQITVEDLFYNIATRRKALKNPSEEYGKILEV | |
| | VGRYSVHNAGISFSVKKQGETVADVRTLPNASTVDNIRSIFGNAVSRELIEIGCEDKTLA | |
| | FKMNGYISNANYSVKKCIFLLFINHRLVESTSLRKAIETVYAAYLPKNTHPFLYLSLEISP | |
| | QNVDVNVHPTKHEVHFLHEESILERVQQHIESKLL | |
| MSH2 | MAVQPKETLQLESAAEVGFVRFFQGMPEKPTTTVRLFDRGDFYTAHGEDALLAAREV | 163 |
| | FKTQGVIKYMGPAGAKNLQSVVLSKMNFESFVKDLLLVRQYRVEVYKNRAGNKASK | |
| | ENDWYLAYKASPGNLSQFEDILFGNNDMSASIGVVGVKMSAVDGQRQVGVGYVDSIQ | |
| | RKLGLCEFPDNDQFSNLEALLIQIGPKECVLPGGETAGDMGKLRQIIQRGGILITERKKA | |
| | DFSTKDIYQDLNRLLKGKKGEQMNSAVLPEMENQVAVSSLSAVIKFLELLSDDSNFGQ | |
| | FELTTFDFSQYMKLDIAAVRALNLFQGSVEDTTGSQSLAALLNKCKTPQGQRLVNQWI | |
| | KQPLMDKNRIEERLNLVEAFVEDAELRQTLQEDLLRRFPDLNRLAKKFQRQAANLQD | |
| | CYRLYQGINQLPNVIQALEKHEGKHQKLLLAVFVTPLTDLRSDFSKFQEMIETTLDMD | |
| | QVENHEFLVKPSFDPNLSELREIMNDLEKKMQSTLISAARDLGLDPGKQIKLDSSAQFG | |
| | YYFRVTCKEEKVLRNNKNFSTVDIQKNGVKFTNSKLTSLNEEYTKNKTEYEEAQDAIV | |
| | KEIVNISSGYVEPMQTLNDVLAQLDAVVSFAHVSNGAPVPYVRPAILEKGQGRIILKAS | |
| | RHACVEVQDEIAFIPNDVYFEKDKQMFHIITGPNMGGKSTYIRQTGVIVLMAQIGCFVP | |
| | CESAEVSIVDCILARVGAGDSQLKGVSTFMAEMLETASILRSATKDSLIIIDELGRGTST | |
| | YDGFGLAWAISEYIATKIGAFCMFATHFHELTALANQIPTVNNLHVTALTTEETLTMLY | |
| | QVKKGVCDQSFGIHVAELANFPKHVIECAKQKALELEEFQYIGESQGYDIMEPAAKKC | |
| | YLEREQGEKIIQEFLSKVKQMPFTEMSEENITIKLKQLKAEVIAKNNSFVNEIISRIKVTT | |
| MSH2 | MAVQPKETLQLESAAEVGFVRFFQGMPEKPTTTVRLFDRGDFYTAHGEDALLAAREV | 164 |
| G674A | FKTQGVIKYMGPAGAKNLQSVVLSKMNFESFVKDLLLVRQYRVEVYKNRAGNKASK | |
| | ENDWYLAYKASPGNLSQFEDILFGNNDMSASIGVVGVKMSAVDGQRQVGVGYVDSIQ | |
| | RKLGLCEFPDNDQFSNLEALLIQIGPKECVLPGGETAGDMGKLRQIIQRGGILITERKKA | |
| | DFSTKDIYQDLNRLLKGKKGEQMNSAVLPEMENQVAVSSLSAVIKFLELLSDDSNFGQ | |
| | FELTTFDFSQYMKLDIAAVRALNLFQGSVEDTTGSQSLAALLNKCKTPQGQRLVNQWI | |
| | KQPLMDKNRIEERLNLVEAFVEDAELRQTLQEDLLRRFPDLNRLAKKFQRQAANLQD | |
| | CYRLYQGINQLPNVIQALEKHEGKHQKLLLAVFVTPLTDLRSDFSKFQEMIETTLDMD | |
| | QVENHEFLVKPSFDPNLSELREIMNDLEKKMQSTLISAARDLGLDPGKQIKLDSSAQFG | |
| | YYFRVTCKEEKVLRNNKNFSTVDIQKNGVKFTNSKLTSLNEEYTKNKTEYEEAQDAIV | |
| | KEIVNISSGYVEPMQTLNDVLAQLDAVVSFAHVSNGAPVPYVRPAILEKGQGRIILKAS | |
| | RHACVEVQDEIAFIPNDVYFEKDKQMFHIITGPNMGAKSTYIRQTGVIVLMAQIGCFVP | |
| | CESAEVSIVDCILARVGAGDSQLKGVSTFMAEMLETASILRSATKDSLIIIDELGRGTST | |
| | YDGFGLAWAISEYIATKIGAFCMFATHFHELTALANQIPTVNNLHVTALTTEETLTMLY | |
| | QVKKGVCDQSFGIHVAELANFPKHVIECAKQKALELEEFQYIGESQGYDIMEPAAKKC | |
| | YLEREQGEKIIQEFLSKVKQMPFTEMSEENITIKLKQLKAEVIAKNNSFVNEIISRIKVTT | |
| MSH2 | MAVQPKETLQLESAAEVGFVRFFQGMPEKPTTTVRLFDRGDFYTAHGEDALLAAREV | 165 |
| N671I | FKTQGVIKYMGPAGAKNLQSVVLSKMNFESFVKDLLLVRQYRVEVYKNRAGNKASK | |
| | ENDWYLAYKASPGNLSQFEDILFGNNDMSASIGVVGVKMSAVDGQRQVGVGYVDSIQ | |
| | RKLGLCEFPDNDQFSNLEALLIQIGPKECVLPGGETAGDMGKLRQIIQRGGILITERKKA | |
| | DFSTKDIYQDLNRLLKGKKGEQMNSAVLPEMENQVAVSSLSAVIKFLELLSDDSNFGQ | |
| | FELTTFDFSQYMKLDIAAVRALNLFQGSVEDTTGSQSLAALLNKCKTPQGQRLVNQWI | |
| | KQPLMDKNRIEERLNLVEAFVEDAELRQTLQEDLLRRFPDLNRLAKKFQRQAANLQD | |
| | CYRLYQGINQLPNVIQALEKHEGKHQKLLLAVFVTPLTDLRSDFSKFQEMIETTLDMD | |
| | QVENHEFLVKPSFDPNLSELREIMNDLEKKMQSTLISAARDLGLDPGKQIKLDSSAQFG | |
| | YYFRVTCKEEKVLRNNKNFSTVDIQKNGVKFTNSKLTSLNEEYTKNKTEYEEAQDAIV | |
| | KEIVNISSGYVEPMQTLNDVLAQLDAVVSFAHVSNGAPVPYVRPAILEKGQGRIILKAS | |
| | RHACVEVQDEIAFIPNDVYFEKDKQMFHIITGPIMGGKSTYIRQTGVIVLMAQIGCFVPC | |
| | ESAEVSIVDCILARVGAGDSQLKGVSTFMAEMLETASILRSATKDSLIIIDELGRGTSTY | |
| | DGFGLAWAISEYIATKIGAFCMFATHFHELTALANQIPTVNNLHVTALTTEETLTMLYQ | |
| | VKKGVCDQSFGIHVAELANFPKHVIECAKQKALELEEFQYIGESQGYDIMEPAAKKCY | |
| | LEREQGEKIIQEFLSKVKQMPFTEMSEENITIKLKQLKAEVIAKNNSFVNEIISRIKVTT | |

The system may relate to a 1-sided Replacer 1. Some aspects include a system comprising: (a) at least one RNA-guided endonuclease; (b) at least one guide nucleic acid comprising: (i) a spacer complementary to a genomic locus in a cell, (ii) a scaffold for complexing with the at least one RNA-guided endonuclease, (iii) an optional donor binding site that is at least partially complementary to an integrating nucleic acid, and (iv) a flap binding site that is at least partially identical or complementary to a genomic flap at or adjacent to the genomic locus; and (c) at least one DNA ligase; and (d) the integrating nucleic acid, optionally comprising a guide binding site that is at least partially complementary to the at least one guide nucleic acid, wherein the at least one RNA-guided endonuclease cleaves at least one strand of the genomic locus, and wherein the at least one DNA ligase ligates an end of the integrating nucleic acid to the genomic flap site, thereby replacing a region of the genomic locus with the integrating nucleic acid in the cell. The integrating nucleic acid may comprise a single-stranded DNA.

The system may relate to a 2-sided Replacer 1. Some aspects include a system comprising: (a) at least one RNA-guided endonuclease comprising a first RNA-guided endonuclease and an optional second RNA-guided endonuclease; (b) at least one guide nucleic acid comprising a first guide nucleic acid and a second guide nucleic acid, the first guide nucleic acid comprising: (i) a first spacer complementary to a first region of a genomic locus in a cell, (ii) a first scaffold for complexing with the first RNA-guided endonuclease, and (iii) an optional first donor binding site that at least partially complementary to an integrating nucleic acid, and (iv) a first flap binding site that is at least partially identical or complementary to a first genomic flap at or adjacent to the genomic locus; and the second guide nucleic acid comprising: (i) a second spacer complementary to a second region of the genomic locus in the cell, (ii) a second scaffold for complexing with the first or second RNA-guided endonuclease, (iii) an optional second donor binding site that at least partially complementary to the integrating nucleic acid, and (iv) a second flap binding site that is at least partially identical or complementary to a second genomic flap at or adjacent to the genomic locus; (c) at least one DNA ligase comprising a first DNA ligase and an optional second DNA ligase; and (d) at least one integrating nucleic acid comprising a first strand and a second strand: (i) wherein the first strand comprises an optional first guide binding site that is at least partially complementary to the first guide nucleic acid, and (ii) wherein the second strand comprises an optional second guide binding site that is at least partially complementary to the second guide nucleic acid, wherein the first RNA-guided endonuclease and/or the second RNA-guided endonuclease each cleaves at least one strand of the genomic locus in the cell; and wherein the first DNA ligase ligates an end of the first strand of the integrating nucleic acid to the first genomic flap; and the first or second DNA ligase ligates an end of the second strand of the integrating nucleic acid to the second genomic flap, thereby replacing a region of the genomic locus with the integrating nucleic acid in the cell. The integrating nucleic acid may comprise a double-stranded DNA duplex region. The integrating nucleic acid may comprise a 5′ overhang optionally comprising the first guide binding site. The integrating nucleic acid may comprise a 5′ overhang optionally comprising the second guide binding site.

The system may relate to 1-sided Replacer 2. Some aspects include a system comprising: (a) at least one RNA-guided endonuclease; (b) at least one guide nucleic acid comprising: (i) a spacer complementary to a genomic locus in a cell, (ii) a scaffold for complexing with the at least one RNA-guided endonuclease, and (iii) an optional donor binding site that is at least partially complementary to an integrating nucleic acid; (c) at least one DNA ligase; and (d) the integrating nucleic acid that: (i) comprises an optional guide binding site that is at least partially complementary to the at least one guide nucleic acid, and (ii) comprises a flap binding site that is at least partially identical or complementary to a genomic flap at or adjacent to the genomic locus, wherein the at least one RNA-guided endonuclease cleaves at least one strand of the genomic locus; and wherein the at least one DNA ligase ligates an end of the integrating nucleic acid to the genomic flap, thereby replacing a region of the genomic locus with the integrating nucleic acid in the cell. The integrating nucleic acid may comprise a DNA comprising a 3′ overhang. The 3′ overhang may comprise the guide binding site. The 3′ overhang may comprise the flap binding site. The at least one DNA ligase may ligates a strand of the integrating nucleic acid to the genomic nucleic acid sequence.

The system may relate to 2-sided Replacer 2. Some aspects include a system comprising: (a) at least one RNA-guided endonuclease comprising a first RNA-guided endonuclease and an optional second RNA-guided endonuclease; (b) at least one guide nucleic acid comprising a first guide nucleic acid and a second guide nucleic acid, the first guide nucleic acid comprising: (i) a first spacer complementary to a first region of a genomic locus in a cell, (ii) a first scaffold for complexing with the first RNA-guided endonuclease, and (iii) an optional first donor binding site that at least partially complementary to an integrating nucleic acid; and the second guide nucleic acid comprising: (i) a second spacer complementary to a second region of the genomic locus in the cell, (ii) a second scaffold for complexing with the first or second RNA-guided endonuclease, and (iii) an optional second donor binding site that at least partially complementary to the integrating nucleic acid; and at least one DNA ligase comprising a first DNA ligase and an optional second DNA ligase; and the integrating nucleic acid comprising a first strand and a second strand: wherein the first strand comprises an optional first guide binding site that is at least partially complementary to the first guide nucleic acid; wherein the second strand comprises an optional second binding site that is at least partially complementary to the second guide nucleic acid; wherein the first strand comprises a first flap binding site that is at least partially identical or complementary to a first genomic flap at or adjacent to the genomic locus; and wherein the second strand comprises a second flap binding site that is at least partially identical or complementary to a second genomic flap at or adjacent to the genomic locus; wherein the first RNA-guided endonuclease and/or the second RNA-guided endonuclease each cleaves at least one strand of the genomic locus in the cell; and wherein the first DNA ligase ligates an end of the first strand of the integrating nucleic acid to the first genomic flap; and the first or second DNA ligase ligates an end of the second strand of the integrating nucleic acid to the second genomic flap, thereby replacing a region of the genomic locus with the integrating nucleic acid in the cell. The integrating nucleic acid may comprise a double-stranded DNA duplex region. The double-stranded DNA may comprise a 3′ overhang optionally comprising the first guide binding site, and comprising the first flap binding site. The double stranded DNA may comprise a 3′ overhang optionally comprising the second guide binding site, and comprising the second flap binding site.

In the system, the at least one RNA-guided endonuclease may comprise a Cas protein or a functional fragment thereof. The Cas protein or the functional fragment thereof may comprise nickase activity The at least one RNA-guided endonuclease may comprise a Cas9 nickase or a functional fragment thereof. The at least one DNA ligase may ligates nucleic acids bound to DNA. The at least one DNA ligase may ligates nucleic acids bound to RNA. The at least one DNA ligase may comprise a PBCV-1 DNA ligase. The at least one DNA ligase may be operatively coupled to the at least one RNA-guided endonuclease. The at least one DNA ligase may be fused to the at least one RNA-guided endonuclease as a fusion polypeptide. The at least one RNA-guided endonuclease and the at least one DNA ligase may comprise a heterodimer domain. The at least one RNA-guided endonuclease and the at least one DNA ligase may form a heterodimer via the heterodimer domain. The at least one RNA-guided endonuclease may comprise a linker. The linker may connect the Cas protein or a functional fragment thereof to the heterodimer domain. The at least one RNA-guided endonuclease may comprise a localization signal sequence. The at least one DNA ligase may comprise a localization signal sequence. The localization signal sequence may comprise a nuclear localization sequence (NLS). The a least one RNA-guided endonuclease or the at least one DNA ligase may be directed to nucleus of the cell by the NLS. The at least one integrating nucleic acid may correct at least one genetic mutation in the at least one genomic locus. The at least one integrating nucleic acid may insert a coding sequence. The coding sequence may encode a full length protein. The at least one integrating nucleic acid may insert a non-coding sequence. The non-coding sequence may knock out an endogenous gene. The non-coding sequence may comprise a regulatory element. The system may further include a nuclease. The nuclease may comprise an exonuclease for digesting the genomic flap. The nuclease may comprise a human flap endonuclease 1 (hFEN1), a human exonuclease 5 (hEXO5), a T5 exonuclease, a T7 exonuclease, an exonuclease VIII, a flap endonuclease domain of E. coli Poll, a RecJF, a Lambda exonuclease, a Xni (ExoIXI), a SaFEN (Staphylococcus aureus FEN), a nuclease BAL-31, or a fragment thereof. The heterologous nuclease may comprise an endonuclease for digesting the genomic flap, and the endonuclease may be different from the at least one RNA-guided endonuclease. The at least one RNA-guided endonuclease may comprise at least one additional functional domain. The at least one additional functional domain may comprise a chromatin modifying domain. The at least one additional functional domain may comprise a cell penetrating peptide. The at least one guide nucleic acid may comprise at least one nucleic acid modification. The at least one nucleic acid modification may comprise a modification to a backbone, a sugar, a base, or a combination thereof. The at least one RNA-guided endonuclease may be complexed with the at least one guide nucleic acid. The at least one guide nucleic acid may be complexed with the integrating nucleic acid. The at least one RNA-guided endonuclease, the at least one guide nucleic acid, the at least one at least one DNA ligase, the integrating nucleic acid, or a combination thereof may be encoded by a polynucleotide. The polynucleotide may comprise mRNA. The polynucleotide may comprise a vector. The vector may comprise a viral vector. The at least one RNA-guided endonuclease, the at least one guide nucleic acid, the at least one at least one DNA ligase, the integrating nucleic acid, or a combination thereof may be encapsulated by at least one lipid nanoparticle. The cell may comprise a bacterial cell or a prokaryotic cell. The cell may include a prokaryotic cell. The prokaryotic cell may include a bacterial cell. The editing may be performed in a cytoplasm of the bacterial cell. The cell may include a eukaryotic cell. The eukaryotic cell may include an animal cell or a plant cell. The eukaryotic cell may include a plant cell. The eukaryotic cell may include an animal cell. The eukaryotic cell may comprise a mammalian cell. The editing may be performed in a cytoplasm of the eukaryotic cell. The editing may be performed in a nucleus of the eukaryotic cell. The system, or any aspect of the system, may be included in a composition, or in a cell such as a cell line.

Some aspects relate to a system that includes nucleic acids. The system may include guide nucleic acids, integrating nucleic acids, or a combination thereof. Some aspects relate to a system of nucleic acids. The system may include a system of guide nucleic acids. The system may include a system of integrating nucleic acids. The system of nucleic acids may further include other aspects such as additional nucleic acids or non-nucleic acid components.

The system of nucleic acids may include a guide nucleic acid. The guide nucleic acid may include a spacer. The spacer may be complementary to a region of a locus (e.g. genomic locus) of a target nucleic acid such as a genomic strand. The target nucleic acid may be in a cell. The genomic strand may be in a cell. The target nucleic acid may be in vitro. The guide nucleic acid may include a scaffold. The scaffold may complex with an endonuclease such as an RNA-guided endonuclease. The guide nucleic acid may include a flap binding site. The flap binding site may be complementary or at least partially complementary to a flap such as a genomic flap. The flap binding site may be identical or at least partially identical to a flap such as a genomic flap. The flap may be at the locus. The flap may be adjacent to the locus. The guide nucleic acid may include a donor binding site. The donor binding site may be complementary to an integrating nucleic acid. The donor binding site may be partially complementary to an integrating nucleic acid. The donor binding site may be complementary to a splinting nucleic acid. The donor binding site may be partially complementary to a splinting nucleic acid. Components of the guide nucleic acid may be included in 1 guide nucleic acid. More than one guide nucleic acid may be used. Components of the guide nucleic acid may collectively be included among multiple guide nucleic acids. Components of the guide nucleic acid may split between multiple guide nucleic acids.

The system of nucleic acids may include an integrating nucleic acid. The integrating nucleic acid may include a 5′ end to be ligated. The 5′ end may be ligated. The 5′ end may be ligated to a 3′ terminus. The 3′ terminus may be of a target nucleic acid strand (e.g. a genomic strand). The 3′ terminus may be generated by an endonuclease such as an RNA-guided endonuclease. The integrating nucleic acid may include a 5′ end to be ligated to a 3′ terminus of a genomic strand generated by an RNA-guided endonuclease. Components of the integrating nucleic acid may be included in 1 or 2 complementary strands. Components of the integrating nucleic acid may be included in 1 integrating nucleic acid. More than one integrating nucleic acid may be used. Components of the integrating nucleic acid may collectively be included among multiple integrating nucleic acids. Components of the integrating nucleic acid may split between multiple integrating nucleic acids.

The system of nucleic acids may include a splinting nucleic acid (also referred to as a “splinting strand”). The splinting strand may hybridize to two nucleic acids comprising ends to be ligated. The splinting nucleic acid may include a flap binding site. The flap binding site may be complementary to a flap. The flap binding site may be partially complementary to a flap. The flap binding site may be identical to a flap. The flap binding site may be partially identical to a flap. The flap may be at a locus of a target nucleic acid. The flap may be adjacent to a locus of a target nucleic acid. The flap may be a genomic flap. The locus may be a genomic locus. The flap binding site may be at least partially identical or complementary to a genomic flap at or adjacent to a genomic locus. The splinting nucleic acid may include a guide binding site. The guide binding site may be complementary to a guide nucleic acid. The guide binding site may be partially complementary to a guide nucleic acid. Components of the splinting nucleic acid may be included in 1 splinting nucleic acid. More than one splinting nucleic acid may be used. The splinting nucleic acid may include a donor binding site. The donor binding site may be complementary to an integrating nucleic acid. The donor binding site may be partially complementary to an integrating nucleic acid.

The splinting strand may be or include DNA. The splinting strand may be or include RNA. The splinting nucleic acid may be included as part of an integrating nucleic acid. The splinting nucleic acid may be included as a strand of a double stranded integrating nucleic acid. The splinting nucleic acid may be included as part of a guide nucleic acid.

The system of nucleic acids may include: (a) a guide nucleic acid comprising: (i) a spacer complementary to a region of a genomic locus of a genomic strand, (ii) a scaffold for complexing with RNA-guided endonuclease, (iii) an optional donor binding site that is at least partially complementary to an integrating nucleic acid, and (iv) a flap binding site that is at least partially identical or complementary to a genomic flap at or adjacent to the genomic locus; and (b) an integrating nucleic acid comprising a 5′ end to be ligated to a 3′ terminus of the genomic strand generated by an RNA-guided endonuclease. A component of (i), (ii), (iii), or (iv) may be included in a single guide nucleic acid, or may be split between or collectively included among multiple guide nucleic acids.

The system of nucleic acids may include: (a) a guide nucleic acid comprising (i) a spacer complementary to a region of a genomic locus of a genomic strand, (ii) a scaffold for complexing with an RNA-guided endonuclease, and (iii) an optional donor binding site that is at least partially complementary to a splinting nucleic acid; (b) an integrating nucleic acid comprising a 5′ end to be ligated to a 3′ terminus of the genomic strand generated by an RNA-guided endonuclease; and (c) a splinting nucleic acid comprising a flap binding site that is at least partially identical or complementary to a genomic flap at or adjacent to the genomic locus, and comprising an optional guide binding site that is at least partially complementary to a guide nucleic acid. A component of (i), (ii), or (iii) may be included in a single guide nucleic acid, or may be split between or collectively included among multiple guide nucleic acids.

In some aspects, the system described herein can be delivered into a cell, where one or more of the components of the system can be delivered into the cell together. In some aspects, each component of the system can be delivered into the cell separately. In some aspects, the system can be encoded by a polynucleotide such as a heterologous polynucleotide, where the polynucleotide is delivered into a cell and where the polynucleotide is expressed by the cell to generate the components of the cell. In some aspects, the system can be encoded and delivered into the cell via a polynucleotide comprising mRNA. In some aspects, the system can be encoded and delivered into the cell via a polynucleotide comprising a vector. In some aspects, the vector comprises a viral vector. The system can be encapsulated in a lipid or nanoparticle, or multiple lipids or nanoparticles. In some aspects, the system can be encapsulated in at least one lipid nanoparticle. In some aspects, the system comprises a ribonucleoprotein (RNP). For example, at least one RNA-guided endonuclease described herein (e.g., a Cas9) can be complexed with at least one guide nucleic acid described herein (e.g., forming a CRISPR ribonucleoprotein) for delivery. In some aspects, the system comprises at least one RNP comprising a RNA-guided endonuclease complexed with at least one first guide nucleic acid or with at least one second guide nucleic acid. In some aspects, the system comprises at least one RNP and at least one integrating nucleic acid (e.g., a single-stranded or a double-stranded integrating nucleic acid described herein). In some aspects, the system comprises at least one RNP and at least one integrating nucleic acid. In some aspects, the system comprises at least one RNP and at least one first integrating nucleic acid or at least one second integrating nucleic acid.

In some aspects, the system described herein can modify a genomic locus or gene in a cell. In some aspects, the cell comprises a bacterial cell, an eukaryotic cell, or a plant cell. In some aspects, the system described herein can be formulated into a composition, a pharmaceutical composition, a kit, or a combination thereof. In some aspects, the system described herein can be delivered and propagated in a cell line.

Some aspects include an editing system, comprising an RNA-guided endonuclease, a guide nucleic acid, and an integrating nucleic acid. Some aspects include an editing method, comprising: contacting a target nucleic acid with the editing system and a DNA ligase.

### Pharmaceutical Compositions

Described herein, in some aspects, is a pharmaceutical composition comprising the system or the composition described herein. The pharmaceutical composition may include a pharmaceutically acceptable excipient, carrier, or diluent. The pharmaceutical composition may include a carrier. The pharmaceutical composition may include an excipient. The pharmaceutical composition may be delivered to a subject. The pharmaceutical composition may be delivered to a cell. The pharmaceutical composition may be used in a method disclosed herein.

The pharmaceutical compositions described herein comprise the system, the composition, or the cell contacted with the system or contacted with the composition. The pharmaceutical composition may comprise a composition such as a protein or nucleic acid disclosed herein. The pharmaceutical composition may comprise a cell comprising a composition or system disclosed herein.

A pharmaceutical composition may include a mixture of a pharmaceutical composition, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. In practicing the methods of treatment or use provided herein, therapeutically effective amounts of pharmaceutical compositions described herein are administered to a mammal having a disease, disorder, or condition to be treated. In some aspects, the mammal is a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures.

The pharmaceutical composition may be formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. Pharmaceutical compositions including a pharmaceutical composition are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a pharmaceutical composition as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some aspects, pharmaceutical compositions exist in unsolvated form or in solvated forms with pharmaceutically acceptable solvents such as water, ethanol, and the like. The solvated forms of the pharmaceutical compositions are also considered to be disclosed herein.

In some aspects, a pharmaceutical composition exists as a tautomer. All tautomers are included within the scope of the agents presented herein. As such, it is to be understood that a pharmaceutical composition or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they can be regarded as different isomeric forms of the same compound.

In some aspects, a pharmaceutical composition exists as an enantiomer, diastereomer, or other steroisomeric form. The agents disclosed herein include all enantiomeric, diastereomeric, and epimeric forms as well as mixtures thereof.

In some aspects, pharmaceutical compositions described herein can be prepared as prodrugs. A “prodrug” refers to an agent that is converted into the parent drug in vivo. Prodrugs are often useful because, in some situations, they can be easier to administer than the parent drug. They may, for instance, be bioavailable by oral administration whereas the parent is not. The prodrug may also have improved solubility in pharmaceutical compositions over the parent drug. In certain embodiments, upon in vivo administration, a prodrug is chemically converted to the biologically, pharmaceutically or therapeutically active form of the pharmaceutical composition. In certain embodiments, a prodrug is enzymatically metabolized by one or more steps or processes to the biologically, pharmaceutically or therapeutically active form of the pharmaceutical composition.

### Kits

Described herein, in some aspects, are kits for using the system, the composition, or the pharmaceutical composition described herein. In some aspects, the kits disclosed herein may be used to treat a disease or condition in a subject. In some aspects, the kit comprises an assemblage of materials or components apart from the system, the composition, or the pharmaceutical composition. In some aspects, the kit comprises the components for assaying and selecting for suitable guide nucleic acid or donor strand for treating a disease or a condition. In some aspects, the kit comprises components for performing assays such as enzyme-linked immunosorbent assay (ELISA), single-molecular array (Simoa), PCR, or qPCR. The exact nature of the components configured in the kit depends on its intended purpose. For example, some embodiments are configured for the purpose of treating a disease or condition disclosed herein in a subject. In some aspects, the kit is configured particularly for the purpose of treating mammalian subjects. In some aspects, the kit is configured particularly for the purpose of treating human subjects.

Instructions for use may be included in the kit. In some aspects, the kit comprises instructions for administering the composition to a subject in need thereof. In some aspects, the kit comprises instructions for further engineering the system described herein. In some aspects, the kit comprises instructions for thawing or otherwise restoring biological activity of at least one component of the system, which may have been cryopreserved or lyophilized during storage or transportation. In some aspects, the kit comprises instructions for measuring efficacy for its intended purpose (e.g., therapeutic efficacy if used for treating a subject).

The kit may comprise a system or composition disclosed herein, and a container. The composition may be a pharmaceutical composition.

### Methods

Described herein are methods such as methods of modifying a target nucleic acid. Described herein are methods such as methods of gene editing or gene replacement. The method may include use of any aspect of composition described herein such as an endonuclease, ligase, guide nucleic acid, integrating nucleic acid, system, kit, or pharmaceutical composition.

### Gene Editing or Replacement

Disclosed herein are editing methods such as gene editing methods or nucleic acid editing methods. The editing tools and methods disclosed herein may be useful for genetic enhancement, genetic correction, treatment of a disease, development of research tools, or for disease diagnosis. The methods may be performed for therapeutic, agricultural, industrial, and research purposes. The editing method may include contacting a target nucleic acid with an editing system and a ligase. The target nucleic acid may be double-stranded. The target nucleic acid may include a host or cell genome. The target nucleic acid may include a pathogen genome in a host. The target nucleic acid may be part of a gene, or may include a non-gene or intergenic sequence. The target nucleic acid may reside in a nucleus of a cell. The target nucleic acid may include chromatin, euchromatin, or heterochromatin. The target nucleic acid may comprise DNA. The methods referred to herein as gene editing methods or genome editing methods may be useful for nucleic acid editing without necessarily being limited to editing of a certain gene. The method may include replacing a target nucleic acid sequence with a sequence of an integrating nucleic acid. The method may be performed in vitro. The method may be performed in vivo. The method may be performed in a cell. The editing may be performed without homologous recombination. The editing may be performed without prior insertion into host genome.

Disclosed herein, in some aspects, are editing methods. The method may include editing a nucleic acid. The nucleic acid may be in a cell. The editing may be performed using a DNA ligase. The editing may be performed using a CRISPR protein. The editing may be performed using a CRISPR protein or DNA ligase without any significant chemical interaction with an endogenous factor. The editing may be performed using a CRISPR protein or DNA ligase without any significant chemical interaction with a polymerase such as a DNA polymerase. In some aspects, the editing may be performed using an endonuclease (e.g., a Cas endonuclease) described herein or DNA ligase, where the endonuclease and the DNA ligase are coupled. For example, the endonuclease and the DNA ligase can be covalently coupled as a fusion protein for performing the editing. The method may include editing a nucleic acid in a cell, wherein the editing is performed using a Cas endonuclease without any significant chemical interaction with an endogenous factor or polymerase. The method may include editing a nucleic acid in a cell, wherein the editing is performed using a Cas endonuclease without any significant chemical interaction with endogenous cellular components of NHEJ or HDR. The editing method may exclude polymerization or in-cell synthesis of a nucleic acid. For example, the method may exclude in-cell synthesis from a template on a guide nucleic acid.

The editing may be performed, in some aspects, solely by factors exogenous to the cell. The exogenous factors may be added to the cell or are encoded by a nucleic acid added to the cell. In some aspects, the exogenous factors are added to the cell. In some aspects, the exogenous factors encoded by a nucleic acid added to the cell. The factors may include a Cas endonuclease and a DNA ligase. The Cas endonuclease may be or include a DNA-binding protein.

The editing may include replacing a nucleotide or nucleotide sequence within a target nucleic acid. The editing may include replacing a nucleotide. The editing may include replacing a nucleotide sequence. The nucleotide or nucleotide sequence may be replaced with an integrating nucleic acid. The editing may include replacing a nucleotide or nucleotide sequence of the nucleic acid with an integrating nucleic acid. In some aspects, replacing the nucleotide comprises breaking a phosphodiester bond of the nucleic acid and forming a new phosphodiester bond with the integrating nucleic acid. In some aspects, the replacement is performed at a replacement site within the nucleic acid, without leaving a remaining nick or strand break in the nucleic acid at the replacement site. In some aspects, the editing generates an edited nucleic acid comprising an edited region flanked by phosphodiester bonds to unedited regions of the edited nucleic acid.

Described herein, in some aspects, is a method for correcting a gene or modifying gene expression in a cell. In some aspects, the method comprises contacting the cell with a system or composition described herein. In some aspects, the method comprises delivering a heterologous polynucleotide into the cell, where the heterologous polynucleotide encodes at least one component of system. In some aspects, the system described herein can introduce a donor strand into a genomic locus. In some aspects, the system can introduce the donor strand without the need of endogenous machinery of the cell. In some aspects, the system can introduce the donor strand without the need to synchronize cell cycling. In some aspects, the system can introduce the donor strand in non-dividing cell or slow dividing cell. Such technical aspect can be especially useful for correcting genetic mutation in non-dividing cell or slow dividing cell for treating a disease or condition.

The method may include editing a nucleic acid of a cell. In some embodiments, the cell is quiescent or senescent cell. The cell may be quiescent. The cell may be senescent. In some aspects, the cell is not actively dividing. The cell may have a low dNTP concentration relative to other cells or cell types. Some examples of cells may include a neuron, myocyte, cardiomyocyte, or osteocyte. The cell may include a neuron. The cell may include a myocyte. The cell may include a cardiomyocyte. The cell may include an osteocyte. The cell may include an eye cell.

The cell may include a stem cell such as an embryonic stem cell, or such as an adult stem cell. The cell may be a circulating cell such as a blood cell. The cell may include a bone marrow cell. The cell may be an immune cell. The cell may be an innate immune cell.

The cell may be an airway cell. The cell may be a lung cell. The cell may be a bronchial cell. The cell may be an endothelial cell.

Described herein, in some aspects, is an editing method, comprising: editing a nucleic acid in a cell, wherein the editing is performed using a CRISPR protein (e.g. an RNA-guided endonuclease such as a Cas endonuclease) without any significant chemical interaction with an endogenous factor or polymerase. In some embodiments, the editing is performed solely by factors exogenous to the cell. In some embodiments, the exogenous factors are added to the cell or are encoded by a nucleic acid added to the cell.

In some embodiments, the editing is performed using a DNA ligase. In some embodiments, the editing comprises replacing a nucleotide or nucleotide sequence of the nucleic acid with an integrating nucleic acid. In some embodiments, replacing the nucleotide comprises breaking a phosphodiester bond of the nucleic acid and forming a new phosphodiester bond with the integrating nucleic acid. In some embodiments, the replacement is performed at a replacement site within the nucleic acid, without leaving a nick or strand break in the nucleic acid at the replacement site. In some embodiments, the editing generates an edited nucleic acid comprising an edited region flanked by phosphodiester bonds to unedited regions of the edited nucleic acid.

Some aspects include a method for modifying a cell comprising contacting a cell with a system or composition such as a pharmaceutical composition disclosed herein. In some aspects, the cell is not a dividing cell. The integrating nucleic acid may be inserted into the genomic locus of the cell independent of endogenous non-homologous end joining (NHEJ) and independent of endogenous homology-directed repair (HDR).

In some aspects, described herein is a method for modifying or replacing a nucleotide or nucleotide sequence in a cell by contacting the cell with the system or composition described herein, where the system or composition comprises a guide nucleic acid comprising: a spacer complementary to a region of a genomic locus of a genomic strand; a scaffold for complexing with an endonuclease; an optional donor binding site that is at least partially complementary to an integrating nucleic acid; and a flap binding site that is at least partially identical or complementary to a genomic flap at or adjacent to the genomic locus. In some embodiments, the guide nucleic acid comprises the donor binding site is complexed with the integrating nucleic acid. The complexing between the guide nucleic acid and the integrating nucleic acid can occur in vivo or in vitro. In some embodiments, the flap binding site can be complexed with a genomic flap generated by the endonuclease cleaving the genomic strand. The complexing between the flap binding site and the genomic flap can bring the integrating nucleic acid to close proximity to the cleaved genomic strand. The decreased proximity between the donor nucleic and the cleaved genomic strand can increase editing efficiency, decease off-target effect, or decrease introduction of unwanted mutations such as indels. In such case, the integrating nucleic acid can replace one strand of the cleaved genomic strand, thus editing or correcting the cleaved genomic strand. FIG. 1A-FIG. 1C illustrate the complexing between the guide nucleic acid and the integrating nucleic acid described herein, where the complexing between the guide nucleic acid and the integrating nucleic acid brings the integrating nucleic acid to close proximity to the cleaved genomic strand. In some embodiments, the integrating nucleic acid comprises a 5′ end to be ligated to a 3′ terminus of a genomic strand generated by an endonuclease cleaving the genomic strand. In some embodiments, the integrating nucleic acid comprises a 3′ end to be ligated to a 5′ terminus of a genomic strand generated by an endonuclease cleaving the genomic strand. In some embodiments, the endonuclease can be a fusion protein described herein. For example, the endonuclease can be fused to a DNA ligase described herein, where the endonuclease and DNA ligase fusion can cleave the genomic strand and ligate the integrating nucleic acid to the cleaved genomic strand with increased efficiency.

In some embodiments, the integrating nucleic acid is double stranded or partially double stranded, where the integrating nucleic acid can replace both strands of the cleaved genomic strand. In such case, the integrating nucleic acid can comprise single stranded guide binding site to be complexed with a guide nucleic acid comprising the donor binding site. The guide binding site can locate at 5′ end of the integrating nucleic acid. The guide binding site can locate at 3′ end of the integrating nucleic acid. The guide binding site can locate at both 5′ end and 3′ end of the integrating nucleic acid. FIG. 2A-FIG. 2C illustrate a double stranded integrating nucleic acid comprising the guide binding site at both 5′ end and 3′ end of the integrating nucleic acid, where the integrating nucleic acid can edit and replace the cleaved genomic strand.

In some embodiments, the integrating nucleic acid is double stranded or partially double stranded, where the integrating nucleic acid comprises a flap binding site and a guide binding site. In such case, the guide binding site can complex with the donor binding site of the guide nucleic acid. FIG. 3A illustrates such arrangement, where the integrating nucleic acid (and not the guide nucleic acid) can be complexed with the genomic flap to bring the integrating nucleic acid to close proximity to the cleaved genomic strand. In some embodiments, the donor nucleic comprises two flap binding sites to be complexed with two different genomic flaps. FIG. 4A illustrates such arrangement, where the integrating nucleic acid (and not the guide nucleic acid) can be complexed with the two genomic flaps to bring the integrating nucleic acid to close proximity to the two cleaved genomic strand.

In some embodiments, the integrating nucleic acid comprises the guide binding site, where the guide binding site can be complexed with the donor binding site of the guide nucleic acid. The guide nucleic acid can comprise the flap binding site to be complexed with the genomic flap at the cleaved genomic strand. As shown in FIG. 5A, the guide nucleic acid brings the integrating nucleic acid to close proximity to the cleaved genomic strand for editing and replacing the cleaved genomic strand with the integrating nucleic acid. In some embodiments, the integrating nucleic acid can be double strand and comprises the two guide binding sites to be complexed with two different guide nucleic acids. FIG. 6A illustrates such arrangement, where the two guide nucleic acids bring the integrating nucleic acid to close proximity to two cleaved genomic strands.

In some aspects, described herein is a method for modifying or replacing a nucleotide or nucleotide sequence in a cell by contacting the cell with the system or composition described herein, where the system or composition comprises a guide nucleic acid comprising a spacer complementary to a region of a genomic locus of a genomic strand; a scaffold for complexing with an endonuclease, and an optional donor binding site that is at least partially complementary to a splinting nucleic acid. In some embodiments, the system or composition comprises an integrating nucleic acid, where the integrating nucleic acid can be ligated into the cleaved or nicked genomic strand. In some embodiments, the integrating nucleic acid comprises a 5′ end to be ligated to a 3′ terminus of the genomic strand generated by an endonuclease. In some embodiments, the integrating nucleic acid comprises a 3′ end to be ligated to a 5′ terminus of the genomic strand generated by an endonuclease. In some embodiments, the system or composition comprises a splinting nucleic acid comprising a flap binding site that is at least partially identical or complementary to a genomic flap at or adjacent to the genomic locus, and comprising an optional guide binding site that is at least partially complementary to a guide nucleic acid. In some embodiments, the splinting nucleic acid may include a guide binding site. The guide binding site may be complementary to a guide nucleic acid. The guide binding site may be partially complementary to a guide nucleic acid. The splinting nucleic acid may include a donor binding site. The donor binding site may be complementary to an integrating nucleic acid. The donor binding site may be partially complementary to an integrating nucleic acid. The splinting strand may be or include DNA. The splinting strand may be or include RNA. The splinting nucleic acid may be included as part of an integrating nucleic acid. The splinting nucleic acid may be included as a strand of a double stranded integrating nucleic acid.

In some embodiments, the method described herein decreases proximity between the integrating nucleic acid and the cleaved or nicked site. In some embodiments, the decreased proximity between the integrating nucleic acid and the cleaved or nicked site increases gene editing rate by at least 0.1 fold, 0.2 fold, 0.5 fold, 1.0 fold, 2.0 fold, 5.0 fold, 10.0 fold, or more compared to a gene editing rate without using a composition or a replacer described herein. In some embodiments, the decreased proximity between the integrating nucleic acid and the cleaved or nicked site decreases introduction of unwanted mutation such as indel by at least 0.1 fold, 0.2 fold, 0.5 fold, 1.0 fold, 2.0 fold, 5.0 fold, 10.0 fold, or more compared to a introduction of unwanted mutation without using a composition or a replacer described herein. In some embodiments, the decreased proximity between the integrating nucleic acid and the cleaved or nicked site decreases off-target editing by at least 0.1 fold, 0.2 fold, 0.5 fold, 1.0 fold, 2.0 fold, 5.0 fold, 10.0 fold, or more compared to off-target editing without using a composition or a replacer described herein.

In some aspects, the method edits a gene. In some aspects, the method replaces a gene. In some aspects, the method removes a gene. In some aspects, the method introduces a methylated nucleotide into the target nucleic acid. In some aspects, the method introduces an unmethylated nucleotide into the target nucleic acid.

The method may be used to edit a nucleic acid in a plant cell. Some aspects include enhancing a plant. Some examples of plant enhancement may include editing of a disease susceptibility gene or introducing an herbicide resistance gene. An example of a disease susceptibility gene may include bacterial leaf streak disease susceptibility gene OsSULTR3;6 in rice. An example of introducing an herbicide resistance gene may include editing of acetolactate synthase in potato for herbicide resistance

### Treatment

A method such as a gene editing method may be useful for treatment of a disease or disorder. The disease or disorder may be genetic. The treatment may be of a diseased or damaged cell. The disease may include a genetic disease, cancer, or an infection. The treatment may include administration of a composition disclosed herein to a subject in need thereof. The subject in need may include a subject identified as having a disease or disorder.

The methods described herein may be useful for treating a genetic disease. The genetic disease may be caused by a DNA mutation such as a point mutation, a deletion, an insertion, a duplication, or a repeat, relative to normal non-diseased DNA. The treatment may correct the mutation. Some examples of genetic diseases may include Angelman syndrome, Canavan disease, Charcot-Marie-Tooth disease, color blindness, cri du chat syndrome, cystic fibrosis, DiGeorge syndrome, Duchenne muscular dystrophy, familial hypercholesterolemia, haemochromatosis type 1, hemophilia, neurofibromatosis, phenylketonuria, polycystic kidney disease, Prader-Willi syndrome, sickle cell disease, spinal muscular atrophy, or Tay-Sachs disease. Some examples of diseases that may be treated using a method herein may include sickle cell disease, beta thalassemia, familial hypercholesterolemia (e.g. PCSK9 disruption), alpha I antitrypsin deficiency, phenylketonuria, cystic fibrosis, tyrosinemia, arginase I deficiency, Wilson's disease, a repeat expansion disorder, hemophilia (e.g. insertion of Factor IX at ALB in a hepatocyte), Duchenne muscular dystrophy. Some examples of repeat expansion disorders like Huntington's disease, Amyotrophic lateral sclerosis/frontotemporal dementia, Friedreich ataxia, Fragile X Syndrome. The method may be included in immuno-oncology, such as for T-cell engineering or in cancer treatment.

Two non-limiting examples of genetic diseases for which efficient and precise editing of slowly dividing and nondividing cells is beneficial for therapeutic gene therapy are sickle cell anemia (SCA) and alpha-1 antitrypsin deficiency (AATD). Sickle cell anemia is caused by the E6V missense mutation in the HBB gene resulting in aggregation of mutant beta-globin protein and ‘sickling’ of red blood cells. Autologous gene therapies using hematopoetic stem cells with corrected HBB alleles have been proposed as curative treatments for SCA. While expansion of ex vivo HSC cultures can be induced using cytokine cocktails, HSCs in the human body typically reside in niches within the bone marrow where they exist in a quiescent or slowly dividing state. AATD is most commonly caused by the E366K missense mutation in the SERPINA1 gene which encodes alpha-1 antitrypsin, a serine protease inhibitor secreted by hepatocytes. Mutant AAT is misfolded, forming aggregates in the endoplasmic reticulum of the hepatocytes rather than being secreted, ultimately leading to liver disease. Although hepatocytes possess the ability to rapidly proliferate in response to liver damage, their life cycles are typically spent in a state of quiescence. As such, high efficiency in vivo editing of these two disorders necessitates a novel gene therapy platform which can effectively perform precise edits in nondividing or slowly dividing cells.

Some aspects include a method for treating a disease or condition in subject in need thereof comprising: (a) contacting a cell of the subject with a system or composition such as a pharmaceutical composition disclosed herein; and (b) replacing a genomic locus in a cell with an integrating nucleic acid, thereby treating the disease or condition in the subject. In some aspects, the cell is not a dividing cell. In some aspects, the integrating nucleic acid is inserted into the genomic locus of the cell independent of endogenous non-homologous end joining (NHEJ) and independent of endogenous homology-directed repair (HDR).

In some embodiments, the method described herein decreases proximity between the integrating nucleic acid and the cleaved or nicked site, where the decreased proximity between the integrating nucleic acid and the cleaved or nicked site increases gene editing rate by at least 0.1 fold, 0.2 fold, 0.5 fold, 1.0 fold, 2.0 fold, 5.0 fold, 10.0 fold, or more compared to a gene editing rate without using a composition or a replacer described herein. In some embodiments, the decreased proximity between the integrating nucleic acid and the cleaved or nicked site increases therapeutic efficacy (e.g., by increasing gene editing rate) by at least 0.1 fold, 0.2 fold, 0.5 fold, 1.0 fold, 2.0 fold, 5.0 fold, 10.0 fold, or more compared to a therapeutic efficacy without using a composition or a replacer described herein.

### Delivery

Described herein, in some aspects, are methods of delivering the system described herein to a cell. In some aspects, the method comprises delivering directly or indirectly at least one component of the system to the cell. In some aspects, the method comprises delivering the cell with at least one heterologous polynucleotide, where the cell can then express the at least one component of the system. In some aspects, the at least one heterologous polynucleotide can be delivered into the cell via any of the transfection methods described herein. In some aspects, the at least one heterologous polynucleotide can be delivered into the cell via the use of expression vectors such as viral vectors. In the context of an expression vector, the vector can be readily introduced into the cell described herein by any method in the art. For example, the expression vector can be transferred into the cell by physical, chemical, or biological means.

Physical methods for introducing the oligonucleotide or vector encoding the oligonucleotide into the cell can include calcium phosphate precipitation, lipofection, particle bombardment, microinjection, gene gun, electroporation, and the like. Methods for producing cells comprising vectors and/or exogenous nucleic acids are suitable for methods herein. One method for the introduction of oligonucleotide or vector encoding the oligonucleotide into a host cell is calcium phosphate transfection.

Chemical means for introducing the oligonucleotide or vector encoding the oligonucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An exemplary colloidal system for use as a delivery vehicle in vitro and in vivo is a liposome (e.g., an artificial membrane vesicle). Other methods of state-of-the-art targeted delivery of nucleic acids are available, such as delivery of oligonucleotide or vector encoding the oligonucleotide with targeted nanoparticles or other suitable sub-micron sized delivery system.

In the case where a non-viral delivery system is utilized, an exemplary delivery vehicle is a liposome. The use of lipid formulations is contemplated for the introduction of the oligonucleotide or vector encoding the oligonucleotide into a cell (in vitro, ex vivo or in vivo). In another aspect, the oligonucleotide or vector encoding the oligonucleotide can be associated with a lipid. The oligonucleotide or vector encoding the oligonucleotide associated with a lipid, In some aspects, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, In some aspects, they are present in a bilayer structure, as micelles, or with a “collapsed” structure. Alternately, they may be simply interspersed in a solution, possibly forming aggregates that are not uniform in size or shape. Lipids are fatty substances which are, In some aspects, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, In some aspects, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some aspects, the delivery method comprises conjugating or encapsulating the compositions or the oligonucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the oligonucleotide or vector encoding the oligonucleotide described herein can be packaged and delivered to the cell via extracellular vesicles. The extracellular vesicles can be any membrane-bound particles. In some aspects, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In aspects, the system described herein or the at least one heterologous polynucleotide encoding the system described herein can be delivered into a cell as a vector such as a viral vector. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Leukemia Virus (MoMLV, MMLV, MuLV, or MLV) or the Murine Stem cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

In some cases, the at least one heterologous polynucleotide encoding the system described herein can be administered to the subject in need thereof via the use of the transgenic cells generated by introduction of the at least one heterologous polynucleotide first into allogeneic or autologous cells. In some cases, the cell can be isolated. In some aspects, the cell can be isolated from the subject.

### Subjects and Cells

The methods described herein may involve cells. For example, a composition may be delivered to a cell to edit a nucleic acid in the cell. The aspects delivered to the cell may be heterologous to the cell. “Heterologous” may include anything that does not exist in the cell in its natural state.

Any cell or cell type may be used. Examples of cells or cell types may include stem cells, red blood cells, white blood cells, platelets, nerve cells, neuroglial cells, muscle cells, cartilage cells, bone cells, skin cells, endothelial cells, epithelial cells, fat cells, or sex cells. The cell may include a stem cell. The cell may include a bone cell. The cell may include a blood cell. The cell may include a sperm cell. The cell may include an egg cell. The cell may include a fat cell. The cell may include a nerve cell. The cell may include a muscle cell. The cell may include an endocrine cell. The cell may include an endothelial cell. The cell may include a pancreatic cell.

The cell may be eukaryotic. The cell may be a plant cell. The cell may be an animal cell. The cell may be protozoan. The cell may be a fungal cell. The cell may be prokaryotic. The cell may be a bacterial cell. The cell may be an archaeon cell. The cell may be from a cell line. The cell may be part of a subject. The cell may be separated from a subject. The cell may be an autologous cell of a subject. The cell may be an allogenic cell of a subject.

The cell may include a diseased cell. The cell may include a cancer cell. The cell may be infected. The cell may be damaged. The cell may be a pathogen such as a fungal pathogen.

The methods described herein may involve a subject. For example, a composition may be delivered to the subject. Some aspects of the methods described herein include treatment of the subject. Non-limiting examples of subjects include vertebrates, animals, mammals, dogs, cats, cattle, rodents, mice, rats, primates, monkeys, and humans. The subject may be an invertebrate. The subject may be a arthropod. The subject may be a vertebrate. The subject may be an animal. The subject may be a fish. The subject may be a reptile. The subject may be a mammal. The subject may be a dog. The subject may be a cat. The subject may be a cattle. The subject may be a rodent. The subject may be a mouse. The subject may be a rat. The subject may be a primate. The subject may be a non-human primate. The subject may be a monkey. The subject may be an animal, a mammal, a dog, a cat, cattle, a rodent, a mouse, a rat, a primate, or a monkey. The subject may be a human.

The subject may be a non-animal subject. For example, the subject may include a plant. Examples of plants may include trees, flowers, shrubs, or grasses. The subject may include a crop. Examples of crops may include almond, apricot, apple, artichoke, banana, barley, beet, blackberry, blueberry, broccoli, Brussels sprout, cabbage, cannabis, capsicum, carrot, celery, chard, cherry, citrus, corn, cucurbit, date, fig, garlic, grape, herb, spice, kale, lettuce, oil palm, olive, onion, pea, pear, peach, peanut, papaya, parsnip, pecan, persimmon, plum, pomegranate, potato, quince, radish, raspberry, rose, rice, sloe, sorghum, soybean, spinach, strawberry, sweet potato, tobacco, tomato, turnip greens, walnut, or wheat.

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

Where sequences are provided, nucleic acids containing phosphorothioate bonds between nucleotides are signified with an asterisk (*). 2′-O-methyl nucleotides are signified with a lowercase “m” in front of the nucleotide, for example mC instead of C. The code “/5Phos/” in front of a nucleotide sequence indicates that the sequence is phosphorylated at the 5′ end. Locked nucleic acid (LNA) nucleotides comprising a methylene bridge connecting the 2′ oxygen and 4′ carbon are signified with a “+” in front of the nucleotide, for example +C instead of C.

### EMBODIMENTS

Some aspects include an embodiment as follows:

Embodiment 1. Described herein, in some aspects, is a composition, comprising: a DNA-binding protein coupled to a DNA ligase.

Embodiment 2. The composition of Embodiment 1, wherein the coupling is covalent.

Embodiment 3. The composition of Embodiment 2, comprising a fusion protein comprising the DNA-binding protein and the DNA ligase.

Embodiment 4. The composition of Embodiment 3, wherein the DNA-binding protein is amino (N)-terminal relative to the DNA ligase within the fusion protein.

Embodiment 5. The composition of Embodiment 3, wherein the DNA-binding protein is carboxy (C)-terminal relative to the DNA ligase within the fusion protein.

Embodiment 6. The composition of any one of Embodiments 2-5, wherein the connection comprises a linker comprising 1-100 amino acids.

Embodiment 7. The composition of Embodiment 1, wherein the coupling is non-covalent.

Embodiment 8. The composition of Embodiment 7, wherein the composition comprises a first polypeptide comprising at least part of the DNA-binding protein, and a second polypeptide comprising at least part of the DNA ligase, wherein the first and second polypeptides are non-covalently coupled.

Embodiment 9. The composition of Embodiment 8, wherein the first polypeptide comprises a first heterodimerization domain that binds a second heterodimerization domain, and wherein the second polypeptide comprises the second heterodimerization domain.

Embodiment 10. The composition of Embodiment 9, wherein the heterodimer domains comprise a leucine zipper, PDZ domain, streptavidin, streptavidin binding protein, foldon domain, hydrophobic moiety, or a functional binding fragment thereof.

Embodiment 11. The composition of Embodiment 8, wherein the first polypeptide comprises a first intein that binds a second intein, and wherein the second polypeptide comprises the second intein.

Embodiment 12. The composition of Embodiment 1, wherein the ligase comprises a hairpin binding motif, and wherein the DNA-binding protein and the DNA ligase are coupled with a nucleic acid comprising a scaffold that binds to the DNA-binding protein and a hairpin that binds to the hairpin binding motif.

Embodiment 13. The composition of Embodiment 12, wherein the hairpin binding motif comprises an MS2 coat protein (MCP) peptide, and wherein the hairpin comprises an MS2 hairpin.

Embodiment 14. The composition of Embodiment 1, wherein the DNA-binding protein and the DNA ligase are coupled with a heterobifunctional molecule comprising an endonuclease binding domain and a DNA ligase binding domain.

Embodiment 15. The composition of Embodiment 14, wherein the heterobifunctional molecule comprises a small molecule.

Embodiment 16. Described herein, in some aspects, is a composition comprising a cell containing a DNA-binding protein and a DNA ligase, both of which are heterologous to the cell.

Embodiment 17. The composition of any one of Embodiments 1-16, wherein the DNA-binding protein comprises a class II CRISPR/Cas endonuclease.

Embodiment 18. The composition of any one of Embodiments 1-17, wherein the DNA-binding protein comprises a Cas9 endonuclease.

Embodiment 19. The composition of any one of Embodiments 1-18, wherein the DNA-binding protein comprises a nickase.

Embodiment 20. The composition of any one of Embodiments 1-19, wherein the DNA-binding protein comprises an amino acid sequence at least 80% identical to the amino acid sequence of any one of SEQ ID NOS: 1-13, or a functional fragment thereof.

Embodiment 21. The composition of any one of Embodiments 1-20, wherein the DNA ligase ligates DNA strands base paired to a DNA splint.

Embodiment 22. The composition of any one of Embodiments 1-20, wherein the DNA ligase ligates DNA strands base paired to an RNA splint.

Embodiment 23. The composition of any one of Embodiments 1-22, wherein the DNA ligase comprises an amino acid sequence at least 80% identical to the amino acid sequence of any one of SEQ ID NOS: 55-96, or a functional fragment thereof.

Embodiment 24. The composition of any one of Embodiments 1-23, wherein the DNA-binding protein or the DNA ligase comprises a nuclear localization signal, chromatin modifying domain, cell penetrating peptide, or tag polypeptide.

Embodiment 25. The composition of any one of Embodiments 1-24, further comprising a guide RNA and an integrating nucleic acid.

Embodiment 26. One or more nucleic acids encoding the composition of any one of Embodiments 1-25.

Embodiment 27. A cell comprising the composition of any one of Embodiments 1-25, or comprising the one or more nucleic acids of Embodiment 26.

Embodiment 28. A system of nucleic acids comprising:
a. a guide nucleic acid comprising:
   i. a spacer complementary to a region of a genomic locus of a genomic strand,
   ii. a scaffold for complexing with a DNA-binding protein,
   iii. an optional donor binding site that is at least partially complementary to an integrating nucleic acid, and
   iv. a flap binding site that is at least partially identical or complementary to a genomic flap at or adjacent to the genomic locus; and
b. an integrating nucleic acid comprising a 5′ end to be ligated to a 3′ terminus of the genomic strand generated by a DNA-binding protein.

Embodiment 29. A system of nucleic acids comprising:
a. a guide nucleic acid comprising:
   i. a spacer complementary to a region of a genomic locus of a genomic strand,
   ii. a scaffold for complexing with a DNA-binding protein, and
   iii. an optional donor binding site that is at least partially complementary to a splinting nucleic acid;
b. an integrating nucleic acid comprising a 5′ end to be ligated to a 3′ terminus of the genomic strand generated by a DNA-binding protein; and
c. a splinting nucleic acid comprising a flap binding site that is at least partially identical or complementary to a genomic flap at or adjacent to the genomic locus, and comprising an optional guide binding site that is at least partially complementary to a guide nucleic acid.

Embodiment 30. The system of Embodiment 28 or 29, wherein the genomic strand is in a cell.

Embodiment 31. The system of any one of Embodiments 28-30, wherein the splinting nucleic acid further comprises a donor binding site that is at least partially identical or complementary to a portion of the integrating nucleic acid.

Embodiment 32. The system of any one of Embodiment 28-31, wherein the guide nucleic acid comprises a sequence of linking nucleic acids between the scaffold and the donor binding site.

Embodiment 33. The system of any one of Embodiment 28-32, wherein the guide nucleic acid, the integrating nucleic acid, or the splinting nucleic acid comprises a modified internucleoside linkage.

Embodiment 34. The system of Embodiment 33, wherein the modified internucleoside linkage comprises a phosphorothioate linkage.

Embodiment 35. The system of Embodiment 33 or 34, wherein the modified internucleoside linkage is between any of the 4 terminal nucleosides at a 5′ end or at a 3′ end of the guide nucleic acid or the integrating nucleic acid.

Embodiment 36. The system of any one of Embodiments 28-35, wherein the guide nucleic acid, the integrating nucleic acid, or the splinting nucleic acid comprises a modified nucleoside.

Embodiment 37. The system of Embodiment 36, wherein the modified nucleoside comprises a locked nucleic acid (LNA), a 2′ fluoro, a 2′ O-alkyl, or a combination thereof.

Embodiment 38. The system of Embodiment 36 or 37, wherein the modified nucleoside is any of the 3 terminal nucleosides at a 5′ end or at a 3′ end of the guide nucleic acid or the integrating nucleic acid.

### EXAMPLES

### Example 1. Editing to Convert BFP to GFP by Replacer 1

Components used to edit the blue fluorescent protein (BFP) gene stably integrated into HEK293 cells are co-delivered by lipid nanoparticle (LNP) transfection. The components include chemically synthesized guide RNAs (gRNAs), single-stranded DNA donors, and mRNA encoding protein effectors for Replacer 1 editing including nicking Cas9 (nCas9), a SplintR ligase and nuclear localization sequences (NLS). The gRNAs are synthesized by Agilent, the DNA donors are synthesized by IDT, and the mRNA is synthesized by TriLink or RiboPro. The gRNA, DNA donor, and mRNA are mixed and formulated into lipid nanoparticles prior to delivery to adherent cells in 96well plates. After 48 hours, the cells are detached from the plate by trypsinization and green fluorescent protein (GFP) fluorescence is measured using an Attune NxT flow cytometer to assess the percentage of BFP-to-GFP editing. Following the Replacer 1 editing format, the gRNAs contain a spacer, scaffold, donor binding site (DBS), and flap binding site (FBS). The gRNAs are delivered individually (1-sided Replacer 1) or as pairs with spacers targeting opposite strands of the genomic locus (2-sided Replacer 1). Some of the DBSs contain a mutation in the spacer region or in the protospacer adjacent motif region (SpPAMmut). The gRNAs contain 2′-O-methyl 3′-phosphorothioate nucleotides at the first three and last three positions. The DNA donors are delivered individually (1-sided Replacer 1) or in pairs (2-sided Replacer 1). Some donors have mutations in the spacer or protospacer adjacent motif (PAM) regions (SpPAMmut). Some donors have phosphorothioate bonds at the first three and last three positions. Some donors are recoded with silent mutations that change the nucleotide sequence but retain the amino acid sequence. The DNA donors are phosphorylated on the 5′ end. In some conditions, the gRNAs and donor DNAs are annealed by a thermal cycler annealing reaction prior to LNP formulation. Plasmids can be used in the place of mRNA. Table 12 details this experiment. Sequences corresponding to the names in the table may be found herein.

TABLE 12

| | | | | | | Anneal |
|---|---|---|---|---|---|---|
| Condition | Forward Guide | Reverse Guide | Top Donor | Bottom Donor | Ligase | both sides? |
| 1 | Rep1.BFP. | | Rep1. | | NLS-nCas9- | N/A |
| | FwdGuide | | BFP2GFP. | | linker- | |
| | | | TopDonor. | | SplintR- | |
| | | | 5P | | bpNLS | |
| 2 | | Rep1.BFP. | | Rep1. | NLS-nCas9- | N/A |
| | | RevGuide | | BFP2GFP. | linker- | |
| | | | | BotDonor. | SplintR- | |
| | | | | 5P | bpNLS | |
| 3 | Rep1.BFP. | Rep1.BFP. | Rep1. | Rep1. | NLS-nCas9- | Yes |
| | FwdGuide | RevGuide | BFP2GFP. | BFP2GFP. | linker- | |
| | | | TopDonor. | BotDonor. | SplintR- | |
| | | | 5P | 5P | bpNLS | |
| 4 | Rep1.BFP. | Rep1.BFP. | Rep1. | Rep1. | NLS-nCas9- | No |
| | FwdGuide | RevGuide | BFP2GFP. | BFP2GFP. | linker- | |
| | | | TopDonor. | BotDonor. | SplintR- | |
| | | | 5P | 5P | bpNLS | |
| 5 | Rep1.BFP. | Rep1.BFP. | Rep1. | Rep1. | NLS-nCas9- | Yes |
| | FwdGuide | RevGuide | BFP2GFP. | BFP2GFP. | linker- | |
| | | | TopDonor. | BotDonor. | SplintR- | |
| | | | Recoded.5P | Recoded.5P | bpNLS | |
| 6 | Rep1.BFP. | Rep1.BFP. | Rep1. | Rep1. | NLS-nCas9- | Yes |
| | FwdGuide. | RevGuide. | BFP2GFP. | BFP2GFP. | linker- | |
| | SpPAMmut | SpPAMmut | TopDonor. | BotDonor. | SplintR- | |
| | | | SpPAMmut. | SpPAMmut. | bpNLS | |
| | | | Recoded.5P | Recoded.5P | | |
| 7 | Rep1.BFP. | Rep1.BFP. | Rep1. | Rep1. | NLS-nCas9- | Yes |
| | FwdGuide | RevGuide | BFP2GFP. | BFP2GFP. | linker- | |
| | | | TopDonor. | BotDonor. | SplintR- | |
| | | | Recoded. | Recoded. | bpNLS | |
| | | | 5P.endPhos | 5P.endPhos | | |

### Example 2. Editing to Convert BFP to GFP by Replacer 2

An experiment can be performed similar to Example 1 but adjusted to fit a Replacer 2 format. The ligases used here are T4 ligase, hLIG1(233-919), and hLIG1(119-919). The Replacer 2 gRNA contains a spacer, scaffold, and DBS. The gRNAs are delivered individually (1-sided Replacer 2) or in pairs (2-sided Replacer 2), and the gRNAs contain 2′-O-methyl 3′-phosphorothioate nucleotides at the first three and last three positions. The DNA donors include a FBS and a guide binding site (GBS) that can hybridize to the DBS. Some DNA donors contain SpPAM mutations and some DNA donors have phosphorothioate bonds at the first three and last three positions. Some DNA donors are recoded. The DNA donors are phosphorylated on the 5′ end. The DNA donors are delivered as pairs in the Replacer 2 format. Some of the gRNAs and donor DNAs are annealed prior to LNP formulation. Table 13 details this experiment. Sequences corresponding to the names in the table may be found herein.

TABLE 13

| | | | | | | Anneal |
|---|---|---|---|---|---|---|
| Condition | Forward Guide | Reverse Guide | Top Donor | Bottom Donor | Ligase | both sides? |
| 1 | Rep2.BFP. | | Rep2. | Rep2. | NLS-nCas9- | Yes |
| | FwdGuide | | BFP2GFP. | BFP2GFP. | linker- | |
| | | | TopDonor. | BotDonor. | hLIG1(233- | |
| | | | SpPAMmut. | SpPAMmut. | 919)-bpNLS | |
| | | | 5P | 5P | | |
| 2 | | Rep2.BFP. | Rep2. | Rep2. | NLS-nCas9- | Yes |
| | | RevGuide | BFP2GFP. | BFP2GFP. | linker- | |
| | | | TopDonor. | BotDonor. | hLIG1(233- | |
| | | | SpPAMmut. | SpPAMmut. | 919)-bpNLS | |
| | | | 5P | 5P | | |
| 3 | Rep2.BFP. | Rep2.BFP. | Rep2. | Rep2. | NLS-nCas9- | Yes |
| | FwdGuide | RevGuide | BFP2GFP. | BFP2GFP. | linker- | |
| | | | TopDonor. | BotDonor. | hLIG1(233- | |
| | | | SpPAMmut. | SpPAMmut. | 919)-bpNLS | |
| | | | 5P | 5P | | |
| 4 | Rep2.BFP. | Rep2.BFP. | Rep2. | Rep2. | NLS-nCas9- | No |
| | FwdGuide | RevGuide | BFP2GFP. | BFP2GFP. | linker- | |
| | | | TopDonor. | BotDonor. | hLIG1(233- | |
| | | | SpPAMmut. | SpPAMmut. | 919)-bpNLS | |
| | | | 5P | 5P | | |
| 5 | Rep2.BFP. | Rep2.BFP. | Rep2.BFP2GFP. | Rep2. | NLS-nCas9- | Yes |
| | FwdGuide | RevGuide | TopDonor. | BFP2GFP. | linker- | |
| | | | SpPAMmut. | BotDonor. | hLIG1(233- | |
| | | | Recoded.5P | SpPAMmut. | 919)-bpNLS | |
| | | | | Recoded.5P | | |
| 6 | Rep2.BFP. | Rep2.BFP. | Rep2. | Rep2. | NLS-nCas9- | Yes |
| | FwdGuide | RevGuide | BFP2GFP. | BFP2GFP. | linker- | |
| | | | TopDonor. | BotDonor. | hLIG1(233- | |
| | | | 5P | 5P | 919)-bpNLS | |
| 7 | Rep2.BFP. | Rep2.BFP. | Rep2. | Rep2. | NLS-nCas9- | Yes |
| | FwdGuide | RevGuide | BFP2GFP. | BFP2GFP. | linker- | |
| | | | TopDonor. | BotDonor. | hLIG1(233- | |
| | | | SpPAMmut. | SpPAMmut. | 919)-bpNLS | |
| | | | Recoded. | Recoded. | | |
| | | | 5P.endPhos | 5P.endPhos | | |
| 8 | Rep2.BFP. | | Rep2. | Rep2. | NLS-nCas9- | Yes |
| | FwdGuide | | BFP2GFP. | BFP2GFP. | linker- | |
| | | | TopDonor. | BotDonor. | T4LIG- | |
| | | | SpPAMmut. | SpPAMmut. | bpNLS | |
| | | | 5P | 5P | | |
| 9 | | Rep2.BFP. | Rep2. | Rep2. | NLS-nCas9- | Yes |
| | | RevGuide | BFP2GFP. | BFP2GFP. | linker- | |
| | | | TopDonor. | BotDonor. | T4LIG- | |
| | | | SpPAMmut. | SpPAMmut. | bpNLS | |
| | | | 5P | 5P | | |
| 10 | Rep2.BFP. | Rep2.BFP. | Rep2. | Rep2. | NLS-nCas9- | Yes |
| | FwdGuide | RevGuide | BFP2GFP. | BFP2GFP. | linker- | |
| | | | TopDonor. | BotDonor. | T4LIG- | |
| | | | SpPAMmut. | SpPAMmut. | bpNLS | |
| | | | Recoded.5P | Recoded.5P | | |
| 11 | Rep2.BFP. | | Rep2. | Rep2. | NLS-nCas9- | Yes |
| | FwdGuide | | BFP2GFP. | BFP2GFP. | linker- | |
| | | | TopDonor. | BotDonor. | hLIG1(119- | |
| | | | SpPAMmut. | SpPAMmut. | 919)-bpNLS | |
| | | | 5P | 5P | | |
| 12 | | Rep2.BFP. | Rep2. | Rep2. | NLS-nCas9- | Yes |
| | | RevGuide | BFP2GFP. | BFP2GFP. | linker- | |
| | | | TopDonor. | BotDonor. | hLIG1(119- | |
| | | | SpPAMmut. | SpPAMmut. | 919)-bpNLS | |
| | | | 5P | 5P | | |
| 13 | Rep2.BFP. | Rep2.BFP. | Rep2. | Rep2. | NLS-nCas9- | Yes |
| | FwdGuide | RevGuide | BFP2GFP. | BFP2GFP. | linker- | |
| | | | TopDonor. | BotDonor. | hLIG1(119- | |
| | | | SpPAMmut. | SpPAMmut. | 919)-bpNLS | |
| | | | Recoded.5P | Recoded.5P | | |

### Example 3. Editing to Insert mGL in Front of CBX1 by Replacer 2

An editing experiment can be performed to insert monomeric Green Lantern (mGL) in the genome of HEK293T cells in front of the CBX1 gene such that a fusion protein is formed that exhibits green fluorescence. This fluorescence can be detected by flow cytometry as in Examples 1 and 2. The experiment is conducted in a similar way to Example 2 except that the sequences of the gRNAs and DNA donors are different and enable insertion of mGL into the genome rather than insertion of a sequence that changes blue fluorescent protein (BFP) to green fluorescent protein (GFP). The DNA donors in Example 3 are longer than in Example 2 and are synthesized by GenScript. The DNA donors are phosphorylated on the 5′ end. Table 14 details this experiment. Sequences corresponding to the names in the table may be found herein.

TABLE 14

| | | | | | | Anneal |
|---|---|---|---|---|---|---|
| Condition | Forward Guide | Reverse Guide | Top Donor | Bottom Donor | Ligase | both sides? |
| 1 | Rep2.CBX1. | | Rep2. | Rep2. | NLS-nCas9- | Yes |
| | FwdGuide | | mGL-CBX1. | mGL-CBX1. | linker- | |
| | | | TopDonor. | BotDonor. | hLIG1(233- | |
| | | | SpPAMmut. | SpPAMmut. | 919)-bpNLS | |
| | | | 5P | 5P | | |
| 2 | | Rep2.CBX1. | Rep2. | Rep2. | NLS-nCas9- | Yes |
| | | RevGuide | mGL-CBX1. | mGL-CBX1. | linker- | |
| | | | TopDonor. | BotDonor. | hLIG1(233- | |
| | | | SpPAMmut. | SpPAMmut. | 919)-bpNLS | |
| | | | 5P | 5P | | |
| 3 | Rep2.CBX1. | Rep2.CBX1. | Rep2. | Rep2. | NLS-nCas9- | Yes |
| | FwdGuide | RevGuide | mGL-CBX1. | mGL-CBX1. | linker- | |
| | | | TopDonor. | BotDonor. | hLIG1(233- | |
| | | | SpPAMmut. | SpPAMmut. | 919)-bpNLS | |
| | | | 5P | 5P | | |
| 4 | Rep2.CBX1. | | Rep2. | Rep2. | NLS-nCas9- | Yes |
| | FwdGuide | | mGL-CBX1. | mGL-CBX1. | linker- | |
| | | | TopDonor. | BotDonor. | T4LIG- | |
| | | | SpPAMmut. | SpPAMmut. | bpNLS | |
| | | | 5P | 5P | | |
| 5 | | Rep2.CBX1. | Rep2. | Rep2. | NLS-nCas9- | Yes |
| | | RevGuide | mGL-CBX1. | mGL-CBX1. | linker- | |
| | | | TopDonor. | BotDonor. | T4LIG- | |
| | | | SpPAMmut. | SpPAMmut. | bpNLS | |
| | | | 5P | 5P | | |
| 6 | Rep2.CBX1. | Rep2.CBX1. | Rep2. | Rep2. | NLS-nCas9- | Yes |
| | FwdGuide | RevGuide | mGL-CBX1. | mGL-CBX1. | linker- | |
| | | | TopDonor. | BotDonor. | T4LIG- | |
| | | | SpPAMmut. | SpPAMmut. | bpNLS | |
| | | | 5P | 5P | | |
| 7 | Rep2.CBX1. | | Rep2. | Rep2. | NLS-nCas9- | Yes |
| | FwdGuide | | mGL-CBX1. | mGL-CBX1. | linker- | |
| | | | TopDonor. | BotDonor. | hLIG1(119- | |
| | | | SpPAMmut. | SpPAMmut. | 919)-bpNLS | |
| | | | 5P | 5P | | |
| 8 | | Rep2.CBX1. | Rep2. | Rep2. | NLS-nCas9- | Yes |
| | | RevGuide | mGL-CBX1. | mGL-CBX1. | linker- | |
| | | | TopDonor. | BotDonor. | hLIG1(119- | |
| | | | SpPAMmut. | SpPAMmut. | 919)-bpNLS | |
| | | | 5P | 5P | | |
| 9 | Rep2.CBX1. | Rep2.CBX1. | Rep2. | Rep2. | NLS-nCas9- | Yes |
| | FwdGuide | RevGuide | mGL-CBX1. | mGL-CBX1. | linker- | |
| | | | TopDonor. | BotDonor. | hLIG1(119- | |
| | | | SpPAMmut. | SpPAMmut. | 919)-bpNLS | |
| | | | 5P | 5P | | |

### Example 4. Treatment of a Genetic Disease in a Patient

A human patient with sickle cell disease comes to a physician for treatment. The patient is identified as having a hemoglobin gene mutation. Hematopoietic stem and progenitor cells are collected from the patient's peripheral blood. The cells are edited by contacting the cells' genomes with a nCas9-DNA ligase fusion protein, a gRNA, and a donor DNA that includes a corrected hemoglobin gene. The gRNA recruits the fusion protein to the gene mutation, and the nCas9 nicks the patient's DNA on one side flanking the mutation. The gRNA binds to a genomic flap generated by the nick, and to the donor DNA, and forms an RNA splint for the ligase to ligate the genomic flap to the donor DNA. Another fusion protein nicks the opposite strand of the mutated hemoglobin gene using a second gRNA on the other side of the mutation, and ligates the other side of the donor DNA. The mutated DNA is thus replaced with the donor DNA, and the cell with the donor DNA is transfused back into the patient, thus treating the genetic disease in the patient.

### Example 5. Enhancing a Crop

In a soybean plant, a germ cell is microinjected with an expression vector encoding an nCas9-DNA ligase fusion protein, and with a gRNA and donor DNA encoding an herbicide resistance gene. gRNA recruits the fusion protein to a suitable spot within the soybean genome which doesn't already include a gene. The nCas9 nicks the soybean's DNA on one side flanking the spot. The gRNA also recruits the donor DNA to bind to a genomic flap created by the nick, and the ligase seals the nick using the donor DNA itself as a splint. Another fusion protein nicks the opposite strand of the soybean's DNA on the other side flanking the spot, and ligates the other side of the donor DNA, thus integrating the herbicide resistance gene into the germ cell. The germ cell eventually produces a seed, and the seeds are harvested to grow herbicide resistant soybeans.

### Example 6. In Vitro 1-Sided Replacer 2 Using T4 Ligase

To demonstrate the usefulness of the components and methods described herein for editing nucleic acids, in vitro experiments were performed. The experiments in this example specifically assessed the feasibility of 1-sided Replacer 2. The experiments used a 100 bp, 5′-Cy5-labeled double-stranded DNA (dsDNA) substrate (IDT) that corresponded to the blue fluorescent protein (BFP) target region (see examples 1 and 2), with the site of nicking located in the middle at base pair 50. 5′-phosphorylated dsDNA donors (IDT) containing a variable GBS, 13 nt flap binding site (FBS), and a protospacer adjacent motif (PAM) mutation were used in conjunction with gRNAs (Agilent) containing the corresponding variable DBS. 5′-Cy5-labeled dsDNA substrate and 5′-phosphorylated dsDNA donor were separately annealed using complementary oligonucleotides by heating to 95 C for 5 min followed by slowly cooling to room temperature.

In vitro 1-sided Replacer 2 reactions were performed by first incubating gRNA (30 nM final) and dsDNA donor (30 nM final) with recombinant S. pyogenes nicking Cas9 (Cas9n; IDT; 30 nM final) for 10 min at room temperature, followed by the addition of T4 ligase (NEB; 200 U final), ATP (1 mM final), and 5′-Cy5-labeled dsDNA substrate (3 nM Final). Reactions were carried out in the presence of NEB Buffer 3.1 (1× final) at 37 C for 1 hr (final volume of 10 ul). Reactions were terminated by the addition of 0.5% SDS and 100 ug/ml Proteinase K, and incubated at 37 C for 30 min. Reaction products were then combined with 2× formamide gel loading buffer (90% formamide; 10% glycerol; 0.01% bromophenol blue), denatured at 95° C. for 10 min, and separated by denaturing urea PAGE gel (15% TBE-urea, 55° C., 200 V). DNA products were visualized by Cy5 fluorescence signal using a LI-COR Odyssey CLx imager.

In addition to the intact 100 bp 5′-Cy5-labeled dsDNA substrate, a nicked 5′-Cy5-labeled dsDNA substrate and a final ligation product were included as size controls. The nicked 5′-Cy5-labeled dsDNA control was annealed using two 50mers corresponding to the top strand oligo of the 100 bp 5′-Cy5-labeled dsDNA substrate (a 5′-Cy5-labeled 50mer and a 5′-phosphorylated 50mer) and its complementary 100mer bottom strand oligo. The final ligation product control was annealed and ligated using the 5′-Cy5-labeled 50mer and the bottom 100mer from the nicked control along with the 150 nt top strand donor oligo.

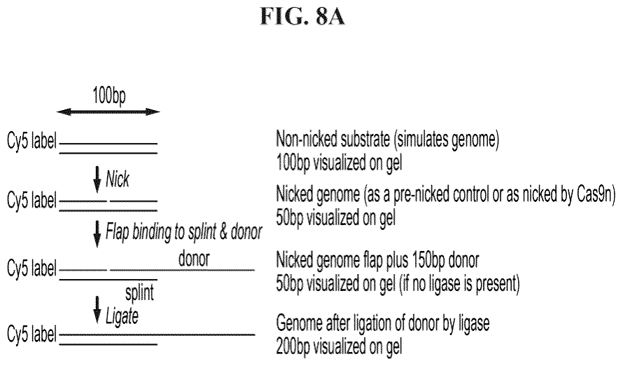

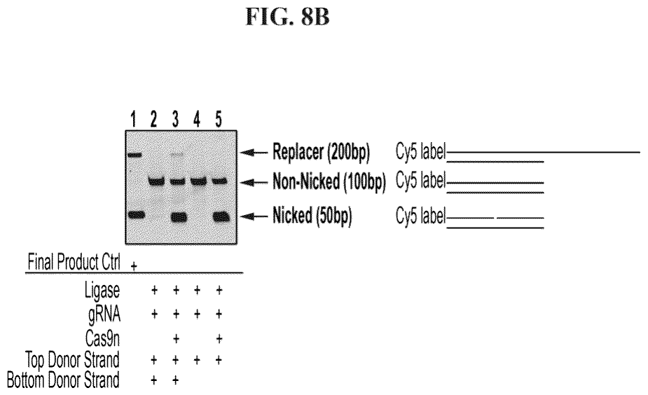

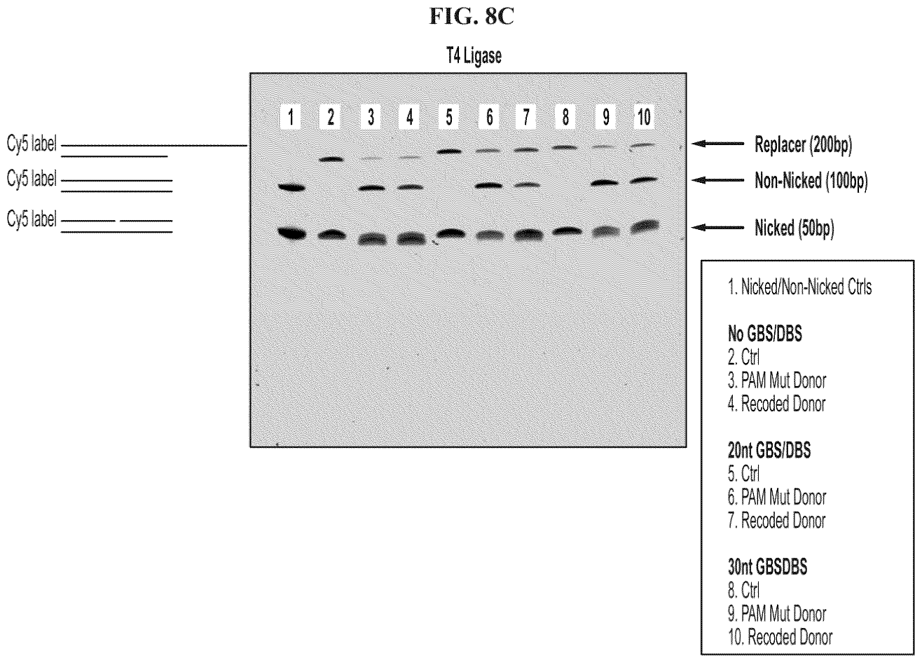

FIG. 8A illustrates an exemplary nicking and ligation pattern of an integrating nucleic acid. FIG. 8B illustrates an exemplary nucleic acid gel showing pattern associated with In Vitro 1-Sided Replacer 2 using 30 nt GBS/DBS and Thermostable T4 Ligase. Using a 30 nt GBS/DBS combination, a donor containing a PAM mutation, and a thermostable T4 ligase (Hi-T4, NEB), we were able to produce a final Replacer product (Lane 3) corresponding to the size of our control product (Lane 1). Replacer products were not detected in the absence of nicking Cas9 (Cas9n) (Lane 2), or in the absence of the bottom donor which serves as the splint (Lanes 4 & 5). FIG. 8C illustrates an exemplary nucleic acid gel showing pattern associated with in vitro 1-Sided Replacer 2 using Variable Length GBS/DBS Combinations and T4 Ligase. Using regular T4 ligase (NEB), we were to produce a final Replacer product corresponding to the size of the control when using multiple GBS/DBS combinations, including No GBS/DBS, 20 nt GBS/DBS, and 30 nt GBS/DBS. Additionally, in this experiment, recoded dsDNA donors containing PAM mutation were more efficient at producing final Replacer products compared to PAM mutant dsDNA donors that were not recoded. The results indicate that a DNA ligase may be used with an RNA-guided endonuclease to edit a target nucleic acid.

### Example 7. Use of 1-Sided Replacer 2 with Nicking Cas9 and Multiple DNA Ligases in Various Coupling Architectures in Mammalian Cells

Components used to edit a blue fluorescent protein (BFP) gene stably integrated into HEK293T cells were co-delivered by lipofectamine 2000 transfection. The components included a chemically synthesized guide RNA (SEQ ID NO: 166, mG*mC*mU*GAAGCACUGCACGCCAUGUUUUAGAGCUAGAAAUAGCAAGUUAAAAUA AGGCUAGUCCGUUAUCGACUUGAAAAAGUCGGACCGAGUCGGUCCAGCUGCGGUAUU GUGGmC*mG*mU) with 2′-O-methyl and phosphorothioate chemical modifications on the 5′ and 3′ ends, an integrating nucleic acid with a 5′ phosphate end modification (SEQ ID NO: 167, /5Phos/cgtaTgtcagggtggtcacGAGgg), a splinting nucleic acid with locked nucleic acid and phosphorothioate modifications (SEQ ID NO: 169, +c*c*+CT+CG+TG+AC+CA+CC+CT+GA+CA+TA+CGGCGTGCAgtgcttACGCCA+CA+AT+A C+CG+CA+G*C*+T), and either a single mRNA encoding nicking Cas9 fused to a ligase, or a pair of mRNAs encoding nicking Cas9 and a ligase.

The integrating nucleic acid and splinting nucleic acid were synthesized by Integrated DNA Technologies (IDT). All mRNAs corresponding to Cas9n (H840A) and all ligases are generated via in vitro transcription (IVT) reactions using the HiScribe T7 High Yield RNA Synthesis Kit (NEB). Coding sequences are cloned into an IVT vector that contains a single copy of the 5′UTR and two copies of the 3′UTR from the human beta globin gene, in addition to a 152 nt polyA tail. Plasmid DNA containing coding sequences are linearized using an XbaI restriction site located immediately downstream of the polyA tail. Linearized plasmids are then purified via phenol:chloroform extraction followed by ethanol precipitation. mRNAs are produced via IVT reactions that contain N1-Methylpseudouridine-5′-Triphosphate (TriLink BioTech) in place of Uridine-Triphosphate, and capped co-transcriptionally with CleanCap Reagent AG (3′ OMe) (TriLink BioTech). IVT reactions are incubated at 37° C. for 2 hours, followed by DNAse I digestion of the template DNA. Finally, mRNA products are purified using LiCl precipitation, quantified (Qubit Fluorometric Quantification; ThermoFisher), and checked for integrity by denaturing gel electrophoresis. “Ligase in trans” refers to Cas9 H840A nickase combined with T4 ligase fused to leucine zipper on its C terminus (T4-LZ, SEQ ID NO: 145). “LZ; C terminal Ligase” refers to Cas9 H840A nickase fused to a leucine zipper on its C terminus (nCas9-LZ, SEQ ID NO: 133) combined with a ligase fused to a leucine zipper on its N terminus for T4 (LZ-T4, SEQ ID NO: 142), SplintR (LZ-SplintR, SEQ ID NO: 141), or hLIG4(1-620) (LZ-hLIG4(1-620), SEQ ID NO: 146). “LZ; N terminal Ligase” refers to Cas9 H840A nickase fused to a leucine zipper on its N terminus (LZ-nCas9, SEQ ID NO: 147) combined with a ligase fused to a leucine zipper on its C terminus for T4 (T4-LZ, SEQ ID NO: 145), SplintR (SplintR-LZ, SEQ ID NO: 148), or hLIG4(1-620) (hLIG4(1-620)-LZ, SEQ ID NO: 149). “Fusion; C terminal Ligase” refers to Cas9 H840A nickase fused to a ligase with the ligase on the C terminus for T4 (nCas9-T4, SEQ ID NO: 131), SplintR (nCas9-SplintR, SEQ ID NO: 129), or hLIG4(1-620) (nCas9-hLIG4(1-620) SEQ ID NO: 150). “Fusion; N terminal Ligase” refers to Cas9 H840A nickase fused to a ligase with the ligase on the N terminus for T4 (T4-nCas9, SEQ ID NO: 151), SplintR (SplintR-nCas9, SEQ ID NO: 152), or hLIG4(1-620) (hLIG4(1-620)-nCas9, (SEQ ID NO: 153). The gRNA contained a spacer, scaffold, and donor binding site. The splinting integrating nucleic acid contained a guide binding site and a flap binding site. The ligating integrating nucleic acid and splinting nucleic acid were partially complementary.

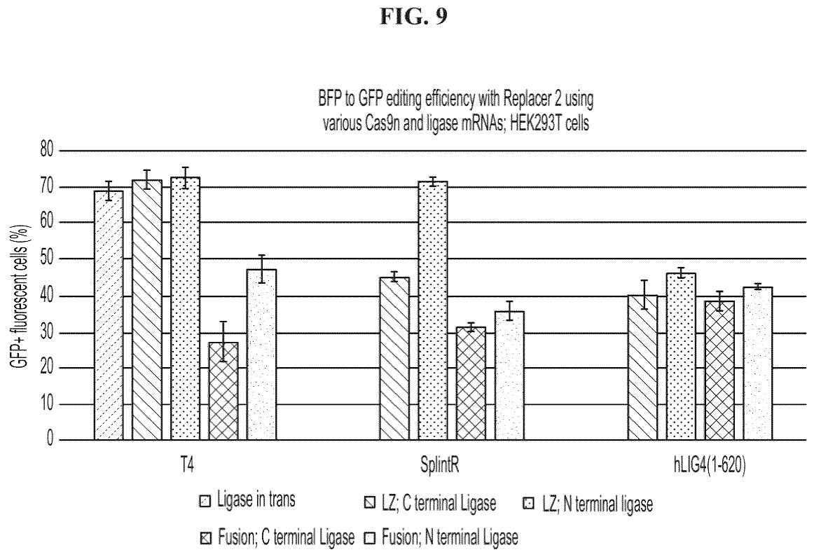

The integrating nucleic acid and splinting nucleic acid were hybridized using an annealing reaction, then mixed with the guide RNA and mRNA and formulated with lipofectamine 2000 in OptiMEM prior to delivery to the adherent HEK293 cells in 96-well plates. After 24-48 hours, the cells were detached with 0.05% Trypsin-EDTA and run through a flow cytometer to measure the percentage of cells expressing green fluorescent protein (GFP), indicating gene editing from BFP to GFP (FIG. 9). Gene editing was observed with T4, SplintR, and hLIG4(1-620) ligases when fused to nCas9, interacting with nCas9 through leucine zippers, or delivered in trans with no leucine zipper interaction.

The results here demonstrate the usefulness of using a DNA ligase with an RNA-guided endonuclease to edit a target nucleic acid in a cell. The experiments in this example specifically demonstrated the feasibility of including 1-sided Replacer 2 components to edit a target nucleic acid in a mammalian cell. This example shows the effectiveness of including a DNA ligase coupled through a heterodimerization domain (here, leucine zippers) to an RNA guided endonuclease (e.g. a nicking Cas9) in nucleic acid editing such as gene editing. This also shows nucleic acid editing is possible in mammalian cells with a DNA ligase fused to an RNA guided endonuclease (e.g. T4 ligase fused to Cas9 H840A nickase), and that nucleic acid editing can be achieved by delivering the DNA ligase and RNA guided endonuclease as separate non-coupled components.

### Example 8. Use of 1-Sided Replacer 2 with Nicking Cas9 and T4 DNA Ligase to Make a Variety of Edits at Multiple Genomic Targets

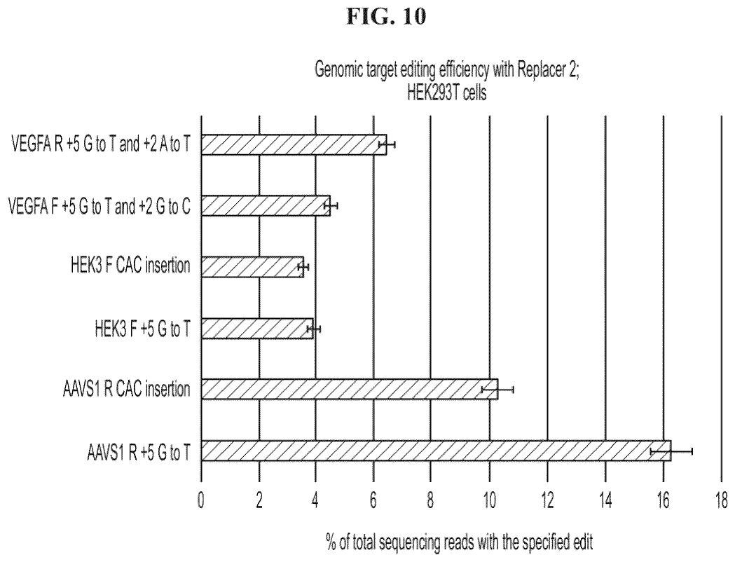

Components used to edit genomic targets in HEK293T cells were co-delivered by lipofectamine 2000 transfection. The components included a chemically synthesized guide with 2′-O-methyl and phosphorothioate chemical modifications on the 5′ and 3′ ends, an integrating nucleic acid with a 5′ phosphate end modification, a splinting nucleic acid with locked nucleic acid and phosphorothioate modifications, an mRNA encoding nicking Cas9 (LZ-nCas9, SEQ ID NO: 147), and an mRNA encoding a ligase (T4-LZ, SEQ ID NO: 145). Target-specific guides, splinting and integrating nucleic acids are listed in Table 15. The integrating nucleic acid and splinting nucleic acid were synthesized by Integrated DNA Technologies (IDT) and both mRNAs were generated via in vitro transcription reactions using the methods described in Example 7. The gRNA contained a spacer, scaffold, and donor binding site. The splinting integrating nucleic acid contained a guide binding site and a flap binding site. The ligating integrating nucleic acid and splinting nucleic acid were partially complementary. The integrating nucleic acid and splinting nucleic acid were hybridized using an annealing reaction, then mixed with the guide RNA and mRNA and formulated with lipofectamine 2000 in OptiMEM prior to delivery to the adherent HEK293 cells in 96-well plates. After 24-48 hours, genomic DNA was extracted from the cells using QuickExtract and genomic targets were amplified using Q5 DNA Polymerase. The PCR program ran at 98 C for 30 seconds, then 35 cycles of 98 C for 5 seconds, 67 C for 20 seconds, and 72 C for 20 seconds, then finally 72 C for 2 minutes. PCR primers are listed in Table 15. PCR products were cleaned up with ExoCIP treatment and submitted for next generation sequencing (NGS) by Azenta using their Amplicon-EZ service. Sequencing reads were merged and aligned to the amplicon of interest, and the percentage total reads that matched the intended edit was calculated (FIG. 10). This example shows the effectiveness of gene editing with 1-sided Replacer 2 in mammalian cells at a variety of genomic targets. The types of edits here include making a single point mutation (HEK3 F+5 G to T), a pair of point mutations (VEGFA R+5 G to T and +2 A to T, VEGFA F+5 G to T and +2 G to C, and AAVS1 R+5 G to T), or a trinucleotide insertion (HEK3 F CAC insertion and AAVS1 R CAC insertion) using 1-sided Replacer 2.

TABLE 15

| Condi- | | | | PCR |
|---|---|---|---|---|
| tion | Guide | Splint | Donor | Primers |
| VEGFA | SEQ ID | SEQ ID | SEQ ID | SEQ ID |
| R +5 | NO: 170 | NO: 174 | NO: 180 | NO: 186 |
| G to T | mC*mA*mC* | +C*C*+TT+ | /5Phos/ | ACACTCTTT |
| and +2 | CCCGGCUC | TC+CA+AA | ATGATG | CCCTACAC |
| A to T | UGGCUAAA | +GC+CC+A | GAATGGG | GACGCTCTT |
| | GGUUUUAG | T+TC+CA+T | CTTT | CCGATCTT |
| | AGCUAGAA | C+ATtagccag | GGAAAGG | GCCGCTCAC |
| | AUAGCAAG | agccggACGC | | TTTGATGT |
| | UUAAAAUA | CA+CA+AT+ | | CT; |
| | AGGCUAGU | AC+CG+C | | SEQ ID |
| | CCGUUAUC | A+G*C*+T | | NO: 187 |
| | GACUUGAA | | | GACTGGAGT |
| | AAAGUCGG | | | TCAGACG |
| | ACCGAGUC | | | TGTGCTCTT |
| | GGUCCAGC | | | CCGATCTG |
| | UGCGGUAU | | | GGGAGAGGG |
| | UGUGGmC* | | | ACACACA |
| | mG*mU | | | GA |
| VEGFA | SEQ ID | SEQ ID | SEQ ID | SEQ ID |
| F +5 | NO: 171 | NO: 175 | NO: 181 | NO: 223 |
| G to T | mG*mA*mU* | +A*C*+AA+ | /5Phos/ | ACACTCTTT |
| and +2 | GUCUGCAG | TG+TG+CC | TCAGT | CCCTACAC |
| G to C | GCCAGAUG | +AT+CT+G | GCTCCA | GACGCTCTT |
| | AGUUUUAG | G+AG+CA+ | GATGGC | CCGATCTT |
| | AGCUAGAA | CT+GAtctgg | ACATTGT | GCCGCTCA |
| | AUAGCAAG | cctgcagaTC | | CTTTGATGT |
| | UUAAAAUA | ATGC+AG+ | | CT; |
| | AGGCUAGU | CC+CG+GA+ | | SEQ ID |
| | CCGUUAUC | AC+C*A*+ | | NO: 224 |
| | GACUUGAA | C | | GACTGGAGT |
| | AAAGUCGG | | | TCAGACG |
| | ACCGAGUC | | | TGTGCTCTT |
| | GGUCCGUG | | | CCGATCTG |
| | GUUCCGG | | | GGGAGAGGG |
| | GCUGCAmU* | | | ACACACA |
| | mG*mA | | | GA |
| HEK3 | SEQ ID | SEQ ID | SEQ ID | SEQ ID |
| F CAC | NO: 172 | NO: 176 | NO: 182 | NO: 188 |
| inser- | mG*mG*mC* | +G*C*+TT+ | /5Phos/ | ACACTCTTT |
| tion | ccagacuga | CC+TT+TC+ | gtgTGAT | CCCTACAC |
| | gcacgugaG | CT+CT+GC+ | GGCAGAG | GACGCTCTT |
| | UUUUAGAGC | CA+TC+Ac+ | GAA | CCGATCT |
| | UAGAAAUAG | accgtgctcag | AGGAAGC | ccctggcctg |
| | CAAGUUAAA | tctgTCATGC+ | | ggtcaatcc; |
| | AUAAGGCUA | AG+CC+CG+ | | SEQ ID |
| | GUCCGUUA | GA+AC+C* | | NO: 189 |
| | UCGACUUGA | A*+C | | GACTGGAGTT |
| | AAAAGUC | | | CAGACG |
| | GGACCGAGU | | | TGTGCTCTT |
| | CGGUCCG | | | CCGATCTct |
| | UGGUUCCGG | | | gaagggcca |
| | GCUGCAm | | | ggtccctc |
| | U*mG*mA | | | |
| HEK3 | SEQ ID | SEQ ID | SEQ ID | SEQ ID |
| F +5 | NO: 221 | NO: 177 | NO: 183 | NO: 225 |
| G to T | mG*mG*mC* | +A*G*+GG+ | /5Phos/ | ACACTCTTT |
| | ccagacuga | CT+TC+CT+ | TGATTG | CCCTACAC |
| | gcacgugaG | TT+CC+TC+ | CAGAGGA | GACGCTCTT |
| | UUUUAGAGC | TG+CA+AT+ | AAGGA | CCGATCT |
| | UAGAAAUAG | CAcgtgctca | AGCCCT | ccctggcctg |
| | CAAGUUAAA | gtctgTCATG | | ggtcaatcc; |
| | AUAAGGCUA | C+AG+CC+C | | SEQ ID |
| | GUCCGUUA | G+GA+AC+ | | NO: 226 |
| | UCGACUUGA | C*A*+C | | GACTGGAGTT |
| | AAAAGUC | | | CAGACG |
| | GGACCGAGU | | | TGTGCTCTTC |
| | CGGUCCG | | | CGATCTct |
| | UGGUUCCGG | | | gaagggcca |
| | GCUGCAm | | | ggtccctc |
| | U*mG*mA | | | |
| AAVS1 | SEQ ID | SEQ ID | SEQ ID | SEQ ID |
| R CAC | NO: 173 | NO: 178 | NO: 184 | NO: 190 |
| inser- | mG*mC*mG* | +A*T*+TA+ | /5Phos/ | ACACTCTTT |
| tion | acuccugga | GC+AG+AA | gtgCCA | CCCTACAC |
| | aguggccaG | +GT+GG+C | AGGGCC | GACGCTCTT |
| | UUUUAGAGC | C+CT+TG+ | ACTTCT | CCGATCT |
| | UAGAAAUAG | Gc+acccactt | GCTAAT | CGCCGGGAA |
| | CAAGUUAAA | ccaggACGC | | CTGCCG |
| | AUAAGGCUA | CA+CA+AT+ | | CTGGC; |
| | GUCCGUUA | AC+CG+C | | SEQ ID |
| | UCGACUUGA | A+G*C*+T | | NO: 191 |
| | AAAAGUC | | | GACTGGAGTT |
| | GGACCGAGU | | | CAGACG |
| | CGGUCCA | | | TGTGCTCTTC |
| | GCUGCGGUA | | | CGATCT |
| | UUGUGG | | | GAGGAGGCCC |
| | mC*mG*mU | | | TCATCT |
| | | | | GGCG |
| AAVS1 | SEQ ID | SEQ ID | SEQ ID | SEQ ID |
| R +5 | NO: 222 | NO: 179 | NO: 185 | NO: 227 |
| G to T | mG*mC*mG* | +T*C*+CA+ | /5Phos/ | ACACTCTTT |
| | acuccugga | TT+AG+CA+ | CCAAT | CCCTACAC |
| | aguggccaG | GA+AG+T | GGCCACT | GACGCTCTT |
| | UUUUAGAGC | G+GC+CA+ | TCTGCT | CCGATCT |
| | UAGAAAUAG | TT+GGccac | AATGGA | CGCCGGGAA |
| | CAAGUUAAA | ttccaggACG | | CTGCCG |
| | AUAAGGCUA | CCA+CA+A | | CTGGC; |
| | GUCCGUUA | T+AC+CG+ | | SEQ ID |
| | UCGACUUGA | CA+G*C*+T | | NO: 228 |
| | AAAAGUC | | | GACTGGAGT |
| | GGACCGAGU | | | TCAGACG |
| | CGGUCCA | | | TGTGCTCTT |
| | GCUGCGGUA | | | CCGATCT |
| | UUGUGG | | | GAGGAGGCC |
| | mC*mG*mU | | | CTCATCT |
| | | | | GGCG |

### Example 9. Use of 2-Sided Replacer 2 with Nicking Cas9 and T4 DNA Ligase to Make Deletions and Sequence Replacements at Multiple Genomic Targets

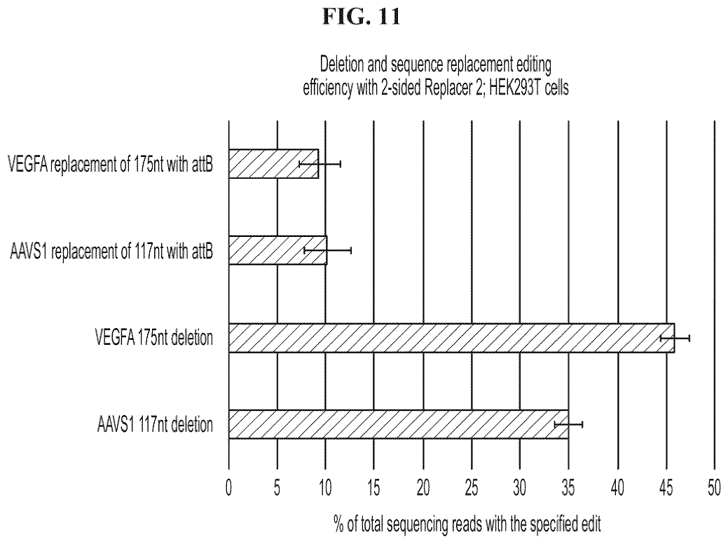

Components used to edit genomic targets in HEK293T cells were co-delivered by lipofectamine 2000 transfection. The components included two chemically synthesized guides with 2′-O-methyl and phosphorothioate chemical modifications on the 5′ and 3′ ends, two integrating nucleic acids with a 5′ phosphate end modification, two splinting nucleic acids with locked nucleic acid and phosphorothioate modifications, an mRNA encoding nicking Cas9 (LZ-nCas9, SEQ ID NO: 147), and an mRNA encoding a ligase (T4-LZ, SEQ ID NO: 145). For both “VEGFA replacement of 175 nt with attB” and “VEGFA 175 nt deletion”, the two guide RNAs used were VEGFA_R (SEQ ID NO: 170) and VEGFA_F (SEQ ID NO: 171). For both “AAVS1 replacement of 117 nt with attB” and “AAVS1 117 nt deletion”, the two guide RNAs used were AAVS1_R (SEQ ID NO: 173) and AAVS1_F (SEQ ID NO: 192, mG*mC*mU*ggccccccaccgccccaGUUUUAGAGCUAGAAAUAGCAAGUUAAAAUAAGGCUA GUCCGUUAUCGACUUGAAAAAGUCGGACCGAGUCGGUCCGUGGUUCCGGGCUGCAmU*mG*mA). For “VEGFA replacement of 175 nt with attB”, the splinting nucleic acids used were SEQ ID NO: 193 (+g*g*+ag+ac+cg+cc+gt+cg+tc+ga+ca+ag+cctctggcctgcagaTCATGC+AG+CC+CG+GA+AC+C*A*+C) and SEQ ID NO: 194 (+g*g*+cg+gt+ct+cc+gt+cg+tc+ag+ga+tc+attagccagagccggACGCCA+CA+AT+AC+CG+CA+G*C*+T), and the integrating nucleic acids used were SEQ ID NO: 195 (/5Phos/ggcttgtcgacgacggcggtctcc) and SEQ ID NO: 196 (/5Phos/atgatcctgacgacggagaccgcc). For “VEGFA 175 nt deletion”, the splinting nucleic acids used were SEQ ID NO: 197 (+C*C*+GT+CT+GC+AC+AC+CC+CG+GC+TC+TG+GC+TAtctggcctgcagaTCATGC+AG+CC+CG+GA+AC+C*A*+C) and SEQ ID NO: 198 (+G*C*+TC+AC+TT+TG+AT+GT+CT+GC+AG+GC+CA+GAtagccagagccggACGCCA+CA+AT+AC+CG+CA+G*C*+T), and the integrating nucleic acids used were SEQ ID NO: 199 (/5Phos/TAGCCAGAGCCGGGGTGTGCAGACGG) and SEQ ID NO: 200 (/5Phos/TCTGGCCTGCAGACATCAAAGTGAGC). For “AAVS1 replacement of 117 nt with attB”, the splinting nucleic acids used were SEQ ID NO: 201 (+g*g*+ag+ac+cg+cc+gt+cg+tc+ga+ca+ag+ccggcggtgggTCATGC+AG+CC+CG+GA+AC+C*A*+C) and SEQ ID NO: 202 (+g*g*+cg+gt+ct+cc+gt+cg+tc+ag+ga+tc+atccacttccaggACGCCA+CA+AT+AC+CG+CA+G*C*+T), and the integrating nucleic acids used were SEQ ID NO: 195 and SEQ ID NO: 196. For “AAVS1 117 nt deletion”, the splinting nucleic acids used were SEQ ID NO: 203 (+C*G*+GG+GC+AC+AG+CG+AC+TC+CT+GG+AA+GT+GGggcggtgggTCATGC+AG+CC+C G+GA+AC+C*A*+C) and SEQ ID NO: 204 (+G*G*+AA+CT+GC+CG+CT+GG+CC+CC+CC+AC+CG+CCccacttccaggACGCCA+CA+AT+A C+CG+CA+G*C*+T), and the integrating nucleic acids used were SEQ ID NO: 205 (/5Phos/CCACTTCCAGGAGTCGCTGTGCCCCG) and SEQ ID NO: 206 (/5Phos/GGCGGTGGGGGGCCAGCGGCAGTTCC). The integrating nucleic acid and splinting nucleic acid were synthesized by Integrated DNA Technologies (IDT) and both mRNAs were generated via in vitro transcription reactions using the methods described in Example 7. The gRNA contained a spacer, scaffold, and donor binding site. The splinting integrating nucleic acid contained a guide binding site and a flap binding site. There were two pairs of ligating integrating nucleic acid and splinting nucleic acid, and each pair was partially complementary to each other. The integrating nucleic acid and splinting nucleic acid were hybridized using an annealing reaction, then mixed with the guide RNA and mRNA and formulated with lipofectamine 2000 in OptiMEM prior to delivery to the adherent HEK293 cells in 96-well plates. After 24-48 hours, genomic DNA was extracted from the cells using QuickExtract and genomic targets were amplified using Q5 DNA Polymerase. The PCR program ran at 98 C for 30 seconds, then 35 cycles of 98 C for 5 seconds, 67 C for 20 seconds, and 72 C for 20 seconds, then finally 72 C for 2 minutes. PCR primers used for both “VEGFA replacement of 175 nt with attB” and “VEGFA 175 nt deletion” are SEQ ID NO: 186 and SEQ ID NO: 187. PCR primers used for both “AAVS1 replacement of 117 nt with attB” and “AAVS1 117 nt deletion” are SEQ ID NO: 190 and SEQ ID NO: 191. PCR products were cleaned up with ExoCIP treatment and submitted for next generation sequencing (NGS). Sequencing reads were merged and aligned to the amplicon of interest, and the percentage total reads that matched the intended edit was calculated (FIG. 11). This example shows that when Replacer 2 is delivered as 2 full sets of guide RNA, splint, and donor, it can delete an entire region of DNA between the nicking sites on each guide RNA, and optionally replace that region of DNA with a new DNA sequence. Since Replacer is making two separate flaps that can hybridize to each other here, this gene editing mechanism would not rely on the MMR pathway. After an attB sequence is inserted into a targeted site in the genome by Replacer, an entire synthetic gene could be inserted at that attB site if it is delivered with a Bxb1 integrase. Thus, the attB sequence replacement described here could be used for targeted insertion of large 1 kb+ DNA fragments into the genome without double strand break or mismatch repair mediated gene editing.

### Example 10. Use of 1-Sided Replacer 2 with Nicking Cas9 and T4 DNA Ligase to Integrate Methylated DNA into a Genomic Target

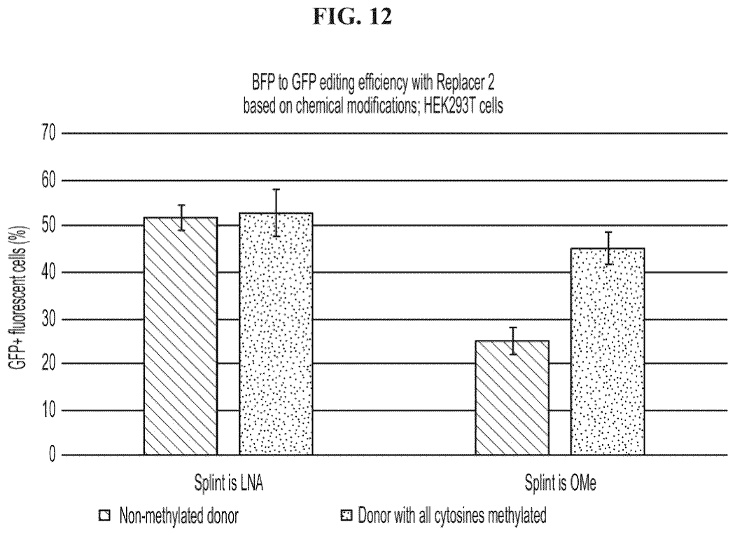

Components used to edit genomic targets in HEK293T cells were co-delivered by lipofectamine 2000 transfection. The components included a chemically synthesized guide with 2′-O-methyl and phosphorothioate chemical modifications on the 5′ and 3′ ends (SEQ ID NO: 166), an integrating nucleic acid, a splinting nucleic acid, an mRNA encoding nicking Cas9 (LZ-nCas9, SEQ ID NO: 147), and an mRNA encoding a ligase (T4-LZ, SEQ ID NO: 145). Conditions with the “non-methylated donor” used an integrating nucleic acid with a 5′ phosphate end modification (SEQ ID NO: 207, /5Phos/CGTATGTCAGGGTGGTCACG). Conditions with the “donor with all cytosines methylated” used an integrating nucleic acid with a 5′ phosphate end modification and methylated cytosines (SEQ ID NO: 207, /5Phos//5Me-dC/gtaTgt/iMe-dC/agggtggt/iMe-dC/a/iMe-dC/G). Conditions under “Splint is LNA” used a splinting nucleic acid with locked nucleic acid and phosphorothioate modifications (SEQ ID NO: 208, +C*g*+tg+ac+ca+cc+ct+ga+cA+TA+CGGCGTGCAgtgcttACGCCA+CA+AT+AC+CG+CA+G*C*+T). Conditions under “Splint is OMe” used a splinting nucleic acid with locked nucleic acid, 2′-O-methyl, and phosphorothioate modifications (SEQ ID NO: 209, mC*g*mUgmacmcamccmctmgamcAmUAmCGGCGTGCAgtgcttACGCCA+CA+AT+AC+CG+CA+G*C*+T). The integrating nucleic acid and splinting nucleic acid were synthesized by Integrated DNA Technologies (IDT) and both mRNAs were generated via in vitro transcription reactions using the methods described in Example 7. The gRNA contained a spacer, scaffold, and donor binding site. The splinting integrating nucleic acid contained a guide binding site and a flap binding site. The ligating integrating nucleic acid and splinting nucleic acid were partially complementary. The integrating nucleic acid and splinting nucleic acid were hybridized using an annealing reaction, then mixed with the guide RNA and mRNA and formulated with lipofectamine 2000 in OptiMEM prior to delivery to the adherent HEK293 cells in 96-well plates. After 24-48 hours, the cells were detached with 0.05% Trypsin-EDTA and run through a flow cytometer to measure the percentage of cells expressing green fluorescent protein (GFP), indicating gene editing from BFP to GFP (FIG. 12). This example shows that methylated DNA can be used in the integrating nucleic acid and does not negatively impact editing efficiency under ideal conditions, when the splint has LNA bases. When the splint has OMe bases instead of LNAs and thus lower affinity to the donor, methylated DNA in the donor boosts efficiency, showing that DNA methylation can improve the system by stabilizing the nucleic acid components. A methylated donor could also be used to specifically introduce DNA methylation into the genome at functional epigenetic sites such as promoters to regulate gene expression. A follow-up experiment could be conducted by performing bisulfite sequencing on the genomic region that Replacer is introducing methylated DNA into to confirm that epigenetic editing has occurred. If Replacer successfully introduces DNA methylation into this genomic region and it is believed that the region's methylation state controls gene expression, quantitative PCR could be conducted to confirm that a gene of interest has reduced mRNA expression after editing.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. An editing method, comprising:
contacting a target nucleic acid in a cell with an endonuclease at a predetermined locus of the target nucleic acid, thereby introducing a nick at the predetermined locus of the target nucleic acid;
introducing a pre-synthesized integrating nucleic acid to the cell;
introducing a pre-synthesized splint nucleic acid to the cell,
wherein the splint nucleic acid comprises a locked nucleic acid; and
ligating a 5′ end of the pre-synthesized integrating nucleic acid to a 3′ end of the nick at the predetermined locus of the target nucleic acid, wherein said ligating is performed by a ligase coupled to the endonuclease, and wherein the ligase and endonuclease are not coupled to a polymerase.

2. The method of claim 1, wherein the endonuclease comprises a class II CRISPR/Cas endonuclease.

3. The method of claim 1, wherein the endonuclease comprises Cas9 nickase.

4. The method of claim 1, further comprising contacting the endonuclease and the predetermined locus of the target nucleic acid with a guide nucleic acid.

5. The method of claim 1, wherein the pre-synthesized integrating nucleic acid comprises a mutation in relation to the target nucleic acid.

6. The method of claim 1, wherein the nick comprises a single phosphodiester strand break in the otherwise double stranded target nucleic acid.

7. The method of claim 1, wherein the target nucleic acid comprises a chromosome of the cell.

8. The method of claim 1, wherein the cell is eukaryotic.

9. An editing system, comprising:
a ligase;
an endonuclease that introduces a nick at a predetermined locus of a target nucleic acid, wherein the ligase is coupled to the endonuclease, wherein the ligase and endonuclease are not coupled to a polymerase;
a pre-synthesized integrating nucleic acid comprising a 5′ end that is ligated by the ligase to a 3′ end of the nick at the predetermined locus of the target nucleic acid; and
a pre-synthesized splint nucleic acid,
wherein the splint nucleic acid comprises a locked nucleic acid.

10. The system of claim 9, wherein the endonuclease comprises a class II CRISPR/Cas endonuclease.

11. The system of claim 9, wherein the endonuclease comprises Cas9 nickase.

12. The system of claim 9, wherein comprising a guide nucleic acid that brings the endonuclease into proximity with the predetermined locus of the target nucleic acid.

13. The system of claim 9, wherein the pre-synthesized integrating nucleic acid comprises a mutation in relation to the target nucleic acid.

14. The system of claim 9, wherein the nick comprises a single phosphodiester strand break in the otherwise double stranded target nucleic acid.

15. The system of claim 9, wherein the system is in a cell, wherein the target nucleic acid comprises a chromosome of the cell.

16. The system of claim 15, wherein the cell is eukaryotic.

17. The method of claim 1, wherein the ligase and endonuclease are fused together, directly or through a linker.

18. The method of claim 1, wherein the ligase is fused to a first dimerization domain, and the endonuclease is fused to a second dimerization domain that binds to the first dimerization domain.

19. The method of claim 1, wherein the introduction of the nick generates a flap comprising the 3′ end of the nick, and wherein the splint nucleic acid binds the flap and the 5′ end of the integrating nucleic acid.

20. The method of claim 19, wherein the splint comprises a strand of the integrating nucleic acid, or comprises a guide nucleic acid that binds to the endonuclease.

21. The method of claim 1, wherein at least part of the splint nucleic acid is not integrated into the target nucleic acid.

22. The system of claim 9, wherein the ligase and endonuclease are fused together, directly or through a linker.

23. The system of claim 9, wherein the ligase is fused to a first dimerization domain, and the endonuclease is fused to a second dimerization domain that binds to the first dimerization domain.

24. The system of claim 9, wherein the introduction of the nick generates a flap comprising the 3′ end of the nick, and the splint nucleic acid binds the flap and the 5′ end of the integrating nucleic acid.

25. The system of claim 24, wherein the splint nucleic acid comprises a strand of the integrating nucleic acid, or comprises a guide nucleic acid that binds to the endonuclease.

26. The system of claim 9, wherein at least part of the pre-synthesized splint nucleic acid is not integrated into the target nucleic acid.

27. An editing method, comprising:
contacting a target nucleic acid in a cell with an endonuclease at a predetermined locus of the target nucleic acid, thereby introducing a nick at the predetermined locus of the target nucleic acid, wherein the endonuclease is contacted with a guide nucleic acid;
introducing a pre-synthesized integrating nucleic acid to the cell;
introducing a pre-synthesized splint nucleic acid to the cell, wherein the pre-synthesized splint nucleic acid comprises a locked nucleic acid, wherein the pre-synthesized splint nucleic acid contacts with the integrating nucleic acid and the guide nucleic acid; and
introducing an exogenous ligase to the cell or introducing a nucleic acid encoding the exogenous ligase to the cell, thereby ligating, via the exogenous ligase, a 5′ end of the pre-synthesized integrating nucleic acid to a 3′ end of the nick at the predetermined locus of the target nucleic acid, wherein neither the endonuclease nor the exogenous ligase is fused to a polymerase.
